(19) United States
(12) Patent Application Publication
(10) Pub. No.: US 2024/0425873 A1
(43) Pub. Date: Dec. 26, 2024

(54) LINKAGE OF A DISTAL PROMOTER TO A GENE OF INTEREST BY GENE EDITING TO MODIFY GENE EXPRESSION

(71) Applicant: LIMAGRAIN EUROPE, Saint Beauzire (FR)

(72) Inventors: Wyatt PAUL, Pont-du-Chateau (FR); Pascual PEREZ, Chanonat (FR); Jean-Philippe PICHON, Clermont Ferrand (FR)

(73) Assignee: LIMAGRAIN EUROPE, Saint Beauzire (FR)

(21) Appl. No.: 18/274,023

(22) PCT Filed: Feb. 1, 2022
(86) PCT No.: PCT/EP2022/052357

§ 371 Date: Jul. 25, 2023

(30) Foreign Application Priority Data
Feb. 2, 2021 (EP) 21305141.0

(51) Int. Cl. C12N 15/82

(52) U.S. Cl. CPC C12N 15/8289; C12N 15/8213; C12N 15/8216

(57) ABSTRACT

Gene editing to link a distal promoter to a gene of interest in order to modify the expression of the gene. The desired gene editing is a deletion or inversion. The invention relates to a method to enhance the expression of a fertility-restorer gene of interest in a plant. The invention also relates to the plant obtained by such methods.

The invention relates to gene editing, preferably in a plant, to link a distal promoter to a gene of interest in order to modify the expression of said gene. The desired gene editing is preferably a deletion or inversion. More specifically, said invention relates to a method to enhance the expression of a fertility-restorer gene of interest in a plant.

### BACKGROUND

The expression pattern and the strength of a promoter are key elements in the expression of genes. Genetic modification approaches highlighted the fact that the use of a strong constitutive promoter to drive the expression of a plant gene was necessary to boost the expression of a gene in order to improve a specific trait of interest. Other possibilities offered by genetic engineering involved the use of a promoter with a different pattern of expression in the plant or a promoter with a different pattern of expression throughout the plant development cycle.

The recent gene editing technologies are offering new possibilities for researchers to modify the expression of genes. It is now possible to activate or repress target genes by using inactivated endonucleases coupled with activator or repressor domains. Some technologies also target promoters to add specific domains within the regulatory regions of genes by homologous recombination. But some of these gene editing technologies are relatively similar to technologies previously used to obtain GMO as they require the insertion of heterologous DNA within the plant genomes.

The use of gene editing to create deletions or inversions in the genome of plants or mammals has already been disclosed. For example, Cai et al. disclose a CRISPR/Cas9-mediated deletion of large genomic fragments in soybean, Durr et al. disclose deletions of gene clusters and non-coding regulatory regions in Arabidopsis using CRISPR/Cas9 for functional genomic studies, and Ordon et al. describe chromosomal deletions in dicotyledonous plants by gene editing. Relative to mammals, Korablev et al. mention deletions, inversions and duplications involving the Contactin-6 gene, using a CRISPR-Cas9 technology, to develop new mouse lines. Li et al. also disclose inversions and duplications of mammalian regulatory DNA elements and gene clusters by CRISPR/Cas9. The use of gene editing to create chromosome inversions has also been described, as in WO2020/117553, but only to reduce the expression of a gene of interest.

Therefore, deletions or inversions into a genome by gene editing have already been disclosed in the prior art. However, none of these deletions and/or inversions aims to operably link a distal endogenous promoter to a gene of interest.

Fertility-restorer (Rf) genes are essential today for the development of hybrids, especially in autogamous species like wheat. To avoid self-pollination, the plant that is used as the female parent has to be male sterile. The male parent is generally a fertility restorer line. The hybrid progeny of the cross will have normal restored pollen fertility.

It is a goal of breeders to move towards hybrid wheat since hybrid varieties usually outperform inbreds. Since wheat is dioecious and largely autogamous, the production of hybrid seed requires systems to facilitate crossing and reduce the cost of hybrid seed production. Such a system is the use of male-sterile ‘female’ plant line crossed to a male fertile line such that all the seed harvested from the female, male-sterile plants will be F1 hybrid seed. Male-sterile plants can be produced using cytoplasmic male sterility (CMS) where the female plant carries ‘defective’ mitochondria that often express novel ORFs leading to the production of no or defective pollen. Use of CMS systems for hybrid seed production requires that the male line used in the hybrid seed production cross carries a nuclear gene or genes that repair the defective mitochondria in the F1. This leads to full male-fertility of the F1 plants that are grown by the farmer. These nuclear genes in the male line are referred to as CMS restorer genes. One potential CMS system for hybrid wheat production is that using T. timopheevii CMS (Bohra A, Jha U C, Adhimoolam P, et al (2016) Cytoplasmic male sterility (CMS) in hybrid breeding in field crops. Plant Cell Rep 35:967-993. doi: 10.1007/s00299-016-1949-3). A drawback of this system is that a combination of several restorer genes (Rf1, Rf3, Rf4 and Rf7) is required to give full male fertility to the F1. For the breeder this makes the system more complex to use since each male line has to be converted to contain 3 or 4 independently segregating restorer genes.

It is thus desirable to identify or create a single effective restorer locus.

In this context, the development of modified organisms which do not require the insertion of heterologous DNA within their genomes is of high interest, notably in plants. A method to obtain hybrid plant, more specifically hybrid wheat, which requires the use of only one fertility restorer gene is also of major importance.

It is therefore the object of this invention to provide a method to meet these needs. Preferably, it is an object of this invention to modify the expression of a gene of interest, more preferably a fertility restorer gene, to obtain hybrid plants useful for the seed industry.

### SUMMARY

The inventors have shown that the expression of a gene of interest can be modified, preferably enhanced, using gene editing to operably link a distal endogenous promoter, or part of an endogenous promoter, to said gene of interest.

Therefore, in a first aspect, it is disclosed herein a method to modify the expression of a gene of interest in an organism, said method comprising the steps of: a) introducing into said organism at least one gene editing system; b) allowing the said gene editing system to perform the desired editing at a target genomic site; and wherein the said gene editing system is designed so that, after the desired editing, the gene of interest is operably linked to an endogenous promoter, or part of an endogenous promoter, said endogenous promoter being distal from a naturally occurring promoter of the gene of interest in the genome of a non-modified organism.

In a specific embodiment, said organism is a eukaryotic organism, provided that the organism is not a human or an animal. In specific embodiments that may be combined with the previous embodiments, the organism is a plant, preferably a polyploid plant, such as wheat, oat, rapeseed, potato, sugar cane, . . . .

In a specific embodiment that may be combined with the previous embodiments, the performance of the desired editing as recited in step b) comprises two or more DNA breaks within said target genomic site.

In a specific embodiment that may be combined with the previous embodiments, the desired editing is a deletion or an inversion, the deletion preferably having a size from 10 kb to 10 Mb and the size of said inversion being from 10 kb to 100 Mb.

In a specific embodiment that may be combined with the previous embodiments, said deletion is a deletion of a genomic region comprising the full naturally occurring promoter or of a genomic region comprising only a part of the naturally occurring promoter. Alternatively, in a specific embodiment that may be combined with the previous embodiments, said inversion is an inversion of a genomic region resulting in the replacement of the naturally occurring promoter by said endogenous promoter or is an inversion of a genomic region resulting in the replacement of a part of the naturally occurring promoter by a part of said endogenous promoter.

In a specific embodiment that may be combined with the previous embodiments, said gene of interest is a fertility-restorer gene, preferably an RFL29 gene, more preferably represented by the coding sequence of SEQ ID NO:14. In another specific embodiment that may be combined with the previous embodiments, said fertility-restorer gene is an RFL79 gene, more preferably represented by SEQ ID NO:58.

In a specific embodiment that may be combined with the previous embodiments, said endogenous promoter drives a higher expression of the gene of interest, changes the pattern of expression of the gene of interest during the development cycle of a plant, changes the spatial pattern of expression of the gene of interest or is a promoter which is activated by biotic or abiotic stress.

In a specific embodiment that may be combined with the previous embodiments, the modification of the expression is an enhancement, said endogenous promoter being notably a strong promoter.

In a specific embodiment that may be combined with the previous embodiments, the gene editing system is designed to maintain the Kozak sequence of the gene of interest.

In a specific embodiment that may be combined with the previous embodiments, the break occurs in a promoter, in an untranslated region, in gene-gene junction region, in exon or in intron, preferably in a promoter or in an untranslated region.

In a specific embodiment that may be combined with the previous embodiments, said gene editing system is chosen among a zinc finger nuclease (ZFN) gene editing system, a transcription activator-like effector nucleases (TALEN) gene editing system, a clustered regularly interspaced short palindromic repeats (CRISPR) gene editing system, or a meganuclease gene editing system, preferably a CRISPR gene editing system. In a more specific embodiment, said gene editing system comprises at least one enzyme chosen among: meganuclease, zinc-finger nuclease, transcription-activator like effector nuclease, CRISPR-nickase or CRISPR-nuclease.

In a specific embodiment that may be combined with the previous embodiments, the CRISPR gene editing system comprises multiple guide sequences capable of hybridizing to multiple target sequences within the target genomic site.

In a specific embodiment that may be combined with the previous embodiments, the method comprises the steps of:
a) identifying an endogenous promoter, distal from a naturally occurring promoter of a gene of interest in a non-modified organism, which is capable of modifying the expression of said gene of interest;
b) identifying the deletion or insertion of a genomic region to be performed in order to operably link said endogenous promoter, or part of an endogenous promoter, to said gene of interest;
c) introducing into said organism at least one gene editing system designed to perform the deletion or insertion identified in step b);
d) allowing said gene-editing system to perform the desired editing at the target genomic site;
e) optionally selecting an organism with the desired editing.

It is also disclosed herein, as a second aspect, a method to enhance the expression of a fertility-restorer gene of interest, in a plant, said method comprising the steps of: a) introducing into said plant, at least one gene-editing system, preferably a CRISPR gene-editing system; b) allowing the said gene-editing system to perform the desired editing at a target genomic site; and wherein the said gene editing system is designed so that, after the desired editing, the fertility-restorer gene of interest is operably linked to an endogenous promoter, or part of an endogenous promoter, said endogenous promoter being distal from a naturally occurring promoter of the fertility-restorer gene of interest in the genome of a non-modified plant.

This second aspect is a particular embodiment of the first aspect, wherein the organism is a plant, more specifically a wheat plant. Consequently, all the specific embodiments disclosed above in relation to the first aspect also apply and may also be combined with this second aspect.

In a specific embodiment that may be combined with the previous embodiments, after the desired editing, the fertility-restorer gene of interest is operably linked to an endogenous promoter, said endogenous promoter being a strong promoter.

In a specific embodiment that may be combined with the previous embodiments, the fertility-restorer gene is RFL29, and preferably said endogenous promoter identified to replace the naturally-occurring promoter of RFL29 is chosen among PK-like promoter (notably PK-like promoter in T. aestivum represented by SEQ ID NO:10), RAE1-like promoter (notably RAE1-like promoter in T. aestivum represented by SEQ ID NO:11) or At5g02240-like promoter (notably At5g02240-like promoter in T. aestivum represented by SEQ ID NO:59).

In another specific embodiment, that may be combined with the previous embodiments, the fertility-restorer gene is RFL79.

In a specific embodiment that may be combined with the previous embodiments, the gene editing system is a CRISPR gene-editing system which is a delivery system comprising and operably configured to deliver into a plant cell, either CRISPR-Cas complex components, or one or more polynucleotide sequences comprising or encoding said components, wherein said CRISPR-Cas complex components, comprises:
(i) at least two guide sequences capable of hybridizing to the two ends of the target genomic site to effect two breaks, thereby allowing a deletion of said target genomic site or an inversion at said target genomic site;
(ii) a CRISPR-associated protein,
   and wherein said CRISPR-Cas complex is operable in a plant cell.

The present disclosure also relates to a plant useful in the above-mentioned methods. It is thus disclosed, in a third aspect, a plant, or a plant part, comprising a gene editing system designed to perform a desired editing at a target genomic site, so that after the desired editing occurred, a gene of interest in the plant is operably linked to an endogenous promoter, or part of an endogenous promoter, said endogenous promoter being distal from a naturally occurring promoter of the gene of interest in the genome of a non-modified plant.

The present disclosure, in a fourth aspect, further relates to the plant as obtained with the above-mentioned methods, as well a method to identify such a plant. Such identification method comprises a step of PCR after the desiring editing was performed, in order to detect bands corresponding to the desired editing, notably inversion or deletion. To evaluate the result of the gene editing, such an identification method can also be used to detect the expression level of the gene of interest, as well as the level of the protein encoded by said gene. A phenotypic analysis can also be performed in order to determine if the modified plant has a fertility restoration phenotype.

The present disclosure, in a fifth aspect, also relates to a method to restore fertility in wheat, comprising a step of crossing a sterile wheat plant with a plant wherein the expression of a fertility-restorer gene has been enhanced according to the above-mentioned methods.

### BRIEF DESCRIPTION OF THE FIGURES

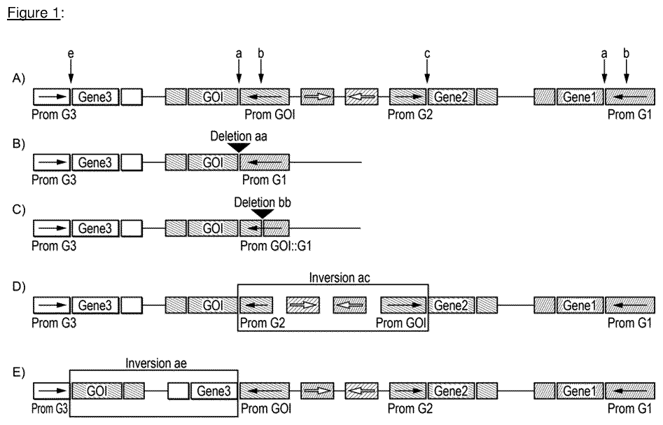

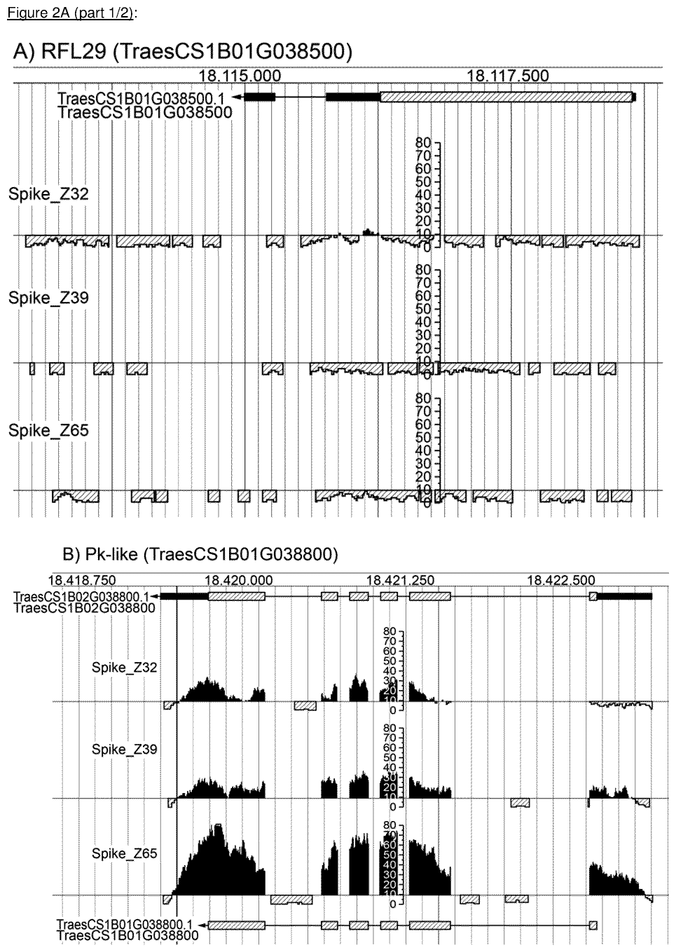

The FIG. 1 shows genomic deletions or inversions to alter the expression of a gene of interest (GOI).

The FIG. 1A presents the genomic context of the target region. GOI stands for Gene Of Interest. a, b, c, e represents the cutting/cleavage sites. Genes 1, 2, 3 are genes in the GOI region. Prom G1, G2, G3 are the promoters of Genes 1, 2, 3 respectively.

The FIG. 1B shows the deletion obtained with a cleavage at both “a” sites. Because of the deletion, the GOI is now under the control of endogenous distal promoter Prom G1 instead of its naturally-occurring promoter.

The FIG. 1C shows the deletion obtained with a cleavage at both “b” sites. Because of the deletion, the GOI is now under the control of a chimeric promoter Prom GOI::G1 comprising a region of its naturally-occurring promoter and a region of endogenous distal promoter Prom G1.

The FIG. 1D shows the inversion obtained with a cleavage at sites “a” and “c”. Because of the inversion, the GOI is now under the control of endogenous distal promoter Prom G2 instead of its naturally-occurring promoter.

The FIG. 1E shows the inversion obtained with a cleavage at sites “a” and “e”. Because of the inversion, the GOI is now under the control of endogenous distal promoter Prom G3 instead of its naturally-occurring promoter.

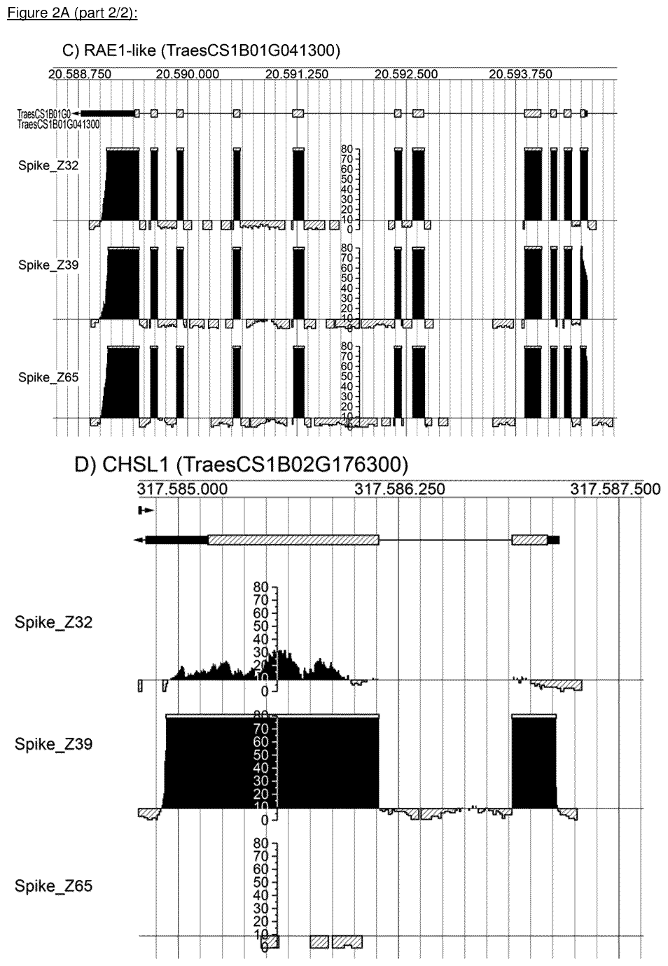

The FIG. 2 shows RNAseq expression data for RFL29, PK-like (or TaPK-like), RAE1-like (or TaRAE1-like) and CHSL1 in wheat spikes.

The FIG. 2A shows the RNAseq expression data for RFL29 (A), PK-like (or TaPK-like) (B), RAE1-like (or TaRAE1-like) (C) and CHSL1 (D) in wheat spikes.

The FIG. 2B shows the expression in Z39 spikes.

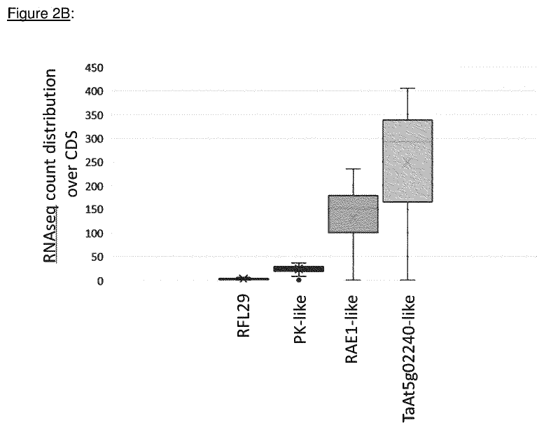

The FIG. 3 shows the procedure of fertility scoring.

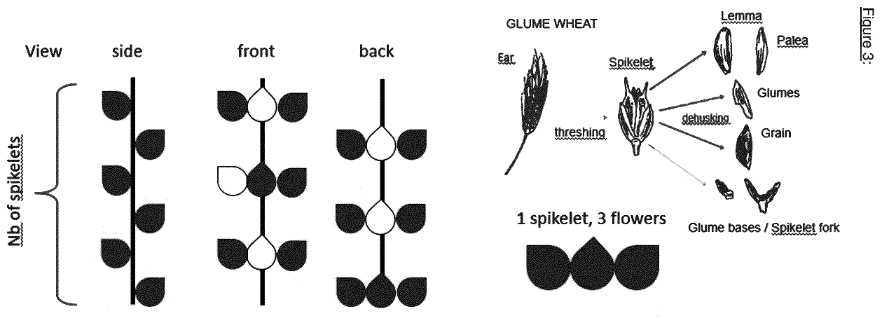

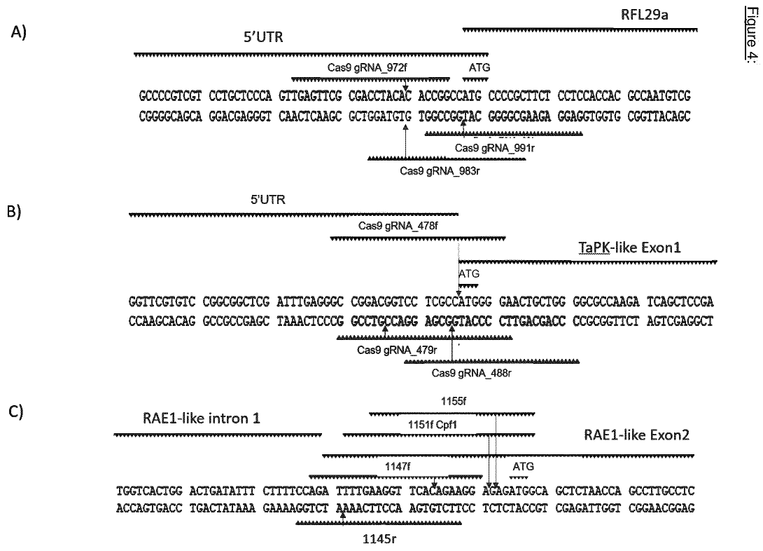

The FIG. 4 shows the candidate gRNA targets for RFL29 to PK-like (or TaPK-like) and RFL29 to RAE1-like (or TaRAE1-like) deletions. The arrows represent the cleavage sites.

The FIG. 4A shows the positions on RFL29 gene.

The FIG. 4B shows the positions on PK-like (or TaPK-like) gene.

The FIG. 4C shows the positions on RAE1-like (or TaRAE1-like) gene.

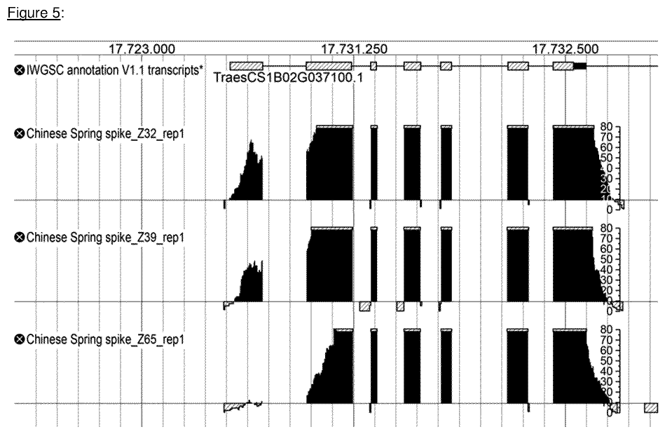

The FIG. 5 shows RNAseq expression data for TaAt5g02240-like in spikes.

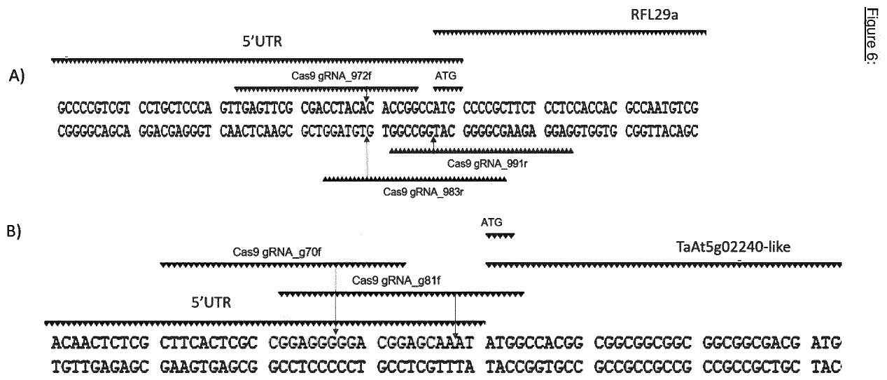

The FIG. 6 shows the candidate gRNA targets for RFL29—TaAt5g02240-like inversion.

The arrows represent the cleavage sites.

The FIG. 6A shows the positions on RFL29 gene.

The FIG. 6B shows the positions on At5g02240-like (or Ta At5g02240-like) gene.

### DETAILED DESCRIPTION

### Organism According to the Present Disclosure

In specific embodiments of the disclosure, the organism is a eukaryotic organism, provided that the organism is not a human or other animal.

Preferably, the organism is a plant. As used herein, the term “plant” or “plants” refer to the entire plant but also plant parts (cells, tissues or organs, seed pods, seeds, severed parts such as roots, leaves, flowers, pollen, etc.), progeny of the plants which retain the distinguishing characteristics of the parents (i.e. the modification of the expression of the gene of interest), such as seed obtained by selfing or crossing, e.g. hybrid seeds (obtained by crossing two inbred parent plants), hybrid plants and plant parts derived there from are encompassed herein, unless otherwise indicated. The plant can be a monocotyledon or a dicotyledonous plant.

In specific embodiments, said plant is a polyploid plant such as wheat, oat, rapeseed, potato, sugar cane, . . . .

As used herein, the term “wheat” refers to a plant of Triticum gender, as for example T. aestivum, T. aethiopicum, T. araraticum, T. boeoticum, T. carthlicum, T. compactum, T. dicoccoides, T. dicoccon, T. durum, T. ispahanicum, T. karamyschevii, T. macha, T. militinae, T. monococcum, T. polonicum, T. spelta, T. sphaerococcum, T. timopheevii, T. turanicum, T. turgidum, T. urartu, T. vavilovii, T. zhukovskyi Faegi. Preferably, the wheat plant is T. aestivum. Wheat plant also refers to Aegilops gender and Triticale.

As used herein, the term “oat” refers to a plant of Avena gender, as for example A. sativa.

As used herein, the term “rapeseed” refers to a plant of Brassica gender, as for example B. napus, B. juncea and B. rapa; preferably B. napus.

As used herein, the term “potato” refers to a plant of Solanum gender, as for example Solanum tuberosum.

As used herein, the term “sugar cane” refers to a plant of Saccharum gender, as for example S. officinarum, S. sponteneum, S. robustum, S. sinense and S. barberi.

### Method to Modify the Expression of a Gene of Interest in an Organism

A first aspect of the present disclosure relates to a method to modify the expression of a gene of interest in an organism, said method comprising the steps of:
a) introducing into said organism at least one gene editing system;
b) allowing the said gene-editing system to perform the desired editing at a target genomic site;
   and wherein the said gene editing system is designed so that, after the desired editing, the gene of interest is operably linked to an endogenous promoter, or part of an endogenous promoter, said endogenous promoter being distal from a naturally occurring promoter of the gene of interest in the genome of a non-modified organism.

As used herein, the term “modify the expression of a gene” refers to an enhancement of the expression, or a modification of an expression pattern. In a preferred embodiment that may be combined with the previous embodiments, said modification is an enhancement. In such a case, the endogenous promoter can be considered as a strong promoter. In a specific embodiment, the gene expression modification can include modification at a precursor mRNA level, at a mature mRNA level or at translation level.

As used herein, the term “enhancement of the expression” means that the gene of interest is more expressed compared to a non-modified organism. Preferably the gene expression is increased by at least 2 fold, preferably between 2 and 100 fold, such as, at least 5, 10, 15, 20, 25, 30, 35, 40, 45, 50, 55, 60, 65, 70, 75, 80, 85, 90, 95 or 100. The term “enhancement of the expression” also means that the gene of interest is expressed when operably linked to the endogenous promoter. Indeed, in this specific embodiment, the gene of interest is not expressed in a non-modified organism, but gene expression of said gene of interest can be detected in a modified organism, after the desired editing. Relative gene expression can be measured by a q-RT-PCR method. Gene expression can also be measured by RNA-Seq.

As used herein, the term “modification of an expression pattern” means that the gene is expressed under different conditions by comparison with a non-modified organism, for example at a different time during the development cycle of a plant, in a different tissue, in presence of biotic or abiotic stress, . . . .

As used herein, the term “gene of interest” refers to an endogenous gene of the organism whose modification of the expression confers a desired characteristic to the organism, such as improved performance in the fields, improved performance in an industrial process, improved nutritional value, or improved reproductive capability. The gene of interest can encode for a protein of interest, but can also encode for a functional RNA of interest, such as antisense RNA, rRNA, tRNA, . . . . The gene of interest can be a gene which is not expressed in a non-modified organism and which becomes expressed when operably linked to an endogenous promoter, or part of an endogenous promoter. The gene of interest can be dominant, recessive or semi-dominant. As used herein “expression of a gene” means that the coding sequence of the gene is transcribed, and optionally translated.

As used herein, the term “operably linked” means that the gene of interest is linked to said endogenous promoter in a manner that allows for expression of the gene of interest.

As used herein, the term “introducing” means that the gene editing system penetrates into the cell of the organism, so that the system can enter the nucleus for targeted gene editing.

As used herein, the term “allowing the said gene-editing system to perform the desired editing at a target genomic site” means that two or more DNA breaks are made within the target genomic site, and that the strand cuts are then repaired. In other words, it also means “making two or more DNA breaks and then selecting for an organism (more specifically a cell) wherein the target genomic site has been edited” (in such edited cell, the gene of interest is operably linked to an endogenous distal promoter, or part of an endogenous distal promoter, whereas in non-edited cell, the gene of interest is operably linked to its naturally occurring promoter).

As used herein, the term “target genomic site” refers to the genomic region wherein the gene editing occurs, i.e. the genomic region between the two cleavage sites, or the genomic region between the most remote cleavage sites if there are more than two cleavage sites. As used herein, the term “cleavage site” corresponds to genomic DNA region which comprises a sequence recognized by a specific enzyme used in gene editing system, such as nickase or nuclease, which cleave the genomic DNA region in one or both strands.

As used herein, the term “gene of interest is operably linked to an endogenous promoter, or part of an endogenous promoter” means that the coding sequence is under the transcriptional control of the endogenous promoter or part of endogenous promoter. In other words, the promoter, or part, controls the expression of the gene of interest. The said promoter sequence does not need to be contiguous with the sequence of the gene of interest, as long as the promoter still controls the expression of the gene (e.g. a transcribed sequence which is not translated, can be interposed between the promoter and the coding sequence of the gene of interest). In a preferred embodiment, after the gene editing occurred, the endogenous promoter is contiguous to the gene of interest.

As used herein, the term “endogenous promoter” means that the promoter is native to the organism.

As used herein, the term “the said promoter distal from a naturally occurring promoter of the gene of interest” means that the endogenous promoter is distant from the gene of interest in a non-modified organism. The distance can be kilobase-sized or megabase-sized between the naturally occurring promoter of the gene of interest and the distal promoter. Said distal promoter can be located in 5′ or 3′ from the naturally occurring promoter. In other words, the endogenous promoter is distal compared to the naturally occurring promoter which is a proximal promoter. As used herein, the term “proximal” refers more specifically to a nucleotide sequence, upstream (5′) to the coding sequence of the gene, generally from 1 base to about 500 base of the start site. The term “from 1 base to 500 base” means every value of this range, even if not explicitly recited, such as 1, 2, 3, 4, 5, 6, 7, 8, 9, 10, 20, 30, 40, 50, 60, 70, 80, 90, 100, 150, 200, 250, 300, 350, 400, 450, 500.

As used herein, the term “distal promoter” refers also to a promoter which is distant from the gene of interest in a non-modified organism, and which regulates the expression of a gene different from the gene of interest in a non-modified organism. It is only after the gene-editing that the said distal promoter regulates the expression of the gene of interest. In other words, in a non-modified organism, the gene of interest is operably linked to its naturally occurring promoter and not to the distal endogenous promoter or part of it, whereas after the gene-editing the gene of interest is operably linked to the distal endogenous promoter or part of it.

In a specific embodiment of the disclosure that may be combined with the previous embodiments, the desired editing is a deletion or an inversion.

As used herein “deletion” means that the genetic material which represents the target genomic site is lost on a chromosome. According to a specific embodiment of the disclosure that may be combined with the previous embodiments, the deletion is a deletion of a genomic region comprising the full naturally occurring promoter. In this embodiment, the full naturally occurring promoter is either replaced by the full distal endogenous promoter or by a functional part of the full distal endogenous promoter. Alternatively, said deletion is a deletion of a genomic region comprising only a part of the naturally occurring promoter. In this last embodiment, the gene editing creates thus a chimeric promoter containing two parts: one part comprising the naturally occurring promoter and one part comprising the endogenous promoter, with the proviso that such chimeric promoter is a functional promoter.

As used herein “inversion” means that the genomic region is reversed end to end within the target region site. In other words, it corresponds to a rearrangement within a single chromosome. According to a specific embodiment of the disclosure that may be combined with the previous embodiments, the inversion is an inversion of a genomic region resulting in the replacement of the naturally occurring promoter by said endogenous promoter. Alternatively, said inversion is an inversion of a genomic region resulting in the replacement of a part of the naturally occurring promoter by a part of said endogenous promoter. In this last embodiment, the gene editing creates thus two chimeric promoters containing two parts: one part comprising the naturally occurring promoter and one part comprising the endogenous promoter. In an embodiment, the two chimeric promoters can be functional and in another embodiment only one chimeric promoter is functional: the one which is operably linked to the gene of interest.

In a specific embodiment that may be combined with the previous embodiments, the size of said deletion is from 10 kb to 10 Mb. In a specific embodiment that may be combined with the previous embodiments, the size of said inversion is from 10 kb to 100 Mb. As used herein, “from 10 kb to 10 Mb” or “from 10 kb to 100 Mb” mean every value of this range, even if not explicitly recited, for example from 100 kb to 10 Mb, from 1000 kb to 10 Mb, from 100 kb to 1 Mb, from 1000 kb to 1 Mb, from 10 kb to 5 Mb, from 100 kb to 5 Mb, from 200 kb to 10 Mb, from 300 kb to 10 Mb, from 400 kb to 10 Mb, from 500 kb to 10 Mb, from 600 kb to 10 Mb, from 700 kb to 10 Mb, from 800 kb to 10 Mb, from 900 kb to 10 Mb, from 200 kb to 5 Mb, from 300 kb to 5 Mb, from 400 kb to 5 Mb, from 500 kb to 5 Mb, from 600 kb to 5 Mb, from 700 kb to 5 Mb, from 800 kb to 5 Mb, from 900 kb to 5 Mb, from 1 Mb to 10 Mb, from 2 Mb to 10 Mb, from 3 Mb to 10 Mb, from 4 Mb to 10 Mb, from 5 Mb to 10 Mb, from 6 Mb to 10 Mb, from 7 Mb to 10 Mb, from 8 Mb to 10 Mb, from 9 Mb to 10 Mb, from 10 kb to 9 Mb, from 10 kb to 8 Mb, from 10 kb to 7 Mb, from 10 kb to 6 Mb, from 10 kb to 5 Mb, from 10 kb to 4 Mb, from 10 kb to 3 Mb, from 10 kb to 2 Mb, from 10 kb to 1 Mb, or from 100 kb to 100 Mb, from 1000 kb to 100 Mb, from 10 kb to 1 Mb, from 10 kb to 10 Mb, from 10 kb to 20 Mb, from 10 kb to 30 Mb, from 10 kb to 40 Mb, from 10 kb to 50 Mb, from 10 kb to 60 Mb, from 10 kb to 70 Mb, from 10 kb to 80 Mb, from 10 kb to 90 Mb, from 100 kb to 20 Mb, from 100 kb to 30 Mb, from 100 kb to 40 Mb, from 100 kb to 50 Mb, from 100 kb to 60 Mb, from 100 kb to 70 Mb, from 100 kb to 80 Mb, from 100 kb to 90 Mb, from 1 Mb to 20 Mb, from 1 Mb to 30 Mb, from 1 Mb to 40 Mb, from 1 Mb to 50 Mb, from 1 Mb to 60 Mb, from 1 Mb to 70 Mb, from 1 Mb to 80 Mb, or from 1 Mb to 90 Mb.

In a specific embodiment of the disclosure that may be combined with the previous embodiments, the endogenous promoter drives a higher expression of the gene of interest, changes the pattern of expression of the gene of interest during the development cycle of a plant, changes the spatial pattern of expression of the gene of interest or is a promoter which is activated by biotic or abiotic stress. Abiotic stress is for example light or drought stress. Biotic stress is for example viral, bacterial or fungal invasion of the plant.

### Gene Editing System Used According to the Present Disclosure

According to the disclosure, a gene editing is performed to create at least two breaks in the genomic targeted site. As used herein, the term “break” refers to a cleavage on both DNA strands. The double-stranded break can result of two separate single-stranded breaks. One double strand break on each side of the target genomic site is necessary to perform the desired editing at the target genomic site. Two single strand breaks (a single strand break on each DNA strand) on each side of the target genomic site are necessary to perform the desired editing at the target genomic site.

In a specific embodiment, the at least two breaks occur in a promoter, in an untranslated region, in gene-gene junction region, in exon or in intron. The term “untranslated region” preferably refers to a 5′UTR region. For example, one of the breaks can occur in front of the initiating Methionine ATG of the coding sequence of the gene of interest. In a specific embodiment, the gene-editing system is designed to maintain the Kozak sequence of the gene of interest.

The gene editing system which can be used are preferably chosen among a zinc finger nuclease (ZFN) gene editing system, a transcription activator-like effector nucleases (TALEN) gene editing system, a clustered regularly interspaced short palindromic repeats (CRISPR) gene editing system, or a meganuclease gene editing system. Such gene editing system comprises at least one enzyme, preferably chosen among: meganuclease, zinc-finger nuclease, transcription-activator like effector nuclease, CRISPR-nickase or CRISPR-nuclease. Such enzymes may be a wild-type protein, or a mutant with the proviso that the enzyme still possesses its nuclease or nickase activity. In a more preferred embodiment, the enzyme is chosen among Cas3, Cas9, Cas12a, or dCas9-Fokl, dCpf1-Fokl, chimeric FENI-Fokl, a nickase Cas9, chimeric dCas9 non-Fokl nuclease and dCpf1 non-Fokl nuclease.

According to the disclosure, the most preferred gene editing system is a CRISPR gene editing system. As used herein, the term “CRISPR gene editing system”, which can also be called “CRISPR-Cas system” in an embodiment refers to a system which relies on two components: (i) a guide sequence which is a specific RNA sequence which hybridize to a target DNA region and direct the enzyme (the ‘CRISPR-associated protein’) to the target DNA region to perform the gene editing, and (ii) the CRISPR-associated protein which is a non-specific nuclease or nickase.

As used herein, the term “guide sequence” refers to a “guide RNA”, also called “gRNA”. Preferably, said guide sequence comprises a crRNA and optionally a tracrRNA. The ‘crispr RNA’, also called crRNA is a 17-20 nucleotide sequence complementary to the target DNA, and a “tracrRNA”, is a binding scaffold for the Cas9 nuclease. For Cas12a, the guide RNA comprises only the crRNA.

The crRNA and the tracrRNA may be present on the same molecule or may be present on two physically distinct molecules. According to the disclosure, the guide sequence may refer to a single guide, so that the crRNA and the tracrRNA form a single molecule.

In specific embodiments of the disclosure, the CRISPR system may comprise multiple guide sequences capable of hybridizing to multiple target sequences within the target genomic site.

Preferably, the said “CRISPR-associated protein” binds to the target DNA region only in presence of a specific sequence, called protospacer adjacent motif (PAM), on the non-targeted DNA strand. The nuclease cuts 3-4 nucleotides upstream of the PAM sequence or around 18-19 nucleotides downstream of the PAM sequence. Therefore, in a preferred embodiment, each end of the target genomic site (i.e. each end of the target DNA region) is adjacent to a Protospacer Adjacent Motif (PAM) recognized by a CRISPR-associated protein, such as 5′NGG3′ (with N representing either A, T, C or G) for Cas9 of Streptococcus pyogenes or T-rich PAM for Cas12a such 5′TTN or 5′TTTN (with N representing either A, T, C or G). In this embodiment, the locations in the genome that can be targeted by the CRISPR-associated proteins are limited by the locations of these PAM sequences.

In specific embodiments of the disclosure that may be combined with the previous embodiments, the CRISPR system may comprise at least one nuclear localization system (NLS). Preferably, said CRISPR-associated protein, more preferably the nuclease, is operably linked to the NLS.

According to the disclosure, several strategies are available to deliver the CRISPR gene editing system: a DNA delivery format, a RNA delivery format or a ribonucleoprotein delivery format.

In the DNA delivery format, the system is provided as one or several DNA molecules which enters the cell and translocates to the nucleus where the sequences encoding for CRISPR-associated protein and the guide sequence are transcribed. After translation of the CRISPR-associated protein said CRISPR-associated protein, preferably a nuclease, and the guide sequence assembles into the cell cytoplasm to form a ribonucleoprotein (RNP) complex. Next, said RNP complex enters the nucleus to perform the desired gene editing.

In the RNA delivery format, the system is provided as one or several RNA molecules. The sequences comprising the guide RNA and the mRNA corresponding to the CRISPR-associated protein are co-transfected into the cell cytoplasm. The mRNA is then translated to produce a CRISPR-associated protein, preferably a nuclease. Then, a RNP complex is formed and it enters the nucleus to perform the desired gene editing.

In the ribonucleoprotein delivery format, a RNP complex comprising a guide RNA and a CRISPR-associated protein are provided. Next, said RNP complex enters the nucleus to perform the desired gene editing.

In a specific embodiment of the disclosure, the CRISPR gene editing system comprises then a delivery system comprising and operably configured to deliver into an eukaryotic cell, either CRISPR-Cas complex components, or one or more polynucleotide sequences comprising or encoding said components, wherein said CRISPR-Cas complex components, comprises:
(i) at least two guide sequences capable of hybridizing to the two ends of the target genomic site to effect two breaks, thereby allowing a deletion of said target genomic site or an inversion at said target genomic site;
(ii) a CRISPR-associated protein,
   and wherein said CRISPR-Cas complex is operable in an eukaryotic cell.

As used herein, the term “CRISPR-Cas complex components” refers to the components required to form a RNP complex (i.e. at least one guide sequence and a functional nuclease or a nickase, preferably a nuclease); Preferably, the CRISPR gene editing system is a CRISPR-Cas vector system encoding a CRISPR-Cas complex, said vector system comprising:
(i) a first regulatory element operably linked to a polynucleotide sequence encoding a first guide sequence, said first guide sequence being capable of hybridizing to a first end of the target genomic site;
(ii) a second regulatory element operably linked to a polynucleotide sequence encoding a second guide sequence, said second guide sequence being capable of hybridizing to a second end of the target genomic site;
(iii) a third regulatory element operably linked to a polynucleotide sequence encoding a CRISPR-associated protein;
   wherein said components (i), (i) and (iii) are located on the same or different vectors of the system, and wherein said CRISPR-Cas complex is operable in an eukaryotic cell.

As used herein, the term “vector” refers to DNA sequence in which it is possible to insert fragments of foreign nucleic acid, the vectors making it possible to introduce foreign DNA into the eukaryotic cell. Examples of vectors are plasmids, cosmids, yeasts, yeast artificial chromosomes (YACs), bacteria artificial chromosomes (BACs), artificial chromosomes derived from the P1 bacteriophage (PACs), and viral vectors such as lentiviral, baculoviral or adeno-viral, adeno-associated viral vectors or plant viral vectors. In a specific embodiment, the CRISPR-Cas complex is delivered via liposomes, nanoparticles, exosomes, microvesicles, or a gene-gun. In a specific embodiment, the CRISPR-Cas complex is delivered in plants via electroporation, Agrobacterium transformation, direct precipitation by means of PEG.

As used herein, the term “regulatory element linked to a polynucleotide sequence” means that the polynucleotide sequence is linked to the regulatory element in a manner that allows for expression of said polynucleotide. Said regulatory element is preferably a promoter. In a specific embodiment, the said vector system can comprise one or several regulatory elements, such as promoter, enhancer, internal ribosomal entry sites, polyadenylation signals, poly U sequences, . . . . Said regulatory elements may direct constitutive expression of the polynucleotide sequence in many types of host cells or only in certain types (i.e. tissue specific regulatory sequences). A tissue-specific promoter may direct expression primarily in a desired tissue of interest or in a particular type of cells. Regulatory elements may also direct expression in a temporal-dependent manner, such as in a cell-cycle dependent or developmental stage-dependent manner, which may or may not also be tissue or cell-type specific.

In other embodiment, the CRISPR gene editing system is a vector system encoding a CRISPR-Cas complex, said vector system comprising:
i) a first regulatory element operably linked to a polynucleotide sequence encoding the first guide sequence and the second guide sequence; and
ii) a second regulatory element operably linked to a polynucleotide sequence encoding a CRISPR-associated protein;
   wherein said components (i) and (ii) are located on the same or different vectors of the system.

In a specific embodiment, the said vector system comprises one or more expression cassettes for driving the expression of one or more guide RNA and CRISPR-associated protein. Each of the expression cassettes comprise a promoter sequence, a polynucleotide sequence encoding for at least one guide sequence, and a terminator.

In a specific embodiment, the disclosure relates to of the disclosure a method to modify the expression of a gene of interest in an organism, as mentioned above, which the steps of:
a) identifying an endogenous promoter, distal from a naturally occurring promoter of the gene of interest in a non-modified organism, which is capable of modifying the expression of said gene of interest;
b) identifying the deletion or insertion of a genomic region to be performed in order to operably link said endogenous promoter, or part of an endogenous promoter, to said gene of interest;
c) introducing into said organism at least one gene-editing system designed to perform the deletion or insertion identified in step b);
d) allowing said gene-editing system to perform the desired editing at the target genomic site;
e) optionally selecting an organism with the desired editing.

In a specific embodiment, some steps can be performed before the step a) mentioned above. Such steps can be:
a step of analyzing the region surrounding the gene of interest to identify the presence of at least one distal promoter, notably the position of at least one distal promoter and/or its orientation, and/or
a step of operably linking the distal promoter to the coding region of said gene of interest, and
a step of selecting the distal promoter as an endogenous promoter capable of being used in step a) if the expression of said gene of interest is modified.

Said “step of analyzing the region surrounding the gene of interest” is any method known in the art.

In a specific embodiment, the disclosure relates to a method to modify the expression of a gene of interest, as mentioned above, wherein said gene of interest is a fertility-restorer gene, preferably RFL29 gene, more preferably represented by SEQ ID NO:14 (RFL29a). In another embodiment, the fertility-restorer gene is RFL79, more preferably represented by SEQ ID NO:58. In another embodiment, the fertility-restorer gene is RFL29b, more preferably represented by SEQ ID NO: 2. Such embodiment corresponds to a second aspect of the disclosure.

### Method to Enhance the Expression of a Fertility-Restorer Gene of Interest in a Plant

In a second aspect, the disclosure then relates to a method to enhance the expression of a fertility-restorer gene of interest in a plant, said method comprising the steps of:
a) introducing into said plant, at least one gene-editing system, preferably a CRISPR gene-editing system;
b) allowing the said gene-editing system to perform the desired editing at a target genomic site;
   and wherein the said gene-editing system is designed so that, after the desired editing, the fertility-restorer gene of interest is operably linked to an endogenous promoter, or part of an endogenous promoter, said endogenous promoter being distal from a naturally occurring promoter of the fertility-restorer gene of interest in the genome of a non-modified plant.

In a specific embodiment, said method aims to enhance the expression of a fertility-restorer gene of interest in a wheat plant.

In a specific embodiment, the desired editing to enhance the expression of a fertility-restorer gene of interest is preferably a deletion or an inversion. SEQ ID NO:14 notably refers to a RFL29a gene in Triticum aestivum (var. Spelt) P1190962. In a specific embodiment, the distal promoter to drive RFL29 is expressed (active) in Z32 and Z39 spikes. In a specific embodiment, the fertility-restorer gene is RFL29 gene and said endogenous promoter identified to replace the naturally-occurring promoter of RFL29 is chosen among PK-like promoter, RAE1-like promoter or At5g02240-like promoter. PK-like promoter is notably represented by SEQ ID NO:10 (TaPK-like promoter), RAE1-like promoter by SEQ ID NO:11 (TaRAE1-like promoter) and At5g02240-like promoter by SEQ ID NO:59 (TaAt5g02240-like promoter).

In another specific embodiment, the fertility-restorer gene is an RFL79 gene, more preferably represented by SEQ ID NO:58 in Triticum aestivum.

In a specific embodiment, said CRISPR gene-editing system is a delivery system comprising and operably configured to deliver into a plant cell, either CRISPR-Cas complex components, or one or more polynucleotide sequences comprising or encoding said components, wherein said CRISPR-Cas complex components, comprises:
(i) at least two guide sequences capable of hybridizing to the two ends of the target genomic site to effect two breaks, thereby allowing a deletion of said target genomic site or an inversion at said target genomic site;
(ii) a CRISPR-associated protein,
   and wherein said CRISPR-Cas complex is operable in a plant cell.

In a specific embodiment of this method, the fertility-restorer gene is a RFL29 gene and the two guide sequences hybridize to the following targets:
991r+478f, represented respectively by the following sequences SEQ ID NO: 35 and SEQ ID NO: 39,
991r+488r, represented respectively by the following sequences SEQ ID NO: 35 and SEQ ID NO: 38,
972f+479r, represented respectively by the following sequences SEQ ID NO: 36 and SEQ ID NO: 37,
983r+479r, represented respectively by the following sequences SEQ ID NO: 34 and SEQ ID NO: 37,
991r+1155f, represented respectively by the following sequences SEQ ID NO: 35 and SEQ ID NO: 42,
991r+1151f, represented respectively by the following sequences SEQ ID NO: 35 and SEQ ID NO: 43,
972f+1147f, represented respectively by the following sequences SEQ ID NO: 36 and SEQ ID NO: 41,
972f+1145r, represented respectively by the following sequences SEQ ID NO: 36 and SEQ ID NO: 40,
983r+1147f, represented respectively by the following sequences SEQ ID NO: 34 and SEQ ID NO: 41,
983r+1145r, represented respectively by the following sequences SEQ ID NO: 34 and SEQ ID NO: 40,
991r+81f, represented respectively by the following sequences SEQ ID NO: 35 and SEQ ID NO: 57,
972f+70f, represented respectively by the following sequences SEQ ID NO: 36 and SEQ ID NO: 56,
983r+70f, represented respectively by the following sequences SEQ ID NO:34 and SEQ ID NO: 56.

In a specific embodiment of this method, the fertility-restorer gene is RFL29 gene and the said CRISPR gene-editing system comprises the following components:
(1) a first guide sequence which hybridizes to SEQ ID NO: 24 (991r) or a polynucleotide sequence comprising or encoding said first guide, and a second guide sequence which hybridizes to SEQ ID NO: 28 (478f) or a polynucleotide sequence comprising or encoding said second guide sequence, and a Cas9 or a polynucleotide sequence encoding said Cas9, or
(2) a first guide which hybridizes to SEQ ID NO: 24 (991r) or a polynucleotide sequence comprising or encoding said first guide, and a second guide which hybridizes to SEQ ID NO: 27 (488r) or a polynucleotide sequence comprising or encoding said second guide sequence, and a Cas9 or a polynucleotide sequence encoding said Cas9, or
(3) a first guide which hybridizes to SEQ ID NO: 25 (972f) or a polynucleotide sequence comprising or encoding said first guide, and a second guide which hybridizes to SEQ ID NO: 26 (479r) or a polynucleotide sequence comprising or encoding said second guide sequence, and a Cas9 or a polynucleotide sequence encoding said Cas9, or
(4) a first guide which hybridizes to SEQ ID NO: 23 (983r) or a polynucleotide sequence comprising or encoding said first guide, and a second guide which hybridizes to SEQ ID NO: 26 (479r) or a polynucleotide sequence comprising or encoding said second guide sequence, and a Cas9 or a polynucleotide sequence encoding said Cas9, or
(5) a first guide which hybridizes to SEQ ID NO: 24 (991r) or a polynucleotide sequence comprising or encoding said first guide, and a second guide which hybridizes to SEQ ID NO: 31 (1155f) or a polynucleotide sequence comprising or encoding second first guide, and a Cas9 or a polynucleotide sequence encoding said Cas9, or
(6) a first guide which hybridizes to SEQ ID NO: 24 (991r) or a polynucleotide sequence comprising or encoding said first guide, and a second guide which hybridizes to SEQ ID NO: 32 (1151f) or a polynucleotide sequence comprising or encoding said second guide sequence, and a Cas9 or a polynucleotide sequence encoding said Cas9, and a Cas12a or a polynucleotide sequence encoding said Cas12a, or
(7) a first guide which hybridizes to SEQ ID NO: 25 (972f) or a polynucleotide sequence comprising or encoding said first guide, and a second guide which hybridizes to SEQ ID NO: 30 (1147f) or a polynucleotide sequence comprising or encoding said second guide sequence, and a Cas9 or a polynucleotide sequence encoding said Cas9, or
(8) a first guide which hybridizes to SEQ ID NO: 25 (972f) or a polynucleotide sequence comprising or encoding said first guide, and a second guide which hybridizes to SEQ ID NO: 29 (1145r) or a polynucleotide sequence comprising or encoding said second guide sequence, and a Cas9 or a polynucleotide sequence encoding said Cas9, or
(9) a first guide which hybridizes to SEQ ID NO: 23 (983r) or a polynucleotide sequence comprising or encoding said first guide, and a second guide which hybridizes to SEQ ID NO: 30 (1147f) or a polynucleotide sequence comprising or encoding said second guide sequence, and a Cas9 or a polynucleotide sequence encoding said Cas9, or
(10) a first guide which hybridizes to SEQ ID NO: 23 (983r), or a polynucleotide sequence comprising or encoding said first guide and a second guide which hybridizes to SEQ ID NO: 29 (1145r) or a polynucleotide sequence comprising or encoding said second guide sequence, a Cas9 or a polynucleotide sequence encoding said Cas9, or
(11) a first guide which hybridizes to SEQ ID NO: 24 (991r) or a polynucleotide sequence comprising or encoding said first guide, and a second guide which hybridizes to SEQ ID NO: 55 (81f) or a polynucleotide sequence comprising or encoding said second guide sequence, and a Cas9 or a polynucleotide sequence encoding said Cas9, or
(12) a first guide which hybridizes to SEQ ID NO: 25 (972f) or a polynucleotide sequence comprising or encoding said first guide, and a second guide which hybridizes to SEQ ID NO: 54 (70f) or a polynucleotide sequence comprising or encoding said second guide sequence, and a Cas9 or a polynucleotide sequence encoding said Cas9, or
(13) a first guide which hybridizes to SEQ ID NO: 23 (983r) or a polynucleotide sequence comprising or encoding said first guide, and a second guide which hybridizes to SEQ ID NO: 54 (70f) or a polynucleotide sequence comprising or encoding said second guide sequence, and a Cas9 or a polynucleotide sequence encoding said Cas9.

### Plant Used in the Methods of the Disclosure or Obtained by Such Methods

The present disclosure also relates to a plant, or a plant part, which comprises a gene-editing system designed to perform a desired editing at a target genomic site, so that after the desired editing, a gene of interest in the plant is operably linked to an endogenous promoter, said endogenous promoter being distal from a naturally occurring promoter of the gene of interest in the genome of a non-modified plant.

The present disclosure further relates to a plant obtained by the method to modify the expression of a gene of interest in an organism as mentioned above, notably the method to enhance the expression of a fertility-restorer gene of interest in a plant. In a specific embodiment, the disclosure relates thus to a wheat plant wherein the RFL29 gene is preferably linked to an endogenous promoter being distal from the naturally occurring promoter of RFL29 gene in the genome of a non-modified plant, such as PK-like promoter, RAE1-like promoter or At5g02240-like promoter.

Several techniques can be used to identify whether a plant was obtained by a method according to the present disclosure, and/or to determine of the gene editing was correctly performed. Any method known in the art may be used. Some suitable methods include, but are not limited to, sequencing, hybridization assays, polymerase chain reaction (PCR), ligase chain reaction (LCR), or combinations thereof. For example, such a method of identification can comprise a step of PCR after the desiring editing was performed, in order to detect bands corresponding to the desired editing, notably inversion or deletion. The step of PCR amplification is generally followed by a sequencing step. Alternatively, the method of identification can comprise a step to determine the expression level of the gene of interest, in order to compare the expression of a gene of interest by comparison with the expression of a gene of interest in a non-modified plant, thereby verifying the modification of the expression of the gene of interest, preferably a higher expression of the gene of interest. In another alternative embodiment, said identification method can comprise a step to determine the level of the expressed protein encoded by the gene of interest, in order to compare said expression level by comparison with the level of the expressed protein encoded by the gene of interest in a non-modified plant, thereby verifying the modification of the expression level of the protein encoded by the gene of interest, preferably a higher level of the protein.

When said plant was obtained by a method to enhance the expression of a fertility-restorer gene of interest, as disclosed above, a further step of phenotypic analysis can be performed in order to determine if said plant has a fertility restoration phenotype.

### A Method to Restore Fertility

As mentioned above, plant having a fertility restoration phenotype can be obtained according to the methods of the disclosure. It is then desirable to cross such plant with another plant, preferably when the plant is wheat.

Therefore, the present disclosure also relates to a method to restore fertility in wheat, comprising a step of crossing a sterile wheat plant with a plant wherein the expression of a fertility-restorer gene has been enhanced, as mentioned above. Preferably the sterile wheat plant is female wheat and the plant with an enhanced fertility restorer gene is male wheat.

SEQUENCES OF THE PRESENT DISCLOSURE

| Sequence | |
|---|---|
| number | Sequence |
| SEQ ID NO: 1_ | atgccccgcttctcctccaccacgccaatgtcgccaccccgcctccgcctccgcctccgactctgcgcccgccactcctcctccacctctcatccctcacgcatctgggatccccacgccgc |
| Traes- | cttcgccgccgcggcacagcgggcgagctctggcacgctcactacggaggacgcacaccacctgtttgacgaattgctgcggcggggcaatcctgtccaggagcgtcccttgaataaatttc |
| CS1B01G038500 | tggctgccctcgcccgcgcgcccgcgtccgcatcctgctgcgatggccccgccctggcagtcaccctcttcggccgtttgtcccgagacgtcggacgacgggggcgcagccaaatgtcttca |
| (RFL29b)CDS | cctatggcgtcctcatggactgctgctgccgcgcttgccgcacagatctggtgctcgccttctttggccgtctcctcaagacgggcctggaggcaaaccaagtcgtcttcaacaccctcctc |
| | aagggcctttgccacacaaagcgggcggatgaggctctggacgtgctgcttcacaggatgcctgagctgggctgcactcctaatgtggtggcgtataacaccgttatccatggcttctttaa |
| | ggaaggccatgtaagcaaggcctgcaatctgttccatgaaatggcgcagcagggcgttaagcctaatgtggtgacatataactcagttattgatgcgctgtgcaaggccagagccatggaca |
| | aggcagaggtggtccttcgtcagatgattgatgatggtgttggacctgataatgtgacgtatagtagcctcatccatggatattcctcttcaggccactggaaggaggcagttagggtattc |
| | aaagagatgacaagtcggagggttacagcagatgtgcatacttacaacatgtttatgacctttctttgcaaacatggaagaagcaaagaagctgcaggaatttttgataccatggctatcaa |
| | gggcctgaaacctgacaacgtttcatatgctattcgccttcatgggtatgccaccgaaggatgcctagttgacatgatcaatctcttcaattccatggcaacacactgcattctacctaact |
| | gtcatatattcaacatactgattaatgcatatgctaaatctgggaagcttgataaggctatgcttatcttcaatgaaatgcagaaacaaggagtgagtccaaatgcagtcacatattcaacc |
| | gtaatacatgcattttgcaagaagggtaggttggatgatgctgtgataaagtttaatcagatgattgatacaggagtacgaccggacgcatctgtttatcgtcccctaatccagggtttttg |
| | tacacatggcgatttggtgaaagcaaaggaatatgttactgaaatgatgaagaaaggtatgcctcctcctgatattatgttcttcagttcaatcatgcagaacctatgcacagaaggaaggg |
| | taacagaagcacgggatatccttgacttgatagtgcacattggtatgaggcctaatgttatcatatttaatttgctgatcggtggatactgcctagtccgcaagatggcagatgcattgaaa |
| | gtatttgatgatatggtgtcatatggtttagaaccttgtaactttacgtatggtatacttattaatggctattgcaaaaatagaaggattgatgacgggcttattctgttcaaagagatgct |
| | gcacaagggacttaaacctacaacttttaattataacgtcatactggatggattatttctggctggacaaactgttgctgcaaaagagaagtttgatgagatggttgaatctggagtaagtg |
| | tgtgcattgatacatactctataattcttggtggactttgtagaaatagctgcagtagcgaagcgatcacccttttccggaaattaagcgcaatgaatgtgaaatttgatattacaattgtc |
| | aatatcattattggtgccttatacagggtcgagagaaaccaagaggctaaggatttgtttgctgctatgccagccaatggcttggttcctaatgctgttacctacaccgtaatgatgacaaa |
| | tcttataaaagaaggttcagtggaagaagctgacaatcttttcttatccatggagaagagcggctgtactgccaactcttgcctgttaaatcatatcatcagaaggttactggaaaaaggag |
| | agatagtcaaggctggaaattatatgtctaaagttgatgcaaagagctactcacttgaagctaaaactgtttcgctgctgatctctctgttttcaaggaaagggaaatatagagaacacatc |
| | aaattgcttcctacaaagtatcagtttctggaagaagcagccacagttgaatag |
| SEQ ID NO: 2_ | mprfssttpmspprlrlrlrlcarhssstshpsriwdphaafaaaaqrassgtlttedahhlfdellrrgnpvqerpinkflaalarapasasccdgpalavtlfgrlsrdvgrrvaqpnvf |
| Traes- | tygvlmdcccracrtdlvlaffgrllktgleanqvvfntllkglchtkradealdvllhrmpelgctpnvvayntvihgffkeghvskacnlfhemaqqgvkpnvvtynsvidalckaramd |
| CS1B01G038500 | kaevvlrqmiddgvgpdnvtysslihgysssghwkeavrvfkemtsrrvtadvhtynmfmtflckhgrskeaagifdtmaikglkpdnvsyairlhgyategclvdminlfnsmathcilpn |
| (RFL29b) | chifnilinayaksgkldkamlifnemqkqgvspnavtystvihafckkgrlddavikfnqmidtgvrpdasvyrpliqgfcthgdlvkakeyvtemmkkgmpppdimffssimqnlctegr |
| | vteardildlivhigmrpnviifnlliggyclvrkmadalkvfddmvsyglepcnftygilingycknrriddglilfkemlhkglkpttfnynvildglflagqtvaakekfdemvesgvs |
| | vcidtysiilgglcrnscsseaitlfrklsamnvkfditivniiigalyrvernqeakdlfaampanglvpnavtytvmmtnlikegsveeadnlflsmeksgctanscllnhiirrllekg |
| | eivkagnymskvdaksysleaktvsllislfsrkgkyrehikllptkyqfleeaatve |
| SEQ ID NO: 3_ | GCACCTTTAGGTCCTAATTCTTTATTAATTGGTTCACCCGAGGTCCTTTTCCACGAGCGCTCGCTGACCAGCACACGTGCCGCATTGACCCATGCCA |
| Traes- | CATCGATAGAGGGTCTAACCGCCAGGCGAGAAGCTTCGCAAAGAGACCCCCCCCCCTCTTAGTGGCACCCTCTCTCCTCTTTCACGGTACTTTGGC |
| CS1B01G038500 | GACATTGGAGGCGAGCGACAGAGGCAACCGGAGGTGATCTGGAGGTAATTTGTGTGGGTGCCCGTGAAGATGCCCCTTCTATGACGGCCAGTGAG |
| (RFL29b) | CAATCCTCCTCCATGTTACCGTAAGAACCCTAATCTTGTCGATGATCTTCTCCTGTAGCTCTTCTAATATGTCATAACTTTGTCATAATCAATAGTTGTA |
| genomic | GTCCTTGATTTACTTTGATTTGCATAACGGTGATACGGACACGGCAAAAAGGACACACCACCAGCGGCAACATACCCGCCGAAGTTGGAGCTTGCTT |
| | CGCCGCGCCGGGGGGGGTGCTGAGGATGTTGCCATGCTCCTCATCTTAGTTGTTTCCTTGCCGAGGAGCTTGAAGTTCCGGCCATGGATAGGTTTA |
| | GGGGACGCGCGCGCCGAGAGAGAGAGCAGATTTGGGGAAGAAATAAAGCCAGGGACTGGGGAATGAGAAGATAATGTTTATAAGGGGTTTTCTGTA |
| | AAAAGAAAGAGCAACCTACGCAGCCCTTCTGTCGATTTGGCATGGGTCAACGCACCACACGAGCAGGTCAACGAGCGCTCGTGAGAAAAAGGACCT |
| | AGGGTAAACCACTTACTAAAGAATTAGGACCTAAAGGTGCATTTTGGACGAAATAAGACTAAAGTGACACCCTGACGAATTTTTTAGGACCTTGAGTG |
| | CATTTAACTCTTTCAAAAAATAAAGATGCTAAAAAAACTAAATGCTTAAAAATACATTTTTCTATGGGAATACTTAAAAATACTAGTATAATTTGATGGCT |
| | TCCTAGTATATTTCTAGGGCTTACAACTAAATTTATATTCTTGGTCTAAATAAAATTATAATCTGATAAATTATGTGATATGAATATAGCCATATGAGACC |
| | ATAATTTAATTTTACCTGCAAAAAGTAAGTAATAGTAGTAGTAGTACTACATACTCCCTCCGTCCGGAAATACTTGTCGAAGAATTTGATGAAAATGGAT |
| | GCATCTAGAACAAGAATACATCTAGATACATCAATCTCCCTGACAAGTATTTCCGAGCGGAGGGAGTACTAGATAATACTCCCTCCGTTCCTAAATAAT |
| | TGTCTTTCTAGCTATCTCAAATAAACTACAACATACGGATGTATGTAGACATGTTTTAGAGTGTAGATTCACTCATTTTGTTCCGTATGTAGTCATTTGT |
| | TGAAATCTCTAGAGAGACAATTATTTAGGAACGGAGGGAGTAAGATAACTACCCTAAAAAAAAAGATAACTGAAGGTTGCCACCTAGCACATTCACAT |
| | TGGTACAACTTGGAAAAGCACAGCCCCGTCGTCCTGCTCCCAGTTGAGTTCGCGACCTACACACCGGCCATGCCCCGCTTCTCCTCCACCACGCCA |
| | ATGTCGCCACCCCGCCTCCGCCTCCGCCTCCGACTCTGCGCCCGCCACTCCTCCTCCACCTCTCATCCCTCACGCATCTGGGATCCCCACGCCGCC |
| | TTCGCCGCCGCGGCACAGCGGGCGAGCTCTGGCACGCTCACTACGGAGGACGCACACCACCTGTTTGACGAATTGCTGCGGCGGGGCAATCCTGT |
| | CCAGGAGCGTCCCTTGAATAAATTTCTGGCTGCCCTCGCCCGCGCGCCCGCGTCCGCATCCTGCTGCGATGGCCCCGCCCTGGCAGTCACCCTCT |
| | TCGGCCGTTTGTCCCGAGACGTCGGACGACGGGTGGCGCAGCCAAATGTCTTCACCTATGGCGTCCTCATGGACTGCTGCTGCCGCGCTTGCCGC |
| | ACAGATCTGGTGCTCGCCTTCTTTGGCCGTCTCCTCAAGACGGGCCTGGAGGCAAACCAAGTCGTCTTCAACACCCTCCTCAAGGGCCTTTGCCAC |
| | ACAAAGCGGGGGGATGAGGCTCTGGACGTGCTGCTTCACAGGATGCCTGAGCTGGGCTGCACTCCTAATGTGGTGGCGTATAACACCGTTATCCAT |
| | GGCTTCTTTAAGGAAGGCCATGTAAGCAAGGCCTGCAATCTGTTCCATGAAATGGCGCAGCAGGGCGTTAAGCCTAATGTGGTGACATATAACTCAG |
| | TTATTGATGCGCTGTGCAAGGCCAGAGCCATGGACAAGGCAGAGGTGGTCCTTCGTCAGATGATTGATGATGGTGTTGGACCTGATAATGTGACGTA |
| | TAGTAGCCTCATCCATGGATATTCCTCTTCAGGCCACTGGAAGGAGGCAGTTAGGGTATTCAAAGAGATGACAAGTCGGAGGGTTACAGCAGATGTG |
| | CATACTTACAACATGTTTATGACCTTTCTTTGCAAACATGGAAGAAGCAAAGAAGCTGCAGGAATTTTTGATACCATGGCTATCAAGGGCCTGAAACCT |
| | GACAACGTTTCATATGCTATTCGCCTTCATGGGTATGCCACCGAAGGATGCCTAGTTGACATGATCAATCTCTTCAATTCCATGGCAACACACTGCAT |
| | TCTACCTAACTGTCATATATTCAACATACTGATTAATGCATATGCTAAATCTGGGAAGCTTGATAAGGCTATGCTTATCTTCAATGAAATGCAGAAACAA |
| | GGAGTGAGTCCAAATGCAGTCACATATTCAACCGTAATACATGCATTTTGCAAGAAGGGTAGGTTGGATGATGCTGTGATAAAGTTTAATCAGATGAT |
| | TGATACAGGAGTACGACCGGACGCATCTGTTTATCGTCCCCTAATCCAGGGTTTTTGTACACATGGCGATTTGGTGAAAGCAAAGGAATATGTTACTG |
| | AAATGATGAAGAAAGGTATGCCTCCTCCTGATATTATGTTCTTCAGTTCAATCATGCAGAACCTATGCACAGAAGGAAGGGTAACAGAAGCACGGGAT |
| | ATCCTTGACTTGATAGTGCACATTGGTATGAGGCCTAATGTTATCATATTTAATTTGCTGATCGGTGGATACTGCCTAGTCCGCAAGATGGCAGATGC |
| | ATTGAAAGTATTTGATGATATGGTGTCATATGGTTTAGAACCTTGTAACTTTACGTATGGTATACTTATTAATGGCTATTGCAAAAATAGAAGGATTGAT |
| | GACGGGCTTATTCTGTTCAAAGAGATGCTGCACAAGGGACTTAAACCTACAACTTTTAATTATAACGTCATACTGGATGGATTATTTCTGGCTGGACAA |
| | ACTGTTGCTGCAAAAGAGAAGTTTGATGAGATGGTTGAATCTGGAGTAAGTGTGTGCATTGATACATACTCTATAATTCTTGGTGGACTTTGTAGAAAT |
| | AGCTGCAGTAGCGAAGCGATCACCCTTTTCCGGAAATTAAGCGCAATGAATGTGAAATTTGATATTACAATTGTCAATATCATTATTGGTGCCTTATAC |
| | AGGGTCGAGAGAAACCAAGAGGCTAAGGATTTGTTTGCTGCTATGCCAGCCAATGGCTTGGTTCCTAATGCTGTTACCTACACCGTAATGATGACAA |
| | ATCTTATAAAAGAAGGTTCAGTGGAAGAAGCTGACAATCTTTTCTTATCCATGGAGAAGAGCGGCTGTACTGCCAACTCTTGCCTGTTAAATCATATCA |
| | TCAGAAGGTTACTGGAAAAAGGAGAGATAGTCAAGGCTGGAAATTATATGTCTAAAGTTGATGCAAAGAGCTACTCACTTGAAGCTAAAACTGTTTCG |
| | CTGCTGATCTCTCTGTTTTCAAGGAAAGGGAAATATAGAGAACACATCAAATTGCTTCCTACAAAGTATCAGTTTCTGGAAGAAGCAGCCACAGTTGA |
| | ATAGTTGGTACATGATATCTGAAATTTAATTTGCATCGCTTGCCATGGGTCCGTCTGCTTGTACAAGAAATGCATTTTCTATTTGTAAATAGGAAGTCA |
| | GTTTAGAACAAGCCATCAGGATGACGCAAAGAGTACAATTCAGTTGCACCACCAATAAAAAGGCAGAACTAGGGCTGCCAAAACAACACTGAATCAA |
| | AACTCAAACAGAAGGAGCAGCAAACTTTTTTTTTTGAAGCTGGACAGTCTGCTAGCCAAACAACTACAGGAGACTGTCAGGCGGGGCATGTAGTGGC |
| | TGGCGTCTAAGCGCCTTTGCTTCTTCCACCATCCATGGCTTAGCCTCACACGGAATCGAGTCAACCAATTCCCGTCGGTTTTGGGTGGCTCCCTTGA |
| | AGATGCAATTGTTTTCAGCGGCCAGATACGCATGGTCAGATTAATCAGCGAGTGTGCCCCTTCTGTCTGGTTCGAGAAAGAATTTGAAGAGCTGAGC |
| | TTGTCCCAGCTGGAACCAGTTTGAGGTTAGTTAATCATAACAGGGTACTAGAGAGGTGTTTTATTGACTGTTGATGTGTAATATGTTATATGCCATCCT |
| | CTTGATGATTACGGTGATCTGTGAAGAGGCAGCATGCAAAAGTCTGAATCACATATGCTTATGTAATTGTGTTATTATTTGTGCAGCTTCTAGACCTTT |
| | TGCCTTGTAGATGGCTACATGGATCTAGTTGTAGCAATCTGTAACTGTTAGTGTTTTGTATATGCTGGCATTGATGATTAGGGTGACTTGTGAAGCATG |
| | CAGAAGTCTGAATCATACATGCTTGTCTAGTTATTTTTGGGGCTCTCTCTTTGCCATGTACATGTTTTATGGATGCAGTTGTAGTCTGTATGTGCTGGC |
| | ATTTAATATGAGGGCTGGTCTGCAATTTTTCTGTTTTGATCGATACAGCGCCATGTTGAGTTTAACCCTGTAACTTGGCTTAACTAGATCATGCTGGTG |
| | TTATCTCTAGGAATCACAATATCAATACTCTCTGTTGTTGCACAAACTGACGCAGATCAATGTCCAGAGGTAATATGGTTCTGCCCGTAATTTTCATCG |
| | ACTGTCAGCATGTTCCTTTTTTATTTAGTACTCTGTATATCACGGCGGTATGACTATAATAAGGATATACGGCTGGTGCATTTTGAGCTTCTCTGTTTCT |
| | TGGGATAAAGCTGATAAATTTCTATTCTTGTTGGCTTTGGTAGTATGAACAGAAAGCAGGCAGTAATAAACTGATATGTGGTACTATTTTGTTAGTGAA |
| | ACAGGGTCCTTTTTATTATCTATTTTATCCAGCAATAGTGTTCCATAAAATTATAGATCGGTTCATTCATCTCATATGGATTTCGGTGAGCTTCGCCCGT |
| | CCTTGGTACATTTTGTTACTATAGAACAATTGGTGTGCGCTTTTTTTCTTTTGGCTGTACGTTATAGAACAACAAAACCTGTTTATTTCATTAATTCATTG |
| | TCTAATTTATTTTCTTACGAGAGCTCAGAAGTTCAGCATTTTATTTTATCTTGTTCCTTCCGCGTAATTTTTCATTTACTCACTGAGTAGTACAGGTCTTG |
| | GACCTGAAATTCTTGTACTAATTTAGTAGAACCATTCTTCGAGAAACACTCCAAAAATGGTTTGTCTTGGGTCTGAATCATATTGTCTCTGAACAAACA |
| | GGTGTGACTCATGCATGGAAAGATCCCCAAGAAACATTGCACATTTGGTAGATATGTCTATGCATGAATACTATTTTTTGAAAAAGAATTTAGTAAATAT |
| | TTTGGTAGCATTTTTTATACGTATGATGCTTTTTCTTCTTCCCGAAATAAGGGTTTTGATTTTTGAATTTTTGTAAAGCAACATGGTTTGTAGTACGTACG |
| | AAAGAACGCTAGTGTGTTTTCGCGATCGTAAGAATGTTATAAACTGTTTTCCTGGGTCATGTAGGTGTAAGCTATAGAAGGAATTCAATATCGTCCTAA |
| | ACATCCTTGGCAGTGGCCAGTGAAAGTCTTGGACCTGAAATCATACCAGTACTATTTTTCTGAAAGAGAACCCATTCTTGTATTCCATAAGTTGGCTG |
| | GTGCCTGGGCATAATTGTCTAATCATATAGTACTATTTGCATCTCATAATTCCATTCAGTTTTGCACTATAGCTGAACTGAAGGAGTACCTAGCACAAA |
| | ATATGTATGACTGTATTTCAGATCTTGCAATCGCAGCTGTAATGGAGTAGCACACCCTGTAGCAGCATATGGATGTACTCTGAATGTTGGGACTCTTT |
| | GAAATGATAAATTGGTCTATTCTGGGAGCAGATTTTTTCCGGCTTTCGGAGATTCATTGATCTTTTTTTTTCAGAAAGTACAGTTGTAAGTTATAATCAA |
| | ACATCCCGAACAAACCTCACTTGTACATGTAAGAATGCACTATGAATCAGTGGAAAGTAGATTTGTGGTGAACCAAAATAGCCAAGGAATAGGTTCAC |
| | TTTCCTCCCTCCTTATGTCTAAAGTTGATGCAAAGAGCTACTCACTTGAAGCTAAAACTGTTTCGCTGCTGATCTCTCTCTTTTCAGGGAAAGGGAAAT |
| | ACAGAGAACAGATAAGATTGCTCCCCGCAAAGTATCAGTTTCGCGGAGAATGAGCTACAGTTGAATGGTTTTACATGATATCTGAACTTAACTCCTCA |
| | GAAGGTAAGGTTGAATTATTGTTTGCCATGAGTTTATCTGCTTGTACAACAAGTGCAATTTCCATATGTAAATAGGAAGGCAGTTGAGAACAAGCCATC |
| | AGGATGACGCAAAGAGTACAATTCAACTGTAGGACCAGCAATTGTTTTGAAGTTGTATGTCTGCTAGTAGAACACCTACGGGAGAGATTCAGGCGGC |
| | CGGGCATGTAGTGGCACCTGTGTAAGTCCCTTTTCTGCTGCCAGCATCCATGGCTTGGCCTCACAGGGAATGGAGTCAACCAATTTCTCGGCGTAG |
| | CTGTTGCAAATGTTTTCTCAGCCACCAGATACACCAAAGTTTCAGAGTAATCAGCGAGCGTGTCTCTTTTGTCTGGTTCGAGAAAGAATTTGAAAACG |
| | CTGAGCAGCAACAGGTGCTTGACCAGCTTGTCCCTGCCGAAGCCAGTTTGAGGTTAGTTAATCATAACAGGGTACTAGACAGGTGTTTTGTTGACTG |
| | TTGATGGGTAATACTCCTATGTTATATGCCAGCCACTTGATGAATAGGGTGATCTGTGAATCACATATGCTTATGTAATTGTGTTATTATTTGTGCAGCT |
| | TCTAGACCTTCTGCCTTGTATGGCTACATGGATCACGTTGTAGCAGTCTGTGACTGTGACTGTTAGTGTTTTGTATATGCTGGCATTGATTAATTTAGG |
| | GCCGGGCTCCAAATTATTTGTTTTGATCGATTCAGCACCATGTTGAGTTTAACCCTGTAACTTGGCTTAACCAGATCAATATCAGGGGCCTCGGCATT |
| | TTTAAACGTTGAATTAAACATACTCTGTGGCATATCAATGTTCAGAGGTAATATGGTTCTGTCCATAATTTCATCGACTGTCAGCATGTTCCTTTGTTAT |
| | TCAATACTCTGTATATCATGCCGGTATGACTATAATAAGGATATAGTATGTTTGATGCATTTTGAACTTCTCTATTCCGTCCGATAGAGGTGATAAATTC |
| | TCTATTCTTTTTGGCTTTGGTAGTATGAACAGTAAGCAGGCAGTATATATTAAACTGATATGTGGTACTATTTTGTTAATGAAACAG |
| SEQ ID NO: 4_ | atggggaactgctggggcgccaagatcagctccgacacctcctcctcctcctccccttcagggacgaattccaagtatgctactaggaatggggcggccttgagcagctccagcagctatgc |
| ZTraes- | acggagcgagggtgagattctggagtcagccaatgtcaaggccttctctttcaatgagctgaggaccgccacgagaaacttccgtccggacagtgtgctaggcgagggagggttcgggtcag |
| CS1B01G038800 | tcttcaaggggtggatcgatgagaagaccctcaccccaactaagccaggcaccgggatggtcattgctgtaaagaagcttaaccaggaaagctatcagggccatagggaatggctggctgaa |
| (TaPK-like) | gtgaattaccttggacaactatcgcacccgaatcttgtaaagctcgttgggtactgtgtcgaagacgaacagcggcttctcgtctacgagttcatgccccgtgggagtttagagaatcatct |
| CDS | attcaggaggagtacacatttccagccgctctcctggaaccttcggatgaaaattgcccatggagcagcaaaagggctcgcgtttctccacagtgacaaggccaaagtcatctaccgcgatt |
| | tcaaaacctctaatatcctcctagatgcgaactatgacgcaaagctctcagatttcggcctggcgaaggacggaccgacgggtgacaagagccatgtgtccacaagggtgatggggacatat |
| | gggtatgctgcaccagaataccttgcaacaggccacctgaccacgaagagcgacgtgtacagcttcggcgtggtcctcctggagatgctctcggggcgtcgcgcggtggacaagaaccggcc |
| | gaccggcgagcacaacctggtggagtgggcgcggccgtacctgacaagcaagcggcgcatcttccgcgtcctggacccccgtctggggggcagtactccctcgccaaggcccagaaggcggc |
| | gtcgctggcgctgcagtgcctctcggtggactcgaggaacaggccgagcatggagcaggtcgtcgtggtgctggagcagctccacgacgccaaggagggagggaacagccctcgcccgcagc |
| | tgcagagaaagccgagcagcaaccggggcgtgaacggctcgagatcgtcgtcgaccaaggggaacaacaagaagcccgcctcaccgagaccggtttga |
| SEQ ID NO: 5_ | mgncwgakissdtssssspsgtnskyatrngaalsssssyasaasvprsegeilesanvkafsfnelrtatrnfrpdsvlgeggfgsvfkgwidektltptkpgtgmviavkklnqesyqgh |
| Traes- | rewlaevnylgqlshpnlvklvgycvedeqrllvyefmprgslenhlfrrsthfqplswnlrmkiahgaakglaflhsdkakviyrdfktsnilldanydaklsdfglakdgptgdkshvst |
| CS1B01G038800 | rvmgtygyaapeylatghlttksdvysfgvvllemlsgrravdknrptgehnlvewarpyltskrrifrvldprlggqyslakaqkaaslalqclsvdsrnrpsmeqvvvvleqlhdakegg |
| (TaPK-like) | nsprpqlqrkpssnrgvngsrssstkgnnkkpasprpv |
| SEQ ID NO: 6_ | CACGGAGTGACAAATCCCAGTCTCGATCCGCATAAAACAATAGATACTTTCGGAGATACCTGTAGTGCACCTTTATAGTCACCCAATTACGTTGTGAC |
| Traes- | GTTTGATACACCCAAAGCACTCCTACGGTATCCAGGAGTTACACGCTCTCATGGTCAAAGGAAGAGATACTTGACATTGGCAAAACTCTAGCAAATGA |
| CS1B01G038800 | ACTACACGATCTTTTGTGCTAGTCTTAGGATTGGGTCTTGTCCATCACATCATTCTCCTAATGATGTGATCCCGTTATCAACGACATCCAATGTCCATA |
| (TaPK-like) | GCCAGGAAACCATGACTATCTGTTGATCACAACGAGCTAGTCAACTAGAGGCTCACTAGGGACATATTGTGGTCTATGTATTCACACATGTATTACTA |
| genomic | TTTCCGGATAATACAGTTATAGCATGAATAAAAGACAATTATCATGAACAAGGAAATATAATAATAATACTTTTATTATTTCCTCTAGGGCATATTTCCAA |
| | CAAAAGTGTCGCAGGAAATAGAAAGTAGTATCCTACGGTATGTATTGCATTTATTAGTTGTAGGCTCAACTAAAAGCACTACTGCGTGGTGACAACCT |
| | AAGACTAGCCACAGTGAGAGTAACTTTAGTAGTAACATCGAGTCCAACTCAGCAAATTTGCTTATGTGGCAATGAGTTAATGAGGAGAGATGTAATAC |
| | AAGTAACTTAGCTAATTACTGTAACACTACATCTCCCAATGCATTATGAGGGGGTGTTTGGTTCAGAAGTCCTAGGACTTTTTCTAGTCCCAGGGACTA |
| | ATAAAAAAAAACTCTCTACTAGAGTCTTTTTCTAGTCCATGCATAAAAAGTCCCTTCCGTTTGGTTCATAGGAACTTTTTAGGGACCTTTTCTAGTCTCT |
| | GAGACTAAAAAATCTCTGAACCAAACACCCCCTGAGTCTATAACCTAATAAATGAATCTTTGTATGTTACCATACTTATGTTACTACCCATAGCCTAGAA |
| | ACGTGTGTATGTTACTCTCCATAGTGGTTAGTCTAATACGTGACTTTAAACATCTCTCTGCCCAAGTAGCTGCCACATAAGCAGATGGGCGTCTTAATA |
| | TAATAATATAGAAATGTATCGGGGTAATATAATACTGTGGCGATAGGCTTGGGTTAATTTCTTTAGTCCCGCCGATAAGCTCGATTGGTCAGTGCTGG |
| | TGCCCCTCTCGACTCCACACACACACACACAGAGGGCGAAAAATAAGGGCACACGAGGCCACGTCACGTGGGGCCCCCATGACCCACACGCGGGC |
| | CCGCGCGCCATCGTGTCGACGCGCCGCGAGCAGAGACGGCCGGCGAGGCCAACCCGAACCACCACTCCGCGCGAGCCGCCCCTCCCTTTCTCTC |
| | GCCTTTTCGGCCCNNNNNNNNNNNNNNNNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN |
| | NNNNNNNNNNNNNNTAGCGGTAGTGTATCGGGTTCGCCTCCCTCCCCCTCCCCCTGCCTGCACTGTCCCATCGCTCGCTCGCTCGCTTTCTCGCCC |
| | GCTCGGTGGCGGTCGTGGGGGAGAAAGTTGCCGGCCGGCCAGCGCACGGGGAAAAGGCGCACATACTTTCTTTCTTTCTTTCCACCGCCGCCCTC |
| | CTCCTCCTCCCGGAGGAGTTCCGATTCCCCCTCTCTTTCTCTCTCTTCTTTCTCTCTCTCTTTCTTGCTCTGCTCTGGAAGCAGGGGGTCCTGATTTG |
| | GGCGCGGGCGCCGTCCGCCGTGATTCGGTTCGGCTGATTTGGGAGGTCTGCTCGTTTGATCCGGGTTCGCTGATTGGTTGATTGATTGATTGGTTG |
| | GTTCATCTCCGGCGGCTCGATTTGAGGGCCGGTCGGCTTGGTCGCCATGGGGAACTGCTGGGGCGCCAAGATCAGCTCCGACACCTCCTCCTCCT |
| | CCTCCCCTTCAGGTGCGTGCGTCGTCCTCCCTCCCTCTCCTCATGCTACTATTTTGCAAAGATTTTTAATACTACGTACCAGCGGTACGACTAGTTCA |
| | TAACTAATCTGTGCCCAGAGGGTCGAATAAATCCTGAATGAAAAATCTGGTGGTTTTTGGTTTGCGGCGACTTCGTGCTCTGCATCCCTTCTCTCCTT |
| | CCCGAAACCAGTCCTCGGTCATGCTGATTCGATTTCGGTCCAATCCAACTGCTTCAACATAGGGCAAACACTAGGTTAGGTTCACTACTGTAGAAACT |
| | TAATCTGGTTCCTACCAAATAGTACTCCCTCCGTAAACTAATATAAGAGTGTTTAGATCCCTATTTTAGTGATCTAAACGCTCTTATATTAGTTTACAGA |
| | GAGAGTATTAAGTAGACTACTCAATAAAGAAATCACTGGTCTGTGTTAACTCTCTACTACTTTGCATCTCCTCACTGTTTTCGGGAACTAAGACTAGAA |
| | CTGGGTTATGCTGATTTCGTTCCGTTTAATCGACTCAAGATTGGAGAGTCACTAAATTCAGTACAGCACAAACTTAATTTGTCTCCCTAGGAAAATTCA |
| | GTCCATTCTTTGGAAAAATCTGTCGCGAGCTTTGACTCTTCCTCTTGACCTGCTTCGTGTGCAAGGGATCATGCCTTTCCGTTTTGGGCCTAGGGGAG |
| | AGGAAGCTTCCGATGTAGTAGTAAAATGCTTCACTTAACTTTATCTGACTTCTCCTCTGATTTTTCTGAGTGTCCAGTTCAAATCAGACATATAGAGGA |
| | AAAGCTCGAATTCTGATGCTACTTTTGGCTACATCCACGTGCTTTTCTTCAAAGTTATTGACTGTCCTGTTTTGGATGTTCAGCCTTCTTTTCTGTAATA |
| | AGCGAAACTAAAGGAAGAAAATACATAGTTAGTCCGACCTAACTTGTCCATGATGAACCTATTTTCTGTTCAATGTTAGGCAGACAAGATAAATGATAA |
| | AGGGACAGCAACCTATTTATTATTCTAAACCGTGATCGAATCATTTCTGAAACTCTGCACCATTGTATGATGATGATGATCAGGGACGAATTCCAAGTA |
| | TGCTACTAGGAATGGGGCGGCCTTGAGCAGCTCCAGCAGCTATGCCTCCGCGGCGTCAGTGCCACGGAGCGAGGGTGAGATTCTGGAGTCAGCCA |
| | ATGTCAAGGCCTTCTCTTTCAATGAGCTGAGGACCGCCACGAGAAACTTCCGTCCGGACAGTGTGCTAGGCGAGGGAGGGTTCGGGTCAGTCTTCA |
| | AGGGGTGGATCGATGAGAAGACCCTCACCCCAACTAAGCCAGGCACCGGGATGGTCATTGCTGTAAAGAAGCTTAACCAGGAAAGCTATCAGGGCC |
| | ATAGGGAATGGCTGGTTAGTGACAATTTTGCCTGCTGGAAATGGGATTTCTTGTTTATTTCAGTTCTGCATTGTGTCTGACATGCTCTTTCTTTTGGGC |
| | GCAGGCTGAAGTGAATTACCTTGGACAACTATCGCACCCGAATCTTGTAAAGCTCGTTGGGTACTGTGTCGAAGACGAACAGCGGCTTCTCGTCTAC |
| | GAGTTCATGCCCCGTGGGAGTTTAGAGAATCATCTATTCAGGAGTAAGCCCCTGGCCTTTTACTTTTCTTATTCGCTGGGTGTTATTCTCCAGTTTTGA |
| | AGGAATGATGCTAAAGTGTTCCAACATTCAGGGAGTACACATTTCCAGCCGCTCTCCTGGAACCTTCGGATGAAAATTGCCCATGGAGCAGCAAAAG |
| | GGCTCGCGTTTCTCCACAGTGACAAGGCCAAAGTCATCTACCGCGATTTCAAAACCTCTAATATCCTCCTAGATGCGGTATATATATCCACAGCCTTG |
| | AAGGATCTTTTCCTTTGCGTCCGCAAAATATCTCTATCGGTCGATCTGACAGTCACGCAATTTCTTCTTCAGAACTATGACGCAAAGCTCTCAGATTTC |
| | GGCCTGGCGAAGGACGGACCGACGGGTGACAAGAGCCATGTGTCCACAAGGGTGATGGGGACATATGGGTATGCTGCACCAGAATACCTTGCAAC |
| | AGGTCGGTTGATCATTCCACTTTCAGAATCCTCAGCTGCGCAAATATAAGATATATTGCTGAAAATCTGACCACGCCTACCTGAATCGTGATTGCGTT |
| | GTCACTCTGGTTGTGTCATCGACCAAGATGCCGAACATAACCAATTTTAGGCTACAACCTTTCAATCATCATCTGCACAAATATCTTGCCCGAAAATCT |
| | AACCACACCTACCTGAATCATGATTGCGTTCTCACTCTGGTTGCGTCATCGACCAAGATGCCAAACTTAACTAATTTTTCGGCTAGAACCTTTCAGTCC |
| | TCATCTGCACAAATATCTTGCTGAAAATCTAAACACACCTACCTGAATCGCGAGTGCATTGTCACCCTGGTTGCGTCATCAACCATCATGCCGAACTT |
| | AAACTAATTTTCCTGCTGCAATATTTCCATGATCAGGCCACCTGACCACGAAGAGCGACGTGTACAGCTTCGGCGTGGTCCTCCTGGAGATGCTCTC |
| | GGGGCGTCGCGCGGTGGACAAGAACCGGCCGACCGGCGAGCACAACCTGGTGGAGTGGGCGCGGCCGTACCTGACAAGCAAGCGGCGCATCTT |
| | CCGCGTCCTGGACCCCCGTCTGGGGGGGCAGTACTCCCTCGCCAAGGCCCAGAAGGCGGCGTCGCTGGCGCTGCAGTGCCTCTCGGTGGACTCG |
| | AGGAACAGGCCGAGCATGGAGCAGGTCGTCGTGGTGCTGGAGCAGCTCCACGACGCCAAGGAGGGAGGGAACAGCCCTCGCCCGCAGCTGCAGA |
| | GAAAGCCGAGCAGCAACCGGGGCGTGAACGGCTCGAGATCGTCGTCGACCAAGGGGAACAACAAGAAGCCCGCCTCACCGAGACCGGTTTGACC |
| | GTGCGAGTGGGATCACATAGCAGAATCTACTAGAGAGATGCGTCAAGGTGTAATGTATGAGTGGTGATCTGCGAAGAGGCAGCGGATTTGTGAATG |
| | TTTTGTGCCCGTGTTGTAGGGATGTATCTTTGCAGCTTCTAGTGTTGGTATGTACAGCTTGTATCTATCCGGTTGTAGCTAGTCTGTATGTAGTCCTTT |
| | TGTGTATGCCTGATTGCTTTGGTTCTGACTTAAGGTCCAGAGGTTTTGATCTGATTTAGATGCATGCACCCTGTTAAACCAAGACCTTAAAATATGAAG |
| | ATGAACAGTGTAAAGTACATATTTGTCACTTCATTTATCAAATGTGAATGAACAATCATATTGGTGTTTTAGCAGAGCAGGCCATGACTTCCATGGGTC |
| | AACTGTATCTTTTCCCTTAGGGATGTTGACCATTTTGTTCATATCATACAGTACTAGTAGTATATGTTTCATTTTTTTTATGAAAAAAGTAGTATATGTTA |
| | CAGATAATGTTGGGAAAATCACCTTCTGTATTTTTAATTTCAAACAGTTGGCCACAATGATCTTTTTATTTACTTGGTGGGTTCAGCCAAATTACCATTG |
| | TTTTTTTCAGGGGTTTCAGCCAAATTATTATTATCTGGGCAAGCGTACGGCGGCGGCCCAAGGCAGAGCACGCAGCCCAGCCGCCAAAACAGAGCT |
| | CGCCCTCGCTCCCCTCCGGCCCAAGTGCCGCTCCTCCTCCTCCTCGGTGGACGCCGGCGCCGGTGGATGGCTCCGCCGCGCCAAATCGAGCCCC |
| | TCACGCCGCGAGCCTCTCCTCCTCCTCCTCAGCCTGCAATCCCCGCCGAGCTCCGATCCCCGCCGCCGAGCTCCGATCCGCGGGCGATCGCCGG |
| | GGCGCCGCCAAGATCAGCTCCGGCCCCTCCCGTTCCTTTAGGTGCGCGTTTCTATTCTTCAGCAAAATAGTTTTCTGGCATCCGACCAGGTTAATAC |
| | CCACCGTAACAAACGGACAGGCTATCGCGAATGTTTTGATGAACGATACTAATTGC |
| SEQ ID NO: 7__ | atggcagctctaaccagccttgcctcaaacccgaatccaaacaagtcattcgaggtccttcctaatccgggtgactccctctcaagcctcagttttagcccgaaaagtaatcttcttgtggc |
| Traes- | aacttcctgggataaccaggtgaggtgttgggagataggtaatggtaacagtcagccaaaggcatccatatcacatgatcagccagtgctctgctcagcctggaaagatgatgggactactg |
| CS1B01G041300 | tcttctctggagggtgtgataaacaggtcaaaatgtggcctctgctgtctggtgggcaggctcagacggttgcaatgcatgatgcacctgtcaaggaggtcgcatggatttctcagatgaat |
| (TaRAE1-like) | cttcttgtatccggaagctgggacaagacactaaggtattgggacacaagacagccgaatccagcccatgttcagcaacttcctgatcgttgctacgcacttgctgtgaattatccccttat |
| CDS | gattgtgggaacagctgatcgcaatattgtgatcttcaacttgcagaatcctcagactgagtttaagcgtattcaatcacctctgaaataccagacacggtgcgttgctgcctttccagatc |
| | aacaaggattcctggtgggttccatagaaggaagagttggtgtgcatcatattgatgattcacagcaaagcaaaaacttcacattcaagtgtcacagggaaggaaatgatattttctctgtc |
| | aattcgctcaactttcaccctgttcatcacacgtttgccacagctggatctgatggtgctttcaacttttgggataaagatagcaagcagagacttaaggctttcagtcggtgtcctcaacc |
| | cattccttgcagtagcttcaataatgatggttcaatatttgcttatggggtgtgctatgactggagccgtggcgctgagaaccataatcctgcaaatgcaaagacatccatctatctccaca |
| | gtccccaggaagccgaggtgaaagggaagccaagaatcgcaacggggcggaagtga |
| SEQ ID NO: 8_ | maaltslasnpnpnksfevlpnpgdslsslsfspksnllvatswdnqvrcweigngnsqpkasishdqpvlcsawkddgttvfsggcdkqvkmwpllsggqaqtvamhdapvkevawisqmn |
| Traes- | llvsgswdktlrywdtrqpnpahvqqlpdrcyalavnyplmivgtadrnivifnlqnpqtefkriqsplkyqtrcvaafpdqqgflvgsiegrvgvhhiddsqqsknftfkchregndifsv |
| CS1B01G041300 | nslnfhpvhhtfatagsdgafnfwdkdskqrlkafsrcpqpipcssfnndgsifaygvcydwsrgaenhnpanaktsiylhspqeaevkgkpriatgrk |
| (TaRAE1-like) | |
| SEQ ID NO: 9_ | ggtgaggttatggtatgtacaattttggcgcacaatgacggcgacttccctgatccttcgtcatcgtctccaccgtgttggttttggtgctggcgtgaggcctgaggtatgtctcttggatc |
| Traes- | taatgctcgtctagcgttggtggtttctccttcagagcgatggcccggtaagacgtggccttaatcactccccgcccgtgatgaacatcaggctggcttcgatgatggagaagcatgtgttg |
| CS1B01G041300 | gctcggcatgttctagaggatgaagaaggatgacccaggaattttgatgtaactttattaatattttcttcctataatttgtacttccttttcttagaaaaaaaactattttgtactgctag |
| (TaRAE1-like) | tttgattaatattcgtggcttcacatgcgctggtttggttatataagagggagcatatgggtgttgtctttttatataagaaaaaaaagagcaaaataagccacgcgccgatcaagcctgcg |
| genomic | aactgagagtatacccggccatgggcaacccagactgacccgacccgtcccgactcgaccttgcccacgggccgggcccaggcctagattttgagcccgatggctgggccgacatgttgtat |
| | ttgcgatttaaggaagaggcccggcccaagggccgaggcccgacgggttttttgactgatgggccggggttgggccaaattgttaggcccaacggccgggctcgggcctaagccttttgctt |
| | gcgttcaggctttgataggctcagcccgaagtccacatccacatcaaccacccccgcgtgccgatgaagccattttttggaaggaccttcgcgtgcatccgacgcgacgcgctacaccgaag |
| | cctatactactacatctcgtctcatcgccctcctcccacacgacgactcgagaatcctcggctgaggctccactccgcactcgcaagtcgcacccgcagccgccgccgaccgctccccgtcc |
| | ggcaggtaagccccccgagcccctcctcgccgctccgctcgcgcggactcctagctagctagggttcccggcaccgattccgcggcgctgcactccggcgcaatgctcgcggccgcggacgg |
| | gcccgccgcttcgggggaagctctgatctggtggccggcgctcgctcgctcgctcgcggtctccggagcaaatcggccgcatcgagtggccggagcaagcttaggatagttcgattatttcc |
| | cccatccccgttggtcaggccgagagggccagtttgggcgacctcctggtgattccgctgccgtggtggggattgggtggtcgtggcccgggtgtaggcgtttgtaggtcgagtgagtgttt |
| | ccttccaggttctatgctcacccagcttgtgttatcactgggatccgggcgttcggcaggctaacattcaccggattttggacctgcattgttgcggcgcgtgccgggggatagcgtttatg |
| | ctggctttggcgcggtgattttgtgcgtgcgtgcaaacttttggggctgccttctggtgatctcgctgtcgggtggtgcgagttcatcaagcggtcagggtgtaggcatttgtagtcgagcg |
| | accccagctttcatgcccaccagctcgtattattcggtcgttaaccacgtttaccgaattttggaccgacgtcgttgcggcacgtaccaggggacagcgtttatgctggtttcggtgtggag |
| | attttgtggtttaaacttggggcgcgcttggtttgcgaggctacctcctggtgatttcaccgtcgggtggtgggaatcggatggtcggggtgtaggcatttatagtggagcggaccccttgt |
| | tttatgaccagacatttagccgaaatttggacctacactatcgtggcgtaccaggggatatcatttatgcgggttttggcgcgacactttctttgtcgggattttgtggtgcaaacttaaat |
| | aatgacttccctaatgttaaacactcgttctggcatcgtggactgttggaatcgttctttttcgtagtctgagctactttaagttgttacttatgctctcgtgtgcgtgggagactccacgt |
| | gctgatttctcttacttgcttctcatggtcactggactgatatttcttttccagattttgaaggttcacagaaggagagatggcagctctaaccagccttgcctcaaacccgaatccaaaca |
| | agtcattcgaggtgtgatactttttgtggccatgtctgtcctgcttttatttttttgcgtcttgtcattctgtatatgctacatgttctctgcaatggttgctttcaaatgcaggtccttcc |
| | gtaatccggtgactccctctcaagcctcagttttagcccgaaaagtaatcttcttgtggcaacttcctgggataaccaggtaattttgtttctgctgtaattttcagtgagcttccattatt |
| | gtgatgtatcttactaggaatgtcttttgagttttgtgcaggtgaggtgttgggagataggtaatggtaacagtcagccaaaggcatccatatcacatgatcagccagtacgttctcataca |
| | tgtttctcctgaaagtagtttcatcttacacattagagtggttccttacctgtttacttggtatgatgattgatttgtttccttccatcaggtgctctgctcagcctggaaagatgatggga |
| | ctactgtcttctctggagggtgtgataaacaggtcaaaatgtggcctctgctgtctggtgggcaggctcagacggttgcaatgcatgatgcacctgtcaaggaggtcgcatggatttctcag |
| | atgaatcttcttgtatccggaagctgggacaagacactaaggtgcaagaacaatattcctctccaccctcagtattctttttacatggaaattcagcgcatatcacccatatctgatcccaa |
| | attgttatttatggctgttttgcttttgggaattcgtgttgtttgtggtatcctttctgtacttcatgtgttctgcattaatgttcacacattggctaaccaaatattgtagaagttctatc |
| | tcatcctctttatatttgcactcattttgtatgccttctttgatcctgaagagttttctactacttgttgaatcatactgccctattcttatcaaaggttgtgcactttctgggtatcatct |
| | gtactatttatggctgtagtgaatttttcttatggcagaagtttttatagaagtaactgttgatgcagacagttactatattaaatgtacattatgtgattagtgttagaaatgttcgtttc |
| | gagttttagcatcatctttatgaagattatttatgaaacatcaagttagggtctggctgtcagtttaaacatgagcacagattgagcataggcgataatcatggttggtaggaggccaggga |
| | ccgggatccttgtgggacctgccggcgtacccgtgatagcagaaggggtgcaagggacagggcactggaactaaggcaggcagaaacagagaaataaagattggggattagactttgtagcc |
| | aattcataaaaaaattggttgcatcattttgtagaggttggccttaacatatgactaaaaaacaaaaacaaaccaagactcttccctaaactgaataccgcagatactcaagacaccatata |
| | gggccacgggctcaggctgaagtcatctaggtctcagacatgccaaaacagactgggacaaggacaagctgaaactgactagtactaggtttcccgggtcaactaccgttcagctggaggtc |
| | tgaatgcgtggtgccttctctgatacatggatctcatgtggcacttctgttgtgtatttgtcccagtctgcttattttcctttgtctgtatcatgctgagactaatacatttgggtcgtgca |
| | tttgatcttcttctcattacaacttttagctctgatcctgatgacttgtttctagtctgcatcaatgcttatcatcatactattgtaggtattgggacacaagacagccgaatcctgcccat |
| | gttcagcaacttcctgatcgttgctacgcacttgctgtgaattatccccttatgattgtgggaacagctgatcgcaatattgtgatcttcaacttgcagaatcctcaggtaattttgcttcc |
| | taaccgactatgcattttcaattcttgtggtggtggtgctcatgcttttactgccattgacttatattttgaactgcctgacttaaattcatccctgatgtgattttagactgagtttaagc |
| | gtattcaatcacctctgaaataccagacacggtgcgttgctgcctttccagatcaacaaggattcctggtaatgccatattttcttgtgtcttttactctcagcattacgacaccaaccaaa |
| | acttccgttgtttgaatacataacttgttgttactatgctttgaagtttaatcatatgttcttctctacctatccatcataattatttatgttgcccaattatgttgtttttacctagcttg |
| | agttttcgaccttaaacattgttacaaccttactcgaattagctttagatgttggcctgccatcatttgcagtgtaatgggcgttgttcctttagtgatctcatttcaaacttcctacccct |
| | ctattataggataaataaggtatattgattgtgaaatcaaactttactgactataaatactatactatgtgatttatgtgacataaagttgatatcattggatttgttttcaaaagtacttt |
| | gttacgattttgataaaatatgagaagaaatttatggccaaagattgaactcgagcagtcaaaatatgccttgtatatctggatagagtgtgtattactaggaatgttaatccaatataaat |
| | gtcattaattgacatctctctagttagcacaaaagctgcataaaagaagaggatgcagttttccttaatgttttgaccaactccagctgtgttgcaaacagtttccttcaccagaactgtag |
| | aacggatcttagcctatagcagttaaagctaatctgaacatgaaatccggctgaagtacaaatagtaaaaccatccaaggcttcaagcaattgtttttgaactacagatctttagatgtcta |
| | tttctgcattttcctattcttcacctgcttgtgggtgttcttatagtgggtcgtcgtttcatcatgggctttgaccaatcaatttttgctgttacacaatgatctttttctacatcaatgaa |
| | ctgcctatattggataaagctggtcgatacctggcaatagttcttgttttaattattttcttgttgtttccatattacttgggcaaaacattttttacatgcgactgacacctgattttgtt |
| | tgaaggtgggttccatagaaggaagagttggtgtgcatcatattgatgattcacagcaaagcaaaaacttcacattcaagtgtcacagggaaggaaatgatattttctctgtcaattcgctc |
| | aactttcaccctgtaagatctctatcttgttctctcttgactccatatttatctatgtgccatactgccattcttatatttcattagtccacttctgctattatggaaaatgctaatggcga |
| | cttctgttgagctttagtcactatgtctccatcatgatttgtaaaatactgaaagctcagttcgttgtcttccacaagatgtgttgagctttattcgatacagatacctaccacacttcgcc |
| | taatataataaccactatgcttgaaggcatacatatgttagtaaactactccaaattgatcaagcaatattttgcagaaataaaaatcccaacagattagcttcagtttaattgagcaactc |
| | gaaatcgtacctatttgttacagacacatcgaagagaagcgtttctgcttcttctgttgctaactttggcatcttattcattggattggtatatgcttgggtgatgatactatacatccatt |
| | tagttgagtgtgcttgttggctatatatttcactggaaaatgcaactgactggtgataattgtgaagtatggaaattcagttagtctagttacttatgggatttcatgttatctaccacttg |
| | gcacaggttcatcacacgtttgccacagctggatctgatggtgctttcaacttttgggataaagatagcaagcagagacttaaggtttgtctattccccttttttttttctattttcccctc |
| | ttgttggtttatgttttcttgattggaattcaatgatttcaagatggctctactttgttgtgatctgtggacactgatgatctgcatataaattctttgttgatgcgcatacatagcctgaa |
| | atcatcccgagctcaagctaataaactcactatcctggccatgagcagaaaaaaaaatattagttcataaatgttatcaccaattctgattttcccagctgccttgtcatcctgctgctttt |
| | ggcgtttagtatgcacttctttttattcttgataaaagcatattacatatttattactcaaccgcatggagcctctgcagtaaggtatatgtgtaatttgtcaacagataaccttttagctt |
| | ttaggcatatcatacacagtgttgcattctgacagttgcttacttgcttagcatagagggctatctgtgtgtgcccaagtttattgcttcagttccctcatttgtcatacaccttctggtat |
| | gttgcacataacacattcttacatttcccctacactgcttctatacaggctttcagtcggtgtcctcaacccattccttgcagtagcttcaataatgatggttcaatatttgcttatggggt |
| | aagtcaagtgatgcttttgagagtgtcttgttatcaccatgtttttcagtccggtcagagtttagatatgttacatgcctgtcatttttcctgcaccttatttaatatacggaagaaatcag |
| | ctcgacaaataattcatgttccttttgaactgctatcttgtcgtgtcctctgaatccaagttctgaatgcaaccgtaattatcattgctggggagcaggtgtgctatgactggagccgtggc |
| | gctgagaaccataatcctgcaaatgcaaagacatccatctatctccacagtccccaggtagtttccgagtgctgatatcgccataaaaagaattcttgtattttcgacatcatttgagatcc |
| | attcctgtttttataaattgttattatgttatcttttggcactcaacttgcatcattctggacaggaagccgaggtgaaagggaagccaagaatcgcaacggggcggaagtgatggtggcat |
| | cctgcctgccgaaaagtcgatagttgtgcaagagatgatgtatattcaagggccttgtttcgctcgggccttgccttgacgtgtcctctggatgttcttacttggttcggccgcctggtaga |
| | tgaaacgcctactcccagcaaccagtgataggttacttagccgtaggtcgtagcacatagccgtttatgtaatgcatagttgtccgaaggaatgtttccccgtgttcttgtgttgtgatata |
| | gtcaggtcagcgcgtagtcttttgaaaaggagaaagccttcggtgtcttgtaagtggtgtagatctttggaaatatgggcatgatattcggtggagttattctgaaaaagatatcccggttt |
| | cggtttcagcgaatgcaagctagatttatggggaaggttgtagttagcaccatgctgcaaatgcatgcggcattcggaggatttacagtttgaatcttcctgtttatccaagtctaggattc |
| | ccaccttcctagtaccattgagcacgttagtttgaattttttttgtttattatggttcagaacttccgattcaaaaaaatctgatttattttacgcgaatatatggatgtttaatcaatata |
| | cggttttgtatgtgagatataaagaagggccaaagacatgcaaacaatgggcatttctttgtgctggatcttttgtgttcttatctgcactgcaccatcaaacgaaaattaccaggtgcgtg |
| | cagaacgacgtgtaaagaaaacacccaacttttccgaaacttccaaatgccattttcgagctgttcaacatagtgtgcttcgaaaatccgttcttcagtacagtaccagccacgtagcaatg |
| | gctccacagaagcctcattgcttacgagctttatgccgtgaacttgcatagattcagttcgacaacccaattgcataagcttgcgatctttttatgccgttaacttcaaaatagattcggcg |
| | caacacgactcaatagccacgacacgggcaaagactagggcatcatcaaacctaacgcaatatagcattggtgtcggtacttaatttacagggttttattgcatgaccaaatcacaggtgcg |
| | gcataaaattacagtcgcggagagaaagcgaacccagtttcacagcaagagagtagcagactagttaacaaacgtaagagaacccagcttcaagaatctaaacacaggtaaagaccacattg |
| | tttcagcaccaagttcccgtccttccttccacgtgagaacccgacccggtcctagtcacagctccttcagacatcgaacacatcatcatacatgtcggcgtcccagtccgcgtcgtcgatat |
| | aatcctccatgtcatcaaactcggcatcctcgtcgtcgtcggtgaccacgtccagcagatcatcgtacatgtcgacctcgatgtcggaaccgccggggtagtcatcactgccagcgaggtaa |
| | gcccggtacttgaagtcggagatggggtccccggggagcctgaggttcccgatcctggcgcacttggatctcagggcatcgtccatctggaggttgcagcagcggcgcacgtcgagggactc |
| | gaggcgggggcagtggtcgaggatcgccgccagcccggcgttggtcaggttgttgccgatcagctggaggtcccggagctcgggcatgctgctcgcgatccctagcacacccgtgtcatcat |
| | ccatgccctcaaagactgcaaaccccctttggaggaccgtccagcgctcgttcagccgaaaggatttcaggtgtgggcaggctctgccgactgactcgcacacgttgctgttcagtgagcag |
| | aatgtgatctccagctcctcaagctgaggaaagcccttgactgcatctgccagcgcctcattggatacttcatcacacaggcttagttgaaggctcttcagcgagggtgccctgcattgaga |
| | catggcaagttcttatctaacacaagcatttaagctaatgccaaacccaatcatgctttccaagctcaggacaagaaataacactagaacaacagaattgattgaaataaccatgtgaaagt |
| | aaaacattccatgctgttagattcaccatagtcttcaaataagtctaactccagtcacatgcaaatgaacttcagcacgcagttctaaacaaaatacgacatgacttgatactgtgtctgaa |
| | ctccacatatcacatctagtcaaaccattagaagaagacgacaacgagccaggtgttaggtggtcaagaagacaagaagtgggatcaatttatttcaatgcaccaaaagatcaacacaacga |
| | taagaaaatagcaactctgactagcactacgcttaccaagcaaacaaatggccacaagaatatggagttatgggcagcttaggggttttgcttcagtgagggggatataacagttgcaatac |
| | cataaaagaaagtagctagtaatagttttacatatttatcgtttcactgcatagttgttgatttgattgttaatactatgcgatacagatatacaccagtggtaattggatgcacattcagt |
| | aaggcccttaatttttttgtatgcaaggaaatgttgtagtggtggctccaacagtataatccataagaaccaattcgagtaaatgatgatttgaacccaggtagtgggcccacccaacatgt |
| | aagttaactacatgaagagtgcaggcacactaaaacattcatcctgaagtacatgagttattttggaaacattctgatggattgttggataaaagcgtgaaagtttccaaggttgcaagttt |
| | ctgtatgatctaaatgcacgagaggactgtgtacaagtgaaaggtactggaagatgcatgaaccaaaaacagttacaggaaggttaacagagaagtctgcagctaccaaaggcaggattgtc |
| | agaaatgccgaattgcatcatgcatcaatcttgcagtgaaactgcctacatctttatcagaaaaaaatggtactgaaccatcgaagtgatcatttggttttctacagacctttacagcaagt |
| | aatttgctggaaaccaacaaaaaagttcagatggccctcccccctgtaaagtctccatgctgagactcacctatccttgtatgttttagggattggtcggtgccaatgttatatccaagagc |
| | acaatgatgcccccattctgactattagtgggaacaggccaattccctccaggaaaacaaaccttcagtcagtgtgctactctatatacattttccgggttgctccacatacattatgtccc |
| | aacgctacggagtagatagaacttagcaaggcacattgagtcacacaaagacatagcactggagactcatgtgatctgttccaacttgcaagcattttactgttgagtagtaccattttacc |
| | ccatccaatttcgttaggttatgaaaatgtgtgtttttttcaacctgtcaaatcaaacgtcagaaaggtccatttccaacttcagaatacagacttattttaagttaaagaccttccccatg |
| | aattttaatttaccagcagcgccagcaaccgttcgtttaactgaatgaataaactaagcgccgcatccacaaattgggggaaaaaaaaccccccactgtatgccagaaatgggcatgtgaaa |
| | caggaaggggagagccgtcgcgaacaacctgccggagatgtagctgaggagggcgtcggtgacgaaggtgtcggcgcagaaggactggagcgtgccggcggcgcggtcgacggcggcgcggg |
| | ccatggcctcggcctcctcggtctcgaggatgtccccgtggtgggtcatgtcgaggcggcgccacagcgcggggtcgccgtcggcgaggcggcgccaggcgcggcacgcccgcccggccccc |
| | gtcaggatg |
| SEQ ID NO: 10_ | ttgtcccgccgatgataagctcggttggcgactggtccatgcccctccggactccacacagccaggcgaattggtgagtctgcaactgctgagctggacacaagtaagtcataaatgcgacg |
| TaPK-like | caacaaaaaatgacgccacgaaagttgggacgttttctcaattatcccggaatattataatagcatgttgtacgcgtacctcatgtagccggaaaacatctacagccgggcaccccaaactc |
| promoter region | tatcatacgccccgacggacggccaagttagtgaccggtcacgaaagactgacctagacgggtaacttaaacgggtctcaagcgctcaggctaaccggcgcccctcatatccagtcccaaat |
| | gtagggcggataagggggcgcccaggagcatcgaggcgcgtccatcacgtcggtcctggcccacgatggcccacccgcctccatatatattcttctgcatccgctgcctgggccaaacacta |
| | tccacttcactcccctacgccacccaaactcctatccgacgatcacgagccttctccggtttgacgtgtagcagattcgacgacgaccagtacagatctgtcgactggggtgtcaacccgtg |
| | cggtttggagaaggaaatggccatccgcattgcactttgccatttccggaggacaacgcccttccgacgacaggatctcccagcgcgagtccattgcgtcggtgcatcgggcgctcagatcc |
| | tctggtgctggatcatcgtggtcctctaggcagccttccaatctcccttccccatcactggtccgtcggagtacgagtcctatcgagggtcagtgtgcccgccgtgggaagaagaggatgag |
| | ggcggctgaggtcagcgccttgctgccgacatggcggcggcggaggcggctgatacaatggcacgggcggccgcagaggaggaaggcatccgcgtccgcattgtaaagaagcggcagcggag |
| | gaacacgcgcgtccctcgtccgagagcataatcggcgatccgtgccattgccggactgccttccaaggaggagaaggaggacaacgatgggtcagatagctctggcgatgagcagacttgac |
| | tcgattcgtactgcctctttgatcggtacttgcgcgagaagaacgacaagggcaccaagaagggcaaagacagacatggattgatcttcttcatagctagcatgtacgtaggttaaacatgc |
| | ccaatttttgatagtccgatgtcatgtcctgatgtcgtagtaggaagatgtgtgtaatgcatcgtttatgtagttgcatgagtttgtatggatttgagataaggtaattaagatgttcgatt |
| | gtgagaagggaaatttaaggcgtgaccaacccgtgttcacggatgcgcctggacgcatcgctgaccggatcgtgtcgacgcgcgcgagcaaaggcggccggcgaggccaaccccaaccacca |
| | ctccgcccgcgccgcccctccctttctcccgccttttcggcccccatcaatggctccctccacctccttctagaagccgcctgccccaccacccgtgctagcggtagtgtatcgggttcccc |
| | ttccttccttcctctcccctccctgcctgcactgtcccatcgctcgctttctcgcccgctccgtggcggtggtggtggtgcgtggtgggggagaaagttggaggcgggggaaaggagacgca |
| | aagcggccacccaaaaaggcacatatactttctttctttccaccgccgccctcctcctcctggtcctggaggagttccgatcccccccccccccccccctctctcttctttctctctctttc |
| | ttgctctgctctggaagcaggggagggagggggttccgatttgggcggggaggcactgttcgcggtgactcggttcggctgatttgggaggtcttctcgtttgatccgggctcgctgattga |
| | ttgattggttggttggttcgtctccggcggctcgatttgagggtcggtcggtccggcgagtgagtcgccatg |
| SEQ ID NO: 11_ | gacggctcaatggtgtatttttcccgtcgtgcgtcttcatcggtgaggttatggtatgtacaattttggcgcacaatgacggcgacttccctgatccttcgtcatcgtctccaccgtgttga |
| TaRAE1-like | ttttggtgctggcgtgaggcctgaggtatgtctcttggatctaatgctcgtctagcgttggtggtttctccttcagagcgatggcccggtaagacgtggccttaatcactccccgcccgtga |
| promoter region | tgaacatcaggctggcttcgatgatggagaagcatgtgttggctcggcatgttctagaggatgaagaaggatgacccagggattttgatgtaactttattaatattttcttcctataatttg |
| | tacttccttttcttagaaaaaaactattttgtactgctagtttgattaatactcgtggcttcacatgcgctggtttggttatataagagggagcatatgggtgttgtctttttatataagaa |
| | aaaaaagagcaaaataagccacgcgccgatcaagcctgcgaactgagtgtatacccggccatgggcaacccagactgacccgacccatcccgaaaaagctcgacccgtcccgactcgacctt |
| | gcccacgggccgggcccaggcctagattttgagcccgatggctgggccgacatgttgtatttgcgatttaaggaagaggcccggcccaagggccgaggcccgacgggttttttgactgatgg |
| | gccggggttgggccagattattaggcccaacggccgggccgggccgggctcgggcctaagccttttgcttgcgttcaggctttgataggctcagcccgaagtccacatccacatcaaccacc |
| | cccgcgtgccgatgaagccatttctttggaaggacctgcgcgtgcatccgacgcgacgcgctacaccgaagcctatactactacatctcgtctcatcgccctcctcccagacgacgcctcga |
| | gaatcctcggctgaggctccactccgcactcgcaagtcgcacccgcagccgcgccgtccccagcagccgccgccgaccgctccccgtccggcaggtaagccccccgagcccctcctcgccgc |
| | tccgctcgcgcggactcctagctagctagggttcccggcaccgattccgcggcgctgcactccggcgcaatgctcgcggccgcggacgggcccgccgcttcgggggaagctctgatctggtg |
| | gccggcgctcgctcgctcgctcgcggtctccggagcaaatcggccgcatcgagtggccggagcaagcttaggatagttcgattatttcccccatccccgttggtcaggccgagagggccagt |
| | ttgggcgacctcctggtgattccgctgccgtggtggggattgggtggtcgtggcccgggtgtaggcgtttgcaggtggagtgagtgtttccttccaggttctatgctcacgcagctcgtgtt |
| | attgctgggatccgggcgttcggcaggctaacattcaccggattttggacctgcattgttgcggcgcgtgccaggggatggcgtttatgctggctttggcgtggtgattttgtggagttgag |
| | tggtcaggttgtaggcatttgtagttgagtgatcccagctttcatgcccaccagcttgtattattactcgagttggtcgttaaccgtgtttaccgaattttggaccgccgttgttgtggcac |
| | gtaccaggggacagcgtttatgctggtttgcgaggctacctcctggtgatttcactctcgggtggtgggaatcgagtggtcggggtgtaggcatatttgtagtggagcgatcccaggtttta |
| | tgaccagacatttagccgaaatttggacctacactattgtggcacgtaccaggggatagcatttatgcaggttttggcgcgacacttcttttgtcgggattttgtggtgcaaacttaaataa |
| | tgacttccctaatgttgaacaatggttctggtatcgtggactgccgaaaccgttctttttggtagtctgagctactttaagttgttacttatgctctcgtgtgcgtgggagactccacgtgc |
| | tggtttctcttacttgcttctcatggtcatcactggactgtgatatttcttttccagattttgaaggttcacagaaggagagatg |
| SEQ ID NO: 12_ | ttgtcccgccgatgataagctcggttggcgactggtccatgcccctccggactccacacagccaggcgaattggtgagtctgcaactgctgagctggacacaagtaagtcataaatgcgacg |
| pTaPK- | caacaaaaaatgacgccacgaaagttgggacgttttctcaattatcccggaatattataatagcatgttgtacgcgtacctcatgtagccggaaaacatctacagccgggcaccccaaactc |
| like: RFL29a | tatcatacgccccgacggacggccaagttagtgaccggtcacgaaagactgacctagacgggtaacttaaacgggtctcaagcgctcaggctaaccggcgcccctcatatccagtcccaaat |
| chimeric gene | gtagggcggataagggggcgcccaggagcatcgaggcgcgtccatcacgtcggtcctggcccacgatggcccacccgcctccatatatattcttctgcatccgctgcctgggccaaacacta |
| cassette | tccacttcactcccctacgccacccaaactcctatccgacgatcacgagccttctccggtttgacgtgtagcagattcgacgacgaccagtacagatctgtcgactggggtgtcaacccgtg |
| | cggtttggagaaggaaatggccatccgcattgcactttgccatttccggaggacaacgcccttccgacgacaggatctcccagcgcgagtccattgcgtcggtgcatcgggcgctcagatcc |
| | tctggtgctggatcatcgtggtcctctaggcagccttccaatctcccttccccatcactggtccgtcggagtacgagtcctatcgagggtcagtgtgcccgccgtgggaagaagaggatgag |
| | ggcggctgaggtcagcgccttgctgccgacatggcggcggcggaggcggctgatacaatggcacgggcggccgcagaggaggaaggcatccgcgtccgcattgtaaagaagcggcagcggag |
| | gaacacgcgcgtccctcgtccgagagcataatcggcgatccgtgccattgccggactgccttccaaggaggagaaggaggacaacgatgggtcagatagctctggcgatgagcagacttgac |
| | tcgattcgtactgcctctttgatcggtacttgcgcgagaagaacgacaagggcaccaagaagggcaaagacagacatggattgatcttcttcatagctagcatgtacgtaggttaaacatgc |
| | caatttttgatagtccgatgtcatgtcctgatgtcgtagtaggaagatgtgtgtaatgcatcgtttatgtagttgcatgagtttgtatggatttgagataaggtaattaagatgttcgattg |
| | tgagaagggaaatttaaggcgtgaccaacccgtgttcacggatgcgcctggacgcatccgctgaccggatcgtgtcgacgcgcgcgagcaaaggcggccggcgaggccaaccccaaccacca |
| | ctccgcccgcgccgcccctccctttctcccgccttttcggcccccatcaatggctccctccacctccttctagaagccgcctgccccaccacccgtgctagcggtagtgtatcgggttcccc |
| | ttccttccttcctctcccctccctgcctgcactgtcccatcgctcgctttctcgcccgctccgtggcggtggtggtggtgcgtggtgggggagaaagttggaggcgggggaaaggagacgca |
| | aagcggccacccaaaaaggcacatatactttctttctttccaccgccgccctcctcctcctggtcctggaggagttccgatcccccccccccccccccctctctcttctttctctctctttc |
| | ttgctctgctctggaagcaggggagggagggggttccgatttgggcggggaggcactgttcgcggtgactcggttcggctgatttgggaggtcttctcgtttgatccgggctcgctgattga |
| | ttgattggttggttggttcgtctccggcggctcgatttgagggtcggtcggtccggcgagtgagtcgccatgccccgcttctcctccaccacgccaatgtcgccaccccgcctccgcctccg |
| | actctgcgcccgccactcctcctccacctctcatccctcacgcatctgggatccccacgccgccttcgccgccgcggcacagcgggcgagctctggcacgctcactacggaggacgcacacc |
| | acctgtttgacgaattgctgcggcggggcaatcctgtccaggagcgtcccttgaataaatttctggctgccctcgcccgcgcgcccgcgtccgcatcctgctgcgatggccccgccctggca |
| | gtcgccctcttcggccgtttgtcccgagacgtcggacgacgggtggcgcagccaaatgtcttcacctatggcgtcctcatggactgctgctgccgcgcttgccgcacagatctggtgctcgc |
| | cttctttggccgtctcctcaagacgggcctggaggcaaaccaagtcgtcttcaacaccctcctcaagggcctttgccacacaaagcgggcggatgaggctctggacgtgctgcttcacagga |
| | tgcctgagctgggctgcactcctaatgtggtggcgtataacaccgttatccatggcttctttaaggaaggccatgtaagcaaggcctgcaatctgttccatgaaatggcgcagcagggcgtt |
| | aagcctaatgtggtgacatataactcagttatcgatgcgctgtgcaaggccagagccatggacaaggcagaggtggtccttcgtcagatgattgatgatggtgttggacctgataatgtgac |
| | gtatagtagcctcatccatggatattcctcttcaggccactggaaggaggcagttagggtattcaaagagatgacaagtcggagggttacagcagatgtgcatacttacaacatgtttatga |
| | cctttctttgcaaacatggaagaagcaaagaagctgcaggaatttttgataccatggctatcaagggcctgaaacctgacaacgtttcatatgctattctccttcatgggtatgccgccgaa |
| | ggatgcttagttgatatgattaatctcttcaattcaatggaaagagattgtattctacctgactgtcgtatcttcaacatactgattaatgcatatgctaaatctgggaagcttgataaggc |
| | tatgcttatcttcaatgaaatgcagaaacaaggagtgagtccaaatgcagtcacatattcaaccgtaatacatgcattttgcaagaagggtaggttggatgatgctgtgataaagtttaatc |
| | agatgattgatacaggagtacgaccggacgcatctgtttatcgtcccctaatccagggtttttgtacacatggcgatttggtgaaagcaaaggaatatgttactgaaatgatgaagaaaggt |
| | atgcctcctcctgatattatgttcttcagttcaatcatgcagaacctatgcacagaaggaagggtaacagaagcacgggatatccttgacttgatagtgcacattggtatgaggcctaatgt |
| | tatcatatttaatttgctgatcggtggatactgcctagtccgcaagatggcagatgcattgaaagtatttgatgatatggtgtcatatggtttagaaccttgtaactttacgtatggtatac |
| | ttattaatggctattgcaaaaatagaaggattgatgacgggcttattctgttcaaagagatgctgcacaagggacttaaacctacaacttttaattataacgtcatactggatggattattt |
| | ctggctggacaaactgttgctgcaaaagagaagtttgatgagatggttgaatctggagtaagtgtgtgcattgatacatactctataattcttggtggactttgtagaaatagctgcagtag |
| | cgaagcgatcacccttttccggaaattaagcgcaatgaatgtgaaatttgatattacaattgtcaatatcattattggtgccttatacagggtcgagagaaaccaagaggctaaggatttgt |
| | ttgctgctatgccagccaatggcttggttcctaatgctgttacctacaccgtaatgatgacaaatcttataaaagaaggttcagtggaagaagctgacaatcttttcttatccatggagaag |
| | agcggctgtactgccaactcttgcctgttaaatcatatcatcagaaggttactggaaaaaggagagatagtcaaggctggaaattatatgtctaaagttgatgcaaagagctactcacttga |
| | agctaaaactgtttcgctgctgatctctctgttttcaaggaaagggaaatatagagaacacatcaaattgcttcctacaaagtatcagtttctggaagaagcagccacagttgaatagttgg |
| | tacatgatatctgaaatttaatttgcatcgcttgccatgggtccgtctgcttgtacaagaaatgcattttctatttgtaaataggaagtcagtttagaacaagccatcaggatgacgcaaag |
| | agtacaattcagttgcaccaccaataaaaaggcagaactagggctgccaaaacaacactgaatcaaaactcaaacagaaggagcagcaaacttttttttttttgaagctggacagtctgcta |
| | gccaaacaactacaggagactgtcaggggggcatgtagtggctggcgtctaagcgcctttgcttcttccaccatccatggcttagcctcacacggaatcgagtcaaccaattcccgtcggtt |
| | ttgggggctcccttgaagatgcaattgttttcagcggccagatacgcatggtcagattaatcagcgagtgtgccccttctgtctggttcgagaaagaatttgaagagctgagcttgtccctg |
| | ctggaaccagtttgaggttagttaatcataacagggtactagagaggtgttttattgactgttgatgtgtaatatgttatatgccatcctcttgatgattacggtgatctgtgaagaggcag |
| | catgcaaaagtctgaatcacatatgcttatgtaattgtgttattatttgtgcagcttctagaccttttgccttgtagatggctacatggatctagttgtagcaatctgtaactgttagtgtt |
| | ttgtatatgctggcattgatgattagggtgacttgtgaagcatgcagaagtctgaatcgtacatgcttgtctag |
| SEQ ID NO: 13_ | gtggccctgctcatccctggatgttgacggctcaatggtgtatttttcccgtcgtgcgtcttcatcggtgaggttatggtatgtacaattttggcgcacaatgacggcgacttccctgatcc |
| pTaRAE1- | ttcgtcatcgtctccaccgtgttgattttggtgctggcgtgaggcctgaggtatgtctcttggatctaatgctcgtctagcgttggtggtttctccttcagagcgatggcccggtaagacgt |
| like::RFL29a | ggccttaatcactccccgcccgtgatgaacatcaggctggcttcgatgatggagaagcatgtgttggctcggcatgttctagaggatgaagaaggatgacccagggattttgatgtaacttt |
| chimeric gene | attaatattttcttcctataatttgtacttccttttcttagaaaaaaactattttgtactgctagtttgattaatactcgtggcttcacatgcgctggtttggttatataagagggagcata |
| cassette | tgggtgttgtctttttatataagaaaaaaaagagcaaaataagccacgcgccgatcaagcctgcgaactgagtgtatacccggccatgggcaacccagactgacccgacccatcccgaaaaa |
| | gctcgacccgtcccgactcgaccttgcccacgggccgggcccaggcctagattttgagcccgatggctgggccgacatgttgtatttgcgatttaaggaagaggcccggcccaagggccgag |
| | gcccgacgggttttttgactgatgggccggggttgggccagattattaggcccaacggccgggccgggccgggctcgggcctaagccttttgcttgcgttcaggctttgataggctcagccc |
| | gaagtccacatccacatcaaccacccccgcgtgccgatgaagccatttctttggaaggacctgcgcgtgcatccgacgcgacgcgctacaccgaagcctatactactacatctcgtctcatc |
| | gccctcctcccagacgacgcctcgagaatcctcggctgaggctccactccgcactcgcaagtcgcacccgcagccgcgccgtccccagcagccgccgccgaccgctccccgtccggcaggta |
| | agccccccgagcccctcctcgccgctccgctcgcgcggactcctagctagctagggttcccggcaccgattccgcggcgctgcactccggcgcaatgctcgcggccgcggacgggcccgccg |
| | cttcgggggaagctctgatctggtggccggcgctcgctcgctcgctcgcggtctccggagcaaatcggccgcatcgagtggccggagcaagcttaggatagttcgattatttcccccatccc |
| | cgttggtcaggccgagagggccagtttgggcgacctcctggtgattccgctgccgtggtggggattgggtggtcgtggcccgggtgtaggcgtttgcaggtggagtgagtgtttccttccag |
| | gttctatgctcacgcagctcgtgttattgctgggatccgggcgttcggcaggctaacattcaccggattttggacctgcattgttgcggcgcgtgccaggggatggcgtttatgctggcttt |
| | ggcgtggtgattttgtggagttgagtggtcaggttgtaggcatttgtagttgagtgatcccagctttcatgcccaccagcttgtattattactcgagttggtcgttaaccgtgtttaccgaa |
| | ttttggaccgccgttgttgtggcacgtaccaggggacagcgtttatgctggtttgcgaggctacctcctggtgatttcactctcggggggggaatcgagtggtcggggtgtaggcatatttg |
| | tagtggagcgatcccaggttttatgaccagacatttagccgaaatttggacctacactattgtggcacgtaccaggggatagcatttatgcaggttttggcgcgacacttcttttgtcggga |
| | ttttgtggtgcaaacttaaataatgacttccctaatgttgaacaatggttctggtatcgtggactgccgaaaccgttctttttggtagtctgagctactttaagttgttacttatgctctcg |
| | tgtgcgtgggagactccacgtgctggtttctcttacttgcttctcatggtcatcactggactgtgatatttcttttccagattttgaaggttcacagaaggagagatgccccgcttctcctc |
| | caccacgccaatgtcgccaccccgcctccgcctccgactctgcgcccgccactcctcctccacctctcatccctcacgcatctgggatccccacgccgccttcgccgccgcggcacagcggg |
| | cgagctctggcacgctcactacggaggacgcacaccacctgtttgacgaattgctgcggcggggcaatcctgtccaggagcgtcccttgaataaatttctggctgccctcgcccgcgcgccc |
| | gcgtccgcatcctgctgcgatggccccgccctggcagtcgccctcttcggccgtttgtcccgagacgtcggacgacgggggcgcagccaaatgtcttcacctatggcgtcctcatggactgc |
| | tgctgccgcgcttgccgcacagatctggtgctcgccttctttggccgtctcctcaagacgggcctggaggcaaaccaagtcgtcttcaacaccctcctcaagggcctttgccacacaaagcg |
| | ggcggatgaggctctggacgtgctgcttcacaggatgcctgagctgggctgcactcctaatgtggtggcgtataacaccgttatccatggcttctttaaggaaggccatgtaagcaaggcct |
| | gcaatctgttccatgaaatggcgcagcagggcgttaagcctaatgtggtgacatataactcagttatcgatgcgctgtgcaaggccagagccatggacaaggcagaggtggtccttcgtcag |
| | atgattgatgatggtgttggacctgataatgtgacgtatagtagcctcatccatggatattcctcttcaggccactggaaggaggcagttagggtattcaaagagatgacaagtcggagggt |
| | tacagcagatgtgcatacttacaacatgtttatgacctttctttgcaaacatggaagaagcaaagaagctgcaggaatttttgataccatggctatcaagggcctgaaacctgacaacgttt |
| | catatgctattctccttcatgggtatgccgccgaaggatgcttagttgatatgattaatctcttcaattcaatggaaagagattgtattctacctgactgtcgtatcttcaacatactgatt |
| | aatgcatatgctaaatctgggaagcttgataaggctatgcttatcttcaatgaaatgcagaaacaaggagtgagtccaaatgcagtcacatattcaaccgtaatacatgcattttgcaagaa |
| | gggtaggttggatgatgctgtgataaagtttaatcagatgattgatacaggagtacgaccggacgcatctgtttatcgtcccctaatccagggtttttgtacacatggcgatttggtgaaag |
| | caaaggaatatgttactgaaatgatgaagaaaggtatgcctcctcctgatattatgttcttcagttcaatcatgcagaacctatgcacagaaggaagggtaacagaagcacgggatatcctt |
| | gacttgatagtgcacattggtatgaggcctaatgttatcatatttaatttgctgatcggtggatactgcctagtccgcaagatggcagatgcattgaaagtatttgatgatatggtgtcata |
| | tggtttagaaccttgtaactttacgtatggtatacttattaatggctattgcaaaaatagaaggattgatgacgggcttattctgttcaaagagatgctgcacaagggacttaaacctacaa |
| | cttttaattataacgtcatactggatggattatttctggctggacaaactgttgctgcaaaagagaagtttgatgagatggttgaatctggagtaagtgtgtgcattgatacatactctata |
| | attcttggtggactttgtagaaatagctgcagtagcgaagcgatcacccttttccggaaattaagcgcaatgaatgtgaaatttgatattacaattgtcaatatcattattggtgccttata |
| | cagggtcgagagaaaccaagaggctaaggatttgtttgctgctatgccagccaatggcttggttcctaatgctgttacctacaccgtaatgatgacaaatcttataaaagaaggttcagtgg |
| | aagaagctgacaatcttttcttatccatggagaagagcggctgtactgccaactcttgcctgttaaatcatatcatcagaaggttactggaaaaaggagagatagtcaaggctggaaattat |
| | atgtctaaagttgatgcaaagagctactcacttgaagctaaaactgtttcgctgctgatctctctgttttcaaggaaagggaaatatagagaacacatcaaattgcttcctacaaagtatca |
| | gtttctggaagaagcagccacagttgaatagttggtacatgatatctgaaatttaatttgcatcgcttgccatgggtccgtctgcttgtacaagaaatgcattttctatttgtaaataggaa |
| | gtcagtttagaacaagccatcaggatgacgcaaagagtacaattcagttgcaccaccaataaaaaggcagaactagggctgccaaaacaacactgaatcaaaactcaaacagaaggagcagc |
| | aaacttttttttttttgaagctggacagtctgctagccaaacaactacaggagactgtcaggggggcatgtagtggctggcgtctaagcgcctttgcttcttccaccatccatggcttagcc |
| | tcacacggaatcgagtcaaccaattcccgtcggttttgggtggctcccttgaagatgcaattgttttcagcggccagatacgcatggtcagattaatcagcgagtgtgccccttctgtctgg |
| | ttcgagaaagaatttgaagagctgagcttgtccctgctggaaccagtttgaggttagttaatcataacagggtactagagaggtgttttattgactgttgatgtgtaatatgttatatgcca |
| | tcctcttgatgattacggtgatctgtgaagaggcagcatgcaaaagtctgaatcacatatgcttatgtaattgtgttattatttgtgcagcttctagaccttttgccttgtagatggctaca |
| | tggatctagttgtagcaatctgtaactgttagtgttttgtatatgctggcattgatgattagggtgacttgtgaagcatgcagaagtctgaatcgtacatgcttgtctag |
| SEQ ID NO: 14_ | atgccccgcttctcctccaccacgccaatgtcgccaccccgcctccgcctccgactctgcgcccgccactcctcctccacctctcatccctcacgcatctgggatccccacgccgccttcgc |
| RFL29a CDS | cgccgcggcacagcgggcgagctctggcacgctcactacggaggacgcacaccacctgtttgacgaattgctgcggcggggcaatcctgtccaggagcgtcccttgaataaatttctggctg |
| | ccctcgcccgcgcgcccgcgtccgcatcctgctgcgatggccccgccctggcagtcgccctcttcggccgtttgtcccgagacgtcggacgacgggggcgcagccaaatgtcttcacctatg |
| | gcgtcctcatggactgctgctgccgcgcttgccgcacagatctggtgctcgccttctttggccgtctcctcaagacgggcctggaggcaaaccaagtcgtcttcaacaccctcctcaagggc |
| | ctttgccacacaaagcgggcggatgaggctctggacgtgctgcttcacaggatgcctgagctgggctgcactcctaatgtggtggcgtataacaccgttatccatggcttctttaaggaagg |
| | ccatgtaagcaaggcctgcaatctgttccatgaaatggcgcagcagggcgttaagcctaatgtggtgacatataactcagttatcgatgcgctgtgcaaggccagagccatggacaaggcag |
| | aggtggtccttcgtcagatgattgatgatggtgttggacctgataatgtgacgtatagtagcctcatccatggatattcctcttcaggccactggaaggaggcagttagggtattcaaagag |
| | atgacaagtcggagggttacagcagatgtgcatacttacaacatgtttatgacctttctttgcaaacatggaagaagcaaagaagctgcaggaatttttgataccatggctatcaagggcct |
| | gaaacctgacaacgtttcatatgctattctccttcatgggtatgccgccgaaggatgcttagttgatatgattaatctcttcaattcaatggaaagagattgtattctacctgactgtcgta |
| | tcttcaacatactgattaatgcatatgctaaatctgggaagcttgataaggctatgcttatcttcaatgaaatgcagaaacaaggagtgagtccaaatgcagtcacatattcaaccgtaata |
| | catgcattttgcaagaagggtaggttggatgatgctgtgataaagtttaatcagatgattgatacaggagtacgaccggacgcatctgtttatcgtcccctaatccagggtttttgtacaca |
| | tggcgatttggtgaaagcaaaggaatatgttactgaaatgatgaagaaaggtatgcctcctcctgatattatgttcttcagttcaatcatgcagaacctatgcacagaaggaagggtaacag |
| | aagcacgggatatccttgacttgatagtgcacattggtatgaggcctaatgttatcatatttaatttgctgatcggtggatactgcctagtccgcaagatggcagatgcattgaaagtattt |
| | gatgatatggtgtcatatggtttagaaccttgtaactttacgtatggtatacttattaatggctattgcaaaaatagaaggattgatgacgggcttattctgttcaaagagatgctgcacaa |
| | gggacttaaacctacaacttttaattataacgtcatactggatggattatttctggctggacaaactgttgctgcaaaagagaagtttgatgagatggttgaatctggagtaagtgtgtgca |
| | ttgatacatactctataattcttggtggactttgtagaaatagctgcagtagcgaagcgatcacccttttccggaaattaagcgcaatgaatgtgaaatttgatattacaattgtcaatatc |
| | attattggtgccttatacagggtcgagagaaaccaagaggctaaggatttgtttgctgctatgccagccaatggcttggttcctaatgctgttacctacaccgtaatgatgacaaatcttat |
| | aaaagaaggttcagtggaagaagctgacaatcttttcttatccatggagaagagcggctgtactgccaactcttgcctgttaaatcatatcatcagaaggttactggaaaaaggagagatag |
| | tcaaggctggaaattatatgtctaaagttgatgcaaagagctactcacttgaagctaaaactgtttcgctgctgatctctctgttttcaaggaaagggaaatatagagaacacatcaaattg |
| | cttcctacaaagtatcagtttctggaagaagcagccacagttgaatag |
| SEQ ID NO: 15_ | mprfssttpmspprlrlrlcarhssstshpsriwdphaafaaaaqrassgtlttedahhlfdellrrgnpvqerpinkflaalarapasasccdgpalavalfgrlsrdvgrrvaqpnvfty |
| RFL29a | gvlmdcccracrtdlvlaffgrilktgleanqvvfntllkglchtkradealdvllhrmpelgctpnvvayntvihgffkeghvskacnlfhemaqqgvkpnvvtynsvidalckaramdka |
| | evvlrqmiddgvgpdnvtysslihgysssghwkeavrvfkemtsrrvtadvhtynmfmtflckhgrskeaagifdtmaikglkpdnvsyaillhgyaaegclvdminlfnsmerdcilpdcr |
| | ifnilinayaksgkldkamlifnemqkqgvspnavtystvihafckkgrlddavikfnqmidtgvrpdasvyrpliqgfcthgdlvkakeyvtemmkkgmpppdimffssimqnlctegrvt |
| | eardildlivhigmrpnviifnlliggyclvrkmadalkvfddmvsyglepcnftygilingycknrriddglilfkemlhkglkpttfnynvildglflagqtvaakekfdemvesgvsvc |
| | idtysiilgglcrnscsseaitlfrklsamnvkfditivniiigalyrvernqeakdlfaampanglvpnavtytvmmtnlikegsveeadnlflsmeksgctanscllnhiirrllekgei |
| | vkagnymskvdaksysleaktvsllislfsrkgkyrehikllptkyqfleeaatve |
| SEQ ID NO: 16_ | tcggatccagctcgtctctcgtcttactcagggtcggtcatcccggtgaccttgcctctggcagcgagggggtgtgcgtgctctatggtgggccttgtgaggcgatggcgcggttgctgcgc |
| RFL29a genomic | aggggtgtgcatgccagcgattgcgcgggccagcttgtgtcggctgcgtgagggtgttggttgagtctgctccaagcgcaagcttgcccggctatggccggccggtgctggcggcgcctatg |
| | ggcgtcgtttcccttgttgaaggcgttgttgtggagttcttcgacctactacaatctcgaatctggttcttcgggcgaaaacctcgccccatctaggtcgggcaacgacgtcgtctgtacgg |
| | tgttttccccctttggggcgttgctttgaagaccggttatcctttcccgtgctacctttgggctggtcgtgcatggtgtcatggttggctggtgctcaaccggtgatgcgagtggttagcgt |
| | tgcgactcgtgcggtgacgaagatggtgatgcgcggagctgggtcgcggcttgtccttctgatatcggcggttcggttcccctttgggtgcgtctgtgcttgtttctcactgtgacagagaa |
| | gttgaagctacatgcggcgggggccctgcggcaatgatgactccatgagggggcttcggcggacgatgctctccgcaggttgcactggactagcttggtgtgaggcttgcaactttctcctg |
| | tgatgctcatcaacaaaatcggagatgtcggtcctcgacgcctgcggccgatcgtttggcttggctgtcgaggtcctcgggtgccgtttgttcggaggggtcttgatgggtcgtcagcgagg |
| | aaatcggagttgcctgctcgtggagaggcgatgacgatgacattgcttgcagcctcgacggtgtcctctcgtcagcagtatgtgtgtgatcttgtcagtgccaggtcggccggagtgctcta |
| | ttcgatgggcgtgttttaattaaccgggcaactctcttcttcttaatcaatgaaaatggcaagtcttttacctcgtttcaaaaagagttaaatgcactggaggtcctaaaggtttcgtcggg |
| | gtgtcactttagtcctatttcttccaaaatgcacctttaggtcctaattctttattaattggttcacccgaggtccttttccacgagcgctagctgaccagcacacgtgccgcattgaccca |
| | cgccacatcgatagagggtctaaccgccaggcgagaagcttcgcaaagagacccccccccctcttagtggcaccctctctcctctttcacggtactttggcgacattggaggcgagcgacag |
| | aggcaaccggaggtgatctggaggtaatttgtgtgggtgcccgtgaagatgccccttctacgacggccagtgagcaatcctcctccatgttaccgtaagaaccctaatcttgtcgacgatct |
| | tctcctgtagctcttctaatatgtcataactttgccataatcaatagttgtagtccttgatttactttgatttgcataacggtgctacggacatggcaaaaaggacacaccaccagcggcaa |
| | catacccgccgaagttggagcttgcttcgccgcaccggcggcggtgctgaggatgttgccatgctcctcatcttagttgtttccttgccgaggagcttgaagttccggccatggataggttt |
| | agggaacgcgcgcgccgagagagagagcagatttggggaagaaataaagccagggactggggaatgagaagataatgtttataaggggttttctgtaaaaagaaagagcaacctacgcggcc |
| | cttctgtcgatttggcatgggtcaacgcaccacacgagcaggtcaacgagcgctcgtgagaaaaaggacctagggtaaaccacttactaaagaattaggacctaaaggtgcattttggacga |
| | aataagactaaagtgacaccctgacgaattttttaggaccttgagtgcatttaactctttcaaaaaataaagatgctaaaaaaactaaatgcttaaaaatacatttttctatgggaatactt |
| | aaaaatactagtataatttgatggcttcctagtatatttctagcgcttacaactaaatttatattcttggtctaaataaaattataatctgataaattatgtgatatgtatatagccatatg |
| | agaccataatttaattttacctgcaaaaagtaagtaatagtaatagtagtactacatactccctccgtccgaaaatacttgtcgaagaatttgatgaaaatggatgcatctagaacaagaat |
| | acatctagatacatcaatctccccgacaagtatttccgaacggagggagtactagataataagataactatccaaaaaaaaaaagataactgaaggttgccacctagcacattcacattggt |
| | acaacttggaaaagcacagccccgtcgtcctgctcccagttgagttcgcgacctacacaccggccatgccccgcttctcctccaccacgccaatgtcgccaccccgcctccgcctccgactc |
| | tgcgcccgccactcctcctccacctctcatccctcacgcatctgggatccccacgccgccttcgccgccgcggcacagcgggcgagctctggcacgctcactacggaggacgcacaccacct |
| | gtttgacgaattgctgcggcggggcaatcctgtccaggagcgtcccttgaataaatttctggctgccctcgcccgcgcgcccgcgtccgcatcctgctgcgatggccccgccctggcagtcg |
| | ccctcttcggccgtttgtcccgagacgtcggacgacgggggcgcagccaaatgtcttcacctatggcgtcctcatggactgctgctgccgcgcttgccgcacagatctggtgctcgccttct |
| | ttggccgtctcctcaagacgggcctggaggcaaaccaagtcgtcttcaacaccctcctcaagggcctttgccacacaaagcgggcggatgaggctctggacgtgctgcttcacaggatgcct |
| | gagctgggctgcactcctaatgtggtggcgtataacaccgttatccatggcttctttaaggaaggccatgtaagcaaggcctgcaatctgttccatgaaatggcgcagcagggcgttaagcc |
| | taatgtggtgacatataactcagttatcgatgcgctgtgcaaggccagagccatggacaaggcagaggtggtccttcgtcagatgattgatgatggtgttggacctgataatgtgacgtata |
| | gtagcctcatccatggatattcctcttcaggccactggaaggaggcagttagggtattcaaagagatgacaagtcggagggttacagcagatgtgcatacttacaacatgtttatgaccttt |
| | ctttgcaaacatggaagaagcaaagaagctgcaggaatttttgataccatggctatcaagggcctgaaacctgacaacgtttcatatgctattctccttcatgggtatgccgccgaaggatg |
| | cttagttgatatgattaatctcttcaattcaatggaaagagattgtattctacctgactgtcgtatcttcaacatactgattaatgcatatgctaaatctgggaagcttgataaggctatgc |
| | ttatcttcaatgaaatgcagaaacaaggagtgagtccaaatgcagtcacatattcaaccgtaatacatgcattttgcaagaagggtaggttggatgatgctgtgataaagtttaatcagatg |
| | attgatacaggagtacgaccggacgcatctgtttatcgtcccctaatccagggtttttgtacacatggcgatttggtgaaagcaaaggaatatgttactgaaatgatgaagaaaggtatgcc |
| | tcctcctgatattatgttcttcagttcaatcatgcagaacctatgcacagaaggaagggtaacagaagcacgggatatccttgacttgatagtgcacattggtatgaggcctaatgttatca |
| | tatttaatttgctgatcggtggatactgcctagtccgcaagatggcagatgcattgaaagtatttgatgatatggtgtcatatggtttagaaccttgtaactttacgtatggtatacttatt |
| | aatggctattgcaaaaatagaaggattgatgacgggcttattctgttcaaagagatgctgcacaagggacttaaacctacaacttttaattataacgtcatactggatggattatttctggc |
| | tggacaaactgttgctgcaaaagagaagtttgatgagatggttgaatctggagtaagtgtgtgcattgatacatactctataattcttggtggactttgtagaaatagctgcagtagcgaag |
| | cgatcacccttttccggaaattaagcgcaatgaatgtgaaatttgatattacaattgtcaatatcattattggtgccttatacagggtcgagagaaaccaagaggctaaggatttgtttgct |
| | gctatgccagccaatggcttggttcctaatgctgttacctacaccgtaatgatgacaaatcttataaaagaaggttcagtggaagaagctgacaatcttttcttatccatggagaagagcgg |
| | ctgtactgccaactcttgcctgttaaatcatatcatcagaaggttactggaaaaaggagagatagtcaaggctggaaattatatgtctaaagttgatgcaaagagctactcacttgaagcta |
| | caaactgtttcgctgctgatctctctgttttcaaggaaagggaaatatagagaacacatcaaattgcttcctacaaagtatcagtttctggaagaagcagccacagttgaatagttggtaca |
| | tgatatctgaaatttaatttgcatcgcttgccatgggtccgtctgcttgtacaagaaatgcattttctatttgtaaataggaagtcagtttagaacaagccatcaggatgacgcaaagagta |
| | caattcagttgcaccaccaataaaaaggcagaactagggctgccaaaacaacactgaatcaaaactcaaacagaaggagcagcaaacttttttttttttgaagctggacagtctgctagcca |
| | aacaactacaggagactgtcaggggggcatgtagtggctggcgtctaagcgctttgcttcttccaccatccatggcttagcctcacacggaatcgagtcaaccaattcccgtcggttttggg |
| | ggctcccttgaagatgcaattgttttcagcggccagatacgcatggtcagattaatcagcgagtgtgccccttctgtctggttcgagaaagaatttgaagagctgagcttgtccctgctgga |
| | accagtttgaggttagttaatcataacagggtactagagaggtgttttattgactgttgatgtgtaatatgttatatgccatcctcttgatgattacggtgatctgtgaagaggcagcatgc |
| | aaaagtctgaatcacatatgcttatgtaattgtgttattatttgtgcagcttctagaccttttgccttgtagatggctacatggatctagttgtagcaatctgtaactgttagtgttttgta |
| | tatgctggcattgatgattagggtgacttgtgaagcatgcagaagtctgaatcatacatgcttgtctagttatttttggagctctctctttgccatgtacatgttttatggatgcagttgta |
| | gtctgtatgtgctggcatttaatatgagggctggtctgcaatttttctgttttgatcgatacagcgccatgttgagtttaaccctgtaacttggcttaactagatcatgctggtgttatctc |
| | taggaatcccaatatcaatactctctgttgttgcacaaactgacgcagatcaatgtccagaggtaatatggttctgcctgtaattttcatcgactgtcagcatgttccttttttatttagta |
| | ctctgtatatcacggcggtatgactataataaggatatacggctggtgcattttgagcttctctgtttcttgggataaagctgataaatttctattcttgttggctttggtagtatgaacag |
| | aaagcaggcagtaata |
| SEQ ID NO: 17_ | atggggaactgctggggcgccaagatcagctccgacacctcctcctcctccccttcagggacgaattccaagtatgctagtaggaatggggcggccttgagcagctccagcagctatgcctc |
| TaPK-like CDS | cgcggcgtcagtgccgcggagcgagggtgagattctggagtcagcaaatgtcaaggcctttaccttcaatgagctgaggaccgccacgagaaacttcaggccggacagtgtgctaggcgagg |
| | gagggtttgggtcagtcttcaaggggtggatcgatgagaagaccctcaccccaaccaagccaggcaccgggatggtcattgctgtaaagaagcttaaccaggaaagctatcagggccatagg |
| | gaatggctggctgaagtgaattaccttggacaactatcgcacccgaatcttgtaaagctcgttgggtactgtgttgaagacgaacagcggcttctcgtctacgagttcatgcctcgtgggag |
| | tttggagaatcatctcttcaggaggagtacacatttccagccgctctcctggaaccttcggatgaaaattgcccatggagcagcaaaagggctcgcatttctccacagtgacaaggccaaag |
| | tcatctaccgcgatttcaaaacctctaatatcctcctagatgcgaactatgacgcaaagctctcagatttcggcctggcgaaggacggaccgacgggtgacaagagccatgtgtccacaagg |
| | gtgatggggacatatgggtatgctgcaccagaataccttgcaacaggccaccttaccacgaagagcgacgtgtacagcttcggcgtggtcctcctggagatgctctcggggcgtcgcgctgt |
| | ggacaagaaccggccgaccggcgagcacaacctggtggagtgggcgcggccgtacctgacaagcaagcggcgcatcttccgcgtcctggatccccgtctagggggcagtactccctcgccaa |
| | ggcccagaaggcggcgtcgctggcgctgcagtgcctctcgatggactcgaggaacaggccgagcatggagcaggtcgtcgtggcgctggagcagctccacgacgccaaggagggagggaaca |
| | gccctcgcccgcagctgcagagaaagccgagcagcaaccggggcgtgaacggctcgagatcgtcgtcgaccaaggggaacaccaagaagcccgcctcaccgagaccggtttga |
| SEQ ID NO: 18_ | mgncwgakissdtsssspsgtnskyasrngaalsssssyasaasvprsegeilesanvkaftfnelrtatrnfrpdsvlgeggfgsvfkgwidektltptkpgtgmviavkklnqesyqghr |
| TaPK-like | ewlaevnylgqlshpnlvklvgycvedeqrllvyefmprgslenhlfrrsthfqplswnlrmkiahgaakglaflhsdkakviyrdfktsnilldanydaklsdfglakdgptgdkshvstr |
| | vmgtygyaapeylatghlttksdvysfgvvllemlsgrravdknrptgehnlvewarpyltskrrifrvldprlggqyslakaqkaaslalqclsmdsrnrpsmeqvvvaleqlhdakeggn |
| | sprpqlqrkpssnrgvngsrssstkgntkkpasprpv* |
| SEQ ID NO: 19_ | ctgtcgtactcgtgacggccatctacacgtataatactcccacttgcacatacgtactatgcacatgtacaacacatgataataaagtttggcaaacattgatcgttgcggggcgagctgca |
| TaPK-like | ggtccatgttcacattgtattttctcagccaagcagaacaatactagaggacatacgccattgacttgacaatttggttaccacccacaaactgtattgaccagatatatacaatatatgta |
| genomic | ataaatgttataaaaagaaatggaaaataataatttcggtgcatgaatcgtgcctcgtccacgaatggtgatactagacgttttcccggctagctagtaaaaaagtaggcgatcggtgtacg |
| | ggtatcgccgtaaccactaatattgtcgcacctacgtatacgcatgtaccggccatattataaacaaaacacacgaagaagaaacggaggcgttgtacggacggcgatcgtgactgcacgat |
| | gtgaggagccgtcccacactctatcgttttcctaccctagccgccgccgtcaccaagtgagtagggcgaggcgactcttttccttcgatctccaccggccggtgcgctggccccctctccct |
| | tcggccgctgctccggcggcaggagggagggggaccccgttcctctgttctgtagttaggttttggatttgtaggtcgtgatgcgtcgtttgggtggtgggagcggcgtccggacgaataaa |
| | tctgcctcaaccccactcccacaccggcggtgtctttcatggaggtgtctcggagttggtggcatcgtgtgtatgtcgatccctcagatcgacagcgttagggttcatggatggtgttggcg |
| | aggcggcggcggcgacgccatcaggtgtttctctccttctgctcggtccccgttgctgtggtgttgtctccgacgtcatggtggagcagggaggttttgtcgttcaggagtatgcgcaggcg |
| | gttgtctacgcaagtgacatctggaagatggagtatcttgtgttgggtccgtggtcgaaggcgggcagttctggtttccttctccgacgtcgtcgtcatgcgagggcgtcagttctgaagtt |
| | cgatggcgtgtccggggacatgttgccccggtctgattctttcaacggctatgattttgcttttggcaaactactttggagatccgtaaagctgctgatcagcgatggagccgcgtcgagct |
| | tgggcgaagatgtgatctgtcttcttaccctttggtcaccgcttcggtggtgtcgaaggaaagggacatgcgctggtattttgcatagaattttcctattttttccgtcatgtatggtcggt |
| | gcttacgtgccttgtaacttgatctttattctatataaatgagacacgtattatcatgaaaaaagaaaacggacggcgatctggcgtggcaatcacgggcagacgaaattgaccgtttggat |
| | ttgaatattcgcttttatatacgtcgaacgactctcgatgctgtaccatcaaatgtactgtataatcggagtagaaaatatactggcacacgagcaactagacataatttttattcgcactt |
| | gttttatactgatatatgttgtggcgatcagttttgtttaatcttttatcccgctgatgataagctcgattggcgactggtccatgcccctccgggctccacacagccaggcgaattcgtgc |
| | tatcatgcaggcagcccgaacaaagaacatgttctcagtctgcaactgctcagccggacccaagtaagtcataaatgcgacgcaacaaaaaatggcgccgcaaaagttgggacgttttctca |
| | gttatcctgaaatattataataacatgcatgttgtatgcgtacctcatgtagcggggaaacatatgcagccgggcaccccaaaccccgtcatacgcccggaccgacgcccaaattagtgacc |
| | ggtcacgaaagactgacccagacgcgtgcctcgaatgggtcttaaacgctcaggctaaccgccacccctcatatctagtcccaaatctaagacggataagggcgcgccgcccaggagcgtcg |
| | gggcgcgtccatcacgttggtcctggctcacgatggcccacccgactccacatccgctgcctgggccaaacactatccacttcacttccctccgccgcccaagctcctatccgccgatcacg |
| | agccttcttcggcttgacgtgtagcgaatccgacgacgaccagtacagatccgttgactagggtgtcaacccgtgcggtttggagaaggaaatggccatccgcattgcacttcgccactccc |
| | gggaggacaacgcccttccgaggggggatctacccaacgcgagtccattgcgtcggtgcatcgggcgctcagattctccggcgctggatcatcgtggtcctctcgggagccttccaatctcc |
| | ctttccccatcatggtccgtcggagtacgattcctatcgggtatcagtgtgcccgccgtgggaagaagaggatgagggcgtctgaggtccgcctcgctgccgacattgcggcagcggaggcg |
| | gctgatgcgatggcaggggcggtcgcaggggaggaagtcatctgcgtccgcattgtaaagaagcggcagcgaacgaacacgcgcgcccctcatccgagagcagaatcggcgggccgtgccat |
| | ggccggactgccctccaaggaggagaaggaggacaacgatgggtcagacagctccgacgatgagcagatttgagtattttcgtagtgcaaaatgtcatagacctgtttaaatacgagtatct |
| | tcctagtgcaaaatgtcgtagacctgtttaaatacgagtatcttcgtagtgcaaaatgtcgtaggaaatagaaatagtatcctacgggtatgtagttgcatttcttactagagtagatcgta |
| | ggctcaactgaaagcactactagtacgtggtggtgataacctaatacgtgactttgaacatgtctctgcccctccctttctctcgccttttcggccccatcaatggctccctccccctcctt |
| | ctagagactagaagcaggccccgcctccacaggtgctagcggtagtgtatcccgctcggtggcggtggtggtgcgtggtgggggagaaagttggaggccggccagcgcacggggaaaaggag |
| | acgcaaagcggccacccaaaaaggcgcacatactttcttttttctttccaccgccgccctccggaggagttccgattccccctctctttctctctcttctttctctctctctctttcttgct |
| | ctgctctggaagcaggggaaggagggaggggcttccgatttgggcgcggaagcactgtccgccgtgattcgattcggctgatttgggaggtctgctcgtttgatttgggttcgctgatttat |
| | tggttgatttgttggttcgtgtccggcggctcgatttgagggccggacggtcctcgccatggggaactgctggggcgccaagatcagctccgacacctcctcctcctccccttcaggtgcgc |
| | gcgtcttcctcgtctcctgctgctattttacaaatgtttttactagtactactatactagcagtactgatctgtgcccataactcgactagtagtactgatctgtgcccacagggccgattg |
| | ctattttctttgcccctcaattttttcacaactcacaagcagccgaataaataagttctgaatgagaaacaagtgttcaaccaaccatttctctcactccaatctgctcatttcgtgctctg |
| | catctcttctctccttcccgaaaccattcttaggttatgctgatttcggcgacaggtttggtcaaagtcaatttcgttctgtttaattggcttatgatagttgagtcactaaattcagtccc |
| | tccttttggaaaaatctgtcgcgagcttggactcttcctcttgacctgcttcatgtgcaaggccaaggaatcatgcctttccgtttatggcctaggggagaggaagcttccgatgtagtaaa |
| | atgcttcacttaactttatctgacttctcctctgatttttctgagtgtccagttcaaatcagacatatagaggaaaagctcgaattctgatgctacttttggctacatccacgtgcttttct |
| | tcaaagttattgactgtcctgttttggatgttcagtcttcttttctgtaataagcgaaactaaaggaagaaaatacatagttagtccgacctaacttgtccatgatggacctattttctgtt |
| | caatgttaggcagacaagataaatgattaagggacatcaacttatttatgctaaaatgtgactgaataatctctgaaagtctgcaccattttatgatgatgatcagggacgaattccaagta |
| | tgctagtaggaatggggcggccttgagcagctccagcagctatgcctccgcggcgtcagtgccgcggagcgagggtgagattctggagtcagcaaatgtcaaggcctttaccttcaatgagc |
| | tgaggaccgccacgagaaacttcaggccggacagtgtgctaggcgagggagggtttgggtcagtcttcaaggggggatcgatgagaagaccctcaccccaaccaagccaggcaccgggatgg |
| | tcattgctgtaaagaagcttaaccaggaaagctatcagggccatagggaatggctggttagtgacaattttgcctgctggaaatgggatttcttgtttatttcagttctgcattgtgtctga |
| | catgctctttcttttgggcgcaggctgaagtgaattaccttggacaactatcgcacccgaatcttgtaaagctcgttgggtactgtgttgaagacgaacagcggcttctcgtctacgagttc |
| | atgcctcgtgggagtttggagaatcatctcttcaggagtaagtgcctggacttttacttttcttattattcgctgggtgttattctccattttttaaggaatgatgctaaagtgttccaaca |
| | ttcagggagtacacatttccagccgctctcctggaaccttcggatgaaaattgcccatggagcagcaaaagggctcgcatttctccacagtgacaaggccaaagtcatctaccgcgatttca |
| | aaacctctaatatcctcctagatgcggtatatatgtccacagcctggaaggatcttttccttcgtgtccgcaaaatatctctatcggttggtctgacagtcacgcaatttcttcttcagaac |
| | tatgacgcaaagctctcagatttcggcctggcgaaggacggaccgacgggtgacaagagccatgtgtccacaagggtgatggggacatatgggtatgctgcaccagaataccttgcaacagg |
| | tcggttgatcattccactttcagaatcctcagctgcgcaaatatcagatatattgccgaaaatctgaccacacctacctgaatcgtgattgcgttgtcactctggttgtgtcatcgaccaag |
| | atgccaaacataaccaattttaggctacaacctttcaatcctcatctgcacaaatatcttgcccgaaaatctaaccacacctacctgaatcatgattgcgttctcactctggttgcgtcatc |
| | gaccaagatgccaaactcaactaatttttcggctagaacctttcaatcatcatctgcacaaatatcttgctgaaaatccaaacacacctacctgaatcgcgagtgcattgtcaccctggttg |
| | cgtcatcaaccatcatgccgaacctaaactaattttcctgctgcaatatttccatgatcaggccaccttaccacgaagagcgacgtgtacagcttcggcgtggtcctcctggagatgctctc |
| | ggggcgtcgcgctgtggacaagaaccggccgaccggcgagcacaacctggtggagtgggcgcggccgtacctgacaagcaagcggcgcatcttccgcgtcctggatccccgtctaggcgggc |
| | agtactccctcgccaaggcccagaaggcggcgtcgctggcgctgcagtgcctctcgatggactcgaggaacaggccgagcatggagcaggtcgtcgtggcgctggagcagctccacgacgcc |
| | aaggagggagggaacagccctcgcccgcagctgcagagaaagccgagcagcaaccggggcgtgaacggctcgagatcgtcgtcgaccaaggggaacaccaagaagcccgcctcaccgagacc |
| | ggtttgaccgtgtgagtgggatcacatagcagaatctagtagagagatgcgtcaaggtgtaatgtatgtatggtataatagggttttagagtggtgatctgcgaagaggcagcggatttgtg |
| | aatgttttgtgcccgtgttgtagggatgtatctttgcagcttctagtgttggtatgtacagcttgtatctatccggttgtagctagtctgtatgtagtccttttgtgtatgcctgattgctt |
| | tggttctgacttaaggtccagaggttttgatctgatttagatgcatgcaccctgttaaaccaagaccttaaaatatgaagatgaacagtgtaaagtacatatttgtcacttcatttatcaaa |
| | tgtgaatgaacaatcatgttggtgttttagcagagcaggccatgacttccatgggtcaactgtatcttttcccttagggatgttgaccattttgttcatatcatacagtactagtagtatat |
| | gtttcatttttttaatgaaaaaagtagtatatgttacagataatgttgggaaaatcaccttctgtatttttaatttcaaacagttggccacaatgatctttttatttacttggtgggttcag |
| | ccaaattaccattgtttttttcaggggtttcagccaaattattattatctgggcaagcgtacggcggcggcccaaggcagagcacgcagcccagccgccaaaacagagctcgccctcgctcc |
| | cctccggcccaagtgccgctcctcctcctcctcggtggacgccggcgccggtggatggctccgccgcgccaaatcgagcccctcacgccgcgagccgctcctcctcctcctcagcctgcaat |
| | ccccgccgagctccgatccccgccgccgagctccgatccgcgggcgatcgccggggcgccgccaagatcagctccggcccctcccgttcc |
| SEQ ID NO: 20_ | atggcagctctaaccagccttgcctcaaacccgaatccaaacaagtcattcgaggtccttcctaatccgggtgactccctctcaagcctcagttttagcccgaaaagtaatcttcttgtggc |
| TaRAE1-like CDS | aacttcctgggataaccaggtgaggtgttgggagataggtaatggtaacagtcagccaaaggcatccatatcacatgatcagccagtgctctgctcagcctggaaagatgatgggactactg |
| | tcttctctggagggtgtgataaacaggtcaaaatgtggcctctgctgtctggtgggcaggctcagacggttgcaatgcatgatgcacctgtcaaggaggtcgcatggatttctcagatgaat |
| | cttcttgtatccggaagctgggacaagacactaaggtattgggacacaagacagccgaatcctgcccatgttcagcaacttcctgatcgttgctacgcacttgctgtgaattatccccttat |
| | gattgtgggaacagctgatcgcaatattgtgatcttcaacttgcagaatcctcagactgagtttaagcgtattcaatcacctctgaaataccagacacggtgcgttgctgcctttccagatc |
| | aacaaggattcctggtgggttccatagaaggaagagttggtgtgcatcatattgatgattcacagcaaagcaaaaacttcacattcaagtgtcacagggaaggaaatgatattttctctgtc |
| | aattcgctcaactttcaccctgttcatcacacgtttgccacagctggatctgatggtgctttcaacttttgggataaagatagcaagcagagacttaaggctttcagtcggtgtcctcaacc |
| | cattccttgcagtagcttcaataatgatggttcaatatttgcttatggggtgtgctatgactggagccatggcgctgagaaccataatcctgcaaatgcaaagacatccatctatctccaca |
| | gtccccaggaagccgaggtgaaagggaagccaagaatcgcaacagggcggaagtga |
| SEQ ID NO: 21_ | maaltslasnpnpnksfevlpnpgdslsslsfspksnllvatswdnqvrcweigngnsqpkasishdqpvlcsawkddgttvfsggcdkqvkmwpllsggqaqtvamhdapvkevawisqmn |
| TaRAE1-like | llvsgswdktlrywdtrqpnpahvqqlpdrcyalavnyplmivgtadrnivifnlqnpqtefkriqsplkyqtrcvaafpdqqgflvgsiegrvgvhhiddsqqsknftfkchregndifsv |
| | nslnfhpvhhtfatagsdgafnfwdkdskqrlkafsrcpqpipcssfnndgsifaygvcydwshgaenhnpanaktsiylhspqeaevkgkpriatgrk* |
| SEQ ID NO: 22_ | tttgtaaaaggctatggggacatttgttttcctctggcgcacagaaatcaagaggtgtcattaattctgaatgaagaagacaatgctatgggctccgcaatacctcgctaacgaaggtgtcg |
| TaRAE1-like | agggggccgagctatcatcagccggatacaggagacgggaggaactcgcccctcaatccgaagacttcaagaagcacagagctaatggaaattatcgacctcgctgttggaggctagttcgg |
| genomic | aggctaatgagggtgtcttggactaaggggtcctcgggctgccgacctatttctcatgggccgtactagtggactgtttgttgttcgtcaggggaagaccggacttcggacgaccccgtgct |
| | ccaaaaggaaaccttcggagacttgacgtatcctccaagtctggcttagtgtgacttgtgtgtttatcccctgttggtaaccgacatatgtaaccctagggacccctggtgtctatataaac |
| | agagggtttagtctgtagaggctagaaccatcatcctactcctatagggtttagttcatctgatctcgtggtagatcaactctgtaatcatcacacaaacacaccaagacaatcaagcagaa |
| | tgtagggttttacctcttcgagagggctcgaacctgggtaaatactatctctaccttccctgttacccatgcatccaagatctacaactcaggaccccctaccgagatctgccggttttgac |
| | accaacacacataaaattgagggtgaccaatcacgtaaagctcttaatagcgatattaatatacgtacacatagcaattgtcgatgaaattgttggnnnnnnnnnnnnnnnnnnnnnnnnnn |
| | nnnnnnnnnnnnnnnnnnnnnnnnnnnnnnnnnnnnnnnnnnnnnnnncctgctcatccctggatgttgacggctcaatggtgtatttttcccgtcgtgcgtcttcatcggtgaggttatgg |
| | tatgtacaattttggcgcacaatgacggcgacttccctgatccttcgtcatcgtctccaccgtgttgattttggtgctggcgtgaggcctgaggtatgtctcttggatctaatgctcgtcta |
| | gcgttggtggtttctccttcagagcgatggcccggtaagacgtggccttaatcactccccgcccgtgatgaacatcaggctggcttcgatgatggagaagcatgtgttggctcggcatgttc |
| | tagaggatgaagaaggatgacccagggattttgatgtaactttattaatattttcttcctataatttgtacttccttttcttagaaaaaaactatttggcttcacatgcgctggtttggtta |
| | tataagagggagcatatgggtgttgtctttttatataagaaaaaaaagagcaaaataagccacgcgccgatcaagcctgcgaactgagtgtatacccggccatgggcaacccagactgaccc |
| | gacccatcccgaaaaagctcgacccgtcccgactcgaccttgcccacgggccgggcccaggcctagattttgagcccgatggctgggccgacatgttgtatttgcgatttaaggaagaggcc |
| | cggcccaagggccgaggcccgacgggttttttgactgatgggccggggttgggccagattattaggcccaacggccgggccgggccgggctcgggcctaagccttttgcttgcgttcaggct |
| | ttgataggctcagcccgaagtccacatccacatcaaccacccccgcgtgccgatgaagccatttctttggaaggacctgcgcgtgcatccgacgcgacgcgctacaccgaagcctatactac |
| | tacatctcgtctcatcgccctcctcccagacgacgcctcgagaatcctcggctgaggctccactccgcactcgcaagtcgcacccgcagccgcgccgtccccagcagccgccgccgaccgct |
| | ccccgtccggcaggtaagccccccgagcccctcctcgccgctccgctcgcgcggactcctagctagctagggttcccggcaccgattccgcggcgctgcactccggcgcaatgctcgcggcc |
| | gcggacgggcccgccgcttcgggggaagctctgatctggtggccggcgctcgctcgctcgctcgcggtctccggagcaaatcggccgcatcgagtggccggagcaagcttaggatagttcga |
| | ttatttcccccatccccgttggtcaggccgagagggccagtttgggcgacctcctggtgattccgctgccgtggtggggattgggtggtcgtggcccgggtgtaggcgtttgtaggtcgagt |
| | gagtgtttccttccaggttctatgctcacccagcttgtgttatcactgggatccgggcgttcggcaggctaacattcactggattttggacctgcattgttgcggcgcgtgccgggggatag |
| | cgtttatgctggctttggcgcggtgattttgtgcgtgcgtgcaaacttttggggctgccttctggtgatctcgctgtcgggtggtgcgagttcatcaagcggtcagggtgtaggcatttgta |
| | gtcgagcgaccccagctttcatgcccaccagctcgtattattcggtcgttaaccacgtttaccgaattttggaccgacgtcgttgcggcacgtaccaggggacagcgtttatgctggtttcg |
| | gtgtggagattttgtggtttaaacttggggcgcgcttggtttgcgaggctacctcctggtgatttcaccgtcgggtggtgggaatcggatggtcggggtgtaggcatttatagtggagcgga |
| | ccccttgttttatgaccagacatttagccgaaatttggacctacactatcgtggcacgtaccaggggatatcatttatgcgggttttggcgcgacactttctttgtcgggattttgtggtgc |
| | aaacttaaataatgacttccctaatgttaaacactcgttctggcatcgtggactgttggaatcgttctttttcgtagtctgagctactttaaggtgttacttatgctctcgtgtgcgtggga |
| | gactccacgtgctgatttctcttacttgcttctcatggtcactggactgatatttcttttccagattttgaaggttcacagaaggagagatggcagctctaaccagccttgcctcaaacccg |
| | aatccaaacaagtcattcgaggtgtgatactttttgtggccatgtctgtcctgcttttatttttttgcgtcttgtcattctgtatatgctacatgttctctgcaatggttgctttcaaatgc |
| | aggtccttcctaatccgggtgactccctctcaagcctcagttttagcccgaaaagtaatcttcttgtggcaacttcctgggataaccaggtaattttgtttctgctgtaattttcagtgagc |
| | ttccattattgtgatgtatcttactaggaatgtcttttgagttttgtgcaggtgaggtgttgggagataggtaatggtaacagtcagccaaaggcatccatatcacatgatcagccagtacg |
| | ttctcatacatgtttctcctgaaagtagtttcatcttacacattagagtggttccttacctgtttacttggtatgatgattgatttgtttccttccatcaggtgctctgctcagcctggaaa |
| | gatgatgggactactgtcttctctggagggtgtgataaacaggtcaaaatgtggcctctgctgtctggtgggcaggctcagacggttgcaatgcatgatgcacctgtcaaggaggtcgcatg |
| | gatttctcagatgaatcttcttgtatccggaagctgggacaagacactaaggtgcaagaacaatattcctctccaccctcagtattctttttacatggaaattcagcgcatatcacccatat |
| | ctgatcccaaattgttatttatggctgttttgcttttgggaattcgtgttgtttgtggtatcctttctgtacttcatgtgttctgcattaatgttcacacattggctaaccaaatattgtag |
| | aagttctatctcatcctctttatatttgcactcattttgtatgccttctttgatcctgaagagttttctactacttgttgaatcatactgccctattcttatcaaaggttgtgcactttctg |
| | ggtatcatctgtactatttatggctgtagtgaatttttcttatggcagaagtttttatagaagtaactgttgatgcagacagttactatattaaatgtacattatgtgattagtgttagaaa |
| | tgttcgtttcgagttttagcatcatctttatgaagattatttatgaaacatcaagttagggtctggctgtcagtttaaacatgagcacagattgagcataggcgataatcatggttggtagg |
| | aggccagggaccgggatccttgtgggacctgccggcgtacccgtgatagcagaaggggtgcaagggacagggcactggaactaaggcaggcagaaacagagaaataaagattggggattaga |
| | ctttgtagccaattcataaaaaaattggttgcatcattttgtagaggttggccttaacatatgactaaaaaacaaaaacaaaccaagactcttccctaaactgaataccgcagatactcaag |
| | acaccatatagggccacgggctcaggctgaagtcatctaggtctcagacatgccaaaacagactgggacaaggacaagctgaaactgactagtactaggtttcccggggcaactaccgttca |
| | gctggaggtctgaatgcgtggtgccttctctgatacatggatctcatgtggcacttctgttgtgtatttgtcccagtctgcttattttcctttgtctgtatcatgctgagactaatacattt |
| | gggtcgtgcattgatcttcttctcattacaacttttagctctgatcccgatgacttgtttctagtctgctatcaatgcttatcatcatactattgtaggtattgggacacaagacagccgaa |
| | tcctgcccatgttcagcaacttcctgatcgttgctacgcacttgctgtgaattatccccttatgattgtgggaacagctgatcgcaatattgtgatcttcaacttgcagaatcctcaggtaa |
| | ttttgcttcctaaccgactatgcattttcaattcttgtggtggtggtgctcatgcttttactgccattgacttatattttgaactgcctgacttaaattcatccctgatgtgattttagact |
| | gagtttaagcgtattcaatcacctctgaaataccagacacggtgcgttgctgcctttccagatcaacaaggattcctggtaatgccatattttcttgtgtcttttactctcagcattacgac |
| | accaaccaaaacttccgttgtttgaatacataacttgttgttactatgctttgaagtttaatcatatgttcttctctacctatcccatcataattatttatgttgcccaattatgttgtttt |
| | tacctagcttgagttttcgaccttaaacattgttacaaccttactcgaattagctttagatgttggcctgccatcatttgcagtgtaatgggcgttgttcctttagtgatctcatttcaaac |
| | ttcctacccctctattataggataaataaggtatattgattgtgaaatcaaactttactgactataaatactatactatgtgatttatgtgacataaagttgatatcattggatttgttttc |
| | aagagtactttgttacgattttgataaaatatgagaagaaatttatggccaaagattgaactcgagcagtcaaaatatgccttgtatatctggatagagtgtgtattactaggaatgttaat |
| | ccaatataaatgtcactaattgacatctctctagttagcacaaaagctgcataaaagaagaggatgcagttttccttaatgttttgaccaactccagctgtgttgcaaacagtttccttcac |
| | cagaactgtagaacggatcttagcctatagcagttaaagctaatctgaacatgaaatccggctgaagtacaaatagtaaaaccatccaaggcttcaagcaattgtttttgaactacagatct |
| | ttatagatgtctatttctgcattttcctattcttcacctgcttgtgggtgttcttatagtgggtcgtcgtttcatcatgggctttgaccaatcaatttttgctgttacacaatgatcttttt |
| | ctacatcaatgaactgcctatattggataaagctggtcgatacctggcaatagttcttgttttaattattttcttgttgtttccatattacttgggcaaaacatttttttacatgcgactga |
| | cacctgattttgtttgaaggtgggttccatagaaggaagagttggtgtgcatcatattgatgattcacagcaaagcaaaaacttcacattcaagtgtcacagggaaggaaatgatattttct |
| | ctgtcaattcgctcaactttcaccctgtaagatctctatcttgttctctcttgactccatatttatctatgtgccatactgccattcttatatttcattagtccacttctgctattatggaa |
| | aatgctaatggcgacttctgttgagctttagtcactatgtctccatcatgatttgtaaaatactgaaagctcagttcgttgtcttccacaagatgtgttgagctttattcaatacagatacc |
| | taccacacttcgcctaatataataaccactatgcttgaaggcatacatatgttagtaaactactccaaattgatcaagcaatattttgcagaaataaaaatcccaacagattagcttcagtt |
| | taattgagcaactcgaaatcgtacctatttgttacagacacatcgaagagaagcgtttctgcttcttcttttgctaactttggcatcttattcattggattggtatatgcttgggtgatgat |
| | actatacatccatttagttgagtgtgcttgttggctatatatttcactggaaaatgcaactgactggtgataattgtgaagtatggaaattcagttagtctagttacttatgggatttcatg |
| | ttatctaccacttggcacaggttcatcacacgtttgccacagctggatctgatggtgctttcaacttttgggataaagatagcaagcagagacttaaggtttgtctattcccgttttttttt |
| | ctattttcccctcttgttggtttatgttttcttgattggaattcaatgatttcaagatggctctactttgttgtgatctgtggacactgatgatctgcatataaattctttgttgatgcgca |
| | tacatagcctgaaatcatcccgagctcaagctaataaactcactatcctggccatgagcagaaaaaaaaatattagttcataaatgttatcaccaattctgattttcccagccgccttgtca |
| | tcctgctgcttttggcgtttagtatgcacttctttttattcttgataaaagcatattacatatttattactcaaccgcatggagcctctgcagtaaggtatatgtgtaatttgtcaacagat |
| | aaccttttagcttttaggcatatcatacacagtgttgcattctgacagttgcttacttgcttagcatagagggctatctgtgtgtgcccaagtttattgcttcagttccctcatttgtcatg |
| | caccttctggtatgttgcacataacacattcttacatttcccctacactgcttctatacaggctttcagtcggtgtcctcaacccattccttgcagtagcttcaataatgatggttcaatat |
| | ttgcttatggggtaagtcaagtgatgcttttgagagtgtcttgttatcaccatgtttttcagtccggtcagagtttagatatgttacatgcctgtcatttttcctgcaccttatttaatata |
| | cggaagaaatcagctcgacaaataattcatgttccttttgaactgctatcttgtcgtgtcctctgaatccaagttctgaatgcaaccgtaattatcattgctggggagcaggtgtgctatga |
| | ctggagccatggcgctgagaaccataatcctgcaaatgcaaagacatccatctatctccacagtccccaggtagtttccgagtgctgatatcgccataaaaagaattcttgtattttcgaca |
| | tcatttgagatccattcctgtttttataaattgttattatgttatcttttggcactcaacttgcatcattctggacaggaagccgaggtgaaagggaagccaagaatcgcaacagggcggaa |
| | gtgatggtggcatcctgcctgccgaaaagtcgatagttgtgcaagagatgatgtatattcaagggccttgtttcgctcgggccttgccttgacgtgtcctctggatgttcttacttggttcg |
| | gccgcctggtagatgaaacgcctactcccagcaaccagtgataggttacttagccgtaggtcgtagcacatagccgtttatgtaatgcatagttgtccgaaggaatgtttccccgtgttctt |
| | gtgttgtgatatagtcaggtcagcgcgtagtcttttgaaaaggagaaagccttcggtgtcttgtaagtggtgtagatctttggaaatatgggcatgatattcggtggagttattctgaaaaa |
| | gatatcccggtttcggtttcagcgaatgcaagctagatttatggggaaggttgtagttagcaccatgctgcaaatgcatgcggcattcggaggatttacggtttgaatcttcctgtttatcc |
| | aagtctaggattcccaccttcctagtaccattgagcacgttagtttgaatttttttttttattatggttcaaaacttccgattcaaaaaaatctgatttattttacgcgaatatatggatgt |
| | ttcatcaatatacggttttgtatgtgagatataaagaagggccaaagacatgcaaacaatgggcatttctttgtgctggatcttttgtgttcttatctgcactgcaccatcaaatgaaaatt |
| | accaggtgcgtgcagaacgacgtgtaaagaaaacacccagcttttccgaaacttccaaatgccattttcgagctgttcaacatagtgtgcttcgaaaatccgttcttcagtacagtaccagc |
| | cacgtagcaatggctccacagaagcctcattgcttgcgagctttatgccgtgaacttgcatagattcagttcgacaacccaattgcataagcttgtgatctttttacgccgttaacttcaaa |
| | atagattcggcgcaacacgactcaatagccacg |
| | acacggg |
| SEQ ID NO: 23_ | GCGGGGCATGGCCGGTGTGTAGG |
| Target 983r | |
| SEQ ID NO: 24_ | GAGGAGAAGCGGGGCATGGCCGG |
| Target 991r | |
| SEQ ID NO: 25_ | TGAGTTCGCGACCTACACACCGG |
| Target 972f | |
| SEQ ID NO: 26_ | TCCCCATGGCGAGGACCGTCCGG |
| Target 479r | |
| SEQ ID NO: 27_ | CCCAGCAGTTCCCCATGGCGAGG |
| Target 488r | |
| SEQ ID NO: 28_ | GCCGGACGGTCCTCGCCATGGGG |
| Target 478f | |
| SEQ ID NO: 29_ | TTCTGTGAACCTTCAAAATCTGG |
| Target 1145r | |
| SEQ ID NO: 30_ | AGATTTTGAAGGTTCACAGAAGG |
| Target 1147f | |
| SEQ ID NO: 31_ | AAGGTTCACAGAAGGAGAGATGG |
| Target 1155f | |
| SEQ ID NO: 32_ | TTTGAAGGTTCACAGAAGGAGAGATGG |
| Target 1151f | |
| SEQ ID NO: 33_ | gaccaagcccgttattctgacagttctggtgctcaacacatttatatttatcaaggagcacattgttactcactgctaggagggaatcgaactaggaatattgatcagaggaactacgagag |
| TaU6 promoter | agctgaagataactgccctctagctctcactgatctgggtcgcatagtgagatgcagcccacgtgagttcagcaacggtctagcgctgggcttttaggcccgcatgatcgggcttttgtcgg |
| | gtggtcgacgtgttcacgattggggagagcaacgcagcagttcctcttagtttagtcccacctcgcctgtccagcagagttctgaccggtttataaactcgcttgctgcatcagactt |
| SEQ ID NO: 34_ | GCGGGGCATGGCCGGTGTGTgttttagagctagaaatagcaagttaaaataaggctagtccgttatcaacttgaaaaagtggcaccgagtcggtgcttttttt |
| gRNA_983r | |
| SEQ ID NO: 35_ | GAGGAGAAGCGGGGCATGGCgttttagagctagaaatagcaagttaaaataaggctagtccgttatcaacttgaaaaagtggcaccgagtcggtgcttttttt |
| gRNA_991r | |
| SEQ ID NO: 36_ | TGAGTTCGCGACCTACACACgttttagagctagaaatagcaagttaaaataaggctagtccgttatcaacttgaaaaagtggcaccgagtcggtgcttttttt |
| gRNA_972f | |
| SEQ ID NO: 37_ | TCCCCATGGCGAGGACCGTCgttttagagctagaaatagcaagttaaaataaggctagtccgttatcaacttgaaaaagtggcaccgagtcggtgcttttttt |
| gRNA_479r | |
| SEQ ID NO: 38_ | CCCAGCAGTTCCCCATGGCGgttttagagctagaaatagcaagttaaaataaggctagtccgttatcaacttgaaaaagtggcaccgagtcggtgcttttttt |
| gRNA_488r | |
| SEQ ID NO: 39_ | GCCGGACGGTCCTCGCCATGgttttagagctagaaatagcaagttaaaataaggctagtccgttatcaacttgaaaaagtggcaccgagtcggtgcttttttt |
| gRNA_478f | |
| SEQ ID NO: 40_ | TTCTGTGAACCTTCAAAATCgttttagagctagaaatagcaagttaaaataaggctagtccgttatcaacttgaaaaagtggcaccgagtcggtgcttttttt |
| gRNA_1145r | |
| SEQ ID NO: 41_ | AGATTTTGAAGGTTCACAGAgttttagagctagaaatagcaagttaaaataaggctagtccgttatcaacttgaaaaagtggcaccgagtcggtgcttttttt |
| gRNA_1147f | |
| SEQ ID NO: 42_ | AAGGTTCACAGAAGGAGAGAgttttagagctagaaatagcaagttaaaataaggctagtccgttatcaacttgaaaaagtggcaccgagtcggtgcttttttt |
| gRNA_1155f | |
| SEQ ID NO: 43_ | taatttctactaagtgtagatAAGGTTCACAGAAGGAGAGATGGttttttt |
| crRNA_1151f | |
| SEQ ID NO: 44_ | gtgcagcgtgacccggtcgtgcccctctctagagataatgagcattgcatgtctaagttataaaaaattaccacatattttttttgtcacacttgtttgaagtgcagtttatctatctttat |
| ZmUbiquitin | acatatatttaaactttactctacgaataatataatctatagtactacaataatatcagtgttttagagaatcatataaatgaacagttagacatggtctaaaggacaattgagtattttga |
| promoter+ 5'UTR | caacaggactctacagttttatctttttagtgtgcatgtgttctcctttttttttgcaaatagcttcacctatataatacttcatccattttattagtacatccatttagggtttagggtta |
| | atggtttttatagactaatttttttagtacatctattttattctattttagcctctaaattaagaaaactaaaactctattttagtttttttatttaataatttagatataaaatagaataa |
| | aataaagtgactaaaaattaaacaaataccctttaagaaattaaaaaaactaaggaaacatttttcttgtttcgagtagataatgccagcctgttaaacgccgtcgacgagtctaacggaca |
| | ccaaccagcgaaccagcagcgtcgcgtcgggccaagcgaagcagacggcacggcatctctgtcgctgcctctggacccctctcgagagttccgctccaccgttggacttgctccgctgtcgg |
| | catccagaaattgcgtggcggagcggcagacgtgagccggcacggcaggcggcctcctcctcctctcacggcaccggcagctacgggggattcctttcccaccgctccttcgctttcccttc |
| | ctcgcccgccgtaataaatagacaccccctccacaccctctttccccaacctcgtgttgttcggagcgcacacacacacaaccagatctcccccaaatccacccgtcggcacctccgcttca |
| | aggtacgccgctcgtcctccccccccccccctctctaccttctctagatcggcgttccggtccatggttagggcccggtagttctacttctgttcatgtttgtgttagatccgtgtttgtgt |
| | tagatccgtgctgctagcgttcgtacacggatgcgacctgtacgtcagacacgttctgattgctaacttgccagtgtttctctttggggaatcctgggatggctctagccgttccgcagacg |
| | ggatcgatttcatgattttttttgtttcgttgcatagggtttggtttgcccttttcctttatttcaatatatgccgtgcacttgtttgtcgggtcatcttttcatgcttttttttgtcttgg |
| | ttgtgatgatgtggtctggttgggcggtcgttctagatcggagtagaattaattctgtttcaaactacctggtggatttattaattttggatctgtatgtgtgtgccatacatattcatagt |
| | tacgaattgaagatgatggatggaaatatcgatctaggataggtatacatgttgatgcgggttttactgatgcatatacagagatgctttttgttcgcttggttgtgatgatgtggtgtggt |
| | tgggcggtcgttcattcgttctagatcggagtagaatactgtttcaaactacctggtgtatttattaattttggaactgtatgtgtgtgtcatacatcttcatagttacgagtttaagatgg |
| | atggaaatatcgatctaggataggtatacatgttgatgtgggttttactgatgcatatacatgatggcatatgcagcatctattcatatgctctaaccttgagtacctatctattataataa |
| | acaagtatgttttataattattttgatcttgatatacttggatgatggcatatgcagcagctatatgtggatttttttagccctgccttcatacgctatttatttgcttggtactgtttctt |
| | ttgtcgatgctcaccctgttgtttggtgttacttctgcag |
| SEQ ID NO: 45_ | atggctcctaagaagaagcgcaaggttgggatccacggggttccggcggctatggacaagaagtacagcatcgggctcgacatcgggacgaacagcgttggggggggtcatcacggacgagt |
| NLS-Cas9-NLS | acaaggttccctccaagaagttcaaggtgctggggaacaccgaccgccactccatcaagaagaacctcatcggggccctcctgttcgacagcggggagaccgctgaggctacgaggctcaag |
| | agaaccgcgaggcgcagatacacgagaaggaagaacaggatctgctacctccaagagatcttctccaacgagatggccaaggttgacgattcattcttccaccgcctggaggagtctttcct |
| | cgtggaggaggacaagaagcacgagcggcatcccatcttcgggaacatcgtcgacgaggttgcctaccacgagaagtaccctacgatctaccatctgcggaagaagctcgtggacagcaccg |
| | ataaggcggacctcagactgatctacctcgccctggcgcacatgatcaagttccgcgggcatttcctgatcgagggggatctcaacccagacaactccgatgttgacaagctgttcatccaa |
| | ctcgtgcagacctacaaccaactcttcgaggagaacccgatcaacgcttcgggggtcgacgctaaggccatcctgtcagcccgcctctcgaagtcacgcaggctggagaacctgatcgcgca |
| | gctcccaggggagaagaagaacgggctgttcgggaacctcatcgctctctctctggggctcacgccgaacttcaagtccaacttcgatctcgccgaggacgcgaagctgcaactcagcaagg |
| | acacctacgacgatgacctcgataacctcctggcgcagatcggggatcaatacgctgacctgttcctcgccgcgaagaacctgtcggacgccatcctcctgtcagatatcctccgcgtgaac |
| | accgagatcacgaaggccccactctctgcgtccatgatcaagcgctacgacgagcaccatcaggatctgaccctcctgaaggcgctggtccgccaacagctcccggagaagtacaaggagat |
| | cttcttcgatcagtcgaagaacgggtacgctgggtacatcgacgggggggcgtcacaagaggagttctacaagttcatcaagccaatcctggagaagatggacgggacggaggagctcctgg |
| | tgaagctcaacagggaggacctcctgcggaagcagagaaccttcgataacgggtccatcccccaccaaatccatctcggggagctgcacgccatcctgagaaggcaagaggacttctaccct |
| | ttcctcaaggataaccgggagaagatcgagaagatcctgaccttcagaatcccatactacgtcgggccgctcgctcgggggaacagcagattcgcctggatgacccgcaagtcggaggagac |
| | catcacgccgtggaacttcgaggaggtggtcgacaagggggcgagcgctcagtcgttcatcgagaggatgaccaacttcgacaagaacctgcccaacgagaaggtgctccctaagcactcgc |
| | tcctgtacgagtacttcaccgtctacaacgagctcacgaaggtgaagtatgtgaccgaggggatgagaaagcctgccttcctgtccggggagcagaagaaggcgatcgtggacctcctgttc |
| | aagaccaaccggaaggtcacggttaagcaactcaaggaggactacttcaagaagatcgagtgcttcgattcggtggagatctcaggggtcgaggacaggttcaacgcctccctcgggaccta |
| | ccacgatctcctgaagatcatcaaggataaggacttcctggacaacgaggagaacgaggacatcctggaggacatcgtgctgaccctcacgctgttcgaggacagggagatgatcgaggagc |
| | gcctgaagacgtacgcccatctcttcgatgacaaggtcatgaagcaactcaagagaagaagatacaccggggggggaggctgtcccgcaagctcatcaacgggatccgggacaagcagtcag |
| | ggaagaccatcctcgacttcctgaagtctgatgggttcgccaacaggaacttcatgcaactgatccacgatgactcactcaccttcaaggaggatatccaaaaggctcaggtgagcgggcag |
| | ggggactcgctgcacgagcatatcgctaacctcgctgggtcccctgcgatcaagaaggggatcctgcagaccgtgaaggttgtggacgagctcgtgaaggtcatggggcggcataagcctga |
| | gaacatcgtcatcgagatggccagagagaaccaaaccacgcagaaggggcaaaagaactctagggagcgcatgaagcgcatcgaggaggggatcaaggagctggggtcccaaatcctcaagg |
| | agcacccagttgagaacacccaactgcagaacgagaagctctacctgtactacctccagaacggggggatatgtatgtggaccaagagctggatatcaaccgcctcagcgattacgacgttg |
| | atcatatcgtgccccagtctttcctgaaggatgactccatcgacaacaaggtcctcaccaggtcggacaagaaccgcgggaagtcagataacgttccatctgaggaggtcgttaagaagatg |
| | aagaactactggaggcagctcctgaacgccaagctgatcacgcaaaggaagttcgacaacctcaccaaggctgagagaggggggctctcagagctggacaaggctgggttcatcaagcggca |
| | gctggtcgagaccagacaaatcacgaagcacgttgcgcaaatcctcgactctcggatgaacacgaagtacgatgagaacgacaagctgatcagggaggtgaaggtcatcaccctgaagtcta |
| | agctcgtctccgacttccgcaaggacttccagttctacaaggttcgcgagatcaacaactaccaccatgcgcatgacgcttacctcaacgctgtggtcgggaccgccctgatcaagaagtac |
| | ccgaagctggagagcgagttcgtgtacggggactacaaggtttacgatgtgcgcaagatgatcgccaagtcggagcaagagatcgggaaggccaccgccaagtacttcttctactcaaacat |
| | catgaacttctttaagaccgagatcacgctggccaacggggagatccggaagagacccctcatcgagaccaacggggagacgggggagatcgtgtgggacaaggggagggatttcgcgaccg |
| | tccgcaaggttctctccatgccccaggtgaacatcgtcaagaagaccgaggtccaaacgggggggttctcaaaggagtctatcctgcctaagcggaacagcgacaagctcatcgccagaaag |
| | aaggactgggatccaaagaagtacggggggttcgacagccctaccgtcgcgtactcggttctggttgtggctaaggttgagaaggggaagtccaagaagctcaagagcgtgaaggagctcct |
| | ggggatcaccatcatggagcgctccagcttcgagaagaacccaatcgacttcctggaggccaaggggtacaaggaggtgaagaaggacctgatcatcaagctcccgaagtactctctcttcg |
| | agctggagaacgggaggaagcgcatgctggcttccgctggggagctccaaaaggggaacgagctcgccctgccatccaagtatgtgaacttcctctacctggcgtcccactacgagaagctc |
| | aaggggagcccggaggacaacgagcaaaagcagctgttcgtcgagcagcacaagcattacctcgacgagatcatcgagcaaatctccgagttctccaagcgcgtgatcctcgcggacgctaa |
| | cctggataaggtcctctccgcttacaacaagcaccgggacaagcccatcagagagcaggccgagaacatcatccatctcttcaccctgacgaacctcggggcccctgctgccttcaagtact |
| | tcgacaccacgatcgatcggaagagatacaccagcacgaaggaggtcctggacgcgaccctcatccaccaatctatcacggggctgtacgagacgagaatcgacctgtcacaactggggggg |
| | gacaagaggccggcggcaaccaagaaggcgggccaggccaagaagaagaagtga |
| SEQ ID NO: 46_ | atggccccgaagaagaagaggaaagtgggcatccacggcgtgcctgcagcaatgtccaagctggagaagttcaccaactgctactccctgagcaagaccctccgcttcaaggccatccccgt |
| NLS-Cas12a-NLS | gggcaagacccaggagaacatcgacaacaagaggctcctggtggaggatgagaagagggcagaggactacaagggcgtcaagaagctgctcgacaggtactacctgtccttcatcaacgacg |
| | tcctccacagcatcaagctgaagaacctcaacaactacatctccctcttcaggaagaagacccgcaccgagaaggagaacaaggagctggagaacctcgagatcaacctgcgcaaggagatc |
| | gccaaggccttcaagggcaacgagggctacaagagcctcttcaagaaggacatcatcgagaccatcctgcctgagttcctcgacgacaaggatgaaatcgccctggtgaactccttcaacgg |
| | cttcaccaccgccttcaccggcttcttcgacaacagggagaacatgttcagcgaggaagccaaatcaacctccatcgccttcaggtgcatcaacgagaacctcacccgctacatctccaaca |
| | tggacatcttcgagaaggtcgacgccatcttcgacaagcacgaggtgcaggagatcaaggagaagatcctgaacagcgactacgacgtggaggacttcttcgagggcgagttcttcaacttc |
| | gtcctcacccaagagggcatcgacgtgtacaacgccatcatcggcggcttcgtcaccgagtccggcgagaagatcaagggcctgaacgagtacatcaacctctacaaccagaagaccaagca |
| | gaagctgccgaagttcaagccactgtacaagcaggtgctctccgacagggagtccctcagcttctacggcgagggctacaccagcgacgaggaagtgctggaggtgttccgcaacaccctca |
| | acaagaacagcgagatcttctccagcatcaagaagctggagaagctgttcaagaacttcgacgagtactccagcgccggcatcttcgtgaagaacggcccggccatctccaccatcagcaag |
| | gacatcttcggcgagtggaacgtcatccgcgacaagtggaacgccgagtacgacgacatccacctgaagaagaaggccgtggtcaccgagaagtacgaggacgacaggcgcaagtccttcaa |
| | gaagatcggctccttcagcctggagcagctccaggagtacgccgacgccgacctgagcgtggtcgagaagctcaaggagatcatcatccagaaggtcgacgagatctacaaggtgtacggct |
| | ccagcgagaagctgttcgacgccgacttcgtgctggagaagtccctcaagaagaacgacgccgtggtcgccatcatgaaagacctgctcgactccgtcaagagcttcgagaactacatcaag |
| | gccttcttcggcgagggcaaggagaccaacagggacgagtccttctacggcgacttcgtcctggcctacgacatcctgctcaaggtggaccacatctacgacgccatcaggaactacgtgac |
| | ccagaagccgtacagcaaggacaagttcaagctctacttccagaacccacagttcatgggcggctgggacaaggacaaggagaccgactacagggccaccatcctgcgctacggcagcaagt |
| | actacctcgccatcatggacaagaagtacgccaagtgcctgcagaagatcgacaaggacgacgtgaacggcaactacgagaagatcaactacaagctgctcccgggcccaaacaagatgctc |
| | cccaaggtcttcttctccaagaagtggatggcctactacaaccctagcgaggacatccagaagatctacaagaacggcaccttcaagaagggcgacatgttcaacctgaacgactgccacaa |
| | gctcatcgacttcttcaaggactccatcagcaggtacccgaagtggtccaacgcctacgacttcaacttcagcgagaccgagaagtacaaggacatcgccggcttctaccgcgaggtcgagg |
| | agcagggctacaaggtgtccttcgagtccgcctccaagaaggaagtggacaagctggtggaggaaggcaagctgtacatgttccagatctacaacaaggacttctccgacaagagccacggc |
| | accccaaacctgcacaccatgtacttcaagctgctgttcgacgagaacaaccacggccagatcaggctctccggcggcgccgagctgttcatgcgtagagccagcctgaagaaagaggagct |
| | ggtggtccacccagcaaacagccctatcgccaacaagaaccccgacaaccctaagaagaccaccaccctgtcctacgacgtgtacaaggacaagaggttcagcgaggaccagtacgagctcc |
| | acatccccatcgccatcaacaagtgccctaagaacatcttcaagatcaacaccgaggtccgcgtgctgctcaagcacgacgacaacccatacgtgatcggcatcgacaggggcgagcgcaac |
| | ctgctctacatcgtggtcgtggacggcaagggcaacatcgtcgagcagtactccctcaacgagatcatcaacaacttcaacggcatcaggatcaagaccgactaccacagcctgctcgacaa |
| | gaaggagaaggagaggttcgaggcccgccagaactggacctccatcgagaacatcaaggagctgaaggccggctacatcagccaggtcgtgcacaagatctgcgagctcgtcgagaagtacg |
| | acgccgtgatcgccctggaggacctcaactccggcttcaagaacagccgcgtcaaggtggagaagcaggtgtaccagaagttcgagaagatgctgatcgacaagctcaactacatggtcgac |
| | aagaagtccaacccctgcgcaaccggcggcgccctgaagggctaccagatcaccaacaagttcgagagcttcaagtccatgagcacccagaacggcttcatcttctacatcccggcctggct |
| | cacctccaagatcgacccaagcaccggcttcgtgaacctgctcaagaccaagtacacctccatcgccgacagcaagaagttcatctccagcttcgacaggatcatgtatgtgcccgaggaag |
| | acctgttcgagttcgccctcgactacaagaacttctcccgcaccgacgccgattacatcaagaagtggaagctgtacagctacggcaacaggatccgcatcttcaggaaccctaagaagaac |
| | aacgtcttcgactgggaggaagtgtgcctgacctccgcctacaaggagctgttcaacaagtacggcatcaactaccagcagggcgacatcagggccctgctctgcgagcagagcgacaaggc |
| | cttctactccagcttcatggccctgatgtccctgatgctccagatgaggaacagcatcaccggccgcaccgacgtcgacttcctcatctccccggtgaagaacagcgacggcatcttctacg |
| | actccaggaactacgaggcccaggagaacgccatcctgccaaagaacgccgacgccaacggcgcctacaacatcgcaaggaaggtgctctgggcaatcggccagttcaagaaggccgaggac |
| | gagaagctggacaaggtgaagatcgccatctccaacaaggagtggctcgagtacgcccagacctctgtcaagcacaagaggccagcagcaaccaagaaggcgggccaggccaagaagaagaa |
| | gggcagctacccgtacgacgtcccagactacgcctacccctacgacgtgcctgactacgcttacccatacgatgtgccagactacgcctga |
| SEQ ID NO: 47_ | gatcgttcaaacatttggcaataaagtttcttaagattgaatcctgttgccggtcttgcgatgattatcatataatttctgttgaattacgttaagcatgtaataattaacatgtaatgcat |
| Nopaline | gacgttatttatgagatgggtttttatgattagagtcccgcaattatacatttaatacgcgatagaaaacaaaatatagcgcgcaaactaggataaattatcgcgcgcggtgtcatctatgt |
| synthase | tactag |
| polyadenylation | |
| sequence | |
| SEQ ID NO: 48_ | ATGGCCACGGCGGCGGCGGCGGCGGCGACGATGCGTCTGTCTTCCCCGTTCAGGGCCCCGCCTCTCCGTCCTCCCTGCCACCGCGTCGTCCCGT |
| Traes- | CGATGCGGGGCCCCAGGCGGGCCGGGCTGGCCGTCTCGGCGGCCGCCGCCGGGTCGCCCCCCACCGTGCTCGTCACCGGCGCCGGAGGACGG |
| CS1B02G037100 | ACAGGCCAAATTGTGTACAAGAAGCTGAAGGAGAGGGCAGACCAGTTTGTGGCCAGAGGGCTGGTCAGGACGCCGGACAGCAAGGGTAAGATAGG |
| (TaAt5g02240- | CGGCGGCGACGACGTGTTCATCGGCGACATCAGGGATCCTGGGAGCATTGCTCCGGCGATTGAGGGCATCGACGCGCTCATCATCCTCACCAGTG |
| like) CDS | GGGTCCCGAAGATGAAGCCCGGGTTCGATCCTAGCAAGGGTGGACGGCCGGAGTTTTACTTTGAGGAAGGGTCTGATCCTGAGCAGGTGGATTGG |
| | ATAGGCCAAAAGAACCAAATTGATGCTGCCAAGAGCATTGGTGTAAAGCAGATAGTTTTGGTTGGATCCATGGGGGGAACAGATATCAACCATCCATT |
| | AAACAAGCTTGGGAATGGGAATATACTGGTGTGGAAACGGAAGGCAGAGCAGTACCTAGCGGACTCTGGTCTACCATACACAATTATAAGGGCTGG |
| | AGGACTACAAGACAAAGATGGTGGGGTGCGTGAGTTGATTGTTGGAAAGGATGACGAGATCTTGAAGACGGAAACAAAAACTATTGCCAGGGCAGA |
| | TGTTGCAGAAGTTTGCATACAGGCCTTGCTATTTGAGGAAGCAAAGTTCAAGGCGTTTGATCTGGCTTCAAAACCTGAAGGTGAAGGAACACCGATG |
| | ACAGATTTTAAGTCTGTTTTTGCACAAATTGCTACTCGCTTCTAA |
| SEQ ID NO: 49_ | MATAAAAAATMRLSSPFRAPPLRPPCHRVVPSMRGPRRAGLAVSAAAAGSPPTVLVTGAGGRTGQIVYKKLKERADQFVARGLVRTPDSKGKIGGGDDVF |
| Traes- | IGDIRDPGSIAPAIEGIDALIILTSGVPKMKPGFDPSKGGRPEFYFEEGSDPEQVDWIGQKNQIDAAKSIGVKQIVLVGSMGGTDINHPLNKLGNGNILVWKRK |
| CS1B02G037100 | AEQYLADSGLPYTIIRAGGLQDKDGGVRELIVGKDDEILKTETKTIARADVAEVCIQALLFEEAKFKAFDLASKPEGEGTPMTDFKSVFAQIATRF |
| (TaAt5g02240- | |
| like) | |
| SEQ ID NO: 50_ | CGCCTTAGATGCCGTCGCCTCCTAGCCATAAGCGTCAGAAGGCGGTATATAGGTTATCTCCGCCGGAAAAAGGGTAAGGTTTGCCTCGAGAATGTTT |
| Traes- | TGTTTGACTATATCCTATGTGGAACCAAGGCTAGGTTTGCATGCAATGAAAAAATGTAATTGTAGGAGGAGAAGGGAGGGTGGTAGATGTGCGCGAC |
| CS1B02G037100 | AACAACACACACGCCATATATATATAGTTGGAGGAGGTGGAAAACAAGGAGGCGATAAGAAAAAGGGGGGGAATATGGAGAATGTTTGAAATAGCGT |
| (TaAt5g02240- | CCAACCCGAGTTTCCATTATTAATTGTCGGCCAAAAAAAGAAGGCATGTTACTCATGATGGTAGTTTCAAGGGAAGTGGAGCCTCCCAGCTAGTTTGA |
| like) genomic | ACACTTAAAAAACAAGCTACAACAGAAAGTAGTTTCAAGGGGAGTGGAGCCTAAAACTTGCATTTAAAAAGGAAATGCGCACACTAAGCCGAAAGTGT |
| | GAACACTTTGTGCCGACATTTCCGAGGAAGTAGGTTTTATGCGCATAACATTTTTTTTGAGACAATTTTATGCGCATAACTAGGGTTGACTGCTGTGG |
| | GGAAGATGAAATTTGAACTGCCATTGTGACGCAAAGATTTGGTGATCCAAATTTGTATATGGACCATGCCTGTAGGTTGGGCTAAGTATACTTTTCGA |
| | AGAAAATAGGCCCATGTTAACTTGGGAAGTGGGCCTAGGCCCACAAACCTGAAATCCCTGTGAAGCCCACATTCACACGCGGGCCCACCGTCGAAG |
| | CGATAAGGAGAGTGACAGCCGCTCAGCCCGCCGGCACACCCGACAACAACTCTCGCTTCACTCGCCGGAGGGGGACGGAGCAAATATGGCCACG |
| | GCGGCGGCGGCGGCGGCGACGATGCGTCTGTCTTCCCCGTTCAGGGCCCCGCCTCTCCGTCCTCCCTGCCACCGCGTCGTCCCGTCGATGCGGG |
| | GCCCCAGGCGGGCCGGGCTGGCCGTCTCGGCGGCCGCCGCCGGGTCGCCCCCCACCGTGCTCGTCACCGGCGCCGGAGGACGGACAGGTAGAT |
| | ACTACACATCCCGCTAGCTTCTTTACCTTCGCGTCCTGTAGCCGCACATTGTTGCACGATGCCGCCTTCTCTTGCTGTTCCAATGGGATTCAGTTTGC |
| | ATTTGTGGGAGAATTTACCGGCGGAGGCGGCGGTTTCTTGGAATCAAGATGCTTCACAACCTATGGAACCTCCTATTGATTTAAAATTTACACTGCCG |
| | TTTACGTGCTGTGATCCGGCGTGCGCCTCTGACCCTTGCGCTGTGGTGACCCAGGCCAAATTGTGTACAAGAAGCTGAAGGAGAGGGCAGACCAGT |
| | TTGTGGCCAGAGGGCTGGTCAGGACGCCGGACAGCAAGGGTAAGATAGGCGGCGGCGACGACGTGTTCATCGGCGACATCAGGGATCCTGGGAG |
| | CATTGCTCCGGCGATTGAGGGCATCGACGCGCTCATCATCCTCACCAGTGGGGTCCCGAAGATGAAGCCCGGGTTCGATCCTAGCAAGGGTGGAC |
| | GGCCGGAGTTTTACTTTGAGGAAGGGTCTGATCCTGAGCAGGTAACCGGCAAAAATCGCTTGGCAATGACATTAGTCACATTAGCAATGACAACTTA |
| | TGCATTTCTTTTAGAGTTAATTAATGTCCTTTGCTGTTTTCCCTTGATTTAAGGTGGATTGGATAGGCCAAAAGAACCAAATTGATGCTGGTAAATCTTC |
| | ACCTTCTTCTTGTTCTGTTAGAGCATTATACAATGTTTAGCATGTTCATTGAAACGATTGATTGGTGAAGCATCGATGTGTCATTTTCTCTCCGGTCGA |
| | CCTTTTTCTTGTCTGCTCTCTAAGTTTCCCATCTTTAATTATCCATGCAGCCAAGAGCATTGGTGTAAAGCAGATAGTTTTGGTTGGATCCATGGGCGG |
| | AACAGATATCAACCATCCATTAAACAAGCTTGGGAATGGGAATATACTGGTATATAGCATGCTCGACGATTTTTGTATATGTTCTAATTCCCTATACTTG |
| | TGTGTGCATAAACATATAATATGTACCATTTTGAAACTTAGAATGGCCAATTGCTCTTCTAATTTGCAGGTGTGGAAACGGAAGGCAGAGCAGTACCTA |
| | GCGGACTCTGGTCTACCATACACAATTATAAGGTTATAACACAGACTCATTCGGTAGTGCTATTCTTGCCATGTTCTGCAAAGATTTTTTTTTTGTCACA |
| | TTTGAGTTATGTAATAGGCTGACAGTCACTGTTTTCTCCAAAGATGCACAGTCATAGAAATAATTTTATATATGTGATGAATGAAGAAAACAACAAAACA |
| | ACAGAAGTGCATTATGGAGTATGAAGCACCACAAAGAACATTAATTTTGTATAAGAAATAAAAAATTCCAATTTATTTCATAGGGACATTAATCGTTGGA |
| | GTATTTTCCACCAACAATCGTGTGCTGATAAAATACCTTTCCCATATCCCATCTCTTTCCAGGGCTGGAGGACTACAAGACAAAGATGGTGGGGTGCG |
| | TGAGTTGATTGTTGGAAAGGATGACGAGATCTTGAAGACGGAAACAAAAACTATTGCCAGGGCAGATGTTGCAGAAGTTTGCATACAGGTACCTTTAT |
| | AGAACAAAACACATGTTTCTGTAGATAAACTACTGCTCAATATTTTTTTCATCATCTACCAAATATATTTCTTGCATCTATTTATGTCTTACTAGCTAGTG |
| | GTCATACCACTGATCCATCTGTGCCTTTCCTGAGTAGGCCTTGCTATTTGAGGAAGCAAAGTTCAAGGCGTTTGATCTGGCTTCAAAACCTGAAGGTG |
| | AAGGAACACCGATGACAGATTTTAAGTCTGTTTTTGCACAAATTGCTACTCGCTTCTAAGAGGACAAGATCATTCTGGCCAATGAAATGGGACAGTAT |
| | CTTTAATTAGGCGATATGGAATTTGTACTGGGTCAGGTTAGTGTTCTGTTTATTAATTGAGGAAATGCAACATTTTATTCTACGCACTATTATTCCATAG |
| | TAAATTTTGTGTTTATCTCTTTTCTTTGTGGTCAA |
| SEQ ID NO: 51_ | ATGGCCACGGCGGCGGCGGCGGCGGCGACGATGCGTCTGTCTTCCCCGTTCAGGGCCCCGCCTCTCCGTCCTCCCTGCCACCGCGTCGTCCCGT |
| TaAt5g02240- | CGATGCGGGGCCCCAGGCGGGCCGGGCTGGCCGTCTCGGCGGCCGCCGCCGGGTCGCCCCCCACCGTGCTCGTCACCGGCGCCGGAGGACGG |
| like CDS | ACAGGCCAAATTGTGTACAAGAAGCTGAAGGAGAGGGCAGACCAGTTTGTGGCCAGAGGGCTGGTCAGGACGCCGGACAGCAAGGGTAAGATAGG |
| | CGGCGGCGACGACGTGTTCATCGGCGACATCAGGGATCCTGGGAGCATTGCTCCGGCGATTGAGGGCATCGACGCGCTCATCATCCTCACCAGTG |
| | GGGTCCCGAAGATGAAGCCCGGGTTCGATCCTAGCAAGGGTGGACGGCCGGAGTTTTACTTTGAGGAAGGGTCTGATCCTGAGCAGGTGGATTGG |
| | ATAGGCCAAAAGAACCAAATTGATGCTGCCAAGAGCATTGGTGTAAAGCAGATAGTTTTGGTTGGATCCATGGGCGGAACAGATATCAACCATCCATT |
| | AAACAAGCTTGGGAATGGGAATATACTGGTGTGGAAACGGAAGGCAGAGCAGTACCTAGCGGACTCTGGGACATTAATCGTTGGAGTATTTTCCACC |
| | AACAATCGTGTGCTGATAAAATACCTTTCCCATATCCCATCTCTTTCCAGGGCTGGAGGACTACAAGACAAAGATGGTGGGGTGCGTGAGTTGATTGT |
| | TGGAAAGGATGACGAGATCTTGAAGACGGAAACAAAAACTATTGCCAGGGCAGATGTTGCAGAAGTTTGCATACAGGCCTTGCTATTTGAGGAAGCA |
| | AAGTTCAAGGCGTTTGATCTGGCTTCAAAACCTGAAGGTGAAGGAACACCGATGACAGATTTTAAGTCTGTTTTTGCACAAATTGCTACTCGCTTCTAA |
| SEQ ID NO: 52_ | MATAAAAAATMRLSSPFRAPPLRPPCHRVVPSMRGPRRAGLAVSAAAAGSPPTVLVTGAGGRTGQIVYKKLKERADQFVARGLVRTPDSKGKIGGGDDVF |
| TaAt5g02240- | IGDIRDPGSIAPAIEGIDALIILTSGVPKMKPGFDPSKGGRPEFYFEEGSDPEQVDWIGQKNQIDAAKSIGVKQIVLVGSMGGTDINHPLNKLGNGNILVWKRK |
| like | AEQYLADSGTLIVGVFSTNNRVLIKYLSHIPSLSRAGGLQDKDGGVRELIVGKDDEILKTETKTIARADVAEVCIQALLFEEAKFKAFDLASKPEGEGTPMTDF |
| | KSVFAQIATRF |
| SEQ ID NO: 53_ | GGAGTCGTGTCTTATTTGGTGGCGTTTGTCGACTATCTCCTGTGTGGAGCCAATGCTTGGCTCGCACGCAAGTCGAAAAGAACTGCAATGGTAGGAG |
| TaAt5g02240- | GAGAAGGGAGAGGGTGTGGTAGATGCGCACGACAACAACAACACACGACAAATATATATAGTTGGAGTAAGTGGAAAACAAGGACGCGACAAGAAA |
| like genomic | GGGGGAGAATGTTCGAAATAGCGTCCAACCTAAGTTCAGAGAATTAATTGTCAACCAAAAATTGGGCAAGGGCTCAGTATACCGGCCTAGAAGTGAA |
| | AATTGCGATCAAAAGTTTGCACCGCCGAAGAAGAGAAAAGCAAAAACTTCTCATATTTTTCAAAGGATTGAACATTTTTTATTGTCATGTTATCCCGTA |
| | GGCAGGATATTGACCTAGCAACTTTGGTGTAGCCTATTGTATTACTCATATTCATCGAGCAGTATATTATTGAAGGGGCTTTTATTGACTCTAAATGTA |
| | GCATTGTTGTAATGCATTTGCATAATTAAGGGTGTGTTTGGTTGTGTGGCTAAGGATAGCCACTTTTTAAGTGGTTGCCATGTGTTTGATTTTAAAATTT |
| | GTGTAGTTCAGGATAGCCATATCCAAGGAATATTCCATAAAATGTGGCACAGCGGAAGCGCCAAAAAGTGATGGGTAAGGTCAACCACCCTATGCTT |
| | GCATGCTCACCTCACCATCTGAATCATTTACAACCTGTTCTGACCCCCTCCCCCAAAGGAATTTTATCAGGCTTTTGGTGAGGGCATTCGCATGTCTG |
| | CACTTGTCATCCAATGAACTGTGACGAATACTAGGGTGTTAGAAAGGAAACGGCAACTCATGAAATATAACCGACAAACATATATATCTATAAATCATG |
| | CAACAAAGAAACCGAAAGATTACAACAAAAACCATATATGGATCTGATCATAAAGGGGCAATTCATCACACGGAAACAAACACACCGAGAATTACATC |
| | AGATGGAACACAATCATGTAGAGCAGCGCATGTGACCATTGATTGAAAAGCAAGGGAGAGAAGGAAAAGCAAGGGAGAGAAGATGCCAACTAGCTA |
| | CTGTTATGGACCCATAGTTCAAAGAGGACTATTCACACATGGTCATGGTGGCAACGAGGTTGCTGGAGATGCATCCGGCGATGATTTATCCCTCCAG |
| | GAAAGTACCGGAAAAAGGTCTCCGGATTGGATCATCGCGAAACAAGTGGTGGCGACAGCAGAAAAATCCTTGATAAACAAATCACGTGAGAGAGCTC |
| | CGAAATTAGGGAGCATACTGAATTTAAAGATACTAGATGATACCCCGCATGTTATTGTGAACTCTGGTCTACCATACACAATTATAAGGTTATAACACA |
| | GACTCATTCGGTAGTGCTATTTGTGGTATGAGACTTATTCTTGCCATGTTCTGCAAAGAATTTTTTTTGTCACATTTGAGTTATGTAATAGGCTGACAGT |
| | CACTGTTTTCTCCAAAGACGCACAGTCATAAAAGTAATTTGAAAAAACTGTGCATTTTGAAAAAAATGTTCGCGGAATCAAAAAATGTTTGTGATTTTAC |
| | AAAAAAATATTCACATATTCAGAAAATGTTGGCGTATTCAGAAAATGTTATGGAATTGAGAAATTTTCTGCAAAGTTTAAAATTTTTCATGTATTTTAAAA |
| | AGGCTTCACAAAATTTAAAAAATGTCCATTCATTACAAAAATGATCCTCGATTCAAAAATGCTCATTCAAATAAAAAAATGTTCATGAATTACAAAATTAA |
| | TTCCCCCAATTTTTAAAAGTGTTTCGCTGATTCAAAACAAATATTCACAGGCTTAAAAGATGTTCACAATTCCTAGAAATGTACACAAATTTGTAAAAAA |
| | TCCTCGTCAATTTAAAAAATGTTCGTCAATTAAAAAAATGTAATGTAGTAGTAGTATGTTGAGTAGTCCAACCGAAGGATATTAATTCTCATCTCAGTCA |
| | TCACTCCTCTTCCTCGGATCACCGGCGATGGCAATGGCATTGTCCCTCCCCACCACGGTCTTGCTCCTCCTCCTCCTCCTCCTCCTCCCCGTCGCCT |
| | TAGATGCCGTCGCCTCCTAGCCATAAGCGTCAGAAGGCGGTATATAGGTTATCTCCGCCGGAAAAAGGGTAAGGTTTGCCTCGAGAATGTTTTGTTT |
| | GACTATATCCTATGTGGAACCAAGGCTAGGTTTGCATGCAATGAAAAAATGTAATTGTAGGAGGAGAAGGGAGGGTGGTAGATGTGCGCGACAACAA |
| | CACACACGCCATATATATATAGTTGGAGGAGGTGGAAAACAAGGAGGCGATAAGAAAAAGGGGGGGGGGGAATATGGAGAATGTTTGAAATAGCGT |
| | CCAACCCGAGTTTCCATTATTAATTGTCGGCCAAAAAAAGAAGGCATGTTACTCATGATGGTAGTTTCAAGGGAAGTGGAGCCTCCCAGCTAGTTTGA |
| | ACACTTAAAAAAACAAGCTACAACAGAAAGTAGTTTCAAGGGGAGTGGAGCCTAAAACTTGCATTTAAAAAGGAAATGCGCACACTAAGCCGAAAGTG |
| | TGAACACTTTGTGCCGACATTTCCGAGGAAGTAGGTTTTATGCGCATAACATTTTTTTGAGACAATTTTATGCGCATAACTAGGGTTGACTGCTGTGG |
| | GGAAGATGAAATTTGAACTGCCATTGTGACGCAAAGATTTGGTGATCCAAATTTGTATATGGACCATGCCTGTAGGTTGGGCTAAGTATACTTTTCGA |
| | AGAAAATAGGCCCATGTTAACTTGGGAAGTGGGCCTAGGCCCACAAACCTGAAATCCCTGTGAAGCCCACATTCACACGCGGGCCCACCGTCGAAG |
| | CGATAAGGAGAGTGACAGCCGCTCAGCCCGCCGGCACACCCGACAACAACTCTCGCTTCACTCGCCGGAGGGGGACGGAGCAAATATGGCCACG |
| | GCGGCGGCGGCGGCGGCGACGATGCGTCTGTCTTCCCCGTTCAGGGCCCCGCCTCTCCGTCCTCCCTGCCACCGCGTCGTCCCGTCGATGCGGG |
| | GCCCCAGGCGGGCCGGGCTGGCCGTCTCGGCGGCCGCCGCCGGGTCGCCCCCCACCGTGCTCGTCACCGGCGCCGGAGGACGGACAGGTAGAT |
| | ACTACACATCCCGCTAGCTTCTTTACCTTCGCGTCCTGTAGCCGCACATTGTTGCACGATGCCGCCTTCTCTTGCTGTTCCAATGGGATTCAGTTTGC |
| | ATTTGTGGGAGAATTTACCGGCGGAGGCGGCGGTTTCTTGGAATCAAGATGCTTCACAACCTATGGAACCTCCTATTGATTTAAAATTTACACTGCCG |
| | TTTACGTGCTGTGATCCGGCGTGCGCCTCTGACCCTTGCGCTGTGGTGACCCAGGCCAAATTGTGTACAAGAAGCTGAAGGAGAGGGCAGACCAGT |
| | TTGTGGCCAGAGGGCTGGTCAGGACGCCGGACAGCAAGGGTAAGATAGGGGGGGGCGACGACGTGTTCATCGGCGACATCAGGGATCCTGGGAG |
| | CATTGCTCCGGCGATTGAGGGCATCGACGCGCTCATCATCCTCACCAGTGGGGTCCCGAAGATGAAGCCCGGGTTCGATCCTAGCAAGGGTGGAC |
| | GGCCGGAGTTTTACTTTGAGGAAGGGTCTGATCCTGAGCAGGTAACCGGCAAAAATCGCTTGGCAATGACATTAGTCACATTAGCAATGACAACTTA |
| | TGCATTTCTTTTAGAGTTAATTAATGTCCTTTGCTGTTTTCCCTTGATTTAAGGTGGATTGGATAGGCCAAAAGAACCAAATTGATGCTGGTAAATCTTC |
| | ACCTTCTTCTTGTTCTGTTAGAGCATTATACAATGTTTAGCATGTTCATTGAAACGATTGATTGGTGAAGCATCGATGTGTCATTTTCTCTCCGGTCGA |
| | CCTTTTTCTTGTCTGCTCTCTAAGTTTCCCATCTTTAATTATCCATGCAGCCAAGAGCATTGGTGTAAAGCAGATAGTTTTGGTTGGATCCATGGGGGG |
| | AACAGATATCAACCATCCATTAAACAAGCTTGGGAATGGGAATATACTGGTATATAGCATGCTCGACGATTTTTGTATATGTTCTAATTCCCTATACTTG |
| | TGTGTGCATAAACATATAATATGTACCATTTTGAAACTTAGAATGGCCAATTGCTCTTCTAATTTGCAGGTGTGGAAACGGAAGGCAGAGCAGTACCTA |
| | GCGGACTCTGGTCTACCATACACAATTATAAGGTTATAACACAGACTCATTCGGTAGTGCTATTCTTGCCATGTTCTGCAAAGATTTTTTTTTTGTCACA |
| | TTTGAGTTATGTAATAGGCTGACAGTCACTGTTTTCTCCAAAGATGCACAGTCATAGAAATAATTTTATATATGTGATGAATGAAGAAAACAACAAAACA |
| | ACAGAAGTGCATTATGGAGTATGAAGCACCACAAAGAACATTAATTTTGTATAAGAAATAAAAAATTCCAATTTATTTCATAGGGACATTAATCGTTGGA |
| | GTATTTTCCACCAACAATCGTGTGCTGATAAAATACCTTTCCCATATCCCATCTCTTTCCAGGGCTGGAGGACTACAAGACAAAGATGGTGGGGTGCG |
| | TGAGTTGATTGTTGGAAAGGATGACGAGATCTTGAAGACGGAAACAAAAACTATTGCCAGGGCAGATGTTGCAGAAGTTTGCATACAGGTACCTTTAT |
| | AGAACAAAACACATGTTTCTGTAGATAAACTACTGCTCAATATTTTTATCATCATCTACCAAATATATTTCTTGCATCTATTTATGTCTTACTAGCTAGTG |
| | GTCATACCACTGATCCATCTGTGCCTTTCCTGAGTAGGCCTTGCTATTTGAGGAAGCAAAGTTCAAGGCGTTTGATCTGGCTTCAAAACCTGAAGGTG |
| | AAGGAACACCGATGACAGATTTTAAGTCTGTTTTTGCACAAATTGCTACTCGCTTCTAAGAGGACAAGATCATTCTGGCCAATGAAATGGGACAGTAT |
| | CTTTAATTAGGCGATATGGAATTTGTACTGGGTCAGGTTAGTGTTCTGTTTATTAATTGAGGAAATGCAACATTTTATTCTACGCACTATTATTCCATAG |
| | TAAATTTTGTGTTTATCTCTTTTCTTTGTGGTCAATGGATTCTGCATAAAATGTTTATGGCCCTTCTGTTACCTGGTTGATCTGTCCTAATTTGGATGTAC |
| | ACACTTAGTATTTGCAGTTAGTTTATTTTGTAACTAAAGGTCGGACATGCTTGGGGTCATGCGGTCATGATGTTCTGGTTGATCTAGTTTTAAGTTAAT |
| | CCATTTGCTAGTTTAAACAACGTTTAGGGTTATCCATGTTGCGTATTTATAGTCCCACCAAATCGTTGGTTAGTATAATCTGGATTGTAAATTTGTTAGG |
| | GCTATAAACACCCAACTTCACATGCTCCTTCTGTAGCAAAAGTAGAAGTGGCATGTCCTCTCCTTCCAGATTGTCTCCGATGCTCTGCCAACAAAACA |
| | ACAACAACAACAACAAC |
| SEQ ID NO: 54_ | CTTCACTCGCCGGAGGGGGACGG |
| Target 70f | |
| SEQ ID NO: 55_ | GGAGGGGGACGGAGCAAATATGG |
| Target 81f | |
| SEQ ID NO: 56_ | CTTCACTCGCCGGAGGGGGAgttttagagctagaaatagcaagttaaaataaggctagtccgttatcaacttgaaaaagtggcaccgagtcggtgcttttttt |
| gRNA_70f | |
| SEQ ID NO: 57_ | GGAGGGGGACGGAGCAAATAgttttagagctagaaatagcaagttaaaataaggctagtccgttatcaacttgaaaaagtggcaccgagtcggtgcttttttt |
| gRNA_81f | |
| SEQ ID NO: 58_ | atgcctcgct tctcctccac cacgccaatg tcgccacccc gcctcctcct ccggctcggc gcccgccact cctcctccac ctctcatccc |
| RFL79 gene | tcacgcatct gggatcccca cgccgccttc gccgctgcga cgcagcgggc gcgctctggc acgctcacca cggaggacgc acaccacctg |
| | tttgatgaat tgctgcggca gggcaatcct gtccaggagc gtcccttgac taactttctg gctgccctcg cccgcgcgcc cgcgtccgca |
| | ttctgcagcg atggccctgc cctggccgtc gccctcttcg gccgtttgtc ccgaggcgcc ggacgacggg tggcgcagcc aaatgtcttc |
| | acctatggcg tcctcatgga ctgctgctgc cgtgcgcgcc gcctggatct agcgatcgcc ttcttcgccc gtctcctcaa gacgggactg |
| | gaggcaaacc aagtcatctt ctgcaccctc ctcaagggac tctgccacgc aaagcgctca gatgaggctt tggacgtggt gcttcacagg |
| | atgcctgagc taggctgcac ccccaacgtg gtggcctata ccacggtcat ccacggcttc ttgaaggaag gccaagtagg caaggcatgc |
| | aatctattcc atggaatggc gcagcagggc gttgcgcctg atgtggtgac atataactcg gttatcgatg cgttgtgcaa ggccagagca |
| | atggacaagg cagagtattt ccttcgtgaa atggttgata atggtgtcgt acctaataat gtgacatata atagcctcat ccatggatat |
| | tcctctttgg gccatcagaa ggaggctgtt agggtgctga aagaaatgac aagacagggt atcataccag atgtcattac ctgcacctca |
| | ctcatgacct tcctttgcaa gaatggaaaa agcaaggaag ctgcagaaat ttttgattca atggccacga agggcctgaa acatgacgcc |
| | gtttcatatg ctattctcct tcatgggtat gccactgaag gatgcttggt tgatatgatt aatctcttca attcgatgga cagagactgt |
| | attctaccta actgtcatat cttcaacata ctgatttatg catatgctaa atctgggaag cttgataagg ctatgcttat atttagagat |
| | atgcagaaac aaggagtgag tgatacagga gtacgacagg caaccttaat ttatggttct tgtaaaaagg gtcggttgga cgatgctatg |
| | ataaagttta atcagatggt tgatacagga gtacgacagg gcacagctgt ttatggttct ctaatccagg gtttttgtac acacggcgat |
| | ttggtgaaag gaaaggaatt ggttactgaa atgatgaaca aaggtatacc tcctcctgac attatgttct tccattcaat catgcagaac |
| | ctatgcacag aaggaagggt agtagaagca cgggatatcc ttggcttgat agcacacata ggtatgaggc ctaatgtttg cacatttaat |
| | atactgattg gtggatactg cctagtccgc aagatggagg atgcctcaaa aatatttcat gatatgatgt catatggttt agaaccttct |
| | ggacttaaac ctacaacttt taattacaac atcatactgg atggattatt tctggctgga cgaactgttg ctgcaaagga aaagtttgat |
| | gagatggttg aatctggagt aagtatgtgc atcagtactt actctatagt tcttcgtgga ctttgtagaa ataattgtag cggcgaagcc |
| | atcacgctat tccagacatt aagcgcaatg gatgtgaaat tcaatattag aattgtcaat atcatgattg atgccttctt cagggttcag |
| | cgaaagcaag aagctaagga tttgtttgct gcaataacag ccaatgggtt ggttgctaat gtttttacct acagcctaat gatgacaaat |
| | cttataaaag aagggtcagt ggaagaggct gacacactct ttttatcgat ggagatgagc ggctgtactt cgaactcgtg gatgttaaat |
| | cttattatca gagggttgct ggaaaaagga gagatagtca aggctggatg ttatatgtct aaagttgatg ccaagagcta ctcacttgaa |
| | gctaaaactg tttcgttgct gatctatctc ttttcaggga aagggaaata cagagaacac ataagattgc tacctacaaa gtatcagttt |
| | ctcgaagaag cagccacagt tgaatggtttgctatatag |
| SEQ ID NO: 59_ | GAGTCGTGTCTTATTTGGTGGCGTTTGTCGACTATCTCCTGTGTGGAGCCAATGCTTGGCTCGCACGCAAGTCGAAAAGAACTGCAATGGTAGGAGG |
| At5g02240-like | AGAAGGGAGAGGGTGTGGTAGATGCGCACGACAACAACAACACACGACAAATATATATAGTTGGAGTAAGTGGAAAACAAGGACGCGACAAGAAAG |
| promoter | GGGGAGAATGTTCGAAATAGCGTCCAACCTAAGTTCAGAGAATTAATTGTCAACCAAAAATTGGGCAAGGGCTCAGTATACCGGCCTAGAAGTGAAA |
| | ATTGCGATCAAAAGTTTGCACCGCCGAAGAAGAGAAAAGCAAAAACTTCTCATATTTTTCAAAGGATTGAACATTTTTTATTGTCATGTTATCCCGTAG |
| | GCAGGATATTGACCTAGCAACTTTGGTGTAGCCTATTGTATTACTCATATTCATCGAGCAGTATATTATTGAAGGGGCTTTTATTGACTCTAAATGTAG |
| | CATTGTTGTAATGCATTTGCATAATTAAGGGTGTGTTTGGTTGTGTGGCTAAGGATAGCCACTTTTTAAGTGGTTGCCATGTGTTTGATTTTAAAATTTG |
| | TGTAGTTCAGGATAGCCATATCCAAGGAATATTCCATAAAATGTGGCACAGCGGAAGCGCCAAAAAGTGATGGGTAAGGTCAACCACCCTATGCTTG |
| | CATGCTCACCTCACCATCTGAATCATTTACAACCTGTTCTGACCCCCTCCCCCAAAGGAATTTTATCAGGCTTTTGGTGAGGGCATTCGCATGTCTGC |
| | ACTTGTCATCCAATGAACTGTGACGAATACTAGGGTGTTAGAAAGGAAACGGCAACTCATGAAATATAACCGACAAACATATATATCTATAAATCATGC |
| | AACAAAGAAACCGAAAGATTACAACAAAAACCATATATGGATCTGATCATAAAGGGGCAATTCATCACACGGAAACAAACACACCGAGAATTACATCA |
| | GATGGAACACAATCATGTAGAGCAGCGCATGTGACCATTGATTGAAAAGCAAGGGAGAGAAGGAAAAGCAAGGGAGAGAAGATGCCAACTAGCTAC |
| | TGTTATGGACCCATAGTTCAAAGAGGACTATTCACACATGGTCATGGTGGCAACGAGGTTGCTGGAGATGCATCCGGCGATGATTTATCCCTCCAGG |
| | AAAGTACCGGAAAAAGGTCTCCGGATTGGATCATCGCGAAACAAGTGGTGGCGACAGCAGAAAAATCCTTGATAAACAAATCACGTGAGAGAGCTCC |
| | GAAATTAGGGAGCATACTGAATTTAAAGATACTAGATGATACCCCGCATGTTATTGTGAACTCTGGTCTACCATACACAATTATAAGGTTATAACACAG |
| | ACTCATTCGGTAGTGCTATTTGTGGTATGAGACTTATTCTTGCCATGTTCTGCAAAGAATTTTTTTTGTCACATTTGAGTTATGTAATAGGCTGACAGTC |
| | ACTGTTTTCTCCAAAGACGCACAGTCATAAAAGTAATTTGAAAAAACTGTGCATTTTGAAAAAAATGTTCGCGGAATCAAAAAATGTTTGTGATTTTACA |
| | AAAAAATATTCACATATTCAGAAAATGTTGGCGTATTCAGAAAATGTTATGGAATTGAGAAATTTTCTGCAAAGTTTAAAATTTTTCATGTATTTTAAAAA |
| | GGCTTCACAAAATTTAAAAAATGTCCATTCATTACAAAAATGATCCTCGATTCAAAAATGCTCATTCAAATAAAAAAATGTTCATGAATTACAAAATTAAT |
| | TCCCCCAATTTTTAAAAGTGTTTCGCTGATTCAAAACAAATATTCACAGGCTTAAAAGATGTTCACAATTCCTAGAAATGTACACAAATTTGTAAAAAAT |
| | CCTCGTCAATTTAAAAAATGTTCGTCAATTAAAAAAATGTAATGTAGTAGTAGTATGTTGAGTAGTCCAACCGAAGGATATTAATTCTCATCTCAGTCAT |
| | CACTCCTCTTCCTCGGATCACCGGCGATGGCAATGGCATTGTCCCTCCCCACCACGGTCTTGCTCCTCCTCCTCCTCCTCCTCCTCCCCGTCGCCTT |
| | AGATGCCGTCGCCTCCTAGCCATAAGCGTCAGAAGGCGGTATATAGGTTATCTCCGCCGGAAAAAGGGTAAGGTTTGCCTCGAGAATGTTTTGTTTG |
| | ACTATATCCTATGTGGAACCAAGGCTAGGTTTGCATGCAATGAAAAAATGTAATTGTAGGAGGAGAAGGGAGGGTGGTAGATGTGCGCGACAACAAC |
| | ACACACGCCATATATATATAGTTGGAGGAGGTGGAAAACAAGGAGGCGATAAGAAAAAGGGGGGGGGGGAATATGGAGAATGTTTGAAATAGCGTC |
| | CAACCCGAGTTTCCATTATTAATTGTCGGCCAAAAAAAGAAGGCATGTTACTCATGATGGTAGTTTCAAGGGAAGTGGAGCCTCCCAGCTAGTTTGAA |
| | CACTTAAAAAAACAAGCTACAACAGAAAGTAGTTTCAAGGGGAGTGGAGCCTAAAACTTGCATTTAAAAAGGAAATGCGCACACTAAGCCGAAAGTGT |
| | GAACACTTTGTGCCGACATTTCCGAGGAAGTAGGTTTTATGCGCATAACATTTTTTTGAGACAATTTTATGCGCATAACTAGGGTTGACTGCTGTGGG |
| | GAAGATGAAATTTGAACTGCCATTGTGACGCAAAGATTTGGTGATCCAAATTTGTATATGGACCATGCCTGTAGGTTGGGCTAAGTATACTTTTCGAA |
| | GAAAATAGGCCCATGTTAACTTGGGAAGTGGGCCTAGGCCCACAAACCTGAAATCCCTGTGAAGCCCACATTCACACGCGGGCCCACCGTCGAAGC |
| | GATAAGGAGAGTGACAGCCGCTCAGCCCGCCGGC |

### BIBLIOGRAPHY

1 Bohra A, Jha U C, Adhimoolam P, et al (2016) Cytoplasmic male sterility (CMS) in hybrid breeding in field crops. Plant Cell Rep 35:967-993. doi: 10.1007/s00299-016-1949-3
2 A chalcone synthase-like gene is highly expressed in the tapetum of both wheat (Triticum aestivum L.) and triticale (xTriticosecale Wittmack). Wu S, O'Leary S J B, Gleddie S, Eudes F, André Laroche A and Robert L S (2008). Plant Cell Rep 27:1441-9.
3 CRISPR/Cas9-Mediated Deletion of Large Genomic Fragments in Soybean. Yupeng Cai, Li Chen, Shi Sun, Cunxiang Wu, Weiwei Yao, Bingjun Jiang, Tianfu Han and Wensheng Hou. Int. J. Mol. Sci. 2018, 19, 3835.
4 Highly efficient heritable targeted deletions of gene clusters and non-coding regulatory regions in Arabidopsis using CRISPR/Cas9; Julius Durr1, Ranjith Papareddy, Keiji Nakajima & Jose Gutierrez-Marcos. Scientific Reports (2018) 8:4443.
5 Generation of megabase-scale deletions, inversions and duplications involving the contactin-6 gene in mice by CRISPR/Cas9 Technology. Alexei N. Korablev, Irina A. Serova and Oleg L. Serov. BMC Genetics 2017, 18(Suppl 1):112.
6 Generation of chromosomal deletions in dicotyledonous plants employing a user-friendly genome editing toolkit. Jana Ordon, Johannes Gantner, Jan Kemna, Lennart Schwalgun, Maik Reschke, Jana Streubel, Jens Boch and Johannes Stuttmann. The Plant Journal (2017) 89, 155-168.
7 Efficient inversions and duplications of mammalian regulatory DNA elements and gene clusters by CRISPR/Cas9. Jinhuan Li, JiaShou, YaGuo, Yuanxiao Tang, YonghuWu, Zhilian Jia, Yanan Zhai, Zhifeng Chen, Quan Xu, and Qiang Wu. Journal of Molecular Cell Biology (2015), 7(4), 284-298.
8 Multiple wheat genomes reveal global variation in modern breeding. Nature. 2020 December; 588(7837):277-283. Walkowiak et al. doi: 10.1038/s41586-020-2961-x. Epub 2020 Nov. 25.
9 The genetic basis of cytoplasmic male sterility and fertility restoration in wheat. Melonek J, Duarte J, Martin J, Beuf L, Murigneux A, Varenne P, Comadran J, Specel S, Levadoux S, Bernath-Levin K, Torney F, Pichon J-P, Perez P and Small I (2021). Nature Communications.

The Examples below are given for illustration purposes only.

### EXAMPLES

### Example 1: Identification of Promoter Candidates to Improve Fertility Restoration

The T. timopheevii CMS restorer gene RF3 has been identified as a PPR protein on Chr1B referred to as RFL29 (TraesCS1B01G038500) (WO2019086510). This gene is present in most wheat lines such as Chinese Spring though its level of expression is low as measured by RNAseq data (FIG. 2).

There are at least 3 RFL29 variants in wheat; RFL29a, RFL29b, RFL29c. RFL29b, present in Chinese Spring is a less effective restorer than the RFL29a allele found in lines such as Spelt (WO2019086510, Walkowiak et al. 2020).

To determine if RFL29-mediated fertility restoration can be improved, RFL29a and RFL29b were placed under the control of the strong ZmUbiquitin promoter and transformed into a wheat line containing T. timopheevii CMS. Full male fertility was observed in single copy T-DNA transformants (Melonek et al. 2021). This indicates that an increase in RFL29 expression would be sufficient to create a single locus restorer.

In this example, inventors search to identify a strong promoter in a gene upstream of RFL29 and perform a genomic deletion such that the strong promoter is brought in front of RFL29 (FIG. 1). A further alternative approach is the generation of a genomic inversion so as to again bring a strong promoter in front of RFL29 (FIG. 1).

RNAseq data from the wheat Chinese Spring was used to identify candidate promoters that could be brought in front of the RFL29 promoter. RFL29 should be expressed in the anther to give restoration, thus candidate promoters should therefore have good expression levels in RNAseq samples from the floral spike. In particular, overexpression of RFL29b from the tapetum-specific ZmMac2 promoter was sufficient to restore fertility to CMS wheat (Melonek et al. 2021). The expression of the wheat tapetum-specific gene TaCHSL1 (Wu et al., 2008) was detected in RNAseq floral spike samples Z32 and most strongly in Z39 with no expression at stage Z65 (FIG. 2D). This indicated that candidate promoters to drive RFL29 should be expressed in Z32 and Z39 spikes. Two candidate genes in the same orientation as RFL29 were identified with good spike expression in Chinese Spring at these stages; TraesCS1B01G041300 (2172 kb upstream of RFL29) and TraesCS1B01 G038800 (304 kb upstream of RFL29).

The predicted protein encoded by TraesCS1B01 G041300 shows 99% identity to a protein from Aegilops tauschii subsp. Tauschii annotated as RAE1-like (accession XP_020173482.1). RAE1 proteins are required for export of mRNA from the nucleus. TraesCS1B01G041300 was thus named RAE1-like. This protein is represented by SEQ ID NO:8.

TraesCS1B01 G038800 shows 98% identity to a protein annotated as probable serine/threonine-protein kinase PBL11 from Aegilops tauschii subsp. Tauschii (accession XP_020157135.1) and was named TaPK-like. This protein is represented by SEQ ID NO:5.

The TaPK-like gene is about 7-fold more expressed than RFL29 in Z39 spikes and RAE1-like about 44 fold more expressed (FIG. 2).

To confirm that the promoter regions of TaPK-like and TaRAE1-like are sufficient to restore fertility when used to drive RFL29 expression, the putative promoter regions of TaPK-like and TaRAE1-like were amplified from the wheat variety Fielder and linked to the coding region of RFL29a (represented by SEQ ID NO:14).

The chimeric pTaPK-like::RFL29a (SEQ ID NO:12) and pTaRAE1-like::RFL29a (SEQ ID NO:13) gene cassettes were cloned into a plant binary vector containing a plant transformation selectable marker forming plasmids pBIOS12569 and pBIOS12526 respectively.

These binary vectors were transformed into agrobacteria, forming strains T11861 and T11844, which were transformed into a Fielder CMS line, a wheat line bearing a CMS sterility trait, by agrobacterial-mediated transformation and the fertility of the transformants scored.

The scoring method is outlined in FIG. 3. A fertility score is calculated as the number of seeds divided by the number of spikelets. Maximum fertility potential corresponds to spikelet number multiplied by 3 flowers. Since each spikelet has 3 flowers that maximum score=3. In the greenhouse, Fielder has a fertility score of between 2.1 to 2.3 whereas the CMS Fielder line is completely sterile.

The transformed TO plants that had the level of fertility of Fielder were classified as fully fertile. Both the pTaPK-like::RFL29a and pTaRAE1-like::RFL29a constructs gave fully fertile plants however the level of restoration was greater for pTaRAE1-like::RFL29a with 92% of TO plants being fully fertile as opposed to 61% for pTaPK-like::RFL29a (Table 1).

TABLE 1
Fertility restoration of pTaPK-like::RFL29a and pTaRAE1-like::RFL29a transformants

| | | | | | Fully | Fully | | |
|---|---|---|---|---|---|---|---|---|
| | | | | Transformed | Fertile | Sterile | Partial | Restoration |
| Construct | Promoter | RFL | Terminator | plants | Plants | plants | Fertility | efficiency |
| T11842 | TaRAE1-like | TaRFL29a | RFL29a | 26 | 24 | 2 | 0 | 92% |
| T11861 | TaPK-like | TaRFL29a | RFL29a | 32 | 19 (+4*) | 7 | 2 | 60% (71*)% |

Fertility restoration of pTaPK-like::RFL29a and pTaRAE1-like::RFL29a transformants. TO plants were phenotyped for fertility as described in FIG. 3.

Plants with the fertility of Wild-type Fielder were classified as fully fertile. All plants are single-copy for the transgene apart for 4 plants from construct pTaPK-like::RFL29a (indicated as *).

### Example 2: Creation of Genomic Deletions to Bring the PK-Like and RAE1-Like Promoters in Front of RFL29

As described in example 1, the promoters of TaPK-like and TaRAE1-like are candidate promoters to boost or replace the natural RFL29 promoter. The wheat line ‘Spelt’ has the favourable RFL29 allele (RFL29a) therefore it is desirable to engineer a strong RFL29 restorer in this or other lines that possess RFL29a. This strong restorer can then be introgressed into any wheat line to create a male line for hybrid seed production.

The deletion could replace a part of the RFL29a promoter by bringing enhancer elements of TaPK-like or TaRAE1-like (FIG. 1C) or alternatively the entire RFL29 promoter and the majority of the 5′UTR region could be replaced (FIG. 1B). In this example the second strategy is adopted such that double strand DNA breaks (DSBs) are created just in front of the initiating Methionine ATG of the coding sequences of RFL29a and TaPK-like or TaRAE1-like.

The Spelt RFL29a (SEQ ID NO: 16), TaPK-like (SEQ ID NO: 19) and TaRAE1-like (SEQ ID NO: 22) genomic sequences were identified by BLASTN analysis of the Spelt genome sequence. The regions in the coding sequences were analysed for sites that could be cleaved using CRISPR Cas9 or Cas12a guide RNAs (gRNAs). FIG. 4 shows the positions of candidate gRNAs and Table 2 suggests combinations to create the desired deletions.

TABLE 2
Pairs of gRNA targets for generation of genomic deletions in the RFL29 region

| | | | | gRNA combinations | | | | | | | | | |
|---|---|---|---|---|---|---|---|---|---|---|---|---|---|
| Gene | Target (5′ to 3′) | Guide | CRISPR | RFL29 del PK-like | | | | RFL29 del RAE1-like | | | | | |
| RFL29 | GCGGGGCATGGCCGGTGTGTAGG | 983r | Cas9 | | | | Y | | | | | Y | Y |
| RFL29 | GAGGAGAAGCGGGGCATGGCCGG | 991r | Cas9 | Y | Y | | | Y | Y | | | | |
| RFL29 | TGAGTTCGCGACCTACACACCGG | 972f | Cas9 | | | Y | | | | Y | Y | | |
| PK-like | TCCCCATGGCGAGGACCGTCCGG | 479r | Cas9 | | | Y | Y | | | | | | |
| PK-like | CCCAGCAGTTCCCCATGGCGAGG | 488r | Cas9 | | Y | | | | | | | | |
| PK-like | GCCGGACGGTCCTCGCCATGGGG | 478f | Cas9 | Y | | | | | | | | | |
| RAE-like | TTCTGTGAACCTTCAAAATCTGG | 1145r | Cas9 | | | | | | | | Y | | Y |
| RAE-like | AGATTTTGAAGGTTCACAGAAGG | 1147f | Cas9 | | | | | | | Y | | Y | |
| RAE-like | AAGGTTCACAGAAGGAGAGATGG | 1155f | Cas9 | | | | | Y | | | | | |
| RAE-like | TTTGAAGGTTCACAGAAGGAGAGATGG | 1151f | Cas12a | | | | | | Y | | | | |

In Table 2, the combinations of gRNAs are read vertically.

Such combinations are designed to maintain the Kozak translational initiation sequences of either TaRFL29a (such as combinations with targets 972f or 983r) or that of TaPK-like (478f or 488r) or of TaRAE1-like (1155f or 1151f).

The selected pairs of gRNAs are constructed such that they are expressed from the wheat U6 promoter (SEQ ID NO: 33) and the gRNA cassettes cloned into a plant binary transformation vector containing a ZmUbiquitin promoter::Cas9 gene cassette. In the case of use of target 1151f, the plant binary vector also contains a Cas12a gene cassette.

These binary vectors are then transformed into the wheat variety Spelt using agrobacterium-mediated transformation. Primary transformants are then screened for the desired deletion using a PCR screen with primers in the coding region of RFL29a combined with primers in the TaPK-like or TaRAE1-like promoters. PCR bands obtained of approximately the expect size for the deletion are sequenced to confirm the junction sequence. The junction sequence should maintain the functions of the TaPK-like or TaRAE1-like promoter and allow the translation of RFL29a. Junctions that lack a Kozak sequence or that remove the ATG of RFL29 or introduce a novel ATG sequence or alter an intron splice junction are less desirable.

Plants with a desirable junction sequence between the TaPK-like promoter and the RFL29a CDS or with the TaRAE1-like promoter and the RFL29a CDS are used as males in crosses to the Fielder CMS line. F1 progeny of these crosses are sown and scored for male fertility.

### Example 3: Creation of Genomic Inversions to Bring the TaAt5g02240-Like Promoter in Front of RFL29

RNAseq data from wheat Chinese Spring spikes were used to identify candidate promoters that could be brought in front of the RFL29 coding region via a genomic inversion. A gene with an opposing orientation to RFL29 was identified with good spike expression in Chinese Spring (FIG. 5).

The predicted protein of this gene (TraesCS1B02G037100) shows 97% identity to a protein from Aegilops tauschii subsp. Tauschii annotated as At5g02240-like (accession XP_020198298.1). TraesCS1B02G037100 was thus named TaAt5g02240-like. This protein is represented by SEQ ID NO:49.

The TaAt5g02240-like gene is about 80-fold more expressed than RFL29 in Z39 spikes.

In this example double strand DNA breaks (DSBs) are created just in front of the initiating Methionine ATG of the coding sequences of RFL29a and TaAt5g02240-like. The Spelt RFL29a (SEQ ID NO:16) and TaAt5g02240-like (SEQ ID NO:50) genomic sequences were identified by BLASTN analysis of the Spelt genome sequence. The regions in from of the coding sequences were analyzed for sites that could be cleaved using CRISPR Cas9 guide RNAs (gRNAs). FIG. 6 shows the positions of candidate gRNAs and Table 3 suggested combinations to create the desired deletions.

TABLE 3
Pairs of gRNA targets for generation of genomic inversions in the RFL29 region

| Gene | Target (5′ to 3′) | Guide | CRISPR | gRNA combination | | |
|---|---|---|---|---|---|---|
| RFL29 | GCGGGGCATGGCCGGTGTGTAGG | 983r | Cas9 | | | Y |
| RFL29 | GAGGAGAAGCGGGGCATGGCCGG | 991r | Cas9 | Y | | |
| RFL29 | TGAGTTCGCGACCTACACACCGG | 972f | Cas9 | | Y | |
| At5g02240-like | CTTCACTCGCCGGAGGGGGACGG | 70f | Cas9 | | Y | Y |
| At5g02240-like | GGAGGGGGACGGAGCAAATATGG | 81f | Cas9 | Y | | |

The combinations of gRNAs are read vertically.

Such combinations are designed to maintain the Kozak translational initiation sequences of either RFL29a (combinations with targets 972f or 983r) or that of TaAt5g02240-like (81f)).

The selected pairs of gRNAs are constructed such that they are expressed from the wheat U6 promoter (SEQ ID NO: 33) and the gRNA cassettes cloned into a plant binary transformation vector containing a ZmUbiquitin promoter::Cas9 gene cassette.

These binary vectors are then transformed into the wheat variety Spelt using agrobacterium-mediated transformation. Primary transformants are then screened for the desired inversion using a PCR screen with primers in the coding region of RFL29a combined with primers in the TaAt5g02240-like promoter or 5′UTR region. PCR bands obtained of approximately the expect size for the deletion are sequenced to confirm the junction sequence. The junction sequence should maintain the functions of the TaAt5g02240-like promoter and allow the translation of RFL29a. Junctions that lack a Kozak sequence or that remove the ATG of RFL29 or introduce a novel ATG sequence or alter an intron splice junction are less desirable.

Plants with a desirable junction sequence between the TaAt5g02240-like promoter and the RFL29a CDS are used as males in crosses to the Fielder CMS line. F1 progeny of these crosses are sown and scored for male fertility.

1-20. (canceled)

21. A method to modify the expression of a gene of interest in an organism, the method comprising the steps of:
a) introducing into the organism at least one gene-editing system;
b) allowing the gene-editing system to perform the desired editing at a target genomic site;
and wherein the gene-editing system is designed so that, after the desired editing, the gene of interest is operably linked to an endogenous promoter, or part of an endogenous promoter, the endogenous promoter being distal from a naturally occurring promoter of the gene of interest in the genome of a non-modified organism.

22. The method to modify the expression of a gene of interest according to claim 21, wherein the organism is an eukaryotic organism, provided that the organism is not a human or other animal.

23. The method to modify the expression of a gene of interest according claim 21, wherein the performance of the desired editing as recited in step b) comprises two or more DNA breaks within the target genomic site.

24. The method to modify the expression of a gene of interest according claim 21, wherein the desired editing is a deletion or an inversion.

25. The method to modify the expression of a gene of interest according to claim 24, wherein:
the deletion is a deletion of a genomic region comprising the full naturally occurring promoter, or is a deletion of a genomic region comprising only a part of the naturally occurring promoter, or
the inversion is an inversion of a genomic region resulting in the replacement of the naturally occurring promoter by the endogenous promoter, or is an inversion of a genomic region resulting in the replacement of a part of the naturally occurring promoter by a part of the endogenous promoter.

26. The method to modify the expression of a gene of interest according to claim 21, wherein the gene of interest is a fertility-restorer gene the gene is chosen among:
RFL29 gene or
RFL79.

27. The method to modify the expression of a gene of interest according to claim 21, wherein the endogenous promoter drives a higher expression of the gene of interest, changes the pattern of expression of the gene of interest during the development cycle of a plant, changes the spatial pattern of expression of the gene of interest or is a promoter which is activated by biotic or abiotic stress.

28. The method to modify the expression of a gene of interest according to claim 21, wherein the modification of the expression is an enhancement, the endogenous promoter being notably a strong promoter.

29. The method to modify the expression of a gene of interest in an organism according to claim 21, wherein the break occurs in a promoter, in an untranslated region, in gene-gene junction region, in exon or in intron.

30. The method to modify the expression of a gene of interest according to claim 21, wherein the editing system is chosen among a zinc finger nuclease (ZFN) gene editing system, a transcription activator-like effector nucleases (TALEN) gene editing system, a clustered regularly interspaced short palindromic repeats (CRISPR) gene editing system, or a meganuclease gene editing system.

31. The method to modify the expression of a gene of interest according to claim 21, wherein the gene-editing system comprises an enzyme chosen among: meganuclease, zinc-finger nuclease, transcription-activator like effector nuclease, CRISPR-nickase or CRISPR-nuclease.

32. The method to modify the expression of a gene of interest in an organism according to claim 21, the method comprising the steps of:
a) identifying an endogenous promoter, distal from a naturally occurring promoter of the gene of interest in a non-modified organism, which is capable of modifying the expression of the gene of interest;
b) identifying the deletion or insertion of a genomic region to be performed in order to operably link the endogenous promoter, or part of an endogenous promoter, to the gene of interest;
c) introducing into the organism at least one gene-editing system designed to perform the deletion or insertion identified in step b);
d) allowing the gene-editing system to perform the desired editing at the target genomic site;
e) optionally selecting an organism with the desired editing.

33. The method to enhance the expression of a fertility-restorer gene of interest, in a plant, the method comprising the steps of:
a) introducing into the plant, at least one gene-editing system;
b) allowing the gene-editing system to perform the desired editing at a target genomic site;
and wherein the gene-editing system is designed so that, after the desired editing, the fertility-restorer gene of interest is operably linked to an endogenous promoter, or part of an endogenous promoter, the endogenous promoter being distal from a naturally occurring promoter of the fertility-restorer gene of interest in the genome of a non-modified plant.

34. The method to enhance the expression of a fertility-restorer gene according to claim 33, wherein the plant is a wheat plant.

35. The method to enhance the expression of a fertility-restorer gene according to claim 33, wherein the gene is RFL29 or RFL79.

36. The method to enhance the expression of a fertility-restorer gene according to claim 33, wherein the desired editing is a deletion or an inversion.

37. A plant, or plant part, comprising a gene-editing system designed to perform a desired editing at a target genomic site, so that after the desired editing, a gene of interest in the plant is operably linked to an endogenous promoter, the endogenous promoter being distal from a naturally occurring promoter of the gene of interest in the genome of a non-modified plant.

38. The plant as obtained according to the method according to claim 33.

39. A method to identify a plant according to claim 38, comprising, after the desiring editing was performed, a step of:
PCR, in order to detect bands corresponding to the desired editing, notably inversion or deletion,
determining the expression level of the gene of interest, in order to compare the expression of a gene of interest by comparison with the expression of a gene of interest in a non-modified plant, thereby verifying the modification of the expression of the gene of interest,
determining the level of the expressed protein encoded by the gene of interest, in order to compare the expression level by comparison with the level of the expressed protein encoded by the gene of interest in a non-modified plant, thereby verifying the modification of the expression level of the protein encoded by the gene of interest,
phenotypic analysis in order to determine if the plant has a fertility restoration phenotype.

40. A method to restore fertility in wheat, comprising a step of crossing a sterile wheat plant with a plant wherein the expression of a fertility-restorer gene has been enhanced, provided that the plant is a plant according to claim 38.


SEQUENCE LISTING (63 sequences)

SEQ ID NO: 1 - DNA, 2373 bp, Triticum aestivum (var. Chinese Spring)
atgccccgcttctcctccaccacgccaatgtcgccaccccgcctccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcaccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttattgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattcgccttcatgggtatgccaccgaaggatgcctagttgacatgatcaatctcttcaattccatggcaacacactgcattctacctaactgtcatatattcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatag

SEQ ID NO: 2 - protein, 790 aa, Triticum aestivum (var. Chinese Spring)
MPRFSSTTPMSPPRLRLRLRLCARHSSSTSHPSRIWDPHAAFAAAAQRASSGTLTTEDAHHLFDELLRRGNPVQERPLNKFLAALARAPASASCCDGPALAVTLFGRLSRDVGRRVAQPNVFTYGVLMDCCCRACRTDLVLAFFGRLLKTGLEANQVVFNTLLKGLCHTKRADEALDVLLHRMPELGCTPNVVAYNTVIHGFFKEGHVSKACNLFHEMAQQGVKPNVVTYNSVIDALCKARAMDKAEVVLRQMIDDGVGPDNVTYSSLIHGYSSSGHWKEAVRVFKEMTSRRVTADVHTYNMFMTFLCKHGRSKEAAGIFDTMAIKGLKPDNVSYAIRLHGYATEGCLVDMINLFNSMATHCILPNCHIFNILINAYAKSGKLDKAMLIFNEMQKQGVSPNAVTYSTVIHAFCKKGRLDDAVIKFNQMIDTGVRPDASVYRPLIQGFCTHGDLVKAKEYVTEMMKKGMPPPDIMFFSSIMQNLCTEGRVTEARDILDLIVHIGMRPNVIIFNLLIGGYCLVRKMADALKVFDDMVSYGLEPCNFTYGILINGYCKNRRIDDGLILFKEMLHKGLKPTTFNYNVILDGLFLAGQTVAAKEKFDEMVESGVSVCIDTYSIILGGLCRNSCSSEAITLFRKLSAMNVKFDITIVNIIIGALYRVERNQEAKDLFAAMPANGLVPNAVTYTVMMTNLIKEGSVEEADNLFLSMEKSGCTANSCLLNHIIRRLLEKGEIVKAGNYMSKVDAKSYSLEAKTVSLLISLFSRKGKYREHIKLLPTKYQFLEEAATVE

SEQ ID NO: 3 - DNA, 7650 bp, Triticum aestivum (var. Chinese Spring)
gcacctttaggtcctaattctttattaattggttcacccgaggtccttttccacgagcgctcgctgaccagcacacgtgccgcattgacccatgccacatcgatagagggtctaaccgccaggcgagaagcttcgcaaagagacccccccccctcttagtggcaccctctctcctctttcacggtactttggcgacattggaggcgagcgacagaggcaaccggaggtgatctggaggtaatttgtgtgggtgcccgtgaagatgccccttctatgacggccagtgagcaatcctcctccatgttaccgtaagaaccctaatcttgtcgatgatcttctcctgtagctcttctaatatgtcataactttgtcataatcaatagttgtagtccttgatttactttgatttgcataacggtgatacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggtttaggggacgcgcgcgccgagagagagagcagatttggggaagaaataaagccagggactggggaatgagaagataatgtttataaggggttttctgtaaaaagaaagagcaacctacgcagcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacatactccctccgtccggaaatacttgtcgaagaatttgatgaaaatggatgcatctagaacaagaatacatctagatacatcaatctccctgacaagtatttccgagcggagggagtactagataatactccctccgttcctaaataattgtctttctagctatctcaaataaactacaacatacggatgtatgtagacatgttttagagtgtagattcactcattttgttccgtatgtagtcatttgttgaaatctctagagagacaattatttaggaacggagggagtaagataactaccctaaaaaaaaagataactgaaggttgccacctagcacattcacattggtacaacttggaaaagcacagccccgtcgtcctgctcccagttgagttcgcgacctacacaccggccatgccccgcttctcctccaccacgccaatgtcgccaccccgcctccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcaccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttattgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattcgccttcatgggtatgccaccgaaggatgcctagttgacatgatcaatctcttcaattccatggcaacacactgcattctacctaactgtcatatattcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatagttggtacatgatatctgaaatttaatttgcatcgcttgccatgggtccgtctgcttgtacaagaaatgcattttctatttgtaaataggaagtcagtttagaacaagccatcaggatgacgcaaagagtacaattcagttgcaccaccaataaaaaggcagaactagggctgccaaaacaacactgaatcaaaactcaaacagaaggagcagcaaacttttttttttgaagctggacagtctgctagccaaacaactacaggagactgtcaggcggggcatgtagtggctggcgtctaagcgcctttgcttcttccaccatccatggcttagcctcacacggaatcgagtcaaccaattcccgtcggttttgggtggctcccttgaagatgcaattgttttcagcggccagatacgcatggtcagattaatcagcgagtgtgccccttctgtctggttcgagaaagaatttgaagagctgagcttgtcccagctggaaccagtttgaggttagttaatcataacagggtactagagaggtgttttattgactgttgatgtgtaatatgttatatgccatcctcttgatgattacggtgatctgtgaagaggcagcatgcaaaagtctgaatcacatatgcttatgtaattgtgttattatttgtgcagcttctagaccttttgccttgtagatggctacatggatctagttgtagcaatctgtaactgttagtgttttgtatatgctggcattgatgattagggtgacttgtgaagcatgcagaagtctgaatcatacatgcttgtctagttatttttggggctctctctttgccatgtacatgttttatggatgcagttgtagtctgtatgtgctggcatttaatatgagggctggtctgcaatttttctgttttgatcgatacagcgccatgttgagtttaaccctgtaacttggcttaactagatcatgctggtgttatctctaggaatcacaatatcaatactctctgttgttgcacaaactgacgcagatcaatgtccagaggtaatatggttctgcccgtaattttcatcgactgtcagcatgttccttttttatttagtactctgtatatcacggcggtatgactataataaggatatacggctggtgcattttgagcttctctgtttcttgggataaagctgataaatttctattcttgttggctttggtagtatgaacagaaagcaggcagtaataaactgatatgtggtactattttgttagtgaaacagggtcctttttattatctattttatccagcaatagtgttccataaaattatagatcggttcattcatctcatatggatttcggtgagcttcgcccgtccttggtacattttgttactatagaacaattggtgtgcgctttttttcttttggctgtacgttatagaacaacaaaacctgtttatttcattaattcattgtctaatttattttcttacgagagctcagaagttcagcattttattttatcttgttccttccgcgtaatttttcatttactcactgagtagtacaggtcttggacctgaaattcttgtactaatttagtagaaccattcttcgagaaacactccaaaaatggtttgtcttgggtctgaatcatattgtctctgaacaaacaggtgtgactcatgcatggaaagatccccaagaaacattgcacatttggtagatatgtctatgcatgaatactattttttgaaaaagaatttagtaaatattttggtagcattttttatacgtatgatgctttttcttcttcccgaaataagggttttgatttttgaatttttgtaaagcaacatggtttgtagtacgtacgaaagaacgctagtgtgttttcgcgatcgtaagaatgttataaactgttttcctgggtcatgtaggtgtaagctatagaaggaattcaatatcgtcctaaacatccttggcagtggccagtgaaagtcttggacctgaaatcataccagtactatttttctgaaagagaacccattcttgtattccataagttggctggtgcctgggcataattgtctaatcatatagtactatttgcatctcataattccattcagttttgcactatagctgaactgaaggagtacctagcacaaaatatgtatgactgtatttcagatcttgcaatcgcagctgtaatggagtagcacaccctgtagcagcatatggatgtactctgaatgttgggactctttgaaatgataaattggtctattctgggagcagattttttccggctttcggagattcattgatctttttttttcagaaagtacagttgtaagttataatcaaacatcccgaacaaacctcacttgtacatgtaagaatgcactatgaatcagtggaaagtagatttgtggtgaaccaaaatagccaaggaataggttcactttcctccctccttatgtctaaagttgatgcaaagagctactcacttgaagctaaaactgtttcgctgctgatctctctcttttcagggaaagggaaatacagagaacagataagattgctccccgcaaagtatcagtttcgcggagaatgagctacagttgaatggttttacatgatatctgaacttaactcctcagaaggtaaggttgaattattgtttgccatgagtttatctgcttgtacaacaagtgcaatttccatatgtaaataggaaggcagttgagaacaagccatcaggatgacgcaaagagtacaattcaactgtaggaccagcaattgttttgaagttgtatgtctgctagtagaacacctacgggagagattcaggcggccgggcatgtagtggcacctgtgtaagtcccttttctgctgccagcatccatggcttggcctcacagggaatggagtcaaccaatttctcggcgtagctgttgcaaatgttttctcagccaccagatacaccaaagtttcagagtaatcagcgagcgtgtctcttttgtctggttcgagaaagaatttgaaaacgctgagcagcaacaggtgcttgaccagcttgtccctgccgaagccagtttgaggttagttaatcataacagggtactagacaggtgttttgttgactgttgatgggtaatactcctatgttatatgccagccacttgatgaatagggtgatctgtgaatcacatatgcttatgtaattgtgttattatttgtgcagcttctagaccttctgccttgtatggctacatggatcacgttgtagcagtctgtgactgtgactgttagtgttttgtatatgctggcattgattaatttagggccgggctccaaattatttgttttgatcgattcagcaccatgttgagtttaaccctgtaacttggcttaaccagatcaatatcaggggcctcggcatttttaaacgttgaattaaacatactctgtggcatatcaatgttcagaggtaatatggttctgtccataatttcatcgactgtcagcatgttcctttgttattcaatactctgtatatcatgccggtatgactataataaggatatagtatgtttgatgcattttgaacttctctattccgtccgatagaggtgataaattctctattctttttggctttggtagtatgaacagtaagcaggcagtatatattaaactgatatgtggtactattttgttaatgaaacag

SEQ ID NO: 4 - DNA, 1215 bp, Triticum aestivum (var. Chinese Spring)
atggggaactgctggggcgccaagatcagctccgacacctcctcctcctcctccccttcagggacgaattccaagtatgctactaggaatggggcggccttgagcagctccagcagctatgcctccgcggcgtcagtgccacggagcgagggtgagattctggagtcagccaatgtcaaggccttctctttcaatgagctgaggaccgccacgagaaacttccgtccggacagtgtgctaggcgagggagggttcgggtcagtcttcaaggggtggatcgatgagaagaccctcaccccaactaagccaggcaccgggatggtcattgctgtaaagaagcttaaccaggaaagctatcagggccatagggaatggctggctgaagtgaattaccttggacaactatcgcacccgaatcttgtaaagctcgttgggtactgtgtcgaagacgaacagcggcttctcgtctacgagttcatgccccgtgggagtttagagaatcatctattcaggaggagtacacatttccagccgctctcctggaaccttcggatgaaaattgcccatggagcagcaaaagggctcgcgtttctccacagtgacaaggccaaagtcatctaccgcgatttcaaaacctctaatatcctcctagatgcgaactatgacgcaaagctctcagatttcggcctggcgaaggacggaccgacgggtgacaagagccatgtgtccacaagggtgatggggacatatgggtatgctgcaccagaataccttgcaacaggccacctgaccacgaagagcgacgtgtacagcttcggcgtggtcctcctggagatgctctcggggcgtcgcgcggtggacaagaaccggccgaccggcgagcacaacctggtggagtgggcgcggccgtacctgacaagcaagcggcgcatcttccgcgtcctggacccccgtctgggcgggcagtactccctcgccaaggcccagaaggcggcgtcgctggcgctgcagtgcctctcggtggactcgaggaacaggccgagcatggagcaggtcgtcgtggtgctggagcagctccacgacgccaaggagggagggaacagccctcgcccgcagctgcagagaaagccgagcagcaaccggggcgtgaacggctcgagatcgtcgtcgaccaaggggaacaacaagaagcccgcctcaccgagaccggtttga

SEQ ID NO: 5 - protein, 404 aa, Triticum aestivum (var. Chinese Spring)
MGNCWGAKISSDTSSSSSPSGTNSKYATRNGAALSSSSSYASAASVPRSEGEILESANVKAFSFNELRTATRNFRPDSVLGEGGFGSVFKGWIDEKTLTPTKPGTGMVIAVKKLNQESYQGHREWLAEVNYLGQLSHPNLVKLVGYCVEDEQRLLVYEFMPRGSLENHLFRRSTHFQPLSWNLRMKIAHGAAKGLAFLHSDKAKVIYRDFKTSNILLDANYDAKLSDFGLAKDGPTGDKSHVSTRVMGTYGYAAPEYLATGHLTTKSDVYSFGVVLLEMLSGRRAVDKNRPTGEHNLVEWARPYLTSKRRIFRVLDPRLGGQYSLAKAQKAASLALQCLSVDSRNRPSMEQVVVVLEQLHDAKEGGNSPRPQLQRKPSSNRGVNGSRSSSTKGNNKKPASPRPV

SEQ ID NO: 6 - DNA, 6090 bp, Triticum aestivum (var. Chinese Spring)
  misc_feature (1389)..(1669): n is a, c, g, or t
cacggagtgacaaatcccagtctcgatccgcataaaacaatagatactttcggagatacctgtagtgcacctttatagtcacccaattacgttgtgacgtttgatacacccaaagcactcctacggtatccaggagttacacgctctcatggtcaaaggaagagatacttgacattggcaaaactctagcaaatgaactacacgatcttttgtgctagtcttaggattgggtcttgtccatcacatcattctcctaatgatgtgatcccgttatcaacgacatccaatgtccatagccaggaaaccatgactatctgttgatcacaacgagctagtcaactagaggctcactagggacatattgtggtctatgtattcacacatgtattactatttccggataatacagttatagcatgaataaaagacaattatcatgaacaaggaaatataataataatacttttattatttcctctagggcatatttccaacaaaagtgtcgcaggaaatagaaagtagtatcctacggtatgtattgcatttattagttgtaggctcaactaaaagcactactgcgtggtgacaacctaagactagccacagtgagagtaactttagtagtaacatcgagtccaactcagcaaatttgcttatgtggcaatgagttaatgaggagagatgtaatacaagtaacttagctaattactgtaacactacatctcccaatgcattatgagggggtgtttggttcagaagtcctaggactttttctagtcccagggactaataaaaaaaaactctctactagagtctttttctagtccatgcataaaaagtcccttccgtttggttcataggaactttttagggaccttttctagtctctgagactaaaaaatctctgaaccaaacaccccctgagtctataacctaataaatgaatctttgtatgttaccatacttatgttactacccatagcctagaaacgtgtgtatgttactctccatagtggttagtctaatacgtgactttaaacatctctctgcccaagtagctgccacataagcagatgggcgtcttaatataataatatagaaatgtatcggggtaatataatactgtggcgataggcttgggttaatttctttagtcccgccgataagctcgattggtcagtgctggtgcccctctcgactccacacacacacacacagagggcgaaaaataagggcacacgaggccacgtcacgtggggcccccatgacccacacgcgggcccgcgcgccatcgtgtcgacgcgccgcgagcagagacggccggcgaggccaacccgaaccaccactccgcgcgagccgcccctccctttctctcgccttttcggcccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntagcggtagtgtatcgggttcgcctccctccccctccccctgcctgcactgtcccatcgctcgctcgctcgctttctcgcccgctcggtggcggtcgtgggggagaaagttgccggccggccagcgcacggggaaaaggcgcacatactttctttctttctttccaccgccgccctcctcctcctcccggaggagttccgattccccctctctttctctctcttctttctctctctctttcttgctctgctctggaagcagggggtcctgatttgggcgcgggcgccgtccgccgtgattcggttcggctgatttgggaggtctgctcgtttgatccgggttcgctgattggttgattgattgattggttggttcatctccggcggctcgatttgagggccggtcggcttggtcgccatggggaactgctggggcgccaagatcagctccgacacctcctcctcctcctccccttcaggtgcgtgcgtcgtcctccctccctctcctcatgctactattttgcaaagatttttaatactacgtaccagcggtacgactagttcataactaatctgtgcccagagggtcgaataaatcctgaatgaaaaatctggtggtttttggtttgcggcgacttcgtgctctgcatcccttctctccttcccgaaaccagtcctcggtcatgctgattcgatttcggtccaatccaactgcttcaacatagggcaaacactaggttaggttcactactgtagaaacttaatctggttcctaccaaatagtactccctccgtaaactaatataagagtgtttagatccctattttagtgatctaaacgctcttatattagtttacagagagagtattaagtagactactcaataaagaaatcactggtctgtgttaactctctactactttgcatctcctcactgttttcgggaactaagactagaactgggttatgctgatttcgttccgtttaatcgactcaagattggagagtcactaaattcagtacagcacaaacttaatttgtctccctaggaaaattcagtccattctttggaaaaatctgtcgcgagctttgactcttcctcttgacctgcttcgtgtgcaagggatcatgcctttccgttttgggcctaggggagaggaagcttccgatgtagtagtaaaatgcttcacttaactttatctgacttctcctctgatttttctgagtgtccagttcaaatcagacatatagaggaaaagctcgaattctgatgctacttttggctacatccacgtgcttttcttcaaagttattgactgtcctgttttggatgttcagccttcttttctgtaataagcgaaactaaaggaagaaaatacatagttagtccgacctaacttgtccatgatgaacctattttctgttcaatgttaggcagacaagataaatgataaagggacagcaacctatttattattctaaaccgtgatcgaatcatttctgaaactctgcaccattgtatgatgatgatgatcagggacgaattccaagtatgctactaggaatggggcggccttgagcagctccagcagctatgcctccgcggcgtcagtgccacggagcgagggtgagattctggagtcagccaatgtcaaggccttctctttcaatgagctgaggaccgccacgagaaacttccgtccggacagtgtgctaggcgagggagggttcgggtcagtcttcaaggggtggatcgatgagaagaccctcaccccaactaagccaggcaccgggatggtcattgctgtaaagaagcttaaccaggaaagctatcagggccatagggaatggctggttagtgacaattttgcctgctggaaatgggatttcttgtttatttcagttctgcattgtgtctgacatgctctttcttttgggcgcaggctgaagtgaattaccttggacaactatcgcacccgaatcttgtaaagctcgttgggtactgtgtcgaagacgaacagcggcttctcgtctacgagttcatgccccgtgggagtttagagaatcatctattcaggagtaagcccctggccttttacttttcttattcgctgggtgttattctccagttttgaaggaatgatgctaaagtgttccaacattcagggagtacacatttccagccgctctcctggaaccttcggatgaaaattgcccatggagcagcaaaagggctcgcgtttctccacagtgacaaggccaaagtcatctaccgcgatttcaaaacctctaatatcctcctagatgcggtatatatatccacagccttgaaggatcttttcctttgcgtccgcaaaatatctctatcggtcgatctgacagtcacgcaatttcttcttcagaactatgacgcaaagctctcagatttcggcctggcgaaggacggaccgacgggtgacaagagccatgtgtccacaagggtgatggggacatatgggtatgctgcaccagaataccttgcaacaggtcggttgatcattccactttcagaatcctcagctgcgcaaatataagatatattgctgaaaatctgaccacgcctacctgaatcgtgattgcgttgtcactctggttgtgtcatcgaccaagatgccgaacataaccaattttaggctacaacctttcaatcatcatctgcacaaatatcttgcccgaaaatctaaccacacctacctgaatcatgattgcgttctcactctggttgcgtcatcgaccaagatgccaaacttaactaatttttcggctagaacctttcagtcctcatctgcacaaatatcttgctgaaaatctaaacacacctacctgaatcgcgagtgcattgtcaccctggttgcgtcatcaaccatcatgccgaacttaaactaattttcctgctgcaatatttccatgatcaggccacctgaccacgaagagcgacgtgtacagcttcggcgtggtcctcctggagatgctctcggggcgtcgcgcggtggacaagaaccggccgaccggcgagcacaacctggtggagtgggcgcggccgtacctgacaagcaagcggcgcatcttccgcgtcctggacccccgtctgggcgggcagtactccctcgccaaggcccagaaggcggcgtcgctggcgctgcagtgcctctcggtggactcgaggaacaggccgagcatggagcaggtcgtcgtggtgctggagcagctccacgacgccaaggagggagggaacagccctcgcccgcagctgcagagaaagccgagcagcaaccggggcgtgaacggctcgagatcgtcgtcgaccaaggggaacaacaagaagcccgcctcaccgagaccggtttgaccgtgcgagtgggatcacatagcagaatctactagagagatgcgtcaaggtgtaatgtatgagtggtgatctgcgaagaggcagcggatttgtgaatgttttgtgcccgtgttgtagggatgtatctttgcagcttctagtgttggtatgtacagcttgtatctatccggttgtagctagtctgtatgtagtccttttgtgtatgcctgattgctttggttctgacttaaggtccagaggttttgatctgatttagatgcatgcaccctgttaaaccaagaccttaaaatatgaagatgaacagtgtaaagtacatatttgtcacttcatttatcaaatgtgaatgaacaatcatattggtgttttagcagagcaggccatgacttccatgggtcaactgtatcttttcccttagggatgttgaccattttgttcatatcatacagtactagtagtatatgtttcattttttttatgaaaaaagtagtatatgttacagataatgttgggaaaatcaccttctgtatttttaatttcaaacagttggccacaatgatctttttatttacttggtgggttcagccaaattaccattgtttttttcaggggtttcagccaaattattattatctgggcaagcgtacggcggcggcccaaggcagagcacgcagcccagccgccaaaacagagctcgccctcgctcccctccggcccaagtgccgctcctcctcctcctcggtggacgccggcgccggtggatggctccgccgcgccaaatcgagcccctcacgccgcgagcctctcctcctcctcctcagcctgcaatccccgccgagctccgatccccgccgccgagctccgatccgcgggcgatcgccggggcgccgccaagatcagctccggcccctcccgttcctttaggtgcgcgtttctattcttcagcaaaatagttttctggcatccgaccaggttaatacccaccgtaacaaacggacaggctatcgcgaatgttttgatgaacgatactaattgc

SEQ ID NO: 7 - DNA, 1032 bp, Triticum aestivum (var. Chinese Spring)
atggcagctctaaccagccttgcctcaaacccgaatccaaacaagtcattcgaggtccttcctaatccgggtgactccctctcaagcctcagttttagcccgaaaagtaatcttcttgtggcaacttcctgggataaccaggtgaggtgttgggagataggtaatggtaacagtcagccaaaggcatccatatcacatgatcagccagtgctctgctcagcctggaaagatgatgggactactgtcttctctggagggtgtgataaacaggtcaaaatgtggcctctgctgtctggtgggcaggctcagacggttgcaatgcatgatgcacctgtcaaggaggtcgcatggatttctcagatgaatcttcttgtatccggaagctgggacaagacactaaggtattgggacacaagacagccgaatccagcccatgttcagcaacttcctgatcgttgctacgcacttgctgtgaattatccccttatgattgtgggaacagctgatcgcaatattgtgatcttcaacttgcagaatcctcagactgagtttaagcgtattcaatcacctctgaaataccagacacggtgcgttgctgcctttccagatcaacaaggattcctggtgggttccatagaaggaagagttggtgtgcatcatattgatgattcacagcaaagcaaaaacttcacattcaagtgtcacagggaaggaaatgatattttctctgtcaattcgctcaactttcaccctgttcatcacacgtttgccacagctggatctgatggtgctttcaacttttgggataaagatagcaagcagagacttaaggctttcagtcggtgtcctcaacccattccttgcagtagcttcaataatgatggttcaatatttgcttatggggtgtgctatgactggagccgtggcgctgagaaccataatcctgcaaatgcaaagacatccatctatctccacagtccccaggaagccgaggtgaaagggaagccaagaatcgcaacggggcggaagtga

SEQ ID NO: 8 - protein, 343 aa, Triticum aestivum (var. Chinese Spring)
MAALTSLASNPNPNKSFEVLPNPGDSLSSLSFSPKSNLLVATSWDNQVRCWEIGNGNSQPKASISHDQPVLCSAWKDDGTTVFSGGCDKQVKMWPLLSGGQAQTVAMHDAPVKEVAWISQMNLLVSGSWDKTLRYWDTRQPNPAHVQQLPDRCYALAVNYPLMIVGTADRNIVIFNLQNPQTEFKRIQSPLKYQTRCVAAFPDQQGFLVGSIEGRVGVHHIDDSQQSKNFTFKCHREGNDIFSVNSLNFHPVHHTFATAGSDGAFNFWDKDSKQRLKAFSRCPQPIPCSSFNNDGSIFAYGVCYDWSRGAENHNPANAKTSIYLHSPQEAEVKGKPRIATGRK

SEQ ID NO: 9 - DNA, 11234 bp, Triticum aestivum (var. Chinese Spring)
ggtgaggttatggtatgtacaattttggcgcacaatgacggcgacttccctgatccttcgtcatcgtctccaccgtgttggttttggtgctggcgtgaggcctgaggtatgtctcttggatctaatgctcgtctagcgttggtggtttctccttcagagcgatggcccggtaagacgtggccttaatcactccccgcccgtgatgaacatcaggctggcttcgatgatggagaagcatgtgttggctcggcatgttctagaggatgaagaaggatgacccaggaattttgatgtaactttattaatattttcttcctataatttgtacttccttttcttagaaaaaaaactattttgtactgctagtttgattaatattcgtggcttcacatgcgctggtttggttatataagagggagcatatgggtgttgtctttttatataagaaaaaaaagagcaaaataagccacgcgccgatcaagcctgcgaactgagagtatacccggccatgggcaacccagactgacccgacccgtcccgactcgaccttgcccacgggccgggcccaggcctagattttgagcccgatggctgggccgacatgttgtatttgcgatttaaggaagaggcccggcccaagggccgaggcccgacgggttttttgactgatgggccggggttgggccaaattgttaggcccaacggccgggctcgggcctaagccttttgcttgcgttcaggctttgataggctcagcccgaagtccacatccacatcaaccacccccgcgtgccgatgaagccatttctttggaaggaccttcgcgtgcatccgacgcgacgcgctacaccgaagcctatactactacatctcgtctcatcgccctcctcccacacgacgactcgagaatcctcggctgaggctccactccgcactcgcaagtcgcacccgcagccgccgccgaccgctccccgtccggcaggtaagccccccgagcccctcctcgccgctccgctcgcgcggactcctagctagctagggttcccggcaccgattccgcggcgctgcactccggcgcaatgctcgcggccgcggacgggcccgccgcttcgggggaagctctgatctggtggccggcgctcgctcgctcgctcgcggtctccggagcaaatcggccgcatcgagtggccggagcaagcttaggatagttcgattatttcccccatccccgttggtcaggccgagagggccagtttgggcgacctcctggtgattccgctgccgtggtggggattgggtggtcgtggcccgggtgtaggcgtttgtaggtcgagtgagtgtttccttccaggttctatgctcacccagcttgtgttatcactgggatccgggcgttcggcaggctaacattcaccggattttggacctgcattgttgcggcgcgtgccgggggatagcgtttatgctggctttggcgcggtgattttgtgcgtgcgtgcaaacttttggggctgccttctggtgatctcgctgtcgggtggtgcgagttcatcaagcggtcagggtgtaggcatttgtagtcgagcgaccccagctttcatgcccaccagctcgtattattcggtcgttaaccacgtttaccgaattttggaccgacgtcgttgcggcacgtaccaggggacagcgtttatgctggtttcggtgtggagattttgtggtttaaacttggggcgcgcttggtttgcgaggctacctcctggtgatttcaccgtcgggtggtgggaatcggatggtcggggtgtaggcatttatagtggagcggaccccttgttttatgaccagacatttagccgaaatttggacctacactatcgtggcgtaccaggggatatcatttatgcgggttttggcgcgacactttctttgtcgggattttgtggtgcaaacttaaataatgacttccctaatgttaaacactcgttctggcatcgtggactgttggaatcgttctttttcgtagtctgagctactttaagttgttacttatgctctcgtgtgcgtgggagactccacgtgctgatttctcttacttgcttctcatggtcactggactgatatttcttttccagattttgaaggttcacagaaggagagatggcagctctaaccagccttgcctcaaacccgaatccaaacaagtcattcgaggtgtgatactttttgtggccatgtctgtcctgcttttatttttttgcgtcttgtcattctgtatatgctacatgttctctgcaatggttgctttcaaatgcaggtccttcctaatccgggtgactccctctcaagcctcagttttagcccgaaaagtaatcttcttgtggcaacttcctgggataaccaggtaattttgtttctgctgtaattttcagtgagcttccattattgtgatgtatcttactaggaatgtcttttgagttttgtgcaggtgaggtgttgggagataggtaatggtaacagtcagccaaaggcatccatatcacatgatcagccagtacgttctcatacatgtttctcctgaaagtagtttcatcttacacattagagtggttccttacctgtttacttggtatgatgattgatttgtttccttccatcaggtgctctgctcagcctggaaagatgatgggactactgtcttctctggagggtgtgataaacaggtcaaaatgtggcctctgctgtctggtgggcaggctcagacggttgcaatgcatgatgcacctgtcaaggaggtcgcatggatttctcagatgaatcttcttgtatccggaagctgggacaagacactaaggtgcaagaacaatattcctctccaccctcagtattctttttacatggaaattcagcgcatatcacccatatctgatcccaaattgttatttatggctgttttgcttttgggaattcgtgttgtttgtggtatcctttctgtacttcatgtgttctgcattaatgttcacacattggctaaccaaatattgtagaagttctatctcatcctctttatatttgcactcattttgtatgccttctttgatcctgaagagttttctactacttgttgaatcatactgccctattcttatcaaaggttgtgcactttctgggtatcatctgtactatttatggctgtagtgaatttttcttatggcagaagtttttatagaagtaactgttgatgcagacagttactatattaaatgtacattatgtgattagtgttagaaatgttcgtttcgagttttagcatcatctttatgaagattatttatgaaacatcaagttagggtctggctgtcagtttaaacatgagcacagattgagcataggcgataatcatggttggtaggaggccagggaccgggatccttgtgggacctgccggcgtacccgtgatagcagaaggggtgcaagggacagggcactggaactaaggcaggcagaaacagagaaataaagattggggattagactttgtagccaattcataaaaaaattggttgcatcattttgtagaggttggccttaacatatgactaaaaaacaaaaacaaaccaagactcttccctaaactgaataccgcagatactcaagacaccatatagggccacgggctcaggctgaagtcatctaggtctcagacatgccaaaacagactgggacaaggacaagctgaaactgactagtactaggtttcccgggtcaactaccgttcagctggaggtctgaatgcgtggtgccttctctgatacatggatctcatgtggcacttctgttgtgtatttgtcccagtctgcttattttcctttgtctgtatcatgctgagactaatacatttgggtcgtgcattgatcttcttctcattacaacttttagctctgatcctgatgacttgtttctagtctgctatcaatgcttatcatcatactattgtaggtattgggacacaagacagccgaatcctgcccatgttcagcaacttcctgatcgttgctacgcacttgctgtgaattatccccttatgattgtgggaacagctgatcgcaatattgtgatcttcaacttgcagaatcctcaggtaattttgcttcctaaccgactatgcattttcaattcttgtggtggtggtgctcatgcttttactgccattgacttatattttgaactgcctgacttaaattcatccctgatgtgattttagactgagtttaagcgtattcaatcacctctgaaataccagacacggtgcgttgctgcctttccagatcaacaaggattcctggtaatgccatattttcttgtgtcttttactctcagcattacgacaccaaccaaaacttccgttgtttgaatacataacttgttgttactatgctttgaagtttaatcatatgttcttctctacctatccatcataattatttatgttgcccaattatgttgtttttacctagcttgagttttcgaccttaaacattgttacaaccttactcgaattagctttagatgttggcctgccatcatttgcagtgtaatgggcgttgttcctttagtgatctcatttcaaacttcctacccctctattataggataaataaggtatattgattgtgaaatcaaactttactgactataaatactatactatgtgatttatgtgacataaagttgatatcattggatttgttttcaaaagtactttgttacgattttgataaaatatgagaagaaatttatggccaaagattgaactcgagcagtcaaaatatgccttgtatatctggatagagtgtgtattactaggaatgttaatccaatataaatgtcattaattgacatctctctagttagcacaaaagctgcataaaagaagaggatgcagttttccttaatgttttgaccaactccagctgtgttgcaaacagtttccttcaccagaactgtagaacggatcttagcctatagcagttaaagctaatctgaacatgaaatccggctgaagtacaaatagtaaaaccatccaaggcttcaagcaattgtttttgaactacagatctttagatgtctatttctgcattttcctattcttcacctgcttgtgggtgttcttatagtgggtcgtcgtttcatcatgggctttgaccaatcaatttttgctgttacacaatgatctttttctacatcaatgaactgcctatattggataaagctggtcgatacctggcaatagttcttgttttaattattttcttgttgtttccatattacttgggcaaaacattttttacatgcgactgacacctgattttgtttgaaggtgggttccatagaaggaagagttggtgtgcatcatattgatgattcacagcaaagcaaaaacttcacattcaagtgtcacagggaaggaaatgatattttctctgtcaattcgctcaactttcaccctgtaagatctctatcttgttctctcttgactccatatttatctatgtgccatactgccattcttatatttcattagtccacttctgctattatggaaaatgctaatggcgacttctgttgagctttagtcactatgtctccatcatgatttgtaaaatactgaaagctcagttcgttgtcttccacaagatgtgttgagctttattcgatacagatacctaccacacttcgcctaatataataaccactatgcttgaaggcatacatatgttagtaaactactccaaattgatcaagcaatattttgcagaaataaaaatcccaacagattagcttcagtttaattgagcaactcgaaatcgtacctatttgttacagacacatcgaagagaagcgtttctgcttcttctgttgctaactttggcatcttattcattggattggtatatgcttgggtgatgatactatacatccatttagttgagtgtgcttgttggctatatatttcactggaaaatgcaactgactggtgataattgtgaagtatggaaattcagttagtctagttacttatgggatttcatgttatctaccacttggcacaggttcatcacacgtttgccacagctggatctgatggtgctttcaacttttgggataaagatagcaagcagagacttaaggtttgtctattccccttttttttttctattttcccctcttgttggtttatgttttcttgattggaattcaatgatttcaagatggctctactttgttgtgatctgtggacactgatgatctgcatataaattctttgttgatgcgcatacatagcctgaaatcatcccgagctcaagctaataaactcactatcctggccatgagcagaaaaaaaaatattagttcataaatgttatcaccaattctgattttcccagctgccttgtcatcctgctgcttttggcgtttagtatgcacttctttttattcttgataaaagcatattacatatttattactcaaccgcatggagcctctgcagtaaggtatatgtgtaatttgtcaacagataaccttttagcttttaggcatatcatacacagtgttgcattctgacagttgcttacttgcttagcatagagggctatctgtgtgtgcccaagtttattgcttcagttccctcatttgtcatacaccttctggtatgttgcacataacacattcttacatttcccctacactgcttctatacaggctttcagtcggtgtcctcaacccattccttgcagtagcttcaataatgatggttcaatatttgcttatggggtaagtcaagtgatgcttttgagagtgtcttgttatcaccatgtttttcagtccggtcagagtttagatatgttacatgcctgtcatttttcctgcaccttatttaatatacggaagaaatcagctcgacaaataattcatgttccttttgaactgctatcttgtcgtgtcctctgaatccaagttctgaatgcaaccgtaattatcattgctggggagcaggtgtgctatgactggagccgtggcgctgagaaccataatcctgcaaatgcaaagacatccatctatctccacagtccccaggtagtttccgagtgctgatatcgccataaaaagaattcttgtattttcgacatcatttgagatccattcctgtttttataaattgttattatgttatcttttggcactcaacttgcatcattctggacaggaagccgaggtgaaagggaagccaagaatcgcaacggggcggaagtgatggtggcatcctgcctgccgaaaagtcgatagttgtgcaagagatgatgtatattcaagggccttgtttcgctcgggccttgccttgacgtgtcctctggatgttcttacttggttcggccgcctggtagatgaaacgcctactcccagcaaccagtgataggttacttagccgtaggtcgtagcacatagccgtttatgtaatgcatagttgtccgaaggaatgtttccccgtgttcttgtgttgtgatatagtcaggtcagcgcgtagtcttttgaaaaggagaaagccttcggtgtcttgtaagtggtgtagatctttggaaatatgggcatgatattcggtggagttattctgaaaaagatatcccggtttcggtttcagcgaatgcaagctagatttatggggaaggttgtagttagcaccatgctgcaaatgcatgcggcattcggaggatttacagtttgaatcttcctgtttatccaagtctaggattcccaccttcctagtaccattgagcacgttagtttgaattttttttgtttattatggttcagaacttccgattcaaaaaaatctgatttattttacgcgaatatatggatgtttaatcaatatacggttttgtatgtgagatataaagaagggccaaagacatgcaaacaatgggcatttctttgtgctggatcttttgtgttcttatctgcactgcaccatcaaacgaaaattaccaggtgcgtgcagaacgacgtgtaaagaaaacacccaacttttccgaaacttccaaatgccattttcgagctgttcaacatagtgtgcttcgaaaatccgttcttcagtacagtaccagccacgtagcaatggctccacagaagcctcattgcttacgagctttatgccgtgaacttgcatagattcagttcgacaacccaattgcataagcttgcgatctttttatgccgttaacttcaaaatagattcggcgcaacacgactcaatagccacgacacgggcaaagactagggcatcatcaaacctaacgcaatatagcattggtgtcggtacttaatttacagggttttattgcatgaccaaatcacaggtgcggcataaaattacagtcgcggagagaaagcgaacccagtttcacagcaagagagtagcagactagttaacaaacgtaagagaacccagcttcaagaatctaaacacaggtaaagaccacattgtttcagcaccaagttcccgtccttccttccacgtgagaacccgacccggtcctagtcacagctccttcagacatcgaacacatcatcatacatgtcggcgtcccagtccgcgtcgtcgatataatcctccatgtcatcaaactcggcatcctcgtcgtcgtcggtgaccacgtccagcagatcatcgtacatgtcgacctcgatgtcggaaccgccggggtagtcatcactgccagcgaggtaagcccggtacttgaagtcggagatggggtccccggggagcctgaggttcccgatcctggcgcacttggatctcagggcatcgtccatctggaggttgcagcagcggcgcacgtcgagggactcgaggcgggggcagtggtcgaggatcgccgccagcccggcgttggtcaggttgttgccgatcagctggaggtcccggagctcgggcatgctgctcgcgatccctagcacacccgtgtcatcatccatgccctcaaagactgcaaaccccctttggaggaccgtccagcgctcgttcagccgaaaggatttcaggtgtgggcaggctctgccgactgactcgcacacgttgctgttcagtgagcagaatgtgatctccagctcctcaagctgaggaaagcccttgactgcatctgccagcgcctcattggatacttcatcacacaggcttagttgaaggctcttcagcgagggtgccctgcattgagacatggcaagttcttatctaacacaagcatttaagctaatgccaaacccaatcatgctttccaagctcaggacaagaaataacactagaacaacagaattgattgaaataaccatgtgaaagtaaaacattccatgctgttagattcaccatagtcttcaaataagtctaactccagtcacatgcaaatgaacttcagcacgcagttctaaacaaaatacgacatgacttgatactgtgtctgaactccacatatcacatctagtcaaaccattagaagaagacgacaacgagccaggtgttaggtggtcaagaagacaagaagtgggatcaatttatttcaatgcaccaaaagatcaacacaacgataagaaaatagcaactctgactagcactacgcttaccaagcaaacaaatggccacaagaatatggagttatgggcagcttaggggttttgcttcagtgagggggatataacagttgcaataccataaaagaaagtagctagtaatagttttacatatttatcgtttcactgcatagttgttgatttgattgttaatactatgcgatacagatatacaccagtggtaattggatgcacattcagtaaggcccttaatttttttgtatgcaaggaaatgttgtagtggtggctccaacagtataatccataagaaccaattcgagtaaatgatgatttgaacccaggtagtgggcccacccaacatgtaagttaactacatgaagagtgcaggcacactaaaacattcatcctgaagtacatgagttattttggaaacattctgatggattgttggataaaagcgtgaaagtttccaaggttgcaagtttctgtatgatctaaatgcacgagaggactgtgtacaagtgaaaggtactggaagatgcatgaaccaaaaacagttacaggaaggttaacagagaagtctgcagctaccaaaggcaggattgtcagaaatgccgaattgcatcatgcatcaatcttgcagtgaaactgcctacatctttatcagaaaaaaatggtactgaaccatcgaagtgatcatttggttttctacagacctttacagcaagtaatttgctggaaaccaacaaaaaagttcagatggccctcccccctgtaaagtctccatgctgagactcacctatccttgtatgttttagggattggtcggtgccaatgttatatccaagagcacaatgatgcccccattctgactattagtgggaacaggccaattccctccaggaaaacaaaccttcagtcagtgtgctactctatatacattttccgggttgctccacatacattatgtcccaacgctacggagtagatagaacttagcaaggcacattgagtcacacaaagacatagcactggagactcatgtgatctgttccaacttgcaagcattttactgttgagtagtaccattttaccccatccaatttcgttaggttatgaaaatgtgtgtttttttcaacctgtcaaatcaaacgtcagaaaggtccatttccaacttcagaatacagacttattttaagttaaagaccttccccatgaattttaatttaccagcagcgccagcaaccgttcgtttaactgaatgaataaactaagcgccgcatccacaaattgggggaaaaaaaaccccccactgtatgccagaaatgggcatgtgaaacaggaaggggagagccgtcgcgaacaacctgccggagatgtagctgaggagggcgtcggtgacgaaggtgtcggcgcagaaggactggagcgtgccggcggcgcggtcgacggcggcgcgggccatggcctcggcctcctcggtctcgaggatgtccccgtggtgggtcatgtcgaggcggcgccacagcgcggggtcgccgtcggcgaggcggcgccaggcgcggcacgcccgcccggcccccgtcaggatg

SEQ ID NO: 10 - DNA, 2024 bp, Triticum aestivum  (var. Fielder)
ttgtcccgccgatgataagctcggttggcgactggtccatgcccctccggactccacacagccaggcgaattggtgagtctgcaactgctgagctggacacaagtaagtcataaatgcgacgcaacaaaaaatgacgccacgaaagttgggacgttttctcaattatcccggaatattataatagcatgttgtacgcgtacctcatgtagccggaaaacatctacagccgggcaccccaaactctatcatacgccccgacggacggccaagttagtgaccggtcacgaaagactgacctagacgggtaacttaaacgggtctcaagcgctcaggctaaccggcgcccctcatatccagtcccaaatgtagggcggataagggggcgcccaggagcatcgaggcgcgtccatcacgtcggtcctggcccacgatggcccacccgcctccatatatattcttctgcatccgctgcctgggccaaacactatccacttcactcccctacgccacccaaactcctatccgacgatcacgagccttctccggtttgacgtgtagcagattcgacgacgaccagtacagatctgtcgactggggtgtcaacccgtgcggtttggagaaggaaatggccatccgcattgcactttgccatttccggaggacaacgcccttccgacgacaggatctcccagcgcgagtccattgcgtcggtgcatcgggcgctcagatcctctggtgctggatcatcgtggtcctctaggcagccttccaatctcccttccccatcactggtccgtcggagtacgagtcctatcgagggtcagtgtgcccgccgtgggaagaagaggatgagggcggctgaggtcagcgccttgctgccgacatggcggcggcggaggcggctgatacaatggcacgggcggccgcagaggaggaaggcatccgcgtccgcattgtaaagaagcggcagcggaggaacacgcgcgtccctcgtccgagagcataatcggcgatccgtgccattgccggactgccttccaaggaggagaaggaggacaacgatgggtcagatagctctggcgatgagcagacttgactcgattcgtactgcctctttgatcggtacttgcgcgagaagaacgacaagggcaccaagaagggcaaagacagacatggattgatcttcttcatagctagcatgtacgtaggttaaacatgccaatttttgatagtccgatgtcatgtcctgatgtcgtagtaggaagatgtgtgtaatgcatcgtttatgtagttgcatgagtttgtatggatttgagataaggtaattaagatgttcgattgtgagaagggaaatttaaggcgtgaccaacccgtgttcacggatgcgcctggacgcatccgctgaccggatcgtgtcgacgcgcgcgagcaaaggcggccggcgaggccaaccccaaccaccactccgcccgcgccgcccctccctttctcccgccttttcggcccccatcaatggctccctccacctccttctagaagccgcctgccccaccacccgtgctagcggtagtgtatcgggttccccttccttccttcctctcccctccctgcctgcactgtcccatcgctcgctttctcgcccgctccgtggcggtggtggtggtgcgtggtgggggagaaagttggaggcgggggaaaggagacgcaaagcggccacccaaaaaggcacatatactttctttctttccaccgccgccctcctcctcctggtcctggaggagttccgatcccccccccccccccccctctctcttctttctctctctttcttgctctgctctggaagcaggggagggagggggttccgatttgggcggggaggcactgttcgcggtgactcggttcggctgatttgggaggtcttctcgtttgatccgggctcgctgattgattgattggttggttggttcgtctccggcggctcgatttgagggtcggtcggtccggcgagtgagtcgccatg

SEQ ID NO: 11 - DNA, 2159 bp, Triticum aestivum  (var. Fielder)
gacggctcaatggtgtatttttcccgtcgtgcgtcttcatcggtgaggttatggtatgtacaattttggcgcacaatgacggcgacttccctgatccttcgtcatcgtctccaccgtgttgattttggtgctggcgtgaggcctgaggtatgtctcttggatctaatgctcgtctagcgttggtggtttctccttcagagcgatggcccggtaagacgtggccttaatcactccccgcccgtgatgaacatcaggctggcttcgatgatggagaagcatgtgttggctcggcatgttctagaggatgaagaaggatgacccagggattttgatgtaactttattaatattttcttcctataatttgtacttccttttcttagaaaaaaactattttgtactgctagtttgattaatactcgtggcttcacatgcgctggtttggttatataagagggagcatatgggtgttgtctttttatataagaaaaaaaagagcaaaataagccacgcgccgatcaagcctgcgaactgagtgtatacccggccatgggcaacccagactgacccgacccatcccgaaaaagctcgacccgtcccgactcgaccttgcccacgggccgggcccaggcctagattttgagcccgatggctgggccgacatgttgtatttgcgatttaaggaagaggcccggcccaagggccgaggcccgacgggttttttgactgatgggccggggttgggccagattattaggcccaacggccgggccgggccgggctcgggcctaagccttttgcttgcgttcaggctttgataggctcagcccgaagtccacatccacatcaaccacccccgcgtgccgatgaagccatttctttggaaggacctgcgcgtgcatccgacgcgacgcgctacaccgaagcctatactactacatctcgtctcatcgccctcctcccagacgacgcctcgagaatcctcggctgaggctccactccgcactcgcaagtcgcacccgcagccgcgccgtccccagcagccgccgccgaccgctccccgtccggcaggtaagccccccgagcccctcctcgccgctccgctcgcgcggactcctagctagctagggttcccggcaccgattccgcggcgctgcactccggcgcaatgctcgcggccgcggacgggcccgccgcttcgggggaagctctgatctggtggccggcgctcgctcgctcgctcgcggtctccggagcaaatcggccgcatcgagtggccggagcaagcttaggatagttcgattatttcccccatccccgttggtcaggccgagagggccagtttgggcgacctcctggtgattccgctgccgtggtggggattgggtggtcgtggcccgggtgtaggcgtttgcaggtggagtgagtgtttccttccaggttctatgctcacgcagctcgtgttattgctgggatccgggcgttcggcaggctaacattcaccggattttggacctgcattgttgcggcgcgtgccaggggatggcgtttatgctggctttggcgtggtgattttgtggagttgagtggtcaggttgtaggcatttgtagttgagtgatcccagctttcatgcccaccagcttgtattattactcgagttggtcgttaaccgtgtttaccgaattttggaccgccgttgttgtggcacgtaccaggggacagcgtttatgctggtttgcgaggctacctcctggtgatttcactctcgggtggtgggaatcgagtggtcggggtgtaggcatatttgtagtggagcgatcccaggttttatgaccagacatttagccgaaatttggacctacactattgtggcacgtaccaggggatagcatttatgcaggttttggcgcgacacttcttttgtcgggattttgtggtgcaaacttaaataatgacttccctaatgttgaacaatggttctggtatcgtggactgccgaaaccgttctttttggtagtctgagctactttaagttgttacttatgctctcgtgtgcgtgggagactccacgtgctggtttctcttacttgcttctcatggtcatcactggactgtgatatttcttttccagattttgaaggttcacagaaggagagatg

SEQ ID NO: 12 - DNA, 5200 bp, Artificial sequence
  pTaPK-like::RFL29a chimeric gene cassette
ttgtcccgccgatgataagctcggttggcgactggtccatgcccctccggactccacacagccaggcgaattggtgagtctgcaactgctgagctggacacaagtaagtcataaatgcgacgcaacaaaaaatgacgccacgaaagttgggacgttttctcaattatcccggaatattataatagcatgttgtacgcgtacctcatgtagccggaaaacatctacagccgggcaccccaaactctatcatacgccccgacggacggccaagttagtgaccggtcacgaaagactgacctagacgggtaacttaaacgggtctcaagcgctcaggctaaccggcgcccctcatatccagtcccaaatgtagggcggataagggggcgcccaggagcatcgaggcgcgtccatcacgtcggtcctggcccacgatggcccacccgcctccatatatattcttctgcatccgctgcctgggccaaacactatccacttcactcccctacgccacccaaactcctatccgacgatcacgagccttctccggtttgacgtgtagcagattcgacgacgaccagtacagatctgtcgactggggtgtcaacccgtgcggtttggagaaggaaatggccatccgcattgcactttgccatttccggaggacaacgcccttccgacgacaggatctcccagcgcgagtccattgcgtcggtgcatcgggcgctcagatcctctggtgctggatcatcgtggtcctctaggcagccttccaatctcccttccccatcactggtccgtcggagtacgagtcctatcgagggtcagtgtgcccgccgtgggaagaagaggatgagggcggctgaggtcagcgccttgctgccgacatggcggcggcggaggcggctgatacaatggcacgggcggccgcagaggaggaaggcatccgcgtccgcattgtaaagaagcggcagcggaggaacacgcgcgtccctcgtccgagagcataatcggcgatccgtgccattgccggactgccttccaaggaggagaaggaggacaacgatgggtcagatagctctggcgatgagcagacttgactcgattcgtactgcctctttgatcggtacttgcgcgagaagaacgacaagggcaccaagaagggcaaagacagacatggattgatcttcttcatagctagcatgtacgtaggttaaacatgccaatttttgatagtccgatgtcatgtcctgatgtcgtagtaggaagatgtgtgtaatgcatcgtttatgtagttgcatgagtttgtatggatttgagataaggtaattaagatgttcgattgtgagaagggaaatttaaggcgtgaccaacccgtgttcacggatgcgcctggacgcatccgctgaccggatcgtgtcgacgcgcgcgagcaaaggcggccggcgaggccaaccccaaccaccactccgcccgcgccgcccctccctttctcccgccttttcggcccccatcaatggctccctccacctccttctagaagccgcctgccccaccacccgtgctagcggtagtgtatcgggttccccttccttccttcctctcccctccctgcctgcactgtcccatcgctcgctttctcgcccgctccgtggcggtggtggtggtgcgtggtgggggagaaagttggaggcgggggaaaggagacgcaaagcggccacccaaaaaggcacatatactttctttctttccaccgccgccctcctcctcctggtcctggaggagttccgatcccccccccccccccccctctctcttctttctctctctttcttgctctgctctggaagcaggggagggagggggttccgatttgggcggggaggcactgttcgcggtgactcggttcggctgatttgggaggtcttctcgtttgatccgggctcgctgattgattgattggttggttggttcgtctccggcggctcgatttgagggtcggtcggtccggcgagtgagtcgccatgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatagttggtacatgatatctgaaatttaatttgcatcgcttgccatgggtccgtctgcttgtacaagaaatgcattttctatttgtaaataggaagtcagtttagaacaagccatcaggatgacgcaaagagtacaattcagttgcaccaccaataaaaaggcagaactagggctgccaaaacaacactgaatcaaaactcaaacagaaggagcagcaaacttttttttttttgaagctggacagtctgctagccaaacaactacaggagactgtcaggcggggcatgtagtggctggcgtctaagcgcctttgcttcttccaccatccatggcttagcctcacacggaatcgagtcaaccaattcccgtcggttttgggtggctcccttgaagatgcaattgttttcagcggccagatacgcatggtcagattaatcagcgagtgtgccccttctgtctggttcgagaaagaatttgaagagctgagcttgtccctgctggaaccagtttgaggttagttaatcataacagggtactagagaggtgttttattgactgttgatgtgtaatatgttatatgccatcctcttgatgattacggtgatctgtgaagaggcagcatgcaaaagtctgaatcacatatgcttatgtaattgtgttattatttgtgcagcttctagaccttttgccttgtagatggctacatggatctagttgtagcaatctgtaactgttagtgttttgtatatgctggcattgatgattagggtgacttgtgaagcatgcagaagtctgaatcgtacatgcttgtctag

SEQ ID NO: 13 - DNA, 5360 bp, Artificial sequence
  pTaRAE1-like::RFL29a chimeric gene cassette
gtggccctgctcatccctggatgttgacggctcaatggtgtatttttcccgtcgtgcgtcttcatcggtgaggttatggtatgtacaattttggcgcacaatgacggcgacttccctgatccttcgtcatcgtctccaccgtgttgattttggtgctggcgtgaggcctgaggtatgtctcttggatctaatgctcgtctagcgttggtggtttctccttcagagcgatggcccggtaagacgtggccttaatcactccccgcccgtgatgaacatcaggctggcttcgatgatggagaagcatgtgttggctcggcatgttctagaggatgaagaaggatgacccagggattttgatgtaactttattaatattttcttcctataatttgtacttccttttcttagaaaaaaactattttgtactgctagtttgattaatactcgtggcttcacatgcgctggtttggttatataagagggagcatatgggtgttgtctttttatataagaaaaaaaagagcaaaataagccacgcgccgatcaagcctgcgaactgagtgtatacccggccatgggcaacccagactgacccgacccatcccgaaaaagctcgacccgtcccgactcgaccttgcccacgggccgggcccaggcctagattttgagcccgatggctgggccgacatgttgtatttgcgatttaaggaagaggcccggcccaagggccgaggcccgacgggttttttgactgatgggccggggttgggccagattattaggcccaacggccgggccgggccgggctcgggcctaagccttttgcttgcgttcaggctttgataggctcagcccgaagtccacatccacatcaaccacccccgcgtgccgatgaagccatttctttggaaggacctgcgcgtgcatccgacgcgacgcgctacaccgaagcctatactactacatctcgtctcatcgccctcctcccagacgacgcctcgagaatcctcggctgaggctccactccgcactcgcaagtcgcacccgcagccgcgccgtccccagcagccgccgccgaccgctccccgtccggcaggtaagccccccgagcccctcctcgccgctccgctcgcgcggactcctagctagctagggttcccggcaccgattccgcggcgctgcactccggcgcaatgctcgcggccgcggacgggcccgccgcttcgggggaagctctgatctggtggccggcgctcgctcgctcgctcgcggtctccggagcaaatcggccgcatcgagtggccggagcaagcttaggatagttcgattatttcccccatccccgttggtcaggccgagagggccagtttgggcgacctcctggtgattccgctgccgtggtggggattgggtggtcgtggcccgggtgtaggcgtttgcaggtggagtgagtgtttccttccaggttctatgctcacgcagctcgtgttattgctgggatccgggcgttcggcaggctaacattcaccggattttggacctgcattgttgcggcgcgtgccaggggatggcgtttatgctggctttggcgtggtgattttgtggagttgagtggtcaggttgtaggcatttgtagttgagtgatcccagctttcatgcccaccagcttgtattattactcgagttggtcgttaaccgtgtttaccgaattttggaccgccgttgttgtggcacgtaccaggggacagcgtttatgctggtttgcgaggctacctcctggtgatttcactctcgggtggtgggaatcgagtggtcggggtgtaggcatatttgtagtggagcgatcccaggttttatgaccagacatttagccgaaatttggacctacactattgtggcacgtaccaggggatagcatttatgcaggttttggcgcgacacttcttttgtcgggattttgtggtgcaaacttaaataatgacttccctaatgttgaacaatggttctggtatcgtggactgccgaaaccgttctttttggtagtctgagctactttaagttgttacttatgctctcgtgtgcgtgggagactccacgtgctggtttctcttacttgcttctcatggtcatcactggactgtgatatttcttttccagattttgaaggttcacagaaggagagatgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatagttggtacatgatatctgaaatttaatttgcatcgcttgccatgggtccgtctgcttgtacaagaaatgcattttctatttgtaaataggaagtcagtttagaacaagccatcaggatgacgcaaagagtacaattcagttgcaccaccaataaaaaggcagaactagggctgccaaaacaacactgaatcaaaactcaaacagaaggagcagcaaacttttttttttttgaagctggacagtctgctagccaaacaactacaggagactgtcaggcggggcatgtagtggctggcgtctaagcgcctttgcttcttccaccatccatggcttagcctcacacggaatcgagtcaaccaattcccgtcggttttgggtggctcccttgaagatgcaattgttttcagcggccagatacgcatggtcagattaatcagcgagtgtgccccttctgtctggttcgagaaagaatttgaagagctgagcttgtccctgctggaaccagtttgaggttagttaatcataacagggtactagagaggtgttttattgactgttgatgtgtaatatgttatatgccatcctcttgatgattacggtgatctgtgaagaggcagcatgcaaaagtctgaatcacatatgcttatgtaattgtgttattatttgtgcagcttctagaccttttgccttgtagatggctacatggatctagttgtagcaatctgtaactgttagtgttttgtatatgctggcattgatgattagggtgacttgtgaagcatgcagaagtctgaatcgtacatgcttgtctag

SEQ ID NO: 14 - DNA, 2367 bp, Triticum aestivum (var. Spelt)
atgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatag

SEQ ID NO: 15 - protein, 788 aa, Triticum aestivum (var. Spelt)
MPRFSSTTPMSPPRLRLRLCARHSSSTSHPSRIWDPHAAFAAAAQRASSGTLTTEDAHHLFDELLRRGNPVQERPLNKFLAALARAPASASCCDGPALAVALFGRLSRDVGRRVAQPNVFTYGVLMDCCCRACRTDLVLAFFGRLLKTGLEANQVVFNTLLKGLCHTKRADEALDVLLHRMPELGCTPNVVAYNTVIHGFFKEGHVSKACNLFHEMAQQGVKPNVVTYNSVIDALCKARAMDKAEVVLRQMIDDGVGPDNVTYSSLIHGYSSSGHWKEAVRVFKEMTSRRVTADVHTYNMFMTFLCKHGRSKEAAGIFDTMAIKGLKPDNVSYAILLHGYAAEGCLVDMINLFNSMERDCILPDCRIFNILINAYAKSGKLDKAMLIFNEMQKQGVSPNAVTYSTVIHAFCKKGRLDDAVIKFNQMIDTGVRPDASVYRPLIQGFCTHGDLVKAKEYVTEMMKKGMPPPDIMFFSSIMQNLCTEGRVTEARDILDLIVHIGMRPNVIIFNLLIGGYCLVRKMADALKVFDDMVSYGLEPCNFTYGILINGYCKNRRIDDGLILFKEMLHKGLKPTTFNYNVILDGLFLAGQTVAAKEKFDEMVESGVSVCIDTYSIILGGLCRNSCSSEAITLFRKLSAMNVKFDITIVNIIIGALYRVERNQEAKDLFAAMPANGLVPNAVTYTVMMTNLIKEGSVEEADNLFLSMEKSGCTANSCLLNHIIRRLLEKGEIVKAGNYMSKVDAKSYSLEAKTVSLLISLFSRKGKYREHIKLLPTKYQFLEEAATVE

SEQ ID NO: 16 - DNA, 6120 bp, Triticum aestivum (var. Spelt)
tcggatccagctcgtctctcgtcttactcagggtcggtcatcccggtgaccttgcctctggcagcgagggggtgtgcgtgctctatggtgggccttgtgaggcgatggcgcggttgctgcgcaggggtgtgcatgccagcgattgcgcgggccagcttgtgtcggctgcgtgagggtgttggttgagtctgctccaagcgcaagcttgcccggctatggccggccggtgctggcggcgcctatgggcgtcgtttcccttgttgaaggcgttgttgtggagttcttcgacctactacaatctcgaatctggttcttcgggcgaaaacctcgccccatctaggtcgggcaacgacgtcgtctgtacggtgttttccccctttggggcgttgctttgaagaccggttatcctttcccgtgctacctttgggctggtcgtgcatggtgtcatggttggctggtgctcaaccggtgatgcgagtggttagcgttgcgactcgtgcggtgacgaagatggtgatgcgcggagctgggtcgcggcttgtccttctgatatcggcggttcggttcccctttgggtgcgtctgtgcttgtttctcactgtgacagagaagttgaagctacatgcggcgggggccctgcggcaatgatgactccatgagggtggcttcggcggacgatgctctccgcaggttgcactggactagcttggtgtgaggcttgcaactttctcctgtgatgctcatcaacaaaatcggagatgtcggtcctcgacgcctgcggccgatcgtttggcttggctgtcgaggtcctcgggtgccgtttgttcggaggggtcttgatgggtcgtcagcgaggaaatcggagttgcctgctcgtggagaggcgatgacgatgacattgcttgcagcctcgacggtgtcctctcgtcagcagtatgtgtgtgatcttgtcagtgccaggtcggccggagtgctctattcgatgggcgtgttttaattaaccgggcaactctcttcttcttaatcaatgaaaatggcaagtcttttacctcgtttcaaaaagagttaaatgcactggaggtcctaaaggtttcgtcggggtgtcactttagtcctatttcttccaaaatgcacctttaggtcctaattctttattaattggttcacccgaggtccttttccacgagcgctagctgaccagcacacgtgccgcattgacccacgccacatcgatagagggtctaaccgccaggcgagaagcttcgcaaagagacccccccccctcttagtggcaccctctctcctctttcacggtactttggcgacattggaggcgagcgacagaggcaaccggaggtgatctggaggtaatttgtgtgggtgcccgtgaagatgccccttctacgacggccagtgagcaatcctcctccatgttaccgtaagaaccctaatcttgtcgacgatcttctcctgtagctcttctaatatgtcataactttgccataatcaatagttgtagtccttgatttactttgatttgcataacggtgctacggacatggcaaaaaggacacaccaccagcggcaacatacccgccgaagttggagcttgcttcgccgcaccggcggcggtgctgaggatgttgccatgctcctcatcttagttgtttccttgccgaggagcttgaagttccggccatggataggtttagggaacgcgcgcgccgagagagagagcagatttggggaagaaataaagccagggactggggaatgagaagataatgtttataaggggttttctgtaaaaagaaagagcaacctacgcggcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagcgcttacaactaaatttatattcttggtctaaataaaattataatctgataaattatgtgatatgtatatagccatatgagaccataatttaattttacctgcaaaaagtaagtaatagtaatagtagtactacatactccctccgtccgaaaatacttgtcgaagaatttgatgaaaatggatgcatctagaacaagaatacatctagatacatcaatctccccgacaagtatttccgaacggagggagtactagataataagataactatccaaaaaaaaaaagataactgaaggttgccacctagcacattcacattggtacaacttggaaaagcacagccccgtcgtcctgctcccagttgagttcgcgacctacacaccggccatgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatagttggtacatgatatctgaaatttaatttgcatcgcttgccatgggtccgtctgcttgtacaagaaatgcattttctatttgtaaataggaagtcagtttagaacaagccatcaggatgacgcaaagagtacaattcagttgcaccaccaataaaaaggcagaactagggctgccaaaacaacactgaatcaaaactcaaacagaaggagcagcaaacttttttttttttgaagctggacagtctgctagccaaacaactacaggagactgtcaggcggggcatgtagtggctggcgtctaagcgcctttgcttcttccaccatccatggcttagcctcacacggaatcgagtcaaccaattcccgtcggttttgggtggctcccttgaagatgcaattgttttcagcggccagatacgcatggtcagattaatcagcgagtgtgccccttctgtctggttcgagaaagaatttgaagagctgagcttgtccctgctggaaccagtttgaggttagttaatcataacagggtactagagaggtgttttattgactgttgatgtgtaatatgttatatgccatcctcttgatgattacggtgatctgtgaagaggcagcatgcaaaagtctgaatcacatatgcttatgtaattgtgttattatttgtgcagcttctagaccttttgccttgtagatggctacatggatctagttgtagcaatctgtaactgttagtgttttgtatatgctggcattgatgattagggtgacttgtgaagcatgcagaagtctgaatcatacatgcttgtctagttatttttggagctctctctttgccatgtacatgttttatggatgcagttgtagtctgtatgtgctggcatttaatatgagggctggtctgcaatttttctgttttgatcgatacagcgccatgttgagtttaaccctgtaacttggcttaactagatcatgctggtgttatctctaggaatcccaatatcaatactctctgttgttgcacaaactgacgcagatcaatgtccagaggtaatatggttctgcctgtaattttcatcgactgtcagcatgttccttttttatttagtactctgtatatcacggcggtatgactataataaggatatacggctggtgcattttgagcttctctgtttcttgggataaagctgataaatttctattcttgttggctttggtagtatgaacagaaagcaggcagtaata

SEQ ID NO: 17 - DNA, 1212 bp, Triticum aestivum (var. Spelt)
atggggaactgctggggcgccaagatcagctccgacacctcctcctcctccccttcagggacgaattccaagtatgctagtaggaatggggcggccttgagcagctccagcagctatgcctccgcggcgtcagtgccgcggagcgagggtgagattctggagtcagcaaatgtcaaggcctttaccttcaatgagctgaggaccgccacgagaaacttcaggccggacagtgtgctaggcgagggagggtttgggtcagtcttcaaggggtggatcgatgagaagaccctcaccccaaccaagccaggcaccgggatggtcattgctgtaaagaagcttaaccaggaaagctatcagggccatagggaatggctggctgaagtgaattaccttggacaactatcgcacccgaatcttgtaaagctcgttgggtactgtgttgaagacgaacagcggcttctcgtctacgagttcatgcctcgtgggagtttggagaatcatctcttcaggaggagtacacatttccagccgctctcctggaaccttcggatgaaaattgcccatggagcagcaaaagggctcgcatttctccacagtgacaaggccaaagtcatctaccgcgatttcaaaacctctaatatcctcctagatgcgaactatgacgcaaagctctcagatttcggcctggcgaaggacggaccgacgggtgacaagagccatgtgtccacaagggtgatggggacatatgggtatgctgcaccagaataccttgcaacaggccaccttaccacgaagagcgacgtgtacagcttcggcgtggtcctcctggagatgctctcggggcgtcgcgctgtggacaagaaccggccgaccggcgagcacaacctggtggagtgggcgcggccgtacctgacaagcaagcggcgcatcttccgcgtcctggatccccgtctaggcgggcagtactccctcgccaaggcccagaaggcggcgtcgctggcgctgcagtgcctctcgatggactcgaggaacaggccgagcatggagcaggtcgtcgtggcgctggagcagctccacgacgccaaggagggagggaacagccctcgcccgcagctgcagagaaagccgagcagcaaccggggcgtgaacggctcgagatcgtcgtcgaccaaggggaacaccaagaagcccgcctcaccgagaccggtttga

SEQ ID NO: 18 - protein, 403 aa, Triticum aestivum (var. Spelt)
MGNCWGAKISSDTSSSSPSGTNSKYASRNGAALSSSSSYASAASVPRSEGEILESANVKAFTFNELRTATRNFRPDSVLGEGGFGSVFKGWIDEKTLTPTKPGTGMVIAVKKLNQESYQGHREWLAEVNYLGQLSHPNLVKLVGYCVEDEQRLLVYEFMPRGSLENHLFRRSTHFQPLSWNLRMKIAHGAAKGLAFLHSDKAKVIYRDFKTSNILLDANYDAKLSDFGLAKDGPTGDKSHVSTRVMGTYGYAAPEYLATGHLTTKSDVYSFGVVLLEMLSGRRAVDKNRPTGEHNLVEWARPYLTSKRRIFRVLDPRLGGQYSLAKAQKAASLALQCLSMDSRNRPSMEQVVVALEQLHDAKEGGNSPRPQLQRKPSSNRGVNGSRSSSTKGNTKKPASPRPV

SEQ ID NO: 19 - DNA, 7170 bp, Triticum aestivum (var. Spelt)
ctgtcgtactcgtgacggccatctacacgtataatactcccacttgcacatacgtactatgcacatgtacaacacatgataataaagtttggcaaacattgatcgttgcggggcgagctgcaggtccatgttcacattgtattttctcagccaagcagaacaatactagaggacatacgccattgacttgacaatttggttaccacccacaaactgtattgaccagatatatacaatatatgtaataaatgttataaaaagaaatggaaaataataatttcggtgcatgaatcgtgcctcgtccacgaatggtgatactagacgttttcccggctagctagtaaaaaagtaggcgatcggtgtacgggtatcgccgtaaccactaatattgtcgcacctacgtatacgcatgtaccggccatattataaacaaaacacacgaagaagaaacggaggcgttgtacggacggcgatcgtgactgcacgatgtgaggagccgtcccacactctatcgttttcctaccctagccgccgccgtcaccaagtgagtagggcgaggcgactcttttccttcgatctccaccggccggtgcgctggccccctctcccttcggccgctgctccggcggcaggagggagggggaccccgttcctctgttctgtagttaggttttggatttgtaggtcgtgatgcgtcgtttgggtggtgggagcggcgtccggacgaataaatctgcctcaaccccactcccacaccggcggtgtctttcatggaggtgtctcggagttggtggcatcgtgtgtatgtcgatccctcagatcgacagcgttagggttcatggatggtgttggcgaggcggcggcggcgacgccatcaggtgtttctctccttctgctcggtccccgttgctgtggtgttgtctccgacgtcatggtggagcagggaggttttgtcgttcaggagtatgcgcaggcggttgtctacgcaagtgacatctggaagatggagtatcttgtgttgggtccgtggtcgaaggcgggcagttctggtttccttctccgacgtcgtcgtcatgcgagggcgtcagttctgaagttcgatggcgtgtccggggacatgttgccccggtctgattctttcaacggctatgattttgcttttggcaaactactttggagatccgtaaagctgctgatcagcgatggagccgcgtcgagcttgggcgaagatgtgatctgtcttcttaccctttggtcaccgcttcggtggtgtcgaaggaaagggacatgcgctggtattttgcatagaattttcctattttttccgtcatgtatggtcggtgcttacgtgccttgtaacttgatctttattctatataaatgagacacgtattatcatgaaaaaagaaaacggacggcgatctggcgtggcaatcacgggcagacgaaattgaccgtttggatttgaatattcgcttttatatacgtcgaacgactctcgatgctgtaccatcaaatgtactgtataatcggagtagaaaatatactggcacacgagcaactagacataatttttattcgcacttgttttatactgatatatgttgtggcgatcagttttgtttaatcttttatcccgctgatgataagctcgattggcgactggtccatgcccctccgggctccacacagccaggcgaattcgtgctatcatgcaggcagcccgaacaaagaacatgttctcagtctgcaactgctcagccggacccaagtaagtcataaatgcgacgcaacaaaaaatggcgccgcaaaagttgggacgttttctcagttatcctgaaatattataataacatgcatgttgtatgcgtacctcatgtagcggggaaacatatgcagccgggcaccccaaaccccgtcatacgcccggaccgacgcccaaattagtgaccggtcacgaaagactgacccagacgcgtgcctcgaatgggtcttaaacgctcaggctaaccgccacccctcatatctagtcccaaatctaagacggataagggcgcgccgcccaggagcgtcggggcgcgtccatcacgttggtcctggctcacgatggcccacccgactccacatccgctgcctgggccaaacactatccacttcacttccctccgccgcccaagctcctatccgccgatcacgagccttcttcggcttgacgtgtagcgaatccgacgacgaccagtacagatccgttgactagggtgtcaacccgtgcggtttggagaaggaaatggccatccgcattgcacttcgccactcccgggaggacaacgcccttccgagggcgggatctacccaacgcgagtccattgcgtcggtgcatcgggcgctcagattctccggcgctggatcatcgtggtcctctcgggagccttccaatctccctttccccatcatggtccgtcggagtacgattcctatcgggtatcagtgtgcccgccgtgggaagaagaggatgagggcgtctgaggtccgcctcgctgccgacattgcggcagcggaggcggctgatgcgatggcaggggcggtcgcaggggaggaagtcatctgcgtccgcattgtaaagaagcggcagcgaacgaacacgcgcgcccctcatccgagagcagaatcggcgggccgtgccatggccggactgccctccaaggaggagaaggaggacaacgatgggtcagacagctccgacgatgagcagatttgagtattttcgtagtgcaaaatgtcatagacctgtttaaatacgagtatcttcctagtgcaaaatgtcgtagacctgtttaaatacgagtatcttcgtagtgcaaaatgtcgtaggaaatagaaatagtatcctacgggtatgtagttgcatttcttactagagtagatcgtaggctcaactgaaagcactactagtacgtggtggtgataacctaatacgtgactttgaacatgtctctgcccctccctttctctcgccttttcggccccatcaatggctccctccccctccttctagagactagaagcaggccccgcctccacaggtgctagcggtagtgtatcccgctcggtggcggtggtggtgcgtggtgggggagaaagttggaggccggccagcgcacggggaaaaggagacgcaaagcggccacccaaaaaggcgcacatactttctttctttctttccaccgccgccctccggaggagttccgattccccctctctttctctctcttctttctctctctctctttcttgctctgctctggaagcaggggaaggagggaggggcttccgatttgggcgcggaagcactgtccgccgtgattcgattcggctgatttgggaggtctgctcgtttgatttgggttcgctgatttattggttgatttgttggttcgtgtccggcggctcgatttgagggccggacggtcctcgccatggggaactgctggggcgccaagatcagctccgacacctcctcctcctccccttcaggtgcgcgcgtcttcctcgtctcctgctgctattttacaaatgtttttactagtactactatactagcagtactgatctgtgcccataactcgactagtagtactgatctgtgcccacagggccgattgctattttctttgcccctcaatttctttcacaactcacaagcagccgaataaataagttctgaatgagaaacaagtgttcaaccaaccatttctctcactccaatctgctcatttcgtgctctgcatctcttctctccttcccgaaaccattcttaggttatgctgatttcggcgacaggtttggtcaaagtcaatttcgttctgtttaattggcttatgatagttgagtcactaaattcagtccctccttttggaaaaatctgtcgcgagcttggactcttcctcttgacctgcttcatgtgcaaggccaaggaatcatgcctttccgtttatggcctaggggagaggaagcttccgatgtagtaaaatgcttcacttaactttatctgacttctcctctgatttttctgagtgtccagttcaaatcagacatatagaggaaaagctcgaattctgatgctacttttggctacatccacgtgcttttcttcaaagttattgactgtcctgttttggatgttcagtcttcttttctgtaataagcgaaactaaaggaagaaaatacatagttagtccgacctaacttgtccatgatggacctattttctgttcaatgttaggcagacaagataaatgattaagggacatcaacttatttatgctaaaatgtgactgaataatctctgaaagtctgcaccattttatgatgatgatcagggacgaattccaagtatgctagtaggaatggggcggccttgagcagctccagcagctatgcctccgcggcgtcagtgccgcggagcgagggtgagattctggagtcagcaaatgtcaaggcctttaccttcaatgagctgaggaccgccacgagaaacttcaggccggacagtgtgctaggcgagggagggtttgggtcagtcttcaaggggtggatcgatgagaagaccctcaccccaaccaagccaggcaccgggatggtcattgctgtaaagaagcttaaccaggaaagctatcagggccatagggaatggctggttagtgacaattttgcctgctggaaatgggatttcttgtttatttcagttctgcattgtgtctgacatgctctttcttttgggcgcaggctgaagtgaattaccttggacaactatcgcacccgaatcttgtaaagctcgttgggtactgtgttgaagacgaacagcggcttctcgtctacgagttcatgcctcgtgggagtttggagaatcatctcttcaggagtaagtgcctggacttttacttttcttattattcgctgggtgttattctccattttttaaggaatgatgctaaagtgttccaacattcagggagtacacatttccagccgctctcctggaaccttcggatgaaaattgcccatggagcagcaaaagggctcgcatttctccacagtgacaaggccaaagtcatctaccgcgatttcaaaacctctaatatcctcctagatgcggtatatatgtccacagcctggaaggatcttttccttcgtgtccgcaaaatatctctatcggttggtctgacagtcacgcaatttcttcttcagaactatgacgcaaagctctcagatttcggcctggcgaaggacggaccgacgggtgacaagagccatgtgtccacaagggtgatggggacatatgggtatgctgcaccagaataccttgcaacaggtcggttgatcattccactttcagaatcctcagctgcgcaaatatcagatatattgccgaaaatctgaccacacctacctgaatcgtgattgcgttgtcactctggttgtgtcatcgaccaagatgccaaacataaccaattttaggctacaacctttcaatcctcatctgcacaaatatcttgcccgaaaatctaaccacacctacctgaatcatgattgcgttctcactctggttgcgtcatcgaccaagatgccaaactcaactaatttttcggctagaacctttcaatcatcatctgcacaaatatcttgctgaaaatccaaacacacctacctgaatcgcgagtgcattgtcaccctggttgcgtcatcaaccatcatgccgaacctaaactaattttcctgctgcaatatttccatgatcaggccaccttaccacgaagagcgacgtgtacagcttcggcgtggtcctcctggagatgctctcggggcgtcgcgctgtggacaagaaccggccgaccggcgagcacaacctggtggagtgggcgcggccgtacctgacaagcaagcggcgcatcttccgcgtcctggatccccgtctaggcgggcagtactccctcgccaaggcccagaaggcggcgtcgctggcgctgcagtgcctctcgatggactcgaggaacaggccgagcatggagcaggtcgtcgtggcgctggagcagctccacgacgccaaggagggagggaacagccctcgcccgcagctgcagagaaagccgagcagcaaccggggcgtgaacggctcgagatcgtcgtcgaccaaggggaacaccaagaagcccgcctcaccgagaccggtttgaccgtgtgagtgggatcacatagcagaatctagtagagagatgcgtcaaggtgtaatgtatgtatggtataatagggttttagagtggtgatctgcgaagaggcagcggatttgtgaatgttttgtgcccgtgttgtagggatgtatctttgcagcttctagtgttggtatgtacagcttgtatctatccggttgtagctagtctgtatgtagtccttttgtgtatgcctgattgctttggttctgacttaaggtccagaggttttgatctgatttagatgcatgcaccctgttaaaccaagaccttaaaatatgaagatgaacagtgtaaagtacatatttgtcacttcatttatcaaatgtgaatgaacaatcatgttggtgttttagcagagcaggccatgacttccatgggtcaactgtatcttttcccttagggatgttgaccattttgttcatatcatacagtactagtagtatatgtttcatttttttaatgaaaaaagtagtatatgttacagataatgttgggaaaatcaccttctgtatttttaatttcaaacagttggccacaatgatctttttatttacttggtgggttcagccaaattaccattgtttttttcaggggtttcagccaaattattattatctgggcaagcgtacggcggcggcccaaggcagagcacgcagcccagccgccaaaacagagctcgccctcgctcccctccggcccaagtgccgctcctcctcctcctcggtggacgccggcgccggtggatggctccgccgcgccaaatcgagcccctcacgccgcgagccgctcctcctcctcctcagcctgcaatccccgccgagctccgatccccgccgccgagctccgatccgcgggcgatcgccggggcgccgccaagatcagctccggcccctcccgttcc

SEQ ID NO: 20 - DNA, 1032 bp, Triticum aestivum (var. Spelt)
atggcagctctaaccagccttgcctcaaacccgaatccaaacaagtcattcgaggtccttcctaatccgggtgactccctctcaagcctcagttttagcccgaaaagtaatcttcttgtggcaacttcctgggataaccaggtgaggtgttgggagataggtaatggtaacagtcagccaaaggcatccatatcacatgatcagccagtgctctgctcagcctggaaagatgatgggactactgtcttctctggagggtgtgataaacaggtcaaaatgtggcctctgctgtctggtgggcaggctcagacggttgcaatgcatgatgcacctgtcaaggaggtcgcatggatttctcagatgaatcttcttgtatccggaagctgggacaagacactaaggtattgggacacaagacagccgaatcctgcccatgttcagcaacttcctgatcgttgctacgcacttgctgtgaattatccccttatgattgtgggaacagctgatcgcaatattgtgatcttcaacttgcagaatcctcagactgagtttaagcgtattcaatcacctctgaaataccagacacggtgcgttgctgcctttccagatcaacaaggattcctggtgggttccatagaaggaagagttggtgtgcatcatattgatgattcacagcaaagcaaaaacttcacattcaagtgtcacagggaaggaaatgatattttctctgtcaattcgctcaactttcaccctgttcatcacacgtttgccacagctggatctgatggtgctttcaacttttgggataaagatagcaagcagagacttaaggctttcagtcggtgtcctcaacccattccttgcagtagcttcaataatgatggttcaatatttgcttatggggtgtgctatgactggagccatggcgctgagaaccataatcctgcaaatgcaaagacatccatctatctccacagtccccaggaagccgaggtgaaagggaagccaagaatcgcaacagggcggaagtga

SEQ ID NO: 21 - protein, 343 aa, Triticum aestivum (var. Spelt)
MAALTSLASNPNPNKSFEVLPNPGDSLSSLSFSPKSNLLVATSWDNQVRCWEIGNGNSQPKASISHDQPVLCSAWKDDGTTVFSGGCDKQVKMWPLLSGGQAQTVAMHDAPVKEVAWISQMNLLVSGSWDKTLRYWDTRQPNPAHVQQLPDRCYALAVNYPLMIVGTADRNIVIFNLQNPQTEFKRIQSPLKYQTRCVAAFPDQQGFLVGSIEGRVGVHHIDDSQQSKNFTFKCHREGNDIFSVNSLNFHPVHHTFATAGSDGAFNFWDKDSKQRLKAFSRCPQPIPCSSFNNDGSIFAYGVCYDWSHGAENHNPANAKTSIYLHSPQEAEVKGKPRIATGRK

SEQ ID NO: 22 - DNA, 9314 bp, Triticum aestivum (var. Spelt)
  misc_feature (830)..(903): n is a, c, g, or t
tttgtaaaaggctatggggacatttgttttcctctggcgcacagaaatcaagaggtgtcattaattctgaatgaagaagacaatgctatgggctccgcaatacctcgctaacgaaggtgtcgagggtggccgagctatcatcagccggatacaggagacgggaggaactcgcccctcaatccgaagacttcaagaagcacagagctaatggaaattatcgacctcgctgttggaggctagttcggaggctaatgagggtgtcttggactaaggggtcctcgggctgccgacctatttctcatgggccgtactagtggactgtttgttgttcgtcaggggaagaccggacttcggacgaccccgtgctccaaaaggaaaccttcggagacttgacgtatcctccaagtctggcttagtgtgacttgtgtgtttatcccctgttggtaaccgacatatgtaaccctagggacccctggtgtctatataaacagagggtttagtctgtagaggctagaaccatcatcctactcctatagggtttagttcatctgatctcgtggtagatcaactctgtaatcatcacacaaacacaccaagacaatcaagcagaatgtagggttttacctcttcgagagggctcgaacctgggtaaatactatctctaccttccctgttacccatgcatccaagatctacaactcaggaccccctaccgagatctgccggttttgacaccaacacacataaaattgagggtgaccaatcacgtaaagctcttaatagcgatattaatatacgtacacatagcaattgtcgatgaaattgttggnnnnnnnnnnnnnnnnnnnnnnnnnnnnnnnnnnnnnnnnnnnnnnnnnnnnnnnnnnnnnnnnnnnnnnnnnncctgctcatccctggatgttgacggctcaatggtgtatttttcccgtcgtgcgtcttcatcggtgaggttatggtatgtacaattttggcgcacaatgacggcgacttccctgatccttcgtcatcgtctccaccgtgttgattttggtgctggcgtgaggcctgaggtatgtctcttggatctaatgctcgtctagcgttggtggtttctccttcagagcgatggcccggtaagacgtggccttaatcactccccgcccgtgatgaacatcaggctggcttcgatgatggagaagcatgtgttggctcggcatgttctagaggatgaagaaggatgacccagggattttgatgtaactttattaatattttcttcctataatttgtacttccttttcttagaaaaaaactatttggcttcacatgcgctggtttggttatataagagggagcatatgggtgttgtctttttatataagaaaaaaaagagcaaaataagccacgcgccgatcaagcctgcgaactgagtgtatacccggccatgggcaacccagactgacccgacccatcccgaaaaagctcgacccgtcccgactcgaccttgcccacgggccgggcccaggcctagattttgagcccgatggctgggccgacatgttgtatttgcgatttaaggaagaggcccggcccaagggccgaggcccgacgggttttttgactgatgggccggggttgggccagattattaggcccaacggccgggccgggccgggctcgggcctaagccttttgcttgcgttcaggctttgataggctcagcccgaagtccacatccacatcaaccacccccgcgtgccgatgaagccatttctttggaaggacctgcgcgtgcatccgacgcgacgcgctacaccgaagcctatactactacatctcgtctcatcgccctcctcccagacgacgcctcgagaatcctcggctgaggctccactccgcactcgcaagtcgcacccgcagccgcgccgtccccagcagccgccgccgaccgctccccgtccggcaggtaagccccccgagcccctcctcgccgctccgctcgcgcggactcctagctagctagggttcccggcaccgattccgcggcgctgcactccggcgcaatgctcgcggccgcggacgggcccgccgcttcgggggaagctctgatctggtggccggcgctcgctcgctcgctcgcggtctccggagcaaatcggccgcatcgagtggccggagcaagcttaggatagttcgattatttcccccatccccgttggtcaggccgagagggccagtttgggcgacctcctggtgattccgctgccgtggtggggattgggtggtcgtggcccgggtgtaggcgtttgtaggtcgagtgagtgtttccttccaggttctatgctcacccagcttgtgttatcactgggatccgggcgttcggcaggctaacattcactggattttggacctgcattgttgcggcgcgtgccgggggatagcgtttatgctggctttggcgcggtgattttgtgcgtgcgtgcaaacttttggggctgccttctggtgatctcgctgtcgggtggtgcgagttcatcaagcggtcagggtgtaggcatttgtagtcgagcgaccccagctttcatgcccaccagctcgtattattcggtcgttaaccacgtttaccgaattttggaccgacgtcgttgcggcacgtaccaggggacagcgtttatgctggtttcggtgtggagattttgtggtttaaacttggggcgcgcttggtttgcgaggctacctcctggtgatttcaccgtcgggtggtgggaatcggatggtcggggtgtaggcatttatagtggagcggaccccttgttttatgaccagacatttagccgaaatttggacctacactatcgtggcacgtaccaggggatatcatttatgcgggttttggcgcgacactttctttgtcgggattttgtggtgcaaacttaaataatgacttccctaatgttaaacactcgttctggcatcgtggactgttggaatcgttctttttcgtagtctgagctactttaaggtgttacttatgctctcgtgtgcgtgggagactccacgtgctgatttctcttacttgcttctcatggtcactggactgatatttcttttccagattttgaaggttcacagaaggagagatggcagctctaaccagccttgcctcaaacccgaatccaaacaagtcattcgaggtgtgatactttttgtggccatgtctgtcctgcttttatttttttgcgtcttgtcattctgtatatgctacatgttctctgcaatggttgctttcaaatgcaggtccttcctaatccgggtgactccctctcaagcctcagttttagcccgaaaagtaatcttcttgtggcaacttcctgggataaccaggtaattttgtttctgctgtaattttcagtgagcttccattattgtgatgtatcttactaggaatgtcttttgagttttgtgcaggtgaggtgttgggagataggtaatggtaacagtcagccaaaggcatccatatcacatgatcagccagtacgttctcatacatgtttctcctgaaagtagtttcatcttacacattagagtggttccttacctgtttacttggtatgatgattgatttgtttccttccatcaggtgctctgctcagcctggaaagatgatgggactactgtcttctctggagggtgtgataaacaggtcaaaatgtggcctctgctgtctggtgggcaggctcagacggttgcaatgcatgatgcacctgtcaaggaggtcgcatggatttctcagatgaatcttcttgtatccggaagctgggacaagacactaaggtgcaagaacaatattcctctccaccctcagtattctttttacatggaaattcagcgcatatcacccatatctgatcccaaattgttatttatggctgttttgcttttgggaattcgtgttgtttgtggtatcctttctgtacttcatgtgttctgcattaatgttcacacattggctaaccaaatattgtagaagttctatctcatcctctttatatttgcactcattttgtatgccttctttgatcctgaagagttttctactacttgttgaatcatactgccctattcttatcaaaggttgtgcactttctgggtatcatctgtactatttatggctgtagtgaatttttcttatggcagaagtttttatagaagtaactgttgatgcagacagttactatattaaatgtacattatgtgattagtgttagaaatgttcgtttcgagttttagcatcatctttatgaagattatttatgaaacatcaagttagggtctggctgtcagtttaaacatgagcacagattgagcataggcgataatcatggttggtaggaggccagggaccgggatccttgtgggacctgccggcgtacccgtgatagcagaaggggtgcaagggacagggcactggaactaaggcaggcagaaacagagaaataaagattggggattagactttgtagccaattcataaaaaaattggttgcatcattttgtagaggttggccttaacatatgactaaaaaacaaaaacaaaccaagactcttccctaaactgaataccgcagatactcaagacaccatatagggccacgggctcaggctgaagtcatctaggtctcagacatgccaaaacagactgggacaaggacaagctgaaactgactagtactaggtttcccggggcaactaccgttcagctggaggtctgaatgcgtggtgccttctctgatacatggatctcatgtggcacttctgttgtgtatttgtcccagtctgcttattttcctttgtctgtatcatgctgagactaatacatttgggtcgtgcattgatcttcttctcattacaacttttagctctgatcccgatgacttgtttctagtctgctatcaatgcttatcatcatactattgtaggtattgggacacaagacagccgaatcctgcccatgttcagcaacttcctgatcgttgctacgcacttgctgtgaattatccccttatgattgtgggaacagctgatcgcaatattgtgatcttcaacttgcagaatcctcaggtaattttgcttcctaaccgactatgcattttcaattcttgtggtggtggtgctcatgcttttactgccattgacttatattttgaactgcctgacttaaattcatccctgatgtgattttagactgagtttaagcgtattcaatcacctctgaaataccagacacggtgcgttgctgcctttccagatcaacaaggattcctggtaatgccatattttcttgtgtcttttactctcagcattacgacaccaaccaaaacttccgttgtttgaatacataacttgttgttactatgctttgaagtttaatcatatgttcttctctacctatcccatcataattatttatgttgcccaattatgttgtttttacctagcttgagttttcgaccttaaacattgttacaaccttactcgaattagctttagatgttggcctgccatcatttgcagtgtaatgggcgttgttcctttagtgatctcatttcaaacttcctacccctctattataggataaataaggtatattgattgtgaaatcaaactttactgactataaatactatactatgtgatttatgtgacataaagttgatatcattggatttgttttcaagagtactttgttacgattttgataaaatatgagaagaaatttatggccaaagattgaactcgagcagtcaaaatatgccttgtatatctggatagagtgtgtattactaggaatgttaatccaatataaatgtcactaattgacatctctctagttagcacaaaagctgcataaaagaagaggatgcagttttccttaatgttttgaccaactccagctgtgttgcaaacagtttccttcaccagaactgtagaacggatcttagcctatagcagttaaagctaatctgaacatgaaatccggctgaagtacaaatagtaaaaccatccaaggcttcaagcaattgtttttgaactacagatctttatagatgtctatttctgcattttcctattcttcacctgcttgtgggtgttcttatagtgggtcgtcgtttcatcatgggctttgaccaatcaatttttgctgttacacaatgatctttttctacatcaatgaactgcctatattggataaagctggtcgatacctggcaatagttcttgttttaattattttcttgttgtttccatattacttgggcaaaacatttttttacatgcgactgacacctgattttgtttgaaggtgggttccatagaaggaagagttggtgtgcatcatattgatgattcacagcaaagcaaaaacttcacattcaagtgtcacagggaaggaaatgatattttctctgtcaattcgctcaactttcaccctgtaagatctctatcttgttctctcttgactccatatttatctatgtgccatactgccattcttatatttcattagtccacttctgctattatggaaaatgctaatggcgacttctgttgagctttagtcactatgtctccatcatgatttgtaaaatactgaaagctcagttcgttgtcttccacaagatgtgttgagctttattcaatacagatacctaccacacttcgcctaatataataaccactatgcttgaaggcatacatatgttagtaaactactccaaattgatcaagcaatattttgcagaaataaaaatcccaacagattagcttcagtttaattgagcaactcgaaatcgtacctatttgttacagacacatcgaagagaagcgtttctgcttcttcttttgctaactttggcatcttattcattggattggtatatgcttgggtgatgatactatacatccatttagttgagtgtgcttgttggctatatatttcactggaaaatgcaactgactggtgataattgtgaagtatggaaattcagttagtctagttacttatgggatttcatgttatctaccacttggcacaggttcatcacacgtttgccacagctggatctgatggtgctttcaacttttgggataaagatagcaagcagagacttaaggtttgtctattcccgtttttttttctattttcccctcttgttggtttatgttttcttgattggaattcaatgatttcaagatggctctactttgttgtgatctgtggacactgatgatctgcatataaattctttgttgatgcgcatacatagcctgaaatcatcccgagctcaagctaataaactcactatcctggccatgagcagaaaaaaaaatattagttcataaatgttatcaccaattctgattttcccagccgccttgtcatcctgctgcttttggcgtttagtatgcacttctttttattcttgataaaagcatattacatatttattactcaaccgcatggagcctctgcagtaaggtatatgtgtaatttgtcaacagataaccttttagcttttaggcatatcatacacagtgttgcattctgacagttgcttacttgcttagcatagagggctatctgtgtgtgcccaagtttattgcttcagttccctcatttgtcatgcaccttctggtatgttgcacataacacattcttacatttcccctacactgcttctatacaggctttcagtcggtgtcctcaacccattccttgcagtagcttcaataatgatggttcaatatttgcttatggggtaagtcaagtgatgcttttgagagtgtcttgttatcaccatgtttttcagtccggtcagagtttagatatgttacatgcctgtcatttttcctgcaccttatttaatatacggaagaaatcagctcgacaaataattcatgttccttttgaactgctatcttgtcgtgtcctctgaatccaagttctgaatgcaaccgtaattatcattgctggggagcaggtgtgctatgactggagccatggcgctgagaaccataatcctgcaaatgcaaagacatccatctatctccacagtccccaggtagtttccgagtgctgatatcgccataaaaagaattcttgtattttcgacatcatttgagatccattcctgtttttataaattgttattatgttatcttttggcactcaacttgcatcattctggacaggaagccgaggtgaaagggaagccaagaatcgcaacagggcggaagtgatggtggcatcctgcctgccgaaaagtcgatagttgtgcaagagatgatgtatattcaagggccttgtttcgctcgggccttgccttgacgtgtcctctggatgttcttacttggttcggccgcctggtagatgaaacgcctactcccagcaaccagtgataggttacttagccgtaggtcgtagcacatagccgtttatgtaatgcatagttgtccgaaggaatgtttccccgtgttcttgtgttgtgatatagtcaggtcagcgcgtagtcttttgaaaaggagaaagccttcggtgtcttgtaagtggtgtagatctttggaaatatgggcatgatattcggtggagttattctgaaaaagatatcccggtttcggtttcagcgaatgcaagctagatttatggggaaggttgtagttagcaccatgctgcaaatgcatgcggcattcggaggatttacggtttgaatcttcctgtttatccaagtctaggattcccaccttcctagtaccattgagcacgttagtttgaattttttttgtttattatggttcaaaacttccgattcaaaaaaatctgatttattttacgcgaatatatggatgtttcatcaatatacggttttgtatgtgagatataaagaagggccaaagacatgcaaacaatgggcatttctttgtgctggatcttttgtgttcttatctgcactgcaccatcaaatgaaaattaccaggtgcgtgcagaacgacgtgtaaagaaaacacccagcttttccgaaacttccaaatgccattttcgagctgttcaacatagtgtgcttcgaaaatccgttcttcagtacagtaccagccacgtagcaatggctccacagaagcctcattgcttgcgagctttatgccgtgaacttgcatagattcagttcgacaacccaattgcataagcttgtgatctttttacgccgttaacttcaaaatagattcggcgcaacacgactcaatagccacgacacggg

SEQ ID NO: 23 - DNA, 23 bp, Triticum aestivum (var. Spelt)
gcggggcatggccggtgtgtagg

SEQ ID NO: 24 - DNA, 23 bp, Triticum aestivum (var. Spelt)
gaggagaagcggggcatggccgg

SEQ ID NO: 25 - DNA, 23 bp, Triticum aestivum (var. Spelt)
tgagttcgcgacctacacaccgg

SEQ ID NO: 26 - DNA, 23 bp, Triticum aestivum (var. Spelt)
tccccatggcgaggaccgtccgg

SEQ ID NO: 27 - DNA, 23 bp, Triticum aestivum (var. Spelt)
cccagcagttccccatggcgagg

SEQ ID NO: 28 - DNA, 23 bp, Triticum aestivum (var. Spelt)
gccggacggtcctcgccatgggg

SEQ ID NO: 29 - DNA, 23 bp, Triticum aestivum (var. Spelt)
ttctgtgaaccttcaaaatctgg

SEQ ID NO: 30 - DNA, 23 bp, Triticum aestivum (var. Spelt)
agattttgaaggttcacagaagg

SEQ ID NO: 31 - DNA, 23 bp, Triticum aestivum (var. Spelt)
aaggttcacagaaggagagatgg

SEQ ID NO: 32 - DNA, 27 bp, Triticum aestivum (var. Spelt)
tttgaaggttcacagaaggagagatgg

SEQ ID NO: 33 - DNA, 362 bp, Triticum aestivum
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagactt

SEQ ID NO: 34 - DNA, 103 bp, Artificial sequence
  gRNA_983r
gcggggcatggccggtgtgtgttttagagctagaaatagcaagttaaaataaggctagtccgttatcaacttgaaaaagtggcaccgagtcggtgcttttttt

SEQ ID NO: 35 - DNA, 103 bp, Artificial sequence
  gRNA_991r
gaggagaagcggggcatggcgttttagagctagaaatagcaagttaaaataaggctagtccgttatcaacttgaaaaagtggcaccgagtcggtgcttttttt

SEQ ID NO: 36 - DNA, 103 bp, Artificial sequence
  gRNA_972f
tgagttcgcgacctacacacgttttagagctagaaatagcaagttaaaataaggctagtccgttatcaacttgaaaaagtggcaccgagtcggtgcttttttt

SEQ ID NO: 37 - DNA, 103 bp, Artificial sequence
  gRNA_479r
tccccatggcgaggaccgtcgttttagagctagaaatagcaagttaaaataaggctagtccgttatcaacttgaaaaagtggcaccgagtcggtgcttttttt

SEQ ID NO: 38 - DNA, 103 bp, Artificial sequence
  gRNA_488r
cccagcagttccccatggcggttttagagctagaaatagcaagttaaaataaggctagtccgttatcaacttgaaaaagtggcaccgagtcggtgcttttttt

SEQ ID NO: 39 - DNA, 103 bp, Artificial sequence
  gRNA_478f
gccggacggtcctcgccatggttttagagctagaaatagcaagttaaaataaggctagtccgttatcaacttgaaaaagtggcaccgagtcggtgcttttttt

SEQ ID NO: 40 - DNA, 103 bp, Artificial sequence
  gRNA_1145r
ttctgtgaaccttcaaaatcgttttagagctagaaatagcaagttaaaataaggctagtccgttatcaacttgaaaaagtggcaccgagtcggtgcttttttt

SEQ ID NO: 41 - DNA, 103 bp, Artificial sequence
  gRNA_1147f
agattttgaaggttcacagagttttagagctagaaatagcaagttaaaataaggctagtccgttatcaacttgaaaaagtggcaccgagtcggtgcttttttt

SEQ ID NO: 42 - DNA, 103 bp, Artificial sequence
  gRNA_1155f
aaggttcacagaaggagagagttttagagctagaaatagcaagttaaaataaggctagtccgttatcaacttgaaaaagtggcaccgagtcggtgcttttttt

SEQ ID NO: 43 - DNA, 51 bp, Artificial sequence
  crRNA_1151f
taatttctactaagtgtagataaggttcacagaaggagagatggttttttt

SEQ ID NO: 44 - DNA, 1992 bp, Zea mays
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 45 - DNA, 4206 bp, Artificial sequence
  NLS-Cas9-NLS
atggctcctaagaagaagcgcaaggttgggatccacggggttccggcggctatggacaagaagtacagcatcgggctcgacatcgggacgaacagcgttgggtgggcggtcatcacggacgagtacaaggttccctccaagaagttcaaggtgctggggaacaccgaccgccactccatcaagaagaacctcatcggggccctcctgttcgacagcggggagaccgctgaggctacgaggctcaagagaaccgcgaggcgcagatacacgagaaggaagaacaggatctgctacctccaagagatcttctccaacgagatggccaaggttgacgattcattcttccaccgcctggaggagtctttcctcgtggaggaggacaagaagcacgagcggcatcccatcttcgggaacatcgtcgacgaggttgcctaccacgagaagtaccctacgatctaccatctgcggaagaagctcgtggacagcaccgataaggcggacctcagactgatctacctcgccctggcgcacatgatcaagttccgcgggcatttcctgatcgagggggatctcaacccagacaactccgatgttgacaagctgttcatccaactcgtgcagacctacaaccaactcttcgaggagaacccgatcaacgcttcgggggtcgacgctaaggccatcctgtcagcccgcctctcgaagtcacgcaggctggagaacctgatcgcgcagctcccaggggagaagaagaacgggctgttcgggaacctcatcgctctctctctggggctcacgccgaacttcaagtccaacttcgatctcgccgaggacgcgaagctgcaactcagcaaggacacctacgacgatgacctcgataacctcctggcgcagatcggggatcaatacgctgacctgttcctcgccgcgaagaacctgtcggacgccatcctcctgtcagatatcctccgcgtgaacaccgagatcacgaaggccccactctctgcgtccatgatcaagcgctacgacgagcaccatcaggatctgaccctcctgaaggcgctggtccgccaacagctcccggagaagtacaaggagatcttcttcgatcagtcgaagaacgggtacgctgggtacatcgacgggggggcgtcacaagaggagttctacaagttcatcaagccaatcctggagaagatggacgggacggaggagctcctggtgaagctcaacagggaggacctcctgcggaagcagagaaccttcgataacgggtccatcccccaccaaatccatctcggggagctgcacgccatcctgagaaggcaagaggacttctaccctttcctcaaggataaccgggagaagatcgagaagatcctgaccttcagaatcccatactacgtcgggccgctcgctcgggggaacagcagattcgcctggatgacccgcaagtcggaggagaccatcacgccgtggaacttcgaggaggtggtcgacaagggggcgagcgctcagtcgttcatcgagaggatgaccaacttcgacaagaacctgcccaacgagaaggtgctccctaagcactcgctcctgtacgagtacttcaccgtctacaacgagctcacgaaggtgaagtatgtgaccgaggggatgagaaagcctgccttcctgtccggggagcagaagaaggcgatcgtggacctcctgttcaagaccaaccggaaggtcacggttaagcaactcaaggaggactacttcaagaagatcgagtgcttcgattcggtggagatctcaggggtcgaggacaggttcaacgcctccctcgggacctaccacgatctcctgaagatcatcaaggataaggacttcctggacaacgaggagaacgaggacatcctggaggacatcgtgctgaccctcacgctgttcgaggacagggagatgatcgaggagcgcctgaagacgtacgcccatctcttcgatgacaaggtcatgaagcaactcaagagaagaagatacaccgggtgggggaggctgtcccgcaagctcatcaacgggatccgggacaagcagtcagggaagaccatcctcgacttcctgaagtctgatgggttcgccaacaggaacttcatgcaactgatccacgatgactcactcaccttcaaggaggatatccaaaaggctcaggtgagcgggcagggggactcgctgcacgagcatatcgctaacctcgctgggtcccctgcgatcaagaaggggatcctgcagaccgtgaaggttgtggacgagctcgtgaaggtcatggggcggcataagcctgagaacatcgtcatcgagatggccagagagaaccaaaccacgcagaaggggcaaaagaactctagggagcgcatgaagcgcatcgaggaggggatcaaggagctggggtcccaaatcctcaaggagcacccagttgagaacacccaactgcagaacgagaagctctacctgtactacctccagaacgggagggatatgtatgtggaccaagagctggatatcaaccgcctcagcgattacgacgttgatcatatcgtgccccagtctttcctgaaggatgactccatcgacaacaaggtcctcaccaggtcggacaagaaccgcgggaagtcagataacgttccatctgaggaggtcgttaagaagatgaagaactactggaggcagctcctgaacgccaagctgatcacgcaaaggaagttcgacaacctcaccaaggctgagagaggggggctctcagagctggacaaggctgggttcatcaagcggcagctggtcgagaccagacaaatcacgaagcacgttgcgcaaatcctcgactctcggatgaacacgaagtacgatgagaacgacaagctgatcagggaggtgaaggtcatcaccctgaagtctaagctcgtctccgacttccgcaaggacttccagttctacaaggttcgcgagatcaacaactaccaccatgcgcatgacgcttacctcaacgctgtggtcgggaccgccctgatcaagaagtacccgaagctggagagcgagttcgtgtacggggactacaaggtttacgatgtgcgcaagatgatcgccaagtcggagcaagagatcgggaaggccaccgccaagtacttcttctactcaaacatcatgaacttctttaagaccgagatcacgctggccaacggggagatccggaagagacccctcatcgagaccaacggggagacgggggagatcgtgtgggacaaggggagggatttcgcgaccgtccgcaaggttctctccatgccccaggtgaacatcgtcaagaagaccgaggtccaaacgggggggttctcaaaggagtctatcctgcctaagcggaacagcgacaagctcatcgccagaaagaaggactgggatccaaagaagtacggggggttcgacagccctaccgtcgcgtactcggttctggttgtggctaaggttgagaaggggaagtccaagaagctcaagagcgtgaaggagctcctggggatcaccatcatggagcgctccagcttcgagaagaacccaatcgacttcctggaggccaaggggtacaaggaggtgaagaaggacctgatcatcaagctcccgaagtactctctcttcgagctggagaacgggaggaagcgcatgctggcttccgctggggagctccaaaaggggaacgagctcgccctgccatccaagtatgtgaacttcctctacctggcgtcccactacgagaagctcaaggggagcccggaggacaacgagcaaaagcagctgttcgtcgagcagcacaagcattacctcgacgagatcatcgagcaaatctccgagttctccaagcgcgtgatcctcgcggacgctaacctggataaggtcctctccgcttacaacaagcaccgggacaagcccatcagagagcaggccgagaacatcatccatctcttcaccctgacgaacctcggggcccctgctgccttcaagtacttcgacaccacgatcgatcggaagagatacaccagcacgaaggaggtcctggacgcgaccctcatccaccaatctatcacggggctgtacgagacgagaatcgacctgtcacaactggggggggacaagaggccggcggcaaccaagaaggcgggccaggccaagaagaagaagtga

SEQ ID NO: 46 - DNA, 3873 bp, Artificial sequence
  NLS-Cas12a-NLS
atggccccgaagaagaagaggaaagtgggcatccacggcgtgcctgcagcaatgtccaagctggagaagttcaccaactgctactccctgagcaagaccctccgcttcaaggccatccccgtgggcaagacccaggagaacatcgacaacaagaggctcctggtggaggatgagaagagggcagaggactacaagggcgtcaagaagctgctcgacaggtactacctgtccttcatcaacgacgtcctccacagcatcaagctgaagaacctcaacaactacatctccctcttcaggaagaagacccgcaccgagaaggagaacaaggagctggagaacctcgagatcaacctgcgcaaggagatcgccaaggccttcaagggcaacgagggctacaagagcctcttcaagaaggacatcatcgagaccatcctgcctgagttcctcgacgacaaggatgaaatcgccctggtgaactccttcaacggcttcaccaccgccttcaccggcttcttcgacaacagggagaacatgttcagcgaggaagccaaatcaacctccatcgccttcaggtgcatcaacgagaacctcacccgctacatctccaacatggacatcttcgagaaggtcgacgccatcttcgacaagcacgaggtgcaggagatcaaggagaagatcctgaacagcgactacgacgtggaggacttcttcgagggcgagttcttcaacttcgtcctcacccaagagggcatcgacgtgtacaacgccatcatcggcggcttcgtcaccgagtccggcgagaagatcaagggcctgaacgagtacatcaacctctacaaccagaagaccaagcagaagctgccgaagttcaagccactgtacaagcaggtgctctccgacagggagtccctcagcttctacggcgagggctacaccagcgacgaggaagtgctggaggtgttccgcaacaccctcaacaagaacagcgagatcttctccagcatcaagaagctggagaagctgttcaagaacttcgacgagtactccagcgccggcatcttcgtgaagaacggcccggccatctccaccatcagcaaggacatcttcggcgagtggaacgtcatccgcgacaagtggaacgccgagtacgacgacatccacctgaagaagaaggccgtggtcaccgagaagtacgaggacgacaggcgcaagtccttcaagaagatcggctccttcagcctggagcagctccaggagtacgccgacgccgacctgagcgtggtcgagaagctcaaggagatcatcatccagaaggtcgacgagatctacaaggtgtacggctccagcgagaagctgttcgacgccgacttcgtgctggagaagtccctcaagaagaacgacgccgtggtcgccatcatgaaagacctgctcgactccgtcaagagcttcgagaactacatcaaggccttcttcggcgagggcaaggagaccaacagggacgagtccttctacggcgacttcgtcctggcctacgacatcctgctcaaggtggaccacatctacgacgccatcaggaactacgtgacccagaagccgtacagcaaggacaagttcaagctctacttccagaacccacagttcatgggcggctgggacaaggacaaggagaccgactacagggccaccatcctgcgctacggcagcaagtactacctcgccatcatggacaagaagtacgccaagtgcctgcagaagatcgacaaggacgacgtgaacggcaactacgagaagatcaactacaagctgctcccgggcccaaacaagatgctccccaaggtcttcttctccaagaagtggatggcctactacaaccctagcgaggacatccagaagatctacaagaacggcaccttcaagaagggcgacatgttcaacctgaacgactgccacaagctcatcgacttcttcaaggactccatcagcaggtacccgaagtggtccaacgcctacgacttcaacttcagcgagaccgagaagtacaaggacatcgccggcttctaccgcgaggtcgaggagcagggctacaaggtgtccttcgagtccgcctccaagaaggaagtggacaagctggtggaggaaggcaagctgtacatgttccagatctacaacaaggacttctccgacaagagccacggcaccccaaacctgcacaccatgtacttcaagctgctgttcgacgagaacaaccacggccagatcaggctctccggcggcgccgagctgttcatgcgtagagccagcctgaagaaagaggagctggtggtccacccagcaaacagccctatcgccaacaagaaccccgacaaccctaagaagaccaccaccctgtcctacgacgtgtacaaggacaagaggttcagcgaggaccagtacgagctccacatccccatcgccatcaacaagtgccctaagaacatcttcaagatcaacaccgaggtccgcgtgctgctcaagcacgacgacaacccatacgtgatcggcatcgacaggggcgagcgcaacctgctctacatcgtggtcgtggacggcaagggcaacatcgtcgagcagtactccctcaacgagatcatcaacaacttcaacggcatcaggatcaagaccgactaccacagcctgctcgacaagaaggagaaggagaggttcgaggcccgccagaactggacctccatcgagaacatcaaggagctgaaggccggctacatcagccaggtcgtgcacaagatctgcgagctcgtcgagaagtacgacgccgtgatcgccctggaggacctcaactccggcttcaagaacagccgcgtcaaggtggagaagcaggtgtaccagaagttcgagaagatgctgatcgacaagctcaactacatggtcgacaagaagtccaacccctgcgcaaccggcggcgccctgaagggctaccagatcaccaacaagttcgagagcttcaagtccatgagcacccagaacggcttcatcttctacatcccggcctggctcacctccaagatcgacccaagcaccggcttcgtgaacctgctcaagaccaagtacacctccatcgccgacagcaagaagttcatctccagcttcgacaggatcatgtatgtgcccgaggaagacctgttcgagttcgccctcgactacaagaacttctcccgcaccgacgccgattacatcaagaagtggaagctgtacagctacggcaacaggatccgcatcttcaggaaccctaagaagaacaacgtcttcgactgggaggaagtgtgcctgacctccgcctacaaggagctgttcaacaagtacggcatcaactaccagcagggcgacatcagggccctgctctgcgagcagagcgacaaggccttctactccagcttcatggccctgatgtccctgatgctccagatgaggaacagcatcaccggccgcaccgacgtcgacttcctcatctccccggtgaagaacagcgacggcatcttctacgactccaggaactacgaggcccaggagaacgccatcctgccaaagaacgccgacgccaacggcgcctacaacatcgcaaggaaggtgctctgggcaatcggccagttcaagaaggccgaggacgagaagctggacaaggtgaagatcgccatctccaacaaggagtggctcgagtacgcccagacctctgtcaagcacaagaggccagcagcaaccaagaaggcgggccaggccaagaagaagaagggcagctacccgtacgacgtcccagactacgcctacccctacgacgtgcctgactacgcttacccatacgatgtgccagactacgcctga

SEQ ID NO: 47 - DNA, 250 bp, Agrobacterium tumefaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactag

SEQ ID NO: 48 - DNA, 903 bp, Triticum aestivum (var. Chinese Spring)
atggccacggcggcggcggcggcggcgacgatgcgtctgtcttccccgttcagggccccgcctctccgtcctccctgccaccgcgtcgtcccgtcgatgcggggccccaggcgggccgggctggccgtctcggcggccgccgccgggtcgccccccaccgtgctcgtcaccggcgccggaggacggacaggccaaattgtgtacaagaagctgaaggagagggcagaccagtttgtggccagagggctggtcaggacgccggacagcaagggtaagataggcggcggcgacgacgtgttcatcggcgacatcagggatcctgggagcattgctccggcgattgagggcatcgacgcgctcatcatcctcaccagtggggtcccgaagatgaagcccgggttcgatcctagcaagggtggacggccggagttttactttgaggaagggtctgatcctgagcaggtggattggataggccaaaagaaccaaattgatgctgccaagagcattggtgtaaagcagatagttttggttggatccatgggcggaacagatatcaaccatccattaaacaagcttgggaatgggaatatactggtgtggaaacggaaggcagagcagtacctagcggactctggtctaccatacacaattataagggctggaggactacaagacaaagatggtggggtgcgtgagttgattgttggaaaggatgacgagatcttgaagacggaaacaaaaactattgccagggcagatgttgcagaagtttgcatacaggccttgctatttgaggaagcaaagttcaaggcgtttgatctggcttcaaaacctgaaggtgaaggaacaccgatgacagattttaagtctgtttttgcacaaattgctactcgcttctaa

SEQ ID NO: 49 - protein, 300 aa, Triticum aestivum (var. Chinese Spring)
MATAAAAAATMRLSSPFRAPPLRPPCHRVVPSMRGPRRAGLAVSAAAAGSPPTVLVTGAGGRTGQIVYKKLKERADQFVARGLVRTPDSKGKIGGGDDVFIGDIRDPGSIAPAIEGIDALIILTSGVPKMKPGFDPSKGGRPEFYFEEGSDPEQVDWIGQKNQIDAAKSIGVKQIVLVGSMGGTDINHPLNKLGNGNILVWKRKAEQYLADSGLPYTIIRAGGLQDKDGGVRELIVGKDDEILKTETKTIARADVAEVCIQALLFEEAKFKAFDLASKPEGEGTPMTDFKSVFAQIATRF

SEQ ID NO: 50 - DNA, 3063 bp, Triticum aestivum (var. Chinese Spring)
cgccttagatgccgtcgcctcctagccataagcgtcagaaggcggtatataggttatctccgccggaaaaagggtaaggtttgcctcgagaatgttttgtttgactatatcctatgtggaaccaaggctaggtttgcatgcaatgaaaaaatgtaattgtaggaggagaagggagggtggtagatgtgcgcgacaacaacacacacgccatatatatatagttggaggaggtggaaaacaaggaggcgataagaaaaagggggggaatatggagaatgtttgaaatagcgtccaacccgagtttccattattaattgtcggccaaaaaaagaaggcatgttactcatgatggtagtttcaagggaagtggagcctcccagctagtttgaacacttaaaaaacaagctacaacagaaagtagtttcaaggggagtggagcctaaaacttgcatttaaaaaggaaatgcgcacactaagccgaaagtgtgaacactttgtgccgacatttccgaggaagtaggttttatgcgcataacattttttttgagacaattttatgcgcataactagggttgactgctgtggggaagatgaaatttgaactgccattgtgacgcaaagatttggtgatccaaatttgtatatggaccatgcctgtaggttgggctaagtatacttttcgaagaaaataggcccatgttaacttgggaagtgggcctaggcccacaaacctgaaatccctgtgaagcccacattcacacgcgggcccaccgtcgaagcgataaggagagtgacagccgctcagcccgccggcacacccgacaacaactctcgcttcactcgccggagggggacggagcaaatatggccacggcggcggcggcggcggcgacgatgcgtctgtcttccccgttcagggccccgcctctccgtcctccctgccaccgcgtcgtcccgtcgatgcggggccccaggcgggccgggctggccgtctcggcggccgccgccgggtcgccccccaccgtgctcgtcaccggcgccggaggacggacaggtagatactacacatcccgctagcttctttaccttcgcgtcctgtagccgcacattgttgcacgatgccgccttctcttgctgttccaatgggattcagtttgcatttgtgggagaatttaccggcggaggcggcggtttcttggaatcaagatgcttcacaacctatggaacctcctattgatttaaaatttacactgccgtttacgtgctgtgatccggcgtgcgcctctgacccttgcgctgtggtgacccaggccaaattgtgtacaagaagctgaaggagagggcagaccagtttgtggccagagggctggtcaggacgccggacagcaagggtaagataggcggcggcgacgacgtgttcatcggcgacatcagggatcctgggagcattgctccggcgattgagggcatcgacgcgctcatcatcctcaccagtggggtcccgaagatgaagcccgggttcgatcctagcaagggtggacggccggagttttactttgaggaagggtctgatcctgagcaggtaaccggcaaaaatcgcttggcaatgacattagtcacattagcaatgacaacttatgcatttcttttagagttaattaatgtcctttgctgttttcccttgatttaaggtggattggataggccaaaagaaccaaattgatgctggtaaatcttcaccttcttcttgttctgttagagcattatacaatgtttagcatgttcattgaaacgattgattggtgaagcatcgatgtgtcattttctctccggtcgacctttttcttgtctgctctctaagtttcccatctttaattatccatgcagccaagagcattggtgtaaagcagatagttttggttggatccatgggcggaacagatatcaaccatccattaaacaagcttgggaatgggaatatactggtatatagcatgctcgacgatttttgtatatgttctaattccctatacttgtgtgtgcataaacatataatatgtaccattttgaaacttagaatggccaattgctcttctaatttgcaggtgtggaaacggaaggcagagcagtacctagcggactctggtctaccatacacaattataaggttataacacagactcattcggtagtgctattcttgccatgttctgcaaagattttttttttgtcacatttgagttatgtaataggctgacagtcactgttttctccaaagatgcacagtcatagaaataattttatatatgtgatgaatgaagaaaacaacaaaacaacagaagtgcattatggagtatgaagcaccacaaagaacattaattttgtataagaaataaaaaattccaatttatttcatagggacattaatcgttggagtattttccaccaacaatcgtgtgctgataaaatacctttcccatatcccatctctttccagggctggaggactacaagacaaagatggtggggtgcgtgagttgattgttggaaaggatgacgagatcttgaagacggaaacaaaaactattgccagggcagatgttgcagaagtttgcatacaggtacctttatagaacaaaacacatgtttctgtagataaactactgctcaatatttttttcatcatctaccaaatatatttcttgcatctatttatgtcttactagctagtggtcataccactgatccatctgtgcctttcctgagtaggccttgctatttgaggaagcaaagttcaaggcgtttgatctggcttcaaaacctgaaggtgaaggaacaccgatgacagattttaagtctgtttttgcacaaattgctactcgcttctaagaggacaagatcattctggccaatgaaatgggacagtatctttaattaggcgatatggaatttgtactgggtcaggttagtgttctgtttattaattgaggaaatgcaacattttattctacgcactattattccatagtaaattttgtgtttatctcttttctttgtggtcaa

SEQ ID NO: 51 - DNA, 960 bp, Triticum aestivum (var. Spelt)
atggccacggcggcggcggcggcggcgacgatgcgtctgtcttccccgttcagggccccgcctctccgtcctccctgccaccgcgtcgtcccgtcgatgcggggccccaggcgggccgggctggccgtctcggcggccgccgccgggtcgccccccaccgtgctcgtcaccggcgccggaggacggacaggccaaattgtgtacaagaagctgaaggagagggcagaccagtttgtggccagagggctggtcaggacgccggacagcaagggtaagataggcggcggcgacgacgtgttcatcggcgacatcagggatcctgggagcattgctccggcgattgagggcatcgacgcgctcatcatcctcaccagtggggtcccgaagatgaagcccgggttcgatcctagcaagggtggacggccggagttttactttgaggaagggtctgatcctgagcaggtggattggataggccaaaagaaccaaattgatgctgccaagagcattggtgtaaagcagatagttttggttggatccatgggcggaacagatatcaaccatccattaaacaagcttgggaatgggaatatactggtgtggaaacggaaggcagagcagtacctagcggactctgggacattaatcgttggagtattttccaccaacaatcgtgtgctgataaaatacctttcccatatcccatctctttccagggctggaggactacaagacaaagatggtggggtgcgtgagttgattgttggaaaggatgacgagatcttgaagacggaaacaaaaactattgccagggcagatgttgcagaagtttgcatacaggccttgctatttgaggaagcaaagttcaaggcgtttgatctggcttcaaaacctgaaggtgaaggaacaccgatgacagattttaagtctgtttttgcacaaattgctactcgcttctaa

SEQ ID NO: 52 - protein, 319 aa, Triticum aestivum (var. Spelt)
MATAAAAAATMRLSSPFRAPPLRPPCHRVVPSMRGPRRAGLAVSAAAAGSPPTVLVTGAGGRTGQIVYKKLKERADQFVARGLVRTPDSKGKIGGGDDVFIGDIRDPGSIAPAIEGIDALIILTSGVPKMKPGFDPSKGGRPEFYFEEGSDPEQVDWIGQKNQIDAAKSIGVKQIVLVGSMGGTDINHPLNKLGNGNILVWKRKAEQYLADSGTLIVGVFSTNNRVLIKYLSHIPSLSRAGGLQDKDGGVRELIVGKDDEILKTETKTIARADVAEVCIQALLFEEAKFKAFDLASKPEGEGTPMTDFKSVFAQIATRF

SEQ ID NO: 53 - DNA, 5510 bp, Triticum aestivum (var. Spelt)
ggagtcgtgtcttatttggtggcgtttgtcgactatctcctgtgtggagccaatgcttggctcgcacgcaagtcgaaaagaactgcaatggtaggaggagaagggagagggtgtggtagatgcgcacgacaacaacaacacacgacaaatatatatagttggagtaagtggaaaacaaggacgcgacaagaaagggggagaatgttcgaaatagcgtccaacctaagttcagagaattaattgtcaaccaaaaattgggcaagggctcagtataccggcctagaagtgaaaattgcgatcaaaagtttgcaccgccgaagaagagaaaagcaaaaacttctcatatttttcaaaggattgaacattttttattgtcatgttatcccgtaggcaggatattgacctagcaactttggtgtagcctattgtattactcatattcatcgagcagtatattattgaaggggcttttattgactctaaatgtagcattgttgtaatgcatttgcataattaagggtgtgtttggttgtgtggctaaggatagccactttttaagtggttgccatgtgtttgattttaaaatttgtgtagttcaggatagccatatccaaggaatattccataaaatgtggcacagcggaagcgccaaaaagtgatgggtaaggtcaaccaccctatgcttgcatgctcacctcaccatctgaatcatttacaacctgttctgaccccctcccccaaaggaattttatcaggcttttggtgagggcattcgcatgtctgcacttgtcatccaatgaactgtgacgaatactagggtgttagaaaggaaacggcaactcatgaaatataaccgacaaacatatatatctataaatcatgcaacaaagaaaccgaaagattacaacaaaaaccatatatggatctgatcataaaggggcaattcatcacacggaaacaaacacaccgagaattacatcagatggaacacaatcatgtagagcagcgcatgtgaccattgattgaaaagcaagggagagaaggaaaagcaagggagagaagatgccaactagctactgttatggacccatagttcaaagaggactattcacacatggtcatggtggcaacgaggttgctggagatgcatccggcgatgatttatccctccaggaaagtaccggaaaaaggtctccggattggatcatcgcgaaacaagtggtggcgacagcagaaaaatccttgataaacaaatcacgtgagagagctccgaaattagggagcatactgaatttaaagatactagatgataccccgcatgttattgtgaactctggtctaccatacacaattataaggttataacacagactcattcggtagtgctatttgtggtatgagacttattcttgccatgttctgcaaagaattttttttgtcacatttgagttatgtaataggctgacagtcactgttttctccaaagacgcacagtcataaaagtaatttgaaaaaactgtgcattttgaaaaaaatgttcgcggaatcaaaaaatgtttgtgattttacaaaaaaatattcacatattcagaaaatgttggcgtattcagaaaatgttatggaattgagaaattttctgcaaagtttaaaatttttcatgtattttaaaaaggcttcacaaaatttaaaaaatgtccattcattacaaaaatgatcctcgattcaaaaatgctcattcaaataaaaaaatgttcatgaattacaaaattaattcccccaatttttaaaagtgtttcgctgattcaaaacaaatattcacaggcttaaaagatgttcacaattcctagaaatgtacacaaatttgtaaaaaatcctcgtcaatttaaaaaatgttcgtcaattaaaaaaatgtaatgtagtagtagtatgttgagtagtccaaccgaaggatattaattctcatctcagtcatcactcctcttcctcggatcaccggcgatggcaatggcattgtccctccccaccacggtcttgctcctcctcctcctcctcctcctccccgtcgccttagatgccgtcgcctcctagccataagcgtcagaaggcggtatataggttatctccgccggaaaaagggtaaggtttgcctcgagaatgttttgtttgactatatcctatgtggaaccaaggctaggtttgcatgcaatgaaaaaatgtaattgtaggaggagaagggagggtggtagatgtgcgcgacaacaacacacacgccatatatatatagttggaggaggtggaaaacaaggaggcgataagaaaaagggggggggggaatatggagaatgtttgaaatagcgtccaacccgagtttccattattaattgtcggccaaaaaaagaaggcatgttactcatgatggtagtttcaagggaagtggagcctcccagctagtttgaacacttaaaaaaacaagctacaacagaaagtagtttcaaggggagtggagcctaaaacttgcatttaaaaaggaaatgcgcacactaagccgaaagtgtgaacactttgtgccgacatttccgaggaagtaggttttatgcgcataacatttttttgagacaattttatgcgcataactagggttgactgctgtggggaagatgaaatttgaactgccattgtgacgcaaagatttggtgatccaaatttgtatatggaccatgcctgtaggttgggctaagtatacttttcgaagaaaataggcccatgttaacttgggaagtgggcctaggcccacaaacctgaaatccctgtgaagcccacattcacacgcgggcccaccgtcgaagcgataaggagagtgacagccgctcagcccgccggcacacccgacaacaactctcgcttcactcgccggagggggacggagcaaatatggccacggcggcggcggcggcggcgacgatgcgtctgtcttccccgttcagggccccgcctctccgtcctccctgccaccgcgtcgtcccgtcgatgcggggccccaggcgggccgggctggccgtctcggcggccgccgccgggtcgccccccaccgtgctcgtcaccggcgccggaggacggacaggtagatactacacatcccgctagcttctttaccttcgcgtcctgtagccgcacattgttgcacgatgccgccttctcttgctgttccaatgggattcagtttgcatttgtgggagaatttaccggcggaggcggcggtttcttggaatcaagatgcttcacaacctatggaacctcctattgatttaaaatttacactgccgtttacgtgctgtgatccggcgtgcgcctctgacccttgcgctgtggtgacccaggccaaattgtgtacaagaagctgaaggagagggcagaccagtttgtggccagagggctggtcaggacgccggacagcaagggtaagataggcggcggcgacgacgtgttcatcggcgacatcagggatcctgggagcattgctccggcgattgagggcatcgacgcgctcatcatcctcaccagtggggtcccgaagatgaagcccgggttcgatcctagcaagggtggacggccggagttttactttgaggaagggtctgatcctgagcaggtaaccggcaaaaatcgcttggcaatgacattagtcacattagcaatgacaacttatgcatttcttttagagttaattaatgtcctttgctgttttcccttgatttaaggtggattggataggccaaaagaaccaaattgatgctggtaaatcttcaccttcttcttgttctgttagagcattatacaatgtttagcatgttcattgaaacgattgattggtgaagcatcgatgtgtcattttctctccggtcgacctttttcttgtctgctctctaagtttcccatctttaattatccatgcagccaagagcattggtgtaaagcagatagttttggttggatccatgggcggaacagatatcaaccatccattaaacaagcttgggaatgggaatatactggtatatagcatgctcgacgatttttgtatatgttctaattccctatacttgtgtgtgcataaacatataatatgtaccattttgaaacttagaatggccaattgctcttctaatttgcaggtgtggaaacggaaggcagagcagtacctagcggactctggtctaccatacacaattataaggttataacacagactcattcggtagtgctattcttgccatgttctgcaaagattttttttttgtcacatttgagttatgtaataggctgacagtcactgttttctccaaagatgcacagtcatagaaataattttatatatgtgatgaatgaagaaaacaacaaaacaacagaagtgcattatggagtatgaagcaccacaaagaacattaattttgtataagaaataaaaaattccaatttatttcatagggacattaatcgttggagtattttccaccaacaatcgtgtgctgataaaatacctttcccatatcccatctctttccagggctggaggactacaagacaaagatggtggggtgcgtgagttgattgttggaaaggatgacgagatcttgaagacggaaacaaaaactattgccagggcagatgttgcagaagtttgcatacaggtacctttatagaacaaaacacatgtttctgtagataaactactgctcaatatttttatcatcatctaccaaatatatttcttgcatctatttatgtcttactagctagtggtcataccactgatccatctgtgcctttcctgagtaggccttgctatttgaggaagcaaagttcaaggcgtttgatctggcttcaaaacctgaaggtgaaggaacaccgatgacagattttaagtctgtttttgcacaaattgctactcgcttctaagaggacaagatcattctggccaatgaaatgggacagtatctttaattaggcgatatggaatttgtactgggtcaggttagtgttctgtttattaattgaggaaatgcaacattttattctacgcactattattccatagtaaattttgtgtttatctcttttctttgtggtcaatggattctgcataaaatgtttatggcccttctgttacctggttgatctgtcctaatttggatgtacacacttagtatttgcagttagtttattttgtaactaaaggtcggacatgcttggggtcatgcggtcatgatgttctggttgatctagttttaagttaatccatttgctagtttaaacaacgtttagggttatccatgttgcgtatttatagtcccaccaaatcgttggttagtataatctggattgtaaatttgttagggctataaacacccaacttcacatgctccttctgtagcaaaagtagaagtggcatgtcctctccttccagattgtctccgatgctctgccaacaaaacaacaacaacaacaacaac

SEQ ID NO: 54 - DNA, 23 bp, Triticum aestivum (var. Spelt)
cttcactcgccggagggggacgg

SEQ ID NO: 55 - DNA, 23 bp, Triticum aestivum (var. Spelt)
ggagggggacggagcaaatatgg

SEQ ID NO: 56 - DNA, 103 bp, Triticum aestivum (var. Spelt)
cttcactcgccggagggggagttttagagctagaaatagcaagttaaaataaggctagtccgttatcaacttgaaaaagtggcaccgagtcggtgcttttttt

SEQ ID NO: 57 - DNA, 103 bp, Triticum aestivum (var. Spelt)
ggagggggacggagcaaatagttttagagctagaaatagcaagttaaaataaggctagtccgttatcaacttgaaaaagtggcaccgagtcggtgcttttttt

SEQ ID NO: 58 - DNA, 2379 bp, Triticum aestivum
atgcctcgcttctcctccaccacgccaatgtcgccaccccgcctcctcctccggctcggcgcccgccactcctcctccacctctcatccctcacgcatctgggatccccacgccgccttcgccgctgcgacgcagcgggcgcgctctggcacgctcaccacggaggacgcacaccacctgtttgatgaattgctgcggcagggcaatcctgtccaggagcgtcccttgactaactttctggctgccctcgcccgcgcgcccgcgtccgcattctgcagcgatggccctgccctggccgtcgccctcttcggccgtttgtcccgaggcgccggacgacgggtggcgcagccaaatgtcttcacctatggcgtcctcatggactgctgctgccgtgcgcgccgcctggatctagcgatcgccttcttcgcccgtctcctcaagacgggactggaggcaaaccaagtcatcttctgcaccctcctcaagggactctgccacgcaaagcgctcagatgaggctttggacgtggtgcttcacaggatgcctgagctaggctgcacccccaacgtggtggcctataccacggtcatccacggcttcttgaaggaaggccaagtaggcaaggcatgcaatctattccatggaatggcgcagcagggcgttgcgcctgatgtggtgacatataactcggttatcgatgcgttgtgcaaggccagagcaatggacaaggcagagtatttccttcgtgaaatggttgataatggtgtcgtacctaataatgtgacatataatagcctcatccatggatattcctctttgggccatcagaaggaggctgttagggtgctgaaagaaatgacaagacagggtatcataccagatgtcattacctgcacctcactcatgaccttcctttgcaagaatggaaaaagcaaggaagctgcagaaatttttgattcaatggccacgaagggcctgaaacatgacgccgtttcatatgctattctccttcatgggtatgccactgaaggatgcttggttgatatgattaatctcttcaattcgatggacagagactgtattctacctaactgtcatatcttcaacatactgatttatgcatatgctaaatctgggaagcttgataaggctatgcttatatttagagatatgcagaaacaaggagtgagcccagatgcattcacatattcaaccttaatacatgcattttgtaaaaagggtcggttggacgatgctatgataaagtttaatcagatggttgatacaggagtacgacagggcacagctgtttatggttctctaatccagggtttttgtacacacggcgatttggtgaaaggaaaggaattggttactgaaatgatgaacaaaggtatacctcctcctgacattatgttcttccattcaatcatgcagaacctatgcacagaaggaagggtagtagaagcacgggatatccttggcttgatagcacacataggtatgaggcctaatgtttgcacatttaatatactgattggtggatactgcctagtccgcaagatggaggatgcctcaaaaatatttcatgatatgatgtcatatggtttagaaccttctaatgttacgtatggtattcttattaatggctattgcaaaaacagaaggattgatgacgggctgattctgttcaaagaaatgttgcgcaagggacttaaacctacaacttttaattacaacatcatactggatggattatttctggctggacgaactgttgctgcaaaggaaaagtttgatgagatggttgaatctggagtaagtatgtgcatcagtacttactctatagttcttcgtggactttgtagaaataattgtagcggcgaagccatcacgctattccagacattaagcgcaatggatgtgaaattcaatattagaattgtcaatatcatgattgatgccttcttcagggttcagcgaaagcaagaagctaaggatttgtttgctgcaataacagccaatgggttggttgctaatgtttttacctacagcctaatgatgacaaatcttataaaagaagggtcagtggaagaggctgacacactctttttatcgatggagatgagcggctgtacttcgaactcgtggatgttaaatcttattatcagagggttgctggaaaaaggagagatagtcaaggctggatgttatatgtctaaagttgatgccaagagctactcacttgaagctaaaactgtttcgttgctgatctatctcttttcagggaaagggaaatacagagaacacataagattgctacctacaaagtatcagtttctcgaagaagcagccacagttgaatggtttgctatatag

SEQ ID NO: 59 - DNA, 2880 bp, Triticum aestivum (var. Chinese Spring)
gagtcgtgtcttatttggtggcgtttgtcgactatctcctgtgtggagccaatgcttggctcgcacgcaagtcgaaaagaactgcaatggtaggaggagaagggagagggtgtggtagatgcgcacgacaacaacaacacacgacaaatatatatagttggagtaagtggaaaacaaggacgcgacaagaaagggggagaatgttcgaaatagcgtccaacctaagttcagagaattaattgtcaaccaaaaattgggcaagggctcagtataccggcctagaagtgaaaattgcgatcaaaagtttgcaccgccgaagaagagaaaagcaaaaacttctcatatttttcaaaggattgaacattttttattgtcatgttatcccgtaggcaggatattgacctagcaactttggtgtagcctattgtattactcatattcatcgagcagtatattattgaaggggcttttattgactctaaatgtagcattgttgtaatgcatttgcataattaagggtgtgtttggttgtgtggctaaggatagccactttttaagtggttgccatgtgtttgattttaaaatttgtgtagttcaggatagccatatccaaggaatattccataaaatgtggcacagcggaagcgccaaaaagtgatgggtaaggtcaaccaccctatgcttgcatgctcacctcaccatctgaatcatttacaacctgttctgaccccctcccccaaaggaattttatcaggcttttggtgagggcattcgcatgtctgcacttgtcatccaatgaactgtgacgaatactagggtgttagaaaggaaacggcaactcatgaaatataaccgacaaacatatatatctataaatcatgcaacaaagaaaccgaaagattacaacaaaaaccatatatggatctgatcataaaggggcaattcatcacacggaaacaaacacaccgagaattacatcagatggaacacaatcatgtagagcagcgcatgtgaccattgattgaaaagcaagggagagaaggaaaagcaagggagagaagatgccaactagctactgttatggacccatagttcaaagaggactattcacacatggtcatggtggcaacgaggttgctggagatgcatccggcgatgatttatccctccaggaaagtaccggaaaaaggtctccggattggatcatcgcgaaacaagtggtggcgacagcagaaaaatccttgataaacaaatcacgtgagagagctccgaaattagggagcatactgaatttaaagatactagatgataccccgcatgttattgtgaactctggtctaccatacacaattataaggttataacacagactcattcggtagtgctatttgtggtatgagacttattcttgccatgttctgcaaagaattttttttgtcacatttgagttatgtaataggctgacagtcactgttttctccaaagacgcacagtcataaaagtaatttgaaaaaactgtgcattttgaaaaaaatgttcgcggaatcaaaaaatgtttgtgattttacaaaaaaatattcacatattcagaaaatgttggcgtattcagaaaatgttatggaattgagaaattttctgcaaagtttaaaatttttcatgtattttaaaaaggcttcacaaaatttaaaaaatgtccattcattacaaaaatgatcctcgattcaaaaatgctcattcaaataaaaaaatgttcatgaattacaaaattaattcccccaatttttaaaagtgtttcgctgattcaaaacaaatattcacaggcttaaaagatgttcacaattcctagaaatgtacacaaatttgtaaaaaatcctcgtcaatttaaaaaatgttcgtcaattaaaaaaatgtaatgtagtagtagtatgttgagtagtccaaccgaaggatattaattctcatctcagtcatcactcctcttcctcggatcaccggcgatggcaatggcattgtccctccccaccacggtcttgctcctcctcctcctcctcctcctccccgtcgccttagatgccgtcgcctcctagccataagcgtcagaaggcggtatataggttatctccgccggaaaaagggtaaggtttgcctcgagaatgttttgtttgactatatcctatgtggaaccaaggctaggtttgcatgcaatgaaaaaatgtaattgtaggaggagaagggagggtggtagatgtgcgcgacaacaacacacacgccatatatatatagttggaggaggtggaaaacaaggaggcgataagaaaaagggggggggggaatatggagaatgtttgaaatagcgtccaacccgagtttccattattaattgtcggccaaaaaaagaaggcatgttactcatgatggtagtttcaagggaagtggagcctcccagctagtttgaacacttaaaaaaacaagctacaacagaaagtagtttcaaggggagtggagcctaaaacttgcatttaaaaaggaaatgcgcacactaagccgaaagtgtgaacactttgtgccgacatttccgaggaagtaggttttatgcgcataacatttttttgagacaattttatgcgcataactagggttgactgctgtggggaagatgaaatttgaactgccattgtgacgcaaagatttggtgatccaaatttgtatatggaccatgcctgtaggttgggctaagtatacttttcgaagaaaataggcccatgttaacttgggaagtgggcctaggcccacaaacctgaaatccctgtgaagcccacattcacacgcgggcccaccgtcgaagcgataaggagagtgacagccgctcagcccgccggc

SEQ ID NO: 60 - DNA, 80 bp, Triticum aestivum
gccccgtcgtcctgctcccagttgagttcgcgacctacacaccggccatgccccgcttctcctccaccacgccaatgtcg

SEQ ID NO: 61 - DNA, 80 bp, Triticum aestivum
ggttcgtgtccggcggctcgatttgagggccggacggtcctcgccatggggaactgctggggcgccaagatcagctccga

SEQ ID NO: 62 - DNA, 80 bp, Triticum aestivum
tggtcactggactgatatttcttttccagattttgaaggttcacagaaggagagatggcagctctaaccagccttgcctc

SEQ ID NO: 63 - DNA, 73 bp, Triticum aestivum
acaactctcgcttcactcgccggagggggacggagcaaatatggccacggcggcggcggcggcggcgacgatg